(19) United States
(12) Patent Application Publication
(10) Pub. No.: US 2014/0323418 A1
(43) Pub. Date: Oct. 30, 2014

(54) SELECTIVE KINASE INHIBITORS

(71) Applicant: PORTOLA PHARMACEUTICALS, INC., South San Francisco, CA (US)

(72) Inventors: Zhaozhong J. Jia, San Mateo, CA (US); Brian Kane, Oakland, CA (US); Qing Xu, Foster City, CA (US); Shawn M. Bauer, Pacifica, CA (US); Yonghong Song, Foster City, CA (US); Anjali Pandey, Fremont, CA (US); Ryan Dick, Brisbane, CA (US)

(73) Assignee: Portola Pharmaceuticals, Inc., South San Francisco, CA (US)

(21) Appl. No.: 14/360,203

(22) PCT Filed: Nov. 23, 2012
(86) PCT No.: PCT/US2012/066470

§ 371 Date: May 22, 2014

Related U.S. Application Data
(60) Provisional application No. 61/563,458, filed on Nov. 23, 2011.

(51) Int. Cl. C07D 239/48; C07D 403/14; C07H 15/26; C07D 401/14; C07D 413/12; C07D 495/04; C07D 471/04; C07D 405/12; C07D 405/14; C07D 417/14; C07D 403/12; C07D 417/12

(52) U.S. Cl. CPC C07D 239/48; C07D 403/12; C07D 403/14; C07H 15/26; C07D 401/14; C07D 417/12; C07D 495/04; C07D 471/04; C07D 405/12; C07D 405/14; C07D 417/14; C07D 413/12
USPC 514/25; 544/324; 514/275; 544/296; 536/17.4; 544/323; 514/210.18; 435/184

(57) ABSTRACT

Provided are pyrimidine compounds for inhibiting of Syk kinase, intermediates used in making such compounds, methods for their preparation, pharmaceutical compositions thereof, methods for inhibition Syk kinase activity, and methods for treating conditions mediated at least in part by Syk kinase activity.

### CROSS-REFERENCES TO RELATED APPLICATIONS

This application claims priority from U.S. Provisional Application 61/563,428, filed Nov. 23, 2011, which is incorporated by reference in its entirety herewith.

### FIELD OF THE INVENTION

In one embodiment, provided are pyrimidine compounds which act as inhibitors of Spleen tyrosine kinase (Syk). Pharmaceutical compositions containing these compounds, methods for their use to treat a condition mediated at least in part by syk activity, and methods for their preparation are also provided.

### BACKGROUND OF THE INVENTION

Protein kinases constitute a large family of structurally related enzymes that are responsible for the control of a variety of signal transduction processes within cells (see, e.g., Hardie and Hanks, The Protein Kinase Facts Book, I and II, Academic Press, San Diego, Calif., 1995). Protein kinases are thought to have evolved from a common ancestral gene due to the conservation of their structure and catalytic function. Almost all kinases contain a similar 250-300 amino acid catalytic domain. The kinases can be categorized into families by the substrates they phosphorylate (e.g., protein-tyrosine, protein-serine/threonine, lipids, etc.). Sequence motifs have been identified that generally correspond to each of these families (see, e.g., Hanks & Hunter, (1995), FASEB J. 9:576-596; Knighton et al., (1991), Science 253:407-414; Hiles et al., (1992), Cell 70:419-429; Kunz et al., (1993), Cell 73:585-596; Garcia-Bustos et al., (1994), EMBO J. 13:2352-2361).

Many diseases are associated with abnormal cellular responses triggered by protein kinase-mediated events. These diseases include autoimmune diseases, inflammatory diseases, bone diseases, metabolic diseases, neurological and neurodegenerative diseases, cancer, cardiovascular diseases, allergies, asthma, alzheimer's disease and hormone-related diseases. As a consequence, there has been substantial efforts in medicinal chemistry to find inhibitors of protein kinases for use as therapeutic agents.

Immunoreceptor tyrosine activation motif (ITAM)-mediated signaling has emerged as a primary event in signaling pathways responsible for human pathologies. ITAM-mediated signaling is responsible for relaying activation signals initiated at classical immune receptors such as T-cell receptors, B-cell receptors, Fc receptors in immune cells and at GPVI and FcγRIIa in platelets to downstream intracellular molecules such as Syk and ZAP-70 (Underhill, D. M and Goodridge, H. S., Trends Immunol., 28:66-73, 2007).

The binding of a ligand to an ITAM-containing receptor triggers signaling events which allows for the recruitment of proteins from a family of nonreceptor tyrosine kinases called the Src family. These kinases phosphorylate tyrosine residues within the ITAM sequence, a region with which the tandem SH2 domains on either Syk or ZAP-70 interact.

Syk, along with Zap-70, is a member of the Syk family of protein tyrosine kinases. The interaction of Syk or ZAP-70 with diphosphorylated ITAM sequences induces a conformation change in the kinases that allows for tyrosine phosphorylation of the kinase itself. Phosphorylated Syk family members activate a multitude of downstream signaling pathway proteins which include Src homology 2 (SH2) domain containing leukocyte-specific phosphoprotein of 76 kDa (SLP-76), Linker of Activation of T-cells (LAT) and PLC (phospholipase C)γ2.

Human pathologies attributed to dysfunctional ITAM-mediated signaling include autoimmune diseases such as rheumatoid arthritis, systemic lupus, multiple sclerosis, hemolytic anemia, immune-thrombocytopenia purpura, and heparin-induced thrombocytopenia and arteriosclerosis. Interestingly, many of the above mentioned diseases are thought to occur through crosslinking of Fc receptors by antibodies which, via Syk, activate a signaling cascade in mast, basophil and other immune cells that result in the release of cell mediators responsible for inflammatory reactions. The release of mediators and the production of cytokines in IgE stimulation-dependent allergic and inflammatory reactions from mast cells and basophiles can be controlled by inhibiting the tyrosine kinase activity of Syk (Rossi, A. B. et al., J Allergy Clin Immunol., 118:749-755, 2006). In immune-thrombocytopenia, antibody bound platelets are cleared by the spleen by an Fc receptor/ITAM/Syk-mediated process (Crow, A. R. et al., Blood, 106:abstract 2165, 2005). Drug-induced thrombocytopenia, caused by heparin-platelet factor 4 immune complexes that activate platelet FcγRIIa, also involve Syk signaling downstream of receptor engagement (Reilly, M. P., Blood, 98:2442-2447, 2001).

Platelet agonists induce inside-out integrin signaling resulting in fibrinogen binding and platelet aggregation. This initiates outside-in signaling which produces further stimulation of platelets. Syk is activated during both phases of integrin signaling, and inhibition of Syk is shown to inhibit platelet adhesion to immobilized proteins (Law, D. A. et al., Blood, 93:2645-2652, 1999). Release of arachidonic acid and serotonin and platelet aggregation induced by collagen are markedly inhibited in platelets derived from Syk deficient mouse (Poole, A. et al., EMBO J., 16:2333-2341, 1997). Thus Syk inhibitors may also possess anticoagulation action.

Because of the role Syk plays in Ig-induced platelet activation, it is likely to be important in arteriosclerosis and restenosis. Arteriosclerosis is a class of diseases characterized by the thickening and hardening of the arterial walls of blood vessels. Although all blood vessels are susceptible to this serious degenerative condition, the aorta and the coronary arteries serving the heart are most often affected. Arteriosclerosis is of profound clinical importance since it can increase the risk of heart attacks, myocardial infarctions, strokes, and aneurysms.

The traditional treatment for arteriosclerosis includes vascular recanalization procedures for less-serious blockages and coronary bypass surgery for major blockages. A serious shortcoming of intravascular procedures is that, in a significant number of treated individuals, some or all of the treated vessels restenose (i.e., re-narrow). For example, restenosis of an atherosclerotic coronary artery after PTCA occurs in 10-50% of patients undergoing this procedure and subsequently requires either further angioplasty or a coronary artery bypass graft. Furthermore, restenosis of an atherosclerotic coronary artery after stenting occurs in 10-20% of patients undergoing this procedure and subsequently requires repeat treatments to maintain adequate blood flow through the affected artery. Restenosis generally occurs in a relatively brief time period, e.g., roughly less than six months, after treatment.

While the exact hormonal and cellular processes promoting restenosis have not been determined, restenosis is thought to be due in part to mechanical injury to the walls of the blood vessels caused by the balloon catheter or other intravascular device. For example, the process of PTCA, in addition to opening the obstructed artery, also injures resident coronary arterial smooth muscle cells (SMCs). In response to this injury, adhering platelets, infiltrating macrophages, leukocytes, or the smooth muscle cells themselves release cell-derived growth factors such as platelet-derived growth factor (PDGF), with subsequent proliferation and migration of medial SMCs through the internal elastic lamina to the area of the vessel intima. Further proliferation and hyperplasia of intimal SMCs and, most significantly, production of large amounts of extracellular matrix over a period of three to six months results in the filling in and narrowing of the vascular space sufficient to significantly obstruct blood flow.

In addition to the role Syk plays in Ig-induced platelet activations, Syk plays a very important role in collagen-mediated signaling. The primary adhesive protein responsible for platelet adhesion and activation is collagen. Collagen is a filamentous protein contained within the fibrotic caps of atheromas which becomes exposed to blood during plaque rupture. Collagen functions initially by binding von Willebrand factor which tethers platelets through binding platelet membrane GPIb. Collagen functions secondarily by engaging the two collagen receptors on platelets, GPVI and integrin α2β1.

GPVI exists in platelet membranes as a complex with FcRγ, an interaction required for the expression of GPVI. Activation of FcγRIIa on platelets results in platelet shape change, secretion and thrombosis. Signaling by the GPVI/FcRγ complex is initiated by tyrosine phosphorylation of the ITAM domain of FCRγ followed by the recruitment of Syk. Activation of GPVI leads to induction of multiple platelet functions including: activation of integrins α2β1 to achieve firm platelet adhesion, and GP IIb-IIIa which mediates platelet aggregation and thrombosis growth; platelet secretion, allowing for the delivery of inflammatory proteins such as CD40L, RANTES and TGFβ to the vessel wall; and the expression of P-selectin which allows for the recruitment of leukocytes. Therefore, it is believed that Syk inhibitors can inhibit thrombotic events mediated by platelet adhesion, activation and aggregation.

It has been reported that the tyrosine phosphorylation of intracellular protein (activation) induced by stimulation of a receptor for IgG antibody, FcγR, and the phagocytosis mediated by FcγR are considerably inhibited in macrophages derived from Syk deficient mouse (Crowley, M. T. et al., J. Exp. Med., 186:1027-1039, 1997). This suggests that Syk has a markedly important role in the FcγR-mediated phagocytosis of macrophages.

It has also been reported that an antisense oligonucleotide of Syk suppresses the apoptosis inhibition of eosinophils induced by GM-CSF (Yousefi, S. et al., J. E. Med., 183:1407-1414, 1996), showing that Syk is essential for the life extending signal of eosinophils caused by GM-CSF and the like. Since life extension of eosinophils is closely related to the transition of diseases into a chronic state in allergic disorders, such as asthma, Syk inhibitors can also serve as therapeutic agents for chronic eosinophilic inflammation.

Syk is important for the activation of B-cells via a B-cell antigen receptor and is involved in the phosphatidylinositol metabolism and increase in the intracellular calcium concentration caused by the antigen receptor stimulation (Hutchcroft, J E. et al., J. Biol. Chem., 267:8613-8619, 1992; and Takata, M. et al., EMBO J., 13:1341-1349, 1994). Thus, Syk inhibitors may be used to control the function of B-cells and are, therefore, expected to serve as therapeutic agents for antibody-related diseases.

Syk binds to a T-cell antigen receptor, quickly undergoes tyrosine phosphorylation through crosslinking of the receptor and synergistically acts upon intracellular signals mediated by Src tyrosine kinases such as Lek (Couture, C. et al., Proc. Natl. Acad. Sci. USA, 91:5301-5305, 1994; and Couture, C. et al., Mol. Cell. Biol., 14:5249-5258, 1994). Syk is present in mature T-cell populations, such as intraepithelial γδ T-cells and naïve αβ T-cells, and has been reported to be capable of phosphorylation of multiple components of the TCR signaling cascade (Latour, S. et. al., Mol Cell Biol., 17:4434-4441, 1997). As a consequence, Syk inhibitors may serve as agents for inhibiting cellular immunity mediated by T-cell antigen receptor.

Recent comparative genomic hybridization studies have identied Syk as another gene important in the pathogenesis of Mantle Cell Lymphoma (MCL) (Chen, R. et al. Journal of Clinical Oncology, 2007 ASCO Annual Meeting Proceedings (Post-Meeting Edition). Vol 25, No 18S (June 20 Supplement), 2007: 8056). MCL represents 5-10% of all non-Hodgkins lymphomas and it is a difficult form of lymphoma to treat. It has the worst prognosis among the B cell lymphomas with median survival of three years. It has been reported that Syk is overexpressed in MCL (Rinaldi, A, et al, Br. J. Haematol., 2006; 132:303-316) and that Syk mediates mTOR (mammalian target of Rapamycin) survival signals in follicular, mantel cell, Burkitt's, and diffuse large B-cell non-Hodgkin's lymphomas (Leseux, L., et. al, Blood, 2006; 108:4156-4162).

Several lines of evidence suggest that many B-cell lymphomas depend upon B-cell receptor (BCR)-mediated survival signals. BCR signaling induces receptor oligomerization and phosphorylation of Igα and β immunoreceptor tyrosine-based activated motifs by SRC family kinases. ITAM phosphorylation results in the recruitment and activation of Syk that initiates downstream events and amplifies the original BCR signal. Given the role of tonic BCR signaling in normal B cell and Syk-dependent survival of non-Hodgkins lymphoma cell lines in vitro (Chen, L., et al, Blood, 2006; 108:3428-3433), Syk inhibition is a promising rational treatment target for certain B-cell lymphomas and chronic lymphocytic leukemia (CLL) (Stefania Gobessi, Luca Laurenti, Pablo Longo, Laura Carsetti, Giuseppe Leone, Dimitar G. Efremov, Constitutive activation of the protein tyrosine kinase Syk in Chronic Lymphocytic Leukemia B-cells, Blood, 2007, 110, Abstract 1123). Recent data shows that administration of a multikinase inhibitor which inhibits Syk, may have significant clinical activity in CLL patients (Friedberg J W et al, Blood 2010; 115(13),).

The oncogenic potential of the spleen tyrosine kinase (Syk) has been described in a number of different settings. Clinically, Syk over-expression is reported in Mantle Cell Lymphoma (Rinaldi, A, et. al, Br. J. Haematol., 2006; 132:303-316) and the TEL-Syk fusion protein (Translocated ETS Leukemia) generated by a chromosomal translocation (t(9;12)(q22;p12)) leads to increased Syk activity and is associated with myelodysplastic syndrome (Kuno, Y., et. al, Blood, 2001; 97:1050-1055). Leukemia is induced in mice by adoptively transferring bone marrow cells that express human TEL-Syk (Wossning, T., JEM, 2006; 203:2829-2840). Further, in mouse primary bone marrow cells, over-expression of Syk results in IL-7 independent growth in culture (Wossning, T., et. al, JEM, 2006; 203:2829-2840). Additional recent studies also suggest that Syk-dependant survival signals may play a role in B-cell malignancies, including DLBCL, mantle cell lymphoma and follicular lymphoma (Gururajan, Jennings et al. 2006; Irish, Czerwinski et al. J Immunol 176(10): 5715-9 (2006). Given the role of tonic BCR signaling in normal B cells and Syk-dependent survival of NHL cell lines in vitro, the specific inhibition of Syk may prove promising for the treatment of certain B-cell lymphomas.

Interestingly, Syk signaling appears to be required for B-cell development and survival in humans and mouse. Inducible loss of the B-cell receptor (Lam, K., et. al, Cell, 1997; 90:1073-1083) or Igα (Kraus, M., et. al, Cell, 2004; 117:787-800) results in loss of peripheral B-cells in mice. Over-expression of the protein tyrosine phosphatase PTP-RO, which is known to negatively regulate Syk activity, inhibits proliferation and induces apoptosis in cell lines derived from non-Hodgkin's lymphomas (Chen, L., et. al, Blood, 2006; 108:3428-3433). Finally, B-cell lymphomas rarely exhibit loss of BCR expression, and anti-idiotype therapy rarely leads to resistance (Kuppers, R. Nat Rev Cancer, 2005; 5:251-262).

Engagement of the antigen-specific B cell receptor (BCR) activates multiple signaling pathways that ultimately regulate the cells activation status, promoting survival and clonal expansion. Signaling through the BCR is made possible by its association with two other members of the immunoglobulin super-family; Igα and Igβ, each bearing an immuno-tyrosine based activation motif (ITAM) (Jumaa, Hendriks et al. Annu Rev Immunol 23: 415-45 (2005). The ITAM domain is directly phosphorylated by Src family kinases in response to BCR engagement. The spleen tyrosine kinase (Syk) docks with and phosphorylates the ITAM, a process that enhances its kinase activity, resulting in Syk autophosphorylation and tyrosine phosphorylation of multiple downstream substrates (Rolli, Gallwitz et al. Mol Cell 10(5): 1057-69 (2002). This signaling pathway is active in B cells beginning at the transition from pro- to pre-B cell stage of development, when the newly formed pre-BCR is expressed. In fact, B cell development arrests at the pro-B cell stage in Syk knockout mice (Cheng, Rowley et al. 1995; Turner, Mee et al. Nature 378(6554): 303-6 (1995). Inducible loss of the B cell receptor (Lam, Kuhn et al. Cell 90(6): 1073-83 (1997) or Igα (Kraus, Alimzhanov et al. Cell 117(6): 787-800 (2004) results in loss of peripheral B cells in mice. Human B cells also appear to require Syk for proliferation and survival. Over-expression of the protein tyrosine phosphatase PTP-RO, a negative regulator of Syk activity, inhibits proliferation and induces apoptosis in cell lines derived from non-Hodgkin's lymphomas (NHL) (Chen, Juszczynski et al. Blood 108(10): 3428-33 (2006). Knock down of Syk by siRNA in the NHL line SUDHL-4 led to a block in the G1/S transition of the cell cycle (Gururajan, Dasu et al. J Immunol 178(1): 111-21 (2007). Together, these data suggest that Syk signaling is required for the development, proliferation, and even survival of human and mouse B cells.

Recently, R406 (Rigel Pharmaceuticals) was reported to inhibit ITAM signaling in response to various stimuli, including FcεR1 and BCR induced Syk activation (Braselmann, Taylor et al. J Pharmacal Exp Ther 319(3): 998-1008 (2006). Interestingly, this ATP-competitive inhibitor of Syk was also active against Flt3, cKit, and JAK kinases, but not against Src kinase (Braselmann, Taylor et al. 2006). Activating mutations to Flt3 are associated with AML and inhibition of this kinase is currently under clinical development (Burnett and Knapper Hematology Am Soc Hematol Educ Program 2007: 429-34 (2007). Over-activation of the tyrosine kinase cKit is also associated with hematologic malignancies, and a target for cancer therapy (Heinrich, Griffith et al. Blood 96(3): 925-32 (2000). Similarly, JAK3 signaling is implicated in leukemias and lymphomas, and is currently exploited as a potential therapeutic target (Heinrich, Griffith et al. 2000). Importantly, the multi-kinase inhibitory activity of R406 attenuates BCR signaling in lymphoma cell lines and primary human lymphoma samples, resulting in apoptosis of the former (Chen, Monti et al. Blood 111(4): 2230-7 (2008). Further, a phase II clinical trial reported favorable results by this compound in refractory NHL and chronic lymphocytic leukemia (Friedberg J W et al, Blood 2010; 115(13)). Although the precise mechanism of action is unclear for R406, the data suggest that inhibition of kinases that mediate survival signaling in lymphocytes is clinically beneficial.

Additional recent studies also suggest that Syk-dependant survival signals may play a role in B-cell malignancies, including DLBCL, mantle cell lymphoma and follicular lymphoma (see e.g., S. Linfengshen et al. Blood, February 2008; 111: 2230-2237; J. M. Irish et al. Blood, 2006; 108: 3135-3142; A. Renaldi et al. Brit J. Haematology, 2006; 132: 303-316; M. Guruoajan et al. J. Immunol, 2006; 176: 5715-5719; L. Laseux et al. Blood, 2006; 108: 4156-4162.

While progress has been made in this field, there remains a need in the art for compounds that inhibit Syk kinase, as well as for methods for treating conditions in a patient, such as restenosis, and/or inflammation that can benefit from such inhibition. Moreover, the availability of compounds that selectively inhibit one of these kinases as compared to other kinases would also be desirable. The present invention satisfies this and other needs.

### BRIEF SUMMARY OF THE INVENTION

The present invention provides novel compounds having activity as inhibitors of Syk activity (also referred to herein as “Syk inhibitors”) as well as to methods for their preparation and use, and to pharmaceutical compositions containing the same.

In one embodiment, provided is a compound of Formula (I):

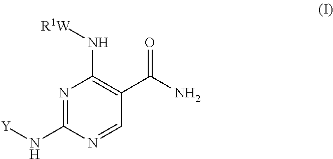

or a pharmaceutically acceptable salt thereof, wherein R¹W and Y are described below.

In another embodiment, provided is a compound of Formula (II):

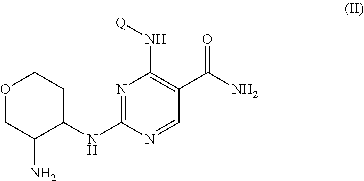

or a pharmaceutically acceptable salt thereof, wherein Q is defined below.

In another embodiment, provided is a compound of Formula (III):

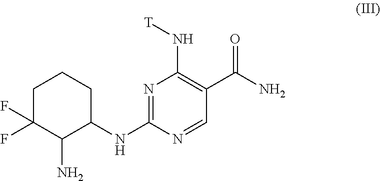

or a pharmaceutically acceptable salt thereof, wherein T is defined below.

In another embodiment, provided is a compound of Formula (IV):

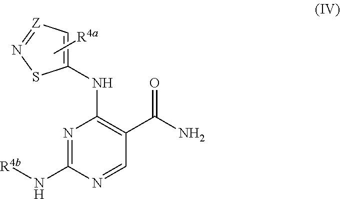

or a pharmaceutically acceptable salt thereof, wherein Z, R^{4a} and R^{4b} are defined below.

In another embodiment, provided is a compound of having the Formula (V):

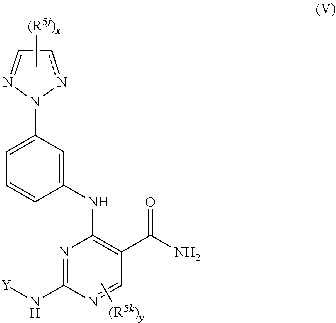

or a pharmaceutically acceptable salt thereof, wherein Y, R^{5j}, R^{5k}, x and y are defined below.

The present invention also provides a pharmaceutical composition comprising a therapeutically effective amount of a compound provided herein, or a pharmaceutical acceptable salt thereof, and a pharmaceutically acceptable carrier and/or diluent.

The compounds of the present invention have utility over a wide range of therapeutic applications, and may be used to treat a variety of conditions, mediated at least in part by Syk activity, in both men and women, as well as a mammal in general (also referred to herein as a “subject”). For example, such conditions include, but are not limited to, those associated with cardiovascular disease, inflammatory disease or autoimmune disease. More specifically, the compounds of the present invention have utility for treating conditions or disorders including, but not limited to: restenosis, inflammation, heparin induced thrombocytopenia, dilated cardiomyopathy, sickle cell disease, atherosclerosis, myocardial infarction, vascular inflammation, unstable angina, acute coronary syndromes, allergy, asthma, rheumatoid arthritis, B-cell mediated diseases such as Non Hodgkin's lymphoma, Crohn's disease, anti-phospholipid syndrome, lupus, psoriasis, multiple sclerosis, and chronic lymphocytic leukemia. Thus, in one embodiment, methods are disclosed which include the administration of an effective amount of a compound provided herein, typically in the form of a pharmaceutical composition, to a subject in need thereof.

The present invention also provides a method for inhibiting the Syk activity of a blood sample comprising contacting said sample with a compound of the present invention.

The present invention further provides compounds in purified forms, as well as chemical intermediates.

These and other aspects, objects, features and advantages of the invention will be apparent upon reference to the following detailed description and figures. To this end, various references are set forth herein which describe in more detail certain background information, procedures, compounds and/or compositions, and are each hereby incorporated by reference in their entirety.

### DETAILED DESCRIPTION OF THE INVENTION

As used herein, the below terms have the following meanings unless specified otherwise:

### 1. Abbreviations and Definitions

The abbreviations used herein are conventional, unless otherwise defined. The following abbreviations are used: ACN=acetonitrile, AcOH=acetic acid, AIBN=azobisisobutyronitrile (also azobisisobutylonitrile), aq.=aqueous, Ar=argon, Boc=t-butylcarboxy, Bz—benzoyl, Bn=benzyl, BOP=benzotriazol-1-yloxytris(dimethylamino)-phosphonium hexafluorophosphate, BPO=benzoyl peroxide, nBuOH=n-butanol, ° C.=degrees celcius, CBr₄=tetrabromomethane, Cbz=benzyloxycarbonyl, mCPBA=m-chloroperoxybenzoic acid, CH₂Cl₂ or DCM=dichloromethane, Cs₂CO₃=cesium carbonate, CuCl₂=copper chloride; DIBAL=diisobutylaluminum hydride, DIEA=Hunig's base or diisopropyl ethylamine, DME=dimethoxy-ethane, DMF=dimethyl formamide, DMSO=dimethyl sulfoxide, DPPA=diphenyl phosphoryl azide, EDC=1-ethyl-3-(3-dimethylaminopropyl)carbodiimide, Et₃N=triethylamine, EtOAc=ethyl acetate, g=gram, HATU=2-(1H 7-azabenzotriazol-1-yl)-1,1,3,3-tetramethyl uronium hexafluorophosphate, HOBT=hydroxybenzotriazole, H₂=hydrogen; H₂O=water; HBr=hydrogen bromide; HCl=hydrogen chloride, HIV=human immunodeficiency virus, HPLC=high pressure liquid chromatography, h=hour, IgE=immunoglobulin E, IC₅₀=The concentration of an inhibitor that is required for 50% inhibition of an enzyme in vitro, IPA=isopropyl alcohol, kg=kilogram, KCN=potassium cyanide, KOH=potassium hydroxide, K₂PO₄=potassium phosphate, LDA=lithium diisopropylamide, LiAlH₄=lithium aluminum hydride=LiOH: lithium hydroxide; MeCN=acetonitrile; MS=Mass Spec, m/z=mass to charge ratio, Ms=methanesulfonyl, MHz=Mega Hertz, MeOH=methanol, MTBE=methyl tert-butyl ether, μM=micromolar, μL=microliter, mg=milligram, mm=millimeter, mM=millimolar, mmol=millimole, mL=milliliter, mOD/min=millioptical density units per minute, min=minute, M=molar, Na₂CO₃=sodium carbonate, ng=nanogram, NaHCO₃=sodium bicarbonate; NaNO₂=sodium nitrite; NaOH=sodium hydroxide; Na₂S₂O₃=sodium thiosulfate; Na₂SO₄=sodium sulfate; NBS=N-bromosuccinimide; NH₄Cl=ammonium chloride; NH₄OAc=ammonium acetate; NaSMe=sodium methylthiolate, NBS=N-bromosuccinamide, n-BuLi=n-butyl lithium, nm=nanometer, nM=nanomolar, N=Normal, NMP=N-methylpyrrolidone, NMR=nuclear magnetic resonance, Pd/C=palladium on carbon, Pd(PPh₃)₄=Tetrakis-(triphenyl-phosphine)-palladium, pM=picomolar, Pin=pinacolato, PEG=polyethylene glycol, PMB=paramethoxybenzyl, PPh₃ or Ph₃P=triphenyl phosphine, psi=pound per square inch, RLV=Raucher leukemia virus, Ra-Ni=Rainey Nickel, rp=reverse phase, sat=saturated, SOCl₂=thionyl chloride, RT=room temperature, TEA=triethylamine, THF=tetrahydrofuran, TFA=trifluoroacetic acid, TLC=thin layer chromatography, TMS=trimethylsilyl, Tf=trifluoromethylsulfonyl and TSC=trisodium citrate.

It is noted here that as used in this specification and the appended claims, the singular forms “a,” “an,” and “the” include plural reference unless the context clearly dictates otherwise.

“Alkyl,” by itself or as part of another substituent, means, unless otherwise stated, a straight or branched chain, fully saturated aliphatic hydrocarbon radical having the number of carbon atoms designated. For example, “C₁₋₈alkyl” refers to a hydrocarbon radical straight or branched, containing from 1 to 8 carbon atoms that is derived by the removal of one hydrogen atom from a single carbon atom of a parent alkane. Alkyl includes branched chain isomers of straight chain alkyl groups such as isopropyl, t-butyl, isobutyl, sec-butyl, and the like. Representative alkyl groups include straight and branched chain alkyl groups having 1, 2, 3, 4, 5, 6, 7, 8, 9, 10, 11 or 12 carbon atoms. Further representative alkyl groups include straight and branched chain alkyl groups having 1, 2, 3, 4, 5, 6, 7 or 8 carbon atoms.

“Alkylene” by itself or as part of another substituent means a divalent radical derived from an alkane, as exemplified by —CH₂CH₂CH₂CH₂—. Typically, an alkylene group will have from 1, 2, 3, 4, 5, 6, 7 or 8 carbon atoms that is derived by the removal of one hydrogen atom from a single carbon atom of a parent alkyl.

“Alkenyl” refers to a linear monovalent hydrocarbon radical or a branched monovalent hydrocarbon radical having the number of carbon atoms indicated in the prefix and containing at least one double bond, but no more than three double bonds. For example, (C₂-C₆)alkenyl is meant to include, ethenyl, propenyl, 1,3-butadienyl and the like.

“Alkynyl” means a linear monovalent hydrocarbon radical or a branched monovalent hydrocarbon radical containing at least one triple bond and having the number of carbon atoms indicated in the prefix. The tem). “alkynyl” is also meant to include those alkyl groups having one triple bond and one double bond. For example, (C₂-C₆)alkynyl is meant to include ethynyl, propynyl and the like.

“Cycloalkyl” or “carbocycle”, by themselves or in combination with other terms, represent, unless otherwise stated, cyclic versions of “alkyl”, “alkenyl” and “alkynyl” in which all ring atoms are carbon. “Cycloalkyl” or “carbocycle” refers to a mono- or polycyclic group. When used in connection with cycloalkyl substituents, the term “polycyclic” refers herein to fused and non-fused alkyl cyclic structures. “Cycloalkyl” or “carbocycle” may form a bridged ring or a spiro ring. The cycloalkyl group may have one or more double or triple bond(s). The term “cycloalkenyl” refers to a cycloalkyl group that has at least one site of alkenyl unsaturation between the ring vertices. The term “cycloalkynyl” refers to a cycloalkyl group that has at least one site of alkynyl unsaturation between the ring vertices. When “cycloalkyl” is used in combination with “alkyl”, as in C₃₋₈cycloalkylC₃₋₈alkylene-, the cycloalkyl portion is meant to have the stated number of carbon atoms (e.g., from three to eight carbon atoms), while the alkylene portion has from one to eight carbon atoms. Examples of cycloalkyl include cyclopentyl, cyclohexyl, 1-cyclohexenyl, 3-cyclohexenyl, cycloheptyl, and the like.

“Aryl” by itself or as part of another substituent refers to a polyunsaturated, aromatic, hydrocarbon group containing from 6 to 14 carbon atoms, which can be a single ring or multiple rings (up to three rings) which are fused together or linked covalently. Thus the phrase includes, but is not limited to, groups such as phenyl, biphenyl, anthracenyl, naphthyl by way of example. Non-limiting examples of aryl groups include phenyl, 1-naphthyl, 2-naphthyl and 4-biphenyl.

The terms “heterocycle”, “heterocyclyl” or “heterocyclic” refer to a saturated or unsaturated non-aromatic cyclic group containing at least one heteroatom and optionally one or more oxo substituents. As used herein, the term “heteroatom” is meant to include oxygen (O), nitrogen (N), sulfur (S) and silicon (Si), wherein the heteroatoms are optionally oxidized, and the nitrogen atom(s) are optionally quaternized. Each heterocycle can be attached at any available ring carbon or heteroatom. Each heterocycle may have one or more rings. When multiple rings are present, they can be fused together or linked covalently. Each heterocycle typically contains 1, 2, 3, 4 or 5, independently selected heteroatoms. Preferably, these groups contain 1, 2, 3, 4, 5, 6, 7, 8, 9 or 10 carbon atoms, 0, 1, 2, 3, 4 or 5 nitrogen atoms, 0, 1 or 2 sulfur atoms and 0, 1 or 2 oxygen atoms. More preferably, these groups contain 1, 2 or 3 nitrogen atoms, 0-1 sulfur atoms and 0-1 oxygen atoms. Non-limiting examples of heterocycle groups include morpholin-3-one, piperazine-2-one, piperazin-1-oxide, pyridine-2-one, piperidine, morpholine, piperazine, isoxazoline, pyrazoline, imidazoline, pyrazol-5-one, pyrrolidine-2,5-dione, imidazolidine-2,4-dione, pyrrolidine, tetrahydroquinolinyl, decahydroquinolinyl, tetrahydrobenzooxazepinyl dihydrodibenzooxepin and the like.

“Heteroaryl” refers to a cyclic or polycyclic aromatic radical that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom or through a carbon atom and can contain 5 to 10 carbon atoms. Non-limiting examples of heteroaryl groups include 1-pyrrolyl, 2-pyrrolyl, 3-pyrrolyl, 1-pyrazolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl and 4-pyrimidyl. If not specifically stated, substituents for each of the above noted aryl and heteroaryl ring systems are selected from the group of acceptable substituents described herein.

“Bicyclic heteroaryl” refers to bicyclic aromatic radical that contain from one to five heteroatoms selected from N, O, and S, wherein the nitrogen and sulfur atoms are optionally oxidized, and the nitrogen atom(s) are optionally quaternized. A bicyclic heteroaryl group can be attached to the remainder of the molecule through a heteroatom or through a carbon atom and can contain 5 to 10 carbon atoms. Non-limiting examples of bicyclic heteroaryl groups include 5-benzothiazolyl, purinyl, 2-benzimidazolyl, benzopyrazolyl, 5-indolyl, azaindole, 1-isoquinolyl, 5-isoquinolyl, 2-quinoxalinyl, 5-quinoxalinyl, 3-quinolyl and 6-quinolyl.

In each of the above embodiments designating a number of atoms e.g. “C₁₋₈” is meant to include all possible embodiments that have one fewer atom. Non-limiting examples include C₁₋₇, C₂₋₈, C₂₋₇, C₃₋₈, C₃₋₇ and the like.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

The term “acyl” refers to the group —C(═O)R^{c} where R^{c} is alkyl, alkenyl, alkynyl, cycloalkyl, cycloalkenyl, aryl, heteroaryl or heterocyclyl. Acyl includes the “acetyl” group —C(═O)CH₃.

“Acylamino-” refers to the group —NR^{a}C(═O)R^{c} where R^{c} is alkyl, alkenyl, alkynyl, cycloalkyl, cycloalkenyl, aryl, heteroaryl or heterocyclyl.

“Alkoxy” refers to —OR^{d} wherein R^{d} is alkyl as defined herein. Representative examples of alkoxy groups include methoxy, ethoxy, t-butoxy, trifluoromethoxy, and the like.

“Alkoxyalkylene” refers to -(alkoxy)(alkylene) wherein alkoxy and alkylene are defined herein.

“Alkoxycarbonylalkylene” refers to the group -alkylene-C(═O)OR^{d} wherein R^{d} is alkyl.

“Alkoxycarbonylamino” refers to to —NR^{a}C(═O)OR^{d} wherein R^{a} is H or alkyl and R^{d} is alkyl.

“Alkoxycarbonylaminoalkylene” refers to to -alkylene-NR^{a}C(═O)OR^{d} wherein R^{a} is H or alkyl R^{d} is alkyl.

“Alkylaminoalkylene” refers to the group -alkyleneNR^{a}R^{d} wherein R^{a} is H or alkyl and R^{d} is alkyl.

“Alkylcarbonyl” refers to the group —C(═O)R^{c} where R^{c} is alkyl.

“Alkylcycloalkyl” refers to the group -cycloalkyl-R^{d}.where R^{d} is alkyl.

“Alkylheterocyclyl” refers to the group -heterocyclyl-R^{d}.where R^{d} is alkyl.

“Alkylsulfonyl” refers to —S(═O)₂R^{e} where R^{e} is alkyl. Alkylsulfonyl groups employed in compounds of the present invention are typically C₁₋₆alkylsulfonyl groups.

“Alkylsulfonylalkylene” refers to -alkylene-S(═O)₂R^{e} where R^{e} is alkyl. Alkylsulfonyl groups employed in compounds of the present invention are typically C₁₋₆alkylsulfonyl groups.

“Alkylthio” refers to —SR^{e} where R^{e} is alkyl.

“Alkylthioalkylene” refers to -(alkylene)SR^{e} where R^{e} is alkyl and alkylene is as defined herein.

“Amino” refers to a monovalent radical —NR^{a}R^{b} or divalent radical —NR^{a}—. The term includes “alkylamino” which refers to the group —NR^{a}R^{b} where R^{a} is alkyl and R^{b} is H or alkyl. The term also includes “acylamino” which refers to the group —NR^{a}R^{b} where at least one R^{a} or R^{b} is aryl. The term also includes “(alkyl)(aryl)amino” which refers to the group —NR^{a}R^{b} where R^{a} is alkyl and R^{b} is aryl. Additionally, for dialkylamino groups, the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

“Aminoalkylene” refers to -alkylene-amino wherein alkylene and amino are as defined herein.

“Aminoalkylenecarbonyl” refers to —C(═O)-alkylene-amino wherein alkylene and amino are as defined herein.

“Aminoalkyleneaminocarbonyl” refers to —C(═O)NR^{a}-alkylene-amino wherein R^{a} is H or alkyl and alkylene and amino are as defined herein.

“Aminocarbonyl” or “aminoacyl” refers to the amide —C(═O)amino wherein amino is as defined herein. The term “alkylaminocarbonyl” refers herein to the group —C(═O)—NR^{a}R^{b} where R^{a} is alkyl and R^{b} is H or alkyl. The term “arylaminocarbonyl” refers herein to the group —C(═O)—NR^{a}R^{b} where R^{a} or R^{b} is aryl.

“Aminocycloalkyl” refers to the group -cycloalkyl-amino, wherein cycloalkyl and amino are as defined herein.

“Aminosulfonyl” refers to —S(O)₂-amino where amino is as defined herein.

“Arylalkoxycarbonylamino” refers to the group —NR^{a}C(═O)O-alkylene-R^{c} wherein R^{a} is H or alkyl and R^{c} is aryl.

“Arylcarbonyl” refers to the group —C(═O)R^{c} where R^{c} is aryl.

“Arylalkylenecarbonyl” refers to the group —C(═O)-alkylene-R^{c} where R^{c} is aryl.

“Arylcarbonylamino” refers to —NR^{a}C(═O)R^{c} wherein R^{c} is aryl.

“Aryloxy” refers to —OR^{d} where R^{d} is aryl. Representative examples of aryloxy groups include phenoxy, naphthoxy, and the like.

“Aryloxyalkylene” refers to —O-alkylene-R^{d} where R^{d} is aryl.

“Azido” refers to the group —N₃.

“Bond” when used a element in a Markush group means that the corresponding group does not exist, and the groups of both sides are directly linked.

“Carbonyl” refers to the divalent group —C(═O)—.

“Carboxy” or “carboxyl” refers to the group —CO₂H.

“Carboxyalkylene” refers to the group -alkylene-CO₂H.

“Cycloalkylalkylene” refers to a radical —R^{x}R^{y} wherein R^{x} is an alkylene group and R^{y} is a cycloalkyl group as defined herein, e.g., cyclopropylmethyl, cyclohexenylpropyl, 3-cyclohexyl-2-methylpropyl, and the like.

“Ester” refers to —C(═O)OR^{d} wherein R^{d} is alkyl, cycloalkyl, aryl, heteroaryl, or heterocyclyl.

“Halo” or “halogen” by themselves or as part of another substituent, mean, unless otherwise stated, a fluorine, chlorine, bromine, or iodine atom. Additionally, terms such as “haloalkylene”, are meant to include alkyl in which one or more hydrogen is substituted with halogen atoms which can be the same or different, in a number ranging from one up to the maximum number of halogens permitted e.g. for alkyl, (2m′+1), where m′ is the total number of carbon atoms in the alkyl group. For example, the term “haloC₁₋₈alkylene” is meant to include trifluoromethyl, 2,2,2-trifluoroethyl, 4-chlorobutyl, 3-bromopropyl, and the like. The term “perhaloalkylene” means, unless otherwise stated, alkyl substituted with (2m′+1) halogen atoms, where m′ is the total number of carbon atoms in the alkyl group. For example, the term. “perhaloC₁₋₈alkylene”, is meant to include trifluoromethyl, pentachloroethyl, 1,1,1-trifluoro-2-bromo-2-chloroethyl, and the like. Additionally, term “haloalkoxy” refers to an alkoxy radical substituted with one or more halogen atoms.

“Heterocyclylalkylene” refers to the -alkylene-R^{c} where R^{c} is heterocyclyl.

“Heteroarylalkylene” refers to the -alkylene-R^{c} where R^{c} is aryl.

“Hydroxy” or “hydroxyl” refers to the group —OH.

“Hydroxycarbonylamino” refers to to —NR^{a}C(═O)OH.

“Hydroxyalkoxy” refers to to -alkoxy-OH wherein alkoxy is as defined herein.

“Hydroxyalkylene” refers to to -alkylene-OH wherein alkylene is as defined herein.

“Nitro” refers to —NO₂.

“Nitroso” refers to the group —NO.

The terms “optional” or “optionally” as used throughout the specification means that the subsequently described event or circumstance may but need not occur, and that the description includes instances where the event or circumstance occurs and instances in which it does not. For example, “heterocyclo group optionally mono- or di-substituted with an alkyl group means that the alkyl may but need not be present, and the description includes situations where the heterocyclo group is mono- or disubstituted with an alkyl group and situations where the heterocyclo group is not substituted with the alkyl group.

“Oxo” refers to the divalent atom ═O.

“Heteroarylsulfinyl” refers to the group —S(═O)—R^{e} where R^{e} is as defined heteroaryl.

“Sulfonyl” refers to the group —S(O)₂—R^{e}.

“Sulfonylamino” refers to —NR^{a}S(═O)₂—R^{e} where R^{a} is selected from the group consisting of hydrogen, alkyl, alkenyl, alkynyl, aryl, cycloalkyl, cycloalkenyl, heteroaryl and heterocyclyl and R^{e} is selected from the group consisting of hydrogen, alkyl, alkenyl, alkynyl, aryl, cycloalkyl, cycloalkenyl, heteroaryl and heterocyclyl.

“Thiol” refers to the group —SH.

Compounds that have the same molecular formula but differ in the nature or sequence of bonding of their atoms or the arrangement of their atoms in space are termed “isomers”. Isomers that differ in the arrangement of their atoms in space are termed “stereoisomers”. “Stereoisomer” and “stereoisomers” refer to compounds that exist in different stereoisomeric forms if they possess one or more asymmetric centers or a double bond with asymmetric substitution and, therefore, can be produced as individual stereoisomers or as mixtures. Stereoisomers include enantiomers and diastereomers. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Calm and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Unless otherwise indicated, the description is intended to include individual stereoisomers as well as mixtures. The methods for the determination of stereochemistry and the separation of stereoisomers are well-known in the art (see discussion in Chapter 4 of ADVANCED ORGANIC CHEMISTRY, 4th edition J. March, John Wiley and Sons, New York, 1992) differ in the chirality of one or more stereocenters.

“Tautomer” refers to alternate forms of a molecule that differ in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups are limited to -substituted aryl-(substituted aryl)-substituted aryl.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl and trityl ethers, as well as alkyl ethers, tetrahydropyranyl ethers, trialkylsilyl ethers (e.g., TMS or TIPPS groups) and allyl ethers.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge, S. M. et al., “Pharmaceutical Salts,” Journal of Pharmaceutical Science, 66:1-19, 1977). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug ester form. “Prodrug”s of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are frequently, but not necessarily, pharmacologically inactive until converted into the active drug. Prodrugs are typically obtained by masking a functional group in the drug believed to be in part required for activity with a progroup (defined below) to form a promoiety which undergoes a transformation, such as cleavage, under the specified conditions of use to release the functional group, and hence the active drug. The cleavage of the promoiety may proceed spontaneously, such as by way of a hydrolysis reaction, or it may be catalyzed or induced by another agent, such as by an enzyme, by light, by acid or base, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

“Progroup” refers to a type of protecting group that, when used to mask a functional group within an active drug to form a promoiety, converts the drug into a prodrug. Progroups are typically attached to the functional group of the drug via bonds that are cleavable under specified conditions of use. Thus, a progroup is that portion of a promoiety that cleaves to release the functional group under the specified conditions of use. As a specific example, an amide promoiety of the formula —NH—C(O)CH₃ comprises the progroup —C(O)CH₃.

A wide variety of progroups, as well as the resultant promoieties, suitable for masking functional groups in the active Syk selective inhibitory compounds to yield prodrugs are well-known in the art. For example, a hydroxyl functional group may be masked as a sulfonate, ester (such as acetate or maleate) or carbonate promoiety, which may be hydrolyzed in vivo to provide the hydroxyl group. An amino functional group may be masked as an amide, carbamate, imine, urea, phosphenyl, phosphoryl or sulfenyl promoiety, which may be hydrolyzed in vivo to provide the amino group. A carboxyl group may be masked as an ester (including methyl, ethyl, pivaloyloxymethyl, silyl esters and thioesters), amide or hydrazide promoiety, which may be hydrolyzed in vivo to provide the carboxyl group. The invention includes those esters and acyl groups known in the art for modifying the solubility or hydrolysis characteristics for use as sustained-release or prodrug formulations. Other specific examples of suitable progroups and their respective promoieties will be apparent to those of skill in the art.

Certain compounds of the present invention can exist in unsolvated forms as well as solvated forms, including hydrated forms. “Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. In general, the solvated forms are equivalent to unsolvated forms and are intended to be encompassed within the scope of the present invention. Certain compounds of the present invention may exist in multiple crystalline or amorphous forms. In general, all physical fauns are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. These isomers can be resolved or asymmetrically synthesized using conventional methods to render the isomers “optically pure”, i.e., substantially free of its other isomers. If, for instance, a particular enantiomer of a compound of the present invention is desired, it may be prepared by asymmetric synthesis, or by derivation with a chrial auxilliary, where the resulting diastereomeric mixture is separated and the auxilliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diasteromers thus formed by fractional crystallization or chromatographic means well known in the art, and subsequent recovery of the pure enantiomers.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Adminsitration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

An “agonist” or “activator” refers to an agent or molecule that binds to a receptor of the invention, stimulates, increases, opens, activates, facilitates, enhances activation or enzymatic activity, sensitizes or up regulates the activity of a receptor of the invention.

An “antagonist” or “inhibitor” refers to an agent or molecule that inhibits or binds to, partially or totally blocks stimulation or activity, decreases, closes, prevents, delays activation or enzymatic activity, inactivates, desensitizes, or down regulates the activity of a receptor of the invention. As used herein, “antagonist” also includes a reverse or inverse agonist.

As used herein, the term “condition or disorder responsive to modulation of Syk” and related terms and phrases refer to a condition or disorder associated with inappropriate, e.g., less than or greater than normal, activity of Syk and at least partially responsive to or affected by modulation of Syk (e.g., Syk antagonist or agonist results in some improvement in patient well-being in at least some patients). Inappropriate functional activity of Syk might arise as the result of expression of Syk in cells which normally do not express the receptor, greater than normal production of Syk, or slower than normal metabolic inactivation or elimination of Syk or its active metabolites, increased expression of Syk or degree of intracellular activation (leading to, e.g., inflammatory and immune-related disorders and conditions) or decreased expression of Syk. A condition or disorder associated with Syk may include a “Syk-mediated condition or disorder”.

As used herein, the phrases “a condition or disorder mediated at least in part by Syk kinase activity”, and related phrases and terms refer to a condition or disorder characterized by inappropriate, e.g., greater than normal, Syk activity. Inappropriate Syk functional activity might arise as the result of Syk expression in cells which normally do not express Syk or increased Syk expression or degree of intracellular activation (leading to, e.g., inflammatory and immune-related disorders and conditions). A condition or disorder mediated at least in part by Syk or JAK kinase activity may be completely or partially mediated by inappropriate Syk functional activity. However, a condition or disorder mediated at least in part by Syk kinase activity is one in which modulation of Syk results in some effect on the underlying condition or disorder (e.g., an Syk antagonist results in some improvement in patient well-being in at least some patients).

The term “inflammation” as used herein refers to infiltration of white blood cells (e.g., leukocytes, monocytes, etc.) into the area being treated for restenosis.

The term “intervention” refers to an action that produces an effect or that is intended to alter the course of a disease process. For example, “vascular intervention” refers to the use of an intravascular procedure such as angioplasty or a stent to open an obstructed blood vessel.

The term “intravascular device” refers to a device useful for a vascular recanalization procedure to restore blood flow through an obstructed blood vessel. Examples of intravascular devices include, without limitation, stents, balloon catheters, autologous venous/arterial grafts, prosthetic venous/arterial grafts, vascular catheters, and vascular shunts.

The term “leukocyte” refers to any of the various blood cells that have a nucleus and cytoplasm, separate into a thin white layer when whole blood is centrifuged, and help protect the body from infection and disease. Examples of leukocytes include, without limitation, neutrophils, eosinophils, basophils, lymphocytes, and monocytes.

The term “mammal” includes, without limitation, humans, domestic animals (e.g., dogs or cats), farm animals (cows, horses, or pigs), monkeys, rabbits, mice, and laboratory animals.

The terms “modulate”, “modulation” and the like refer to the ability of a compound to increase or decrease the function and/or expression of Syk, where such function may include transcription regulatory activity and/or protein-binding. Modulation may occur in vitro or in vivo. Modulation, as described herein, includes the inhibition, antagonism, partial antagonism, activation, agonism or partial agonism of a function or characteristic associated with Syk, either directly or indirectly, and/or the upregulation or downregulation of the expression of Syk, either directly or indirectly. In a preferred embodiment, the modulation is direct. Inhibitors or antagonists are compounds that, e.g., bind to, partially or totally block stimulation, decrease, prevent, inhibit, delay activation, inactivate, desensitize, or downregulate signal transduction. Activators or agonists are compounds that, e.g., bind to, stimulate, increase, open, activate, facilitate, enhance activation, activate, sensitize or upregulate signal transduction. The ability of a compound to inhibit the function of Syk can be demonstrated in a biochemical assay, e.g., binding assay, or a cell-based assay, e.g., a transient transfection assay.

“Modulators” of activity are used to refer to “ligands”, “antagonists” and “agonists” identified using in vitro and in vivo assays for activity and their homologs and mimetics. Modulators include naturally occurring and synthetic ligands, antagonists, agonists, molecules and the like. Assays to identify antagonists and agonists include, e.g., applying putative modulator compounds to cells, in the presence or absence of a receptor of the invention and then determining the functional effects on a receptor of the invention activity. Samples or assays comprising a receptor of the invention that are treated with a potential activator, inhibitor, or modulator are compared to control samples without the inhibitor, activator, or modulator to examine the extent of effect. Control samples (untreated with modulators) are assigned a relative activity value of 100% Inhibition is achieved when the activity value of a receptor of the invention relative to the control is about 80%, optionally 50% or 25-1%. Activation is achieved when the activity value of a receptor of the invention relative to the control is 110%, optionally 150%, optionally 200-500%, or 1000-3000% higher.

“Patient” refers to human and non-human animals, especially mammals. Examples of patients include, but are not limited to, humans, cows, dogs, cats, goats, sheep, pigs and rabbits.

Turning next to the compositions of the invention, the term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

The terms “pharmaceutically effective amount”, “therapeutically effective amount” or “therapeutically effective dose” refers to the amount of the subject compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the symptoms of the condition or disorder being treated. The therapeutically effective amount will vary depending on the compound, the disorder or condition and its severity and the age, weight, etc., of the mammal to be treated.

The term “platelet” refers to a minute, normucleated, disklike cell found in the blood plasma of mammals that functions to promote blood clotting.

The terms “prevent”, “preventing”, “prevention” and grammatical variations thereof as used herein, refers to a method of partially or completely delaying or precluding the onset or recurrence of a disorder or condition and/or one or more of its attendant symptoms or barring a subject from acquiring or reacquiring a disorder or condition or reducing a subject's risk of acquiring or reaquiring a disorder or condition or one or more of its attendant symptoms.

The term “recanalization” refers to the process of restoring flow to or reuniting an interrupted channel of the body, such as a blood vessel.

The term “restenosis” refers to a re-narrowing or blockage of an artery at the same site where treatment, such as an angioplasty or a stent procedure, has been performed.

The phrase “selectively” or “specifically” when referring to binding to a receptor, refers to a binding reaction that is determinative of the presence of the receptor, often in a heterogeneous population of receptors and other biologics. Thus, under designated conditions, the compounds bind to a particular receptor at least two times the background and more typically more than 10 to 100 times background. Specific binding of a compound under such conditions requires a compound that is selected for its specificity for a particular receptor. For example, small organic molecules can be screened to obtain only those compounds that specifically or selectively bind to a selected receptor and not with other receptors or proteins. A variety of assay formats may be used to select compounds that are selective for a particular receptor. For example, High-throughput screening assays are routinely used to select compounds that are selective for a particular a receptor.

As used herein, the term “Sickle cell anemia” refers to an inherited disorder of the red blood cells in which both hemoglobin alleles encode the sickle hemoglobin (S) protein, i.e., the S/S genotype. The presence of abnormal hemoglobin results in the production of unusually shaped cells, which do not survive the usual length of time in the blood circulation. Thus, anemia results. “Anemia” refers to a decrease in the number of red blood cells and/or hemoglobin in the blood.

The term “Sickle cell disease” refers to an inherited disorder of the red blood cells in which one hemoglobin allele encodes the sickle hemoglobin (S) protein, and the other allele encodes another unusual hemoglobin protein, such as hemoglobin (S), (C), (D), (E), and (βThal). Examples of sickle cell disease genotypes include, without limitation, the S/S, S/C, S/D, S/E, and S/βThal genotypes. The most common types of sickle cell disease include sickle cell anemia, sickle-hemoglobin C disease, sickle beta-plus thalassemia, and sickle beta-zero thalassemia.

The “subject” is defined herein to include animals such as mammals, including, but not limited to, primates (e.g., humans), cows, sheep, goats, horses, dogs, cats, rabbits, rats, mice and the like. In preferred embodiments, the subject is a human.

As used herein, the term “Syk” refers to a spleen tyrosine kinase (RefSeq Accession No. P-043405) or a variant thereof that is capable of mediating a cellular response to T-cell receptors in vitro or in vivo. Syk variants include proteins substantially homologous to native Syk, i.e., proteins having one or more naturally or non-naturally occurring amino acid deletions, insertions or substitutions (e.g., Syk derivatives, homologs and fragments). The amino acid sequence of Syk variant preferably is at least about 80% identical to a native Syk, more preferably at least about 90% identical, and most preferably at least about 95% identical.

The term “Syk inhibitor” refers to any agent that inhibits the catalytic activity of spleen tyrosine kinase.

The terms “treat”, “treating”, “treatment” and grammatical variations thereof as used herein, includes partially or completely delaying, alleviating, mitigating or reducing the intensity, progression, or worsening of one or more attendant symptoms of a disorder or condition and/or alleviating, mitigating or impeding one or more causes of a disorder or condition. Treatments according to the invention may be applied preventively, prophylactically, pallatively or remedially.

The term “vessel” refers to any channel for carrying a fluid, such as an artery or vein. For example, a “blood vessel” refers to any of the vessels through which blood circulates in the body. The lumen of a blood vessel refers to the inner open space or cavity of the blood vessel.

### 2. Embodiments of the Invention

a. Compounds

The present invention provides in one embodiment, a compound of Formula (I):

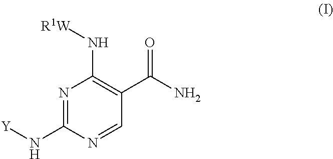

or a pharmaceutically acceptable salt thereof.

In some embodiments, W is selected from the group consisting of

(a) C₃₋₈cycloalkyl, optionally substituted with from 1 to 4 substituents independently selected from the group consisting of C₁₋₈ alkyl, amino, hydroxy, C₁₋₈alkylcarbonyl, aminocarbonyl, C₁₋₈alkoxycarbonylamino, arylC₁₋₈alkoxycarbonylamino, aryl and heterocyclylC₁₋₈alkylene;

(b) C₁₋₈ alkyl, optionally substituted with from 1 to 4 substituents independently selected from the group consisting of amino, oxo, C₁₋₈alkoxy, C₂₋₈alkynyl, cyano, aminocarbonyl, C₁₋₈haloalkylene, hydroxy, halogen, C₃₋₈cycloalkyl, and aryl;

(c) C₁₋₈ alkylC₃₋₈heterocyclyl, optionally substituted with from 1 to 4 substituents independently selected from the group consisting of C₁₋₈alkyl, C₁₋₈alkylcarbonyl, C₁₋₈alkylsulfonyl; and aminocarbonyl;

(d) aryl, optionally substituted with from 1 to 4 substituents independently selected from the group consisting of C₁₋₈alkyl, C₂₋₈alkenyl, C₂₋₈alkynyl, C₁₋₈haloalkylene, carboxy, acyl, acylamino, cyano, amino, aminocarbonyl, aminosulfonyl, sulfonyl, nitro, hydroxy, C₁₋₈alkoxy, aryloxy, halo, sulfonylamino, C₃₋₈cycloalkyl, aryl, heterocyclyl C₁₋₈alkylsulfonyl, C₁₋₈alkylcarbonylheterocyclyl and heteroaryl;

(e) heteroaryl, optionally substituted with from 1 to 4 substituents independently selected from the group consisting of C₁₋₈alkyl, C₁₋₈alkylcarbonyl, aminocarbonyl, C₁₋₈alkoxycarbonyl, amino, C₁₋₈ alkoxycarbonylamino, arylC₁₋₈alkoxycarbonylamino, hydroxy, C₁₋₈ alkoxy, C₁₋₈alkylsulfonyl, oxo, halo, aryl and heterocyclylC₁₋₈alkylene;

(f) C₃₋₈heterocyclyl, optionally substituted with from 1 to 4 substituents independently selected from the group consisting of C₁₋₈alkyl, C₁₋₈alkoxycarbonyl and oxo;

R¹ is selected from the group consisting of H, C₁₋₈ alkyl, amino, aminocarbonyl, hydroxy, C₁₋₈ alkoxy, C₁₋₈ haloalkylene, C₂₋₈ alkenyl, C₂₋₈ alkynyl, oxo, cyano, C₁₋₈ alkoxycarbonyl, C₃₋₈ cycloalkyl, aryl and heterocyclyl; and each heterocyclyl is optionally substituted with from 1 to 4 substituents selected from the group consisting of C₁₋₈ alkyl, halo, oxo, amino, C₁₋₈alkoxy, C₁₋₈alkylcarbonyl, arylC₁₋₈ alkoxycarbonyl, aminocarbonyl, arylC₁₋₈ alkylenecarbonyl and C₁₋₈ alkylsulfonyl;

Y is selected from the group consisting of

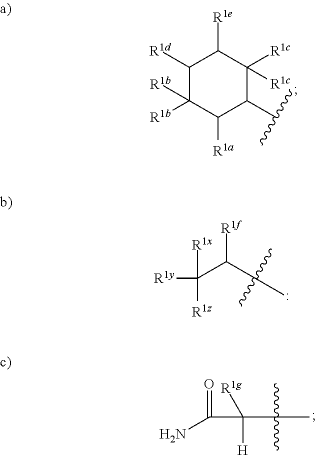

and
d) heterocyclyl, optionally substituted with from 1 to 4 substituents independently selected from the group consisting of C₁₋₈alkyl, C₁₋₈alkenyl, amino, cyanoC₁₋₈alkylene, aminocarbonyl, alkylaminocarbonyl, dialkylaminocarbonyl, oxoC₁₋₈alkylene, hydroxyalkyl, carboxy, haloC₁₋₈alkylene, cyano and oxo and halo
e) phenyl, optionally substituted with from 1 to 4 substituents independently selected from the group consisting of alkyl, alkoxy and halo;
f) pyridyl, optionally substituted with from 1 to 4 substituents independently selected from the group consisting of alkoxy;
g) indinlyl, optionally substituted with from 1 to 4 substituents independently selected from the group consisting of hydroxyl and oxo;
R^{1a} is selected from the group consisting of oxo, hydroxy, alkoxy, NH₂, N₃, triazinyl, HC(O)NH—, NCCH₂NH—, HOCH₂CH₂NH—, R^{1u}OCONH—, R^{1v}NHCH(CH₃)NH—, N⁺(O⁻) H₂, N(O), N(═CH₂), R^{1w}OC(O)NH—, and C₁₋₈alkylC(O)NH—;
R^{1b} is selected from the group consisting of H, hydroxyl, fluoro, combined to form an oxo group, or one R^{1b} is combined with R^{1a} to form a pyridyl ring and the other R^{1b} is null;
R^{1c} is selected from the group consisting of H, fluoro, hydroxyl, alkoxy, benzyloxy;
R^{1d} is independently selected from H, alkoxycarbonyl, alkylaminocarbonyl, dialkylaminocarbonyl, and heterocyclylcarbonyl;
R^{1e} is independently selected from H and aminocarbonyl;
R^{1u} is selected from the group consisting of H, alkyl, and heterocyclyl optionally substituted with one to four substitutents independently selected from the group consisting of oxo, hydroxy, and carboxy;
R^{1v} is a sugar moiety;
R^{1w} is a moiety of formula V attached via a covalent bond at R^{1a} wherein Y is

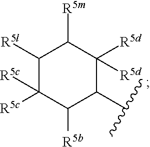

each R^{1f} is selected from the group consisting of H, C₁₋₈alkyl, C₁₋₈haloalkylene, phenyl, C₃₋₈cycloalkyl, hydroxyC₁₋₈alkylene, NH₂, C₁₋₈alkylamino, C₁₋₈ alkoxycarbonylaminoC₁₋₈ alkylene, C₃₋₈cycloalkylC₁₋₈ alkylene, heteroaryl, C₁₋₈alkylthioC₁₋₈ alkylene, C₁₋₈alkylsulfonylC₁₋₈ alkylene, aminocarbonyl, C₁₋₈alkoxyC₁₋₈alkyl, haloC₁₋₈alkylene, aryl and heterocyclyl; wherein the aryl is optionally substituted by hydroxy, C₁₋₈alkoxy, halo or haloC₁₋₈alkylene;

R^{1g} is independently selected from the group consisting of H, C₁₋₈alkyl, C₃₋₈cycloalkyl, and C₃₋₈cycloalkylC₁₋₈ alkylene;

R^{1x} is H, alkyl, haloalkyl or combined with R^{1y} to form a cycloalkyl group;

R^{1y} is selected from the group consisting of H, C₁₋₈alkyl, C₁₋₈alkylamino, amino aminoC₁₋₈alkylene, carboxy, C₁₋₈alkylaminoC₁₋₈alkylene, C₁₋₈alkoxyC₁₋₈alkylene, hydroxyC₁₋₈alkylene; carboxyC₁₋₈alkylene, C₃₋₈cycloalkylC₁₋₈alkylene, aryloxyC₁₋₈alkylene, arylC₁₋₈alkylene, heteroarylC₁₋₈alkylene, and hydroxyC₁₋₈alkoxy; or
R^{1y} may be combined with R^{1f} or R^{1x} and the atoms to which they are attached to form a C₃₋₈ cycloalkyl or heterocyclyl ring optionally substituted with one to three groups independently selected from hydroxy, halo, oxo and amino;

R^{1z} is selected from the group consisting of H, amino, C₁₋₈alkylamino, hydroxycarbonylamino, C₁₋₈alkoxycarbonylamino, arylC₁₋₈alkoxycarbonylamino and hydroxy;

and the wavy line indicates the point of attachment to the rest of the molecule

wherein the wavy line indicates the point of attachment to the rest of the molecule.

In some embodiments, W is C₃₋₈cycloalkyl, optionally substituted with from 1 to 4 substituents independently selected from the group consisting of C₁₋₈ alkyl, amino, hydroxy, C₁₋₈alkylcarbonyl, aminocarbonyl, C₁₋₈alkoxycarbonylamino, arylC₁₋₈alkoxycarbonylamino, aryl and heterocyclylC₁₋₈alkylene.

In some embodiments, W is C₁₋₈ alkyl, optionally substituted with from 1 to 4 substituents independently selected from the group consisting of amino, oxo, C₁₋₈alkoxy, C₂₋₈alkynyl, cyano, aminocarbonyl, C₁₋₈haloalkylene, hydroxy, halogen, C₃₋₈cycloalkyl, and aryl.

In some embodiments, W is C₁₋₈ alkylC₃₋₈heterocyclyl, optionally substituted with from 1 to 4 substituents independently selected from the group consisting of C₁₋₈alkyl, C₁₋₈alkylcarbonyl, C₁₋₈alkylsulfonyl; and aminocarbonyl.

In some embodiments, W is aryl, optionally substituted with from 1 to 4 substituents independently selected from the group consisting of C₁₋₈alkyl, C₂₋₈alkenyl, C₂₋₈alkynyl, C₁₋₈haloalkylene, carboxy, acyl, acylamino, cyano, amino, aminocarbonyl, aminosulfonyl, sulfonyl, nitro, hydroxy, C₁₋₈alkoxy, aryloxy, halo, sulfonylamino, C₃₋₈cycloalkyl, aryl, heterocyclyl C₁₋₈alkylsulfonyl, C₁₋₈alkylcarbonylheterocyclyl and heteroaryl.

In some embodiments, W is heteroaryl, optionally substituted with from 1 to 4 substituents independently selected from the group consisting of C₁₋₈alkyl, C₁₋₈alkylcarbonyl, aminocarbonyl, C₁₋₈alkoxycarbonyl, amino, C₁₋₈ alkoxycarbonylamino, arylC₁₋₈alkoxycarbonylamino, hydroxy, C₁₋₈ alkoxy, C₁₋₈alkylsulfonyl, oxo, halo, aryl and heterocyclylC₁₋₈alkylene.

In some embodiments, W is C₃₋₈heterocyclyl, optionally substituted with from 1 to 4 substituents independently selected from the group consisting of C₁₋₈alkyl, C₁₋₈alkoxycarbonyl and oxo.

In some embodiments, R¹ is H. In some embodiments, R¹ is C₁₋₈ alkyl. In some embodiments, R¹ is amino. In some embodiments, R¹ is aminocarbonyl. In some embodiments, R¹ is hydroxy. In some embodiments, R¹ is C₁₋₈ alkoxy. In some embodiments, R¹ is C₁₋₈ haloalkylene. In some embodiments, R¹ is C₂₋₈ alkenyl. In some embodiments, R¹ is C₂₋₈ alkynyl. In some embodiments, R¹ is oxo. In some embodiments, R¹ is cyano. In some embodiments, R¹ is C₁₋₈ alkoxycarbonyl. In some embodiments, R¹ is C₃₋₈ cycloalkyl. In some embodiments, R¹ is aryl. In some embodiments, R¹ is heterocyclyl. In some embodiments, heterocyclyl is optionally substituted with from 1 to 4 substituents selected from the group consisting of C₁₋₈ alkyl, halo, oxo, amino, C₁₋₈alkoxy, C₁₋₈alkylcarbonyl, arylC₁₋₈alkoxycarbonyl, aminocarbonyl, arylC₁₋₈ alkylenecarbonyl and C₁₋₈alkylsulfonyl.

In some embodiments, Y is

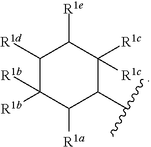

In some embodiments, Y is

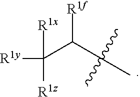

In some embodiments, Y is

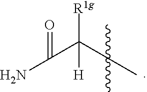

In some embodiments, Y is heterocyclyl, optionally substituted with from 1 to 4 substituents independently selected from the group consisting of C₁₋₈alkyl, C₁₋₈alkenyl, amino, cyanoC₁₋₈alkylene, aminocarbonyl, alkylaminocarbonyl, dialkylaminocarbonyl, oxoC₁₋₈alkylene, hydroxyalkyl, carboxy, haloC₁₋₈alkylene, cyano and oxo and halo.

In some embodiments, Y is phenyl, optionally substituted with from 1 to 4 substituents independently selected from the group consisting of alkyl, alkoxy and halo.

In some embodiments, Y is pyridyl, optionally substituted with from 1 to 4 substituents independently selected from the group consisting of alkoxy.

In some embodiments, Y is indinlyl, optionally substituted with from 1 to 4 substituents independently selected from the group consisting of hydroxyl and oxo.

In some embodiments, R^{1a} is oxo. In some embodiments, R^{1a} is hydroxy. In some embodiments, R^{1a} is alkoxy. In some embodiments, R^{1a} is NH₂. In some embodiments, R^{1a} is N₃. In some embodiments, R^{1a} is triazinyl. In some embodiments, R^{1a} is HC(O)NH—. In some embodiments, R^{1a} is NCCH₂NH—. In some embodiments, R^{1a} is HOCH₂CH₂NH—. In some embodiments, R^{1a} is R^{1u}OCONH—. In some embodiments, R^{1a} is R^{1v}NHCH(CH₃)NH—. In some embodiments, R^{1a} is N⁺(O⁻)H₂. In some embodiments, R^{1a} is N(O). In some embodiments, R^{1a} is N(═CH₂). In some embodiments, R^{1a} is R^{1w}OC(O)NH—. In some embodiments, R^{1a} is C₁₋₈alkylC(O)NH—.

In some embodiments, R^{1b} is H. In some embodiments, R^{1b} is hydroxyl. In some embodiments, R^{1b} is fluoro. In some embodiments, R^{1b} is combined to form an oxo group. In some embodiments, one R^{1b} is combined with R^{1a} to form a pyridyl ring and the other R^{1b} is null.

In some embodiments, R^{1c} is H. In some embodiments, R^{1c} is fluoro. In some embodiments, R^{1c} is hydroxyl. In some embodiments, R^{1c} is alkoxy. In some embodiments, R^{1c} is benzyloxy.

In some embodiments, R^{1d} is H. In some embodiments, R^{1d} is alkoxycarbonyl. In some embodiments, R^{1d} is alkylaminocarbonyl. In some embodiments, R^{1d} is dialkylaminocarbonyl. In some embodiments, R^{1d} is heterocyclylcarbonyl.

In some embodiments, R^{1e} is H. In some embodiments, R^{1e} is aminocarbonyl.

In some embodiments, R^{1u} is selected from the group consisting of H. In some embodiments, R^{1u} is alkyl. In some embodiments, R^{1u} is heterocyclyl. In some embodiments, R^{1u} is optionally substituted with one to four substitutents independently selected from the group consisting of oxo, hydroxy, and carboxy.

In some embodiments, R^{1v} is a sugar moiety.

In some embodiments, R^{1w} is a moiety of formula V attached via a covalent bond at R^{1a} wherein Y is

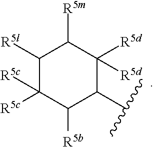

In some embodiments, R^{1f} is H. In some embodiments, R^{1f} is C₁₋₈alkyl. In some embodiments, R^{1f} is C₁₋₈haloalkylene. In some embodiments, R^{1f} is phenyl. In some embodiments, R^{1f} is C₃₋₈cycloalkyl. In some embodiments, R^{1f} is hydroxyC₁₋₈alkylene. In some embodiments, R^{1f} is NH₂. In some embodiments, R^{1f} is C₁₋₈alkylamino. In some embodiments, R^{1f} is C₁₋₈alkoxycarbonylaminoC₁₋₈ alkylene. In some embodiments, R^{1f} is C₃₋₈cycloalkylC₁₋₈ alkylene. In some embodiments, R^{1f} is heteroaryl. In some embodiments, R^{1f} is C₁₋₈alkylthioC₁₋₈ alkylene. In some embodiments, R^{1f} is C₁₋₈alkylsulfonylC₁₋₈ alkylene. In some embodiments, R^{1f} is aminocarbonyl. In some embodiments, R^{1f} is C₁₋₈alkoxyC₁₋₈alkyl. In some embodiments, R^{1f} is haloC₁₋₈alkylene. In some embodiments, R^{1f} is aryl. In some embodiments, R^{1f} is heterocyclyl. In some embodiments, the aryl is optionally substituted by hydroxy, C₁₋₈alkoxy, halo or haloC₁₋₈alkylene.

In some embodiments, R^{1g} is H, C₁₋₈alkyl. In some embodiments, R^{1g} is C₃₋₈cycloalkyl. In some embodiments, is C₃₋₈cycloalkylC₁₋₈ alkylene.

In some embodiments, R^{1x} is H, alkyl, haloalkyl or combined with R^{1y} to form a cycloalkyl group.

In some embodiments, R^{1y} is H. In some embodiments, R^{1y} is C₁₋₈alkyl. In some embodiments, R^{1y} is C₁₋₈alkylamino. In some embodiments, R^{1y} is amino aminoC₁₋₈alkylene. In some embodiments. In some embodiments, R^{1y} is R^{1y} is carboxy. In some embodiments, R^{1y} is C₁₋₈alkylaminoC₁₋₈alkylene. In some embodiments, R^{1y} is C₁₋₈alkoxyC₁₋₈alkylene. In some embodiments, R^{1y} is hydroxyC₁₋₈alkylene. In some embodiments, R^{1y} is carboxyC₁₋₈alkylene. In some embodiments, R^{1y} is C₃₋₈cycloalkylC₁₋₈alkylene. In some embodiments, R^{1y} is aryloxyC₁₋₈alkylene. In some embodiments, R^{1y} is arylC₁₋₈alkylene. In some embodiments, R^{1y} is heteroarylC₁₋₈alkylene. In some embodiments, R^{1y} is hydroxyC₁₋₈alkoxy.

In some embodiments, R^{1y} may be combined with R^{1f} or R^{1x} the atoms to which they are attached to form a C₃₋₈ cycloalkyl or heterocyclyl ring optionally substituted with one to three groups independently selected from hydroxy, halo, oxo and amino.

In some embodiments, R^{1z} is selected from the group consisting of H, amino, C₁₋₈alkylamino, hydroxycarbonylamino, C₁₋₈alkoxycarbonylamino, arylC₁₋₈alkoxycarbonylamino and hydroxy.

In some embodiments, Y is

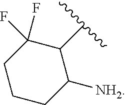 [SMILES: *C1C(N)CCCC1(F)F]

In some embodiments, W is selected from the group consisting of

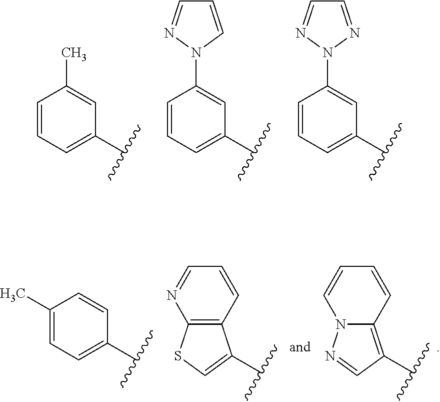

In some embodiments, Y is

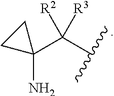

In some embodiments, R² is cycloalkyl. In some embodiments, R² is cycloalkylC₁₋₈alkylene. In some embodiments, R² is C₁₋₈alkyl. In some embodiments, R² is optionally substituted with one or two or three halo substituents.

In some embodiments, R³ is hydrogen or together with R² and the carbon atom to which they are attached to form a cycloalkyl ring optionally substituted with one to three halo substituents.

In some embodiments, Y is selected from the group consisting of

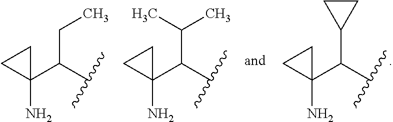

In some embodiments, W is selected from the group consisting of

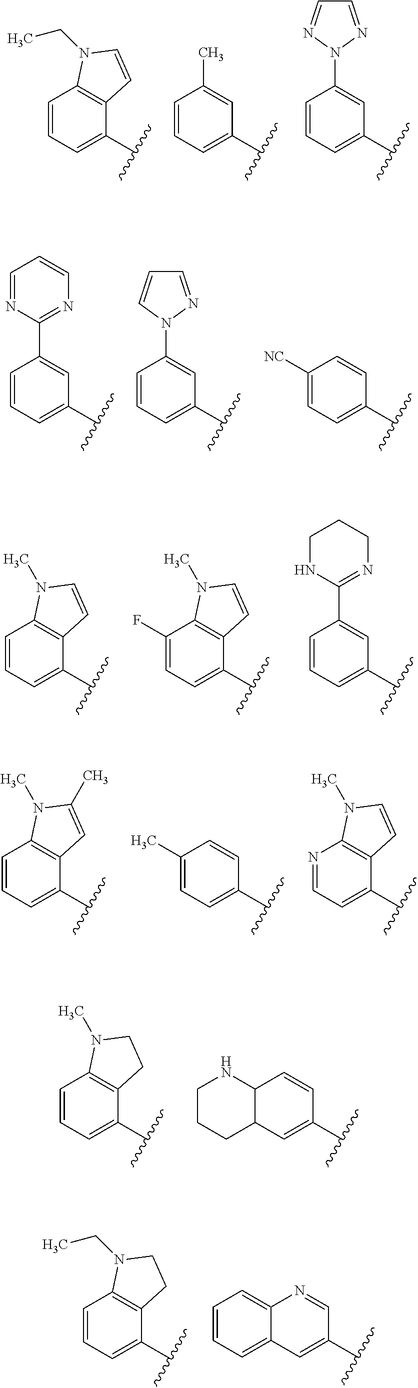

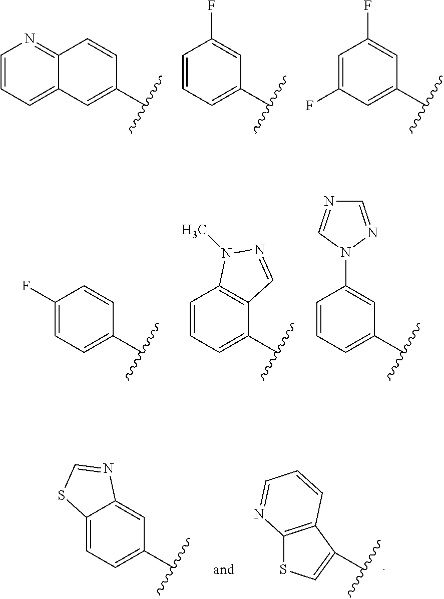

In some embodiments, Y is

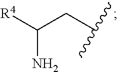

wherein R⁴ is C₁-C₆alkyl optionally substituted with one or two or three halo substituents.

In some embodiments, R⁴ is selected from the group consisting of CHF₂CH₂— and CH₃CF₂—

In some embodiments, W is selected from the group consisting of

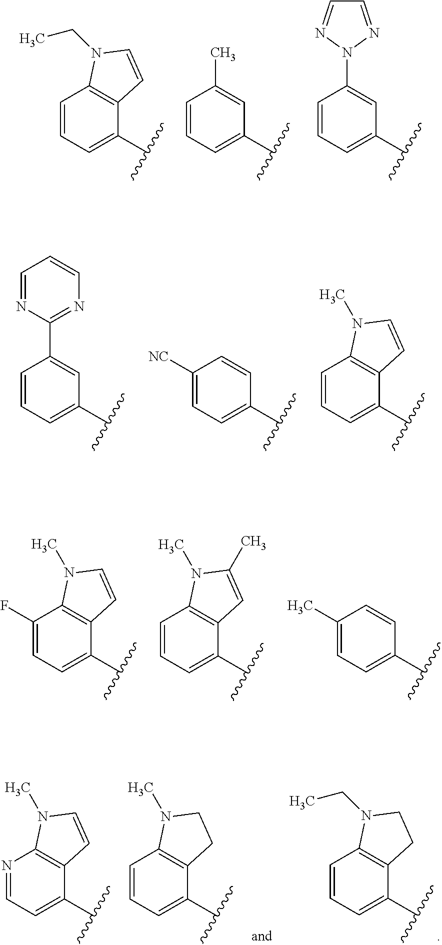

The present invention provides in one embodiment, a compound of Formula (II) or a pharmaceutically acceptable salt thereof:

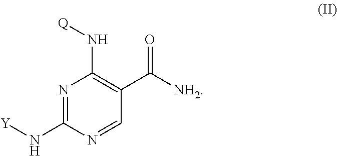

In some embodiments, Q is heteroaryl optionally substituted with one to five R^{3a} groups.

In some embodiments, Q cycloalkyl optionally substituted with one to five R^{3a} groups.

In some embodiments, Q heterocyclyl optionally substituted with one to five R^{3a} groups.

In some embodiments, Q aryl substituted with R^{3b} and optionally substituted with one to four R^{3a} groups.

In some embodiments, R^{3b} is -is selected from the group consisting of C₁₋₈ alkyl, C₃₋₈ cycloalkylC₁₋₈ alkyl, C₁₋₈ alkoxy, C₃₋₈ cycloalkoxy, hydroxyC₁₋₈ alkyl, C₁₋₈ alkoxyalkyl, haloC₁₋₈ alkyl, haloC₁₋₈ alkoxy, amino, C₁₋₈ alkylamino, diC₁₋₈ alkylamino, halo, haloC₁₋₈ alkylaminocarbonyl, C₁₋₈alkylaminocarbonyl, diC₁₋₈ alkylaminocarbonyl, aminocarbonyl, heterocyclylcarbonyl, C₁₋₈ alkylcarbonylamino, C₁₋₈ alkylsulfonyl, aminosulfonyl, C₃₋₈ cycloalkyl, C₁₋₈ alkylcarbonylpiperadinyl, morpholinyl, phenyl, and heteroaryl optionally substituted with one to three R^{3c} groups.

In some embodiments, R^{3a} and R^{3c} are independently selected from the group consisting of C₁₋₈ alkyl, C₂₋₈ alkenyl, C₂₋₈alkynyl, C₃₋₈ cycloalkylC₁₋₈ alkylene, C₁₋₈ alkoxy, C₃₋₈ cycloalkoxy, hydroxyC₁₋₈ alkylene, C₁₋₈ alkoxyalkylene, haloC₁₋₈ alkylene, haloC₁₋₈ alkoxy, amino, hydroxyl, C₁₋₈ alkylamino, diC₁₋₈ alkylamino, oxo, halo, cyano, haloC₁₋₈ alkylaminocarbonyl, C₁₋₈alkylaminocarbonyl, diC₁₋₈ alkylaminocarbonyl, aminocarbonyl, heterocyclylcarbonyl, C₁₋₈ alkylcarbonylamino, C₁₋₈ alkylsulfonyl, aminosulfonyl, C₃₋₈ cycloalkyl, C₁₋₈ alkylcarbonylpiperadinyl, heterocyclyl, phenyl, heteroaryl, heteroarylsulfinyl; C₁₋₈arylalkylene, aminoC₁₋₈alkylene.

In some embodiments, Y is

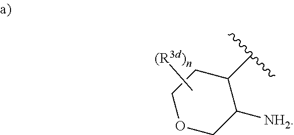

In some embodiments, Y is

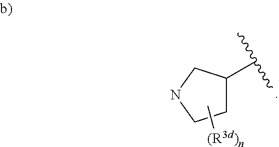

In some embodiments, Y is

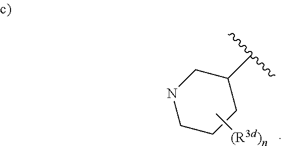

In some embodiments, R^{3d} is independently selected from the group consisting of C₁₋₈alkyl, C₁₋₈alkylcarbonyl, cyanoC₁₋₈alkylene, hydroxyC₁₋₈alkylene, haloC₁₋₈alkylene, halo, and amino, and n is 0, 1, 2, 3, 4, or 5.

In some embodiments, R^{3e} is selected from the group consisting of hydrogen, cycloalkyl, cycloalkylC₁₋₈alkyl, and C₁₋₈alkyl, wherein R^{3e} is optionally substituted with one to five groups independently selected from halo, C₁₋₈alkyl, and amino.

In some embodiments, R^{3f} is hydrogen or together with R^{3e} and the carbon atom to which they are attached to form a cycloalkyl ring.

In some embodiments, R^{3g} is C₁₋₈alkyl optionally substituted with one to three halo substituents.

The present invention provides in one embodiment, a compound of Formula (IIa)

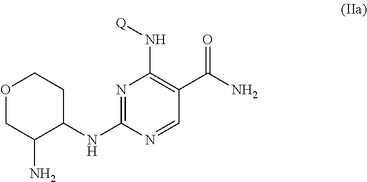

or a pharmaceutically acceptable salt thereof.

In some embodiments, Q is phenyl or heteroaryl optionally substituted with R^{2a}, wherein heteroaryl is selected from the group consisting of pyrimidinyl, indolyl, benzothiazolyl, thieno[2,3-b]pyridinyl, and quinolinyl.

In some embodiments, R^{2a} is independently selected from the group consisting of C₁₋₈alkyl, haloC₁₋₈alkylene, halo, and cyano.

The present invention provides in one embodiment, a compound of Formula (IIb)

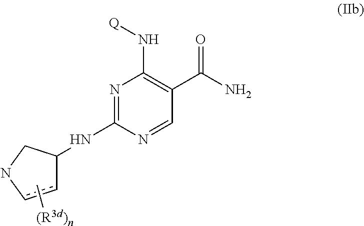

or a pharmaceutically acceptable salt thereof.

In some embodiments, Q is phenyl or heteroaryl optionally substituted with R^{2a}, wherein heteroaryl is selected from the group consisting of triazoyl, pyrimidinyl, indolyl, benzothiazolyl, thieno[2,3-b]pyridinyl, and quinolinyl.

In some embodiments, R^{2a} is independently selected from the group consisting of H, C₁₋₈alkyl, C₁₋₈alkenyl, cyanoC₁₋₈alkylene, aminocarbonyl, alkylaminocarbonyl, dialkylaminocarbonyl, oxoC₁₋₈alkylene, hydroxyalkyl, carboxy, haloC₁₋₈alkylene, cyano and oxo and halo.

In some embodiments, Y is selected from the group consisting of

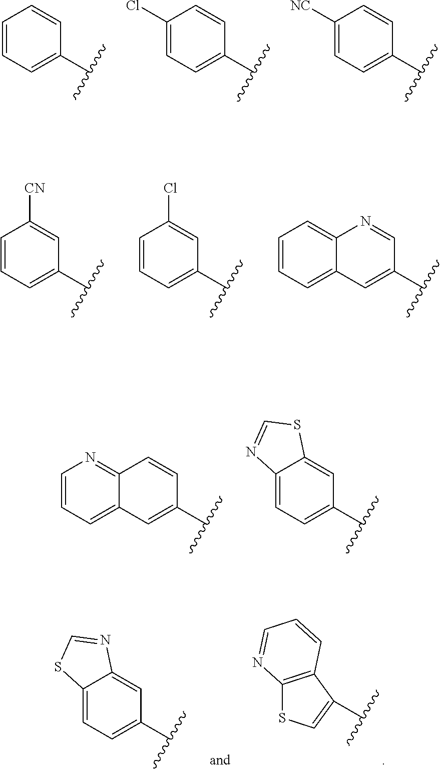

In some embodiments, the compound has Formula (IIIa), (IIIb), (IIIc) or (IIId):

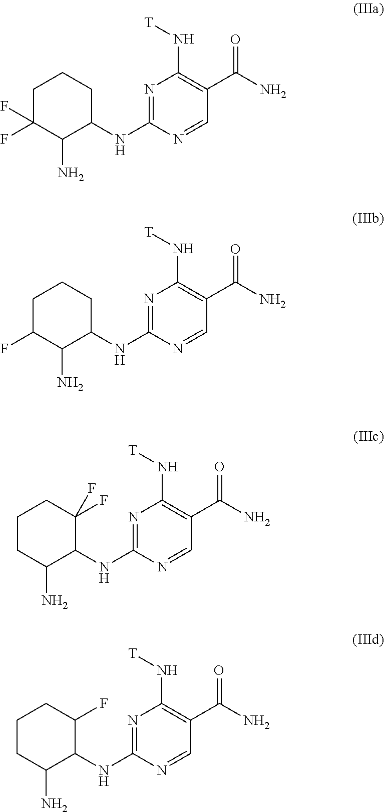

or a pharmaceutically acceptable salt thereof.

In some embodiments, T is selected from the group consisting of phenyl, pyrimidinyl, indolyl, indazoyl, benzothiazolyl, thieno[2,3-b]pyridinyl, pyrazolo[1,5-a]pyridine and quinolinyl, wherein T is optionally substituted with one to three R^{3a} substituents.

In some embodiments, R^{3a} is independently selected from the group consisting of C₁₋₈alkyl, haloC₁₋₈alkylene, halo, cyano, pyrimidyl, and pyrazoyl.

In some embodiments, T is quinolinyl. In some embodiments, T is selected from the group consisting of

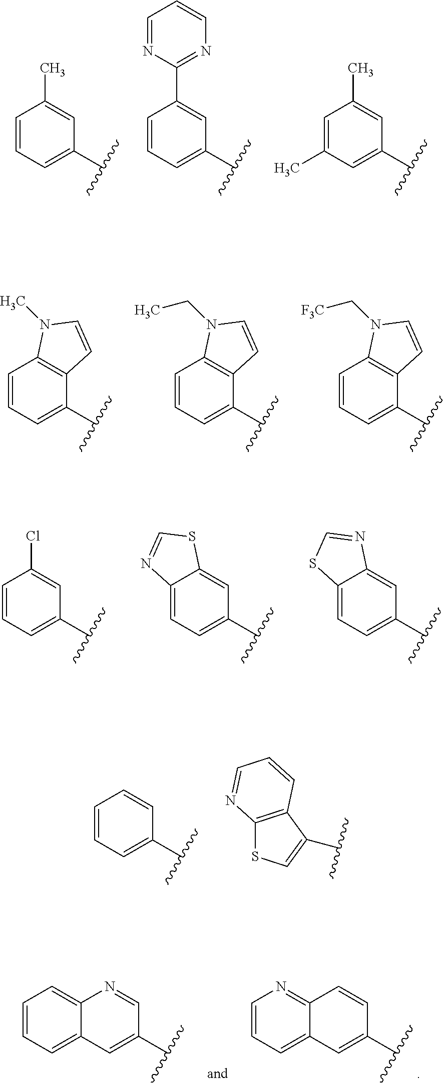

The present invention provides in one embodiment, a compound of Formula (IV)

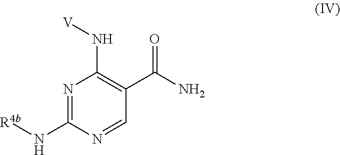

or a pharmaceutically acceptable salt thereof.

In some embodiments, V is phenyl, optionally substituted with from 1 to 4 R^{4a}.

In some embodiments, V is heteroaryl, optionally substituted with from 1 to 4 R^{4a}.

In some embodiments, each R^{4a} is independently selected from the group consisting of C₁₋₈alkyl, C₁₋₈alkoxy, cyano, hydroxyl, oxo, halo, haloC₁₋₈alkyl and heteroaryl.

In some embodiments, R^{4b} is

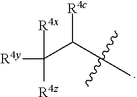

In some embodiments, R^{4b} is

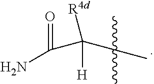

In some embodiments, each R^{4c} is selected from the group consisting of H, C₁₋₈alkyl, C₁₋₈haloalkylene, phenyl, C₃₋₈cycloalkyl, hydroxyC₁₋₈alkylene, NH₂, C₁₋₈alkylamino, C₁₋₈ alkoxycarbonylaminoC₁₋₈alkylene, C₃₋₈cycloalkylC₁₋₈ alkylene, heteroaryl, C₁₋₈alkylthioC₁₋₈ alkylene, C₁₋₈alkylsulfonylC₁₋₈alkylene, aminocarbonyl, C₁₋₈alkoxyC₁₋₈alkyl, haloC₁₋₈alkylene, aryl and heterocyclyl; wherein the aryl is optionally substituted by hydroxy, C₁₋₈alkoxy, halo or haloC₁₋₈alkylene.

In some embodiments, R^{4d} is independently selected from the group consisting of H, C₁₋₈alkyl, C₃₋₈cycloalkyl, and C₃₋₈cycloalkylC₁₋₈ alkylene.

In some embodiments, R^{4x} is H, alkyl, haloalkyl or combined with R^{4y} to form a cycloalkyl group.

In some embodiments, R^{4y} is selected from the group consisting of H, C₁₋₈alkyl, C₁₋₈alkylamino, amino aminoC₁₋₈alkylene, carboxy, C₁₋₈alkylaminoC₁₋₈alkylene, C₁₋₈alkoxyC₁₋₈alkylene, hydroxyC₁₋₈alkylene; carboxyC₁₋₈alkylene, C₃₋₈cycloalkylC₁₋₈alkylene, aryloxyC₁₋₈alkylene, arylC₁₋₈alkylene, heteroarylC₁₋₈alkylene, and hydroxyC₁₋₈alkoxy.

In some embodiments, R^{4y} may be combined with R^{4c} or R^{4X} and the atoms to which they are attached to form a C₃₋₈ cycloalkyl or heterocyclyl ring optionally substituted with one to three groups independently selected from hydroxy, halo, oxo and amino.

In some embodiments, R^{4z} is selected from the group consisting of H, amino, C₁₋₈alkylamino, hydroxycarbonylamino, C₁₋₈alkoxycarbonylamino, arylC₁₋₈alkoxycarbonylamino and hydroxyl.

In some embodiments, the wavy line indicates the point of attachment to the rest of the molecule.

In some embodiments, R^{4b} is cyclohexyl substituted with amino and further optionally substituted with one to three halo substituents. In some embodiments, R^{4b} is

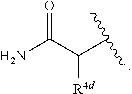

In some embodiments, R^{4b} is C₁₋₈alkyl or haloC₁₋₈alkylene.

In some embodiments, heteroaryl is selected from the group consisting of: thienyl, thiazoyl, thiadiazoyl, isothiazoyl, pyrazoyl, triazoyl, pyrimidinyl, tetrahydroprimidinyl, indolyl, indolinyl, indazoyl, benzothiazolyl, thieno[2,3-b]pyridinyl, pyrazolo[1,5-a]pyridine, 1H-pyrrolo[2,3-b]pyridine, isoquinolinyl, tetrahydroquinolinyl and quinolinyl.

The present invention provides in one embodiment, a compound of Formula (V)

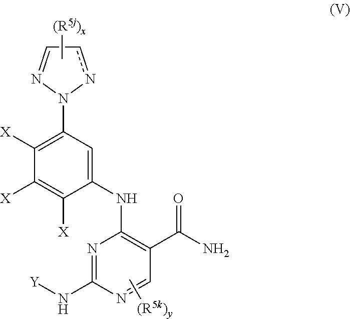

or a pharmaceutically acceptable salt thereof.

In some embodiments, X is independently, H or halogen;

In some embodiments, Y is CH₃CH₂NH—. In some embodiments, Y is (CH₃)₂N—. In some embodiments, Y is CH₂CH(NH₂)CH₂CHCF₂. In some embodiments, Y is

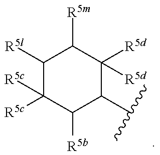

In some embodiments, R^{5b} is oxo. In some embodiments, R^{5b} is hydroxy In some embodiments, R^{5b} is alkoxy In some embodiments, R^{5b} is NH₂. In some embodiments, R^{5b} is N₃. In some embodiments, R^{5b} is triazinyl. In some embodiments, R^{5b} is HC(O)NH—. In some embodiments, R^{5b} is NCCH₂NH—. In some embodiments, R^{5b} is HOCH₂CH₂NH—. In some embodiments, R^{5b} is R^{5x}OCONH—. In some embodiments, R^{5b} is R^{5z}NHCH(CH₃)NH—. In some embodiments, R^{5b} is N⁺(O⁻)H₂. In some embodiments, R^{5b} is N(O). In some embodiments, R^{5b} is N(═CH₂). In some embodiments, R^{5b} is R^{5e}OC(O)NH—In some embodiments, R^{5b} is C₁₋₈alkylC(O)NH—.

In some embodiments, R^{5c} is H. In some embodiments, R^{5c} is hydroxyl. In some embodiments, R^{5c} is fluoro. In some embodiments, R^{5c} is combined to form an oxo group. In some embodiments, one R^{5c} is combined with R^{5b} to form a pyridyl ring and the other R^{5c} is null.

In some embodiments, R^{5d} is H, fluoro. In some embodiments, R^{5d} is hydroxyl. In some embodiments, R^{5d} is alkoxy. In some embodiments, R^{5d} is benzyloxy.

In some embodiments, R^{5e} is of H. In some embodiments, R^{5e} is alkyl. In some embodiments, R^{5e} is heterocyclyl. In some embodiments, R^{5e} is substituted with one to four substitutents independently selected from the group consisting of oxo, hydroxy, and carboxy.

In some embodiments, R^{5f} is selected from the group consisting of hydrogen, hydroxyl and acetoxy.

In some embodiments, R^{5j} is independently selected from oxo, hydroxyl and acetoxy.

In some embodiments, R^{5k} is independently selected from oxo, hydroxyl and acetoxy.

In some embodiments, R^{5l} is independently selected from H, alkoxycarbonyl, alkylaminocarbonyl, dialkylaminocarbonyl, and heterocyclylcarbonyl.

In some embodiments, R^{5m} is independently selected from H and aminocarbonyl.

In some embodiments, R^{5X} is a sugar moiety.

In some embodiments, R^{5z} is a moiety of formula V attached via a covalent bond at R^{5b} wherein Y is

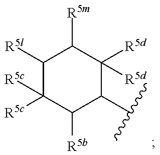

x is 0, 1, or 2;
y is 0, 1, or 2; and
- - - represents a single or double bond;
provided that when R^{5b} is amino and x and y are 0, then R^{5c} and R^{5d} are not both hydrogen.

In some embodiments, wherein at least one of x and y is 1.

In some embodiments, R^{5j} is an oxide attached to one of the triazole nitrogens.

In some embodiments, wherein R^{5k} is an oxide attached to one of the pyrimidine nitrogen.

In some embodiments, Y is

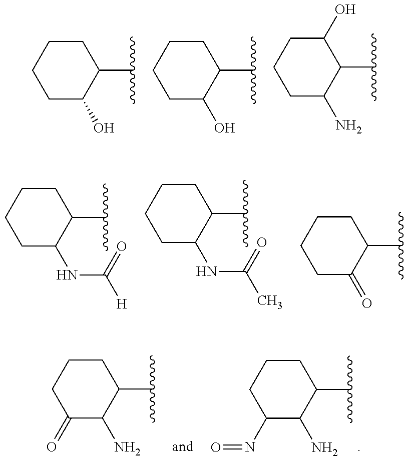

In some embodiments, Y is selected from the group consisting of:

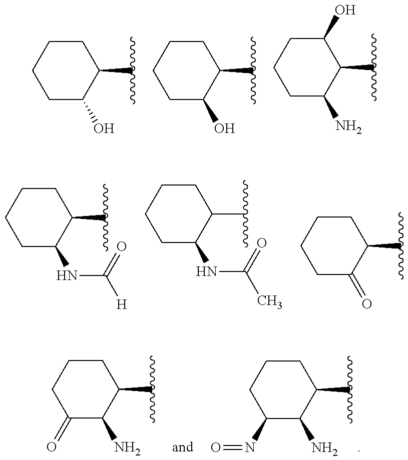

In some embodiments, Y is selected from the group consisting of:

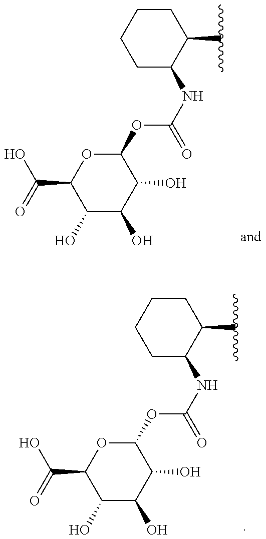 [SMILES: *[C@@H]1CCCC[C@@H]1NC(=O)O[C@@H]1O[C@H](C(=O)O)[C@@H](O)[C@H](O)[C@H]1O ; *[C@@H]1CCCC[C@@H]1NC(=O)O[C@H]1O[C@H](C(=O)O)[C@@H](O)[C@H](O)[C@H]1O]

In some embodiments, R^{5b} is NH₂.

In some embodiments, one of R^{5c} or R^{5d} is hydroxy.

The present invention provides in other embodiments, a compound of the examples.

The present invention provides in other embodiments, a compound of Table 1.

In some embodiments, compounds provided herein are isolated and purified or are synthetic compounds.

It is understood that in another group of embodiments, any of the above embodiments may also be combined with other embodiments listed herein, to form other embodiments of the invention. Similarly, it is understood that in other embodiments, listing of groups includes embodiments wherein one or more of the elements of those groups is not included.

b. Methods of Synthesis

The compounds of the present invention may be prepared by known organic synthesis techniques, including the methods described in more detail in the Examples.

One skilled in the art will recognize that in certain embodiments it may be advantageous to use a protecting group strategy. The protecting group can be removed using methods known to those skilled in the art.

The compounds of the present invention may generally be utilized as the free base. Alternatively, the compounds of this invention may be used in the form of acid addition salts as described below.

c. Inhibition of Syk Kinases

The activity of a specified compound as an inhibitor of a Syk kinase may be assessed in vitro or in vivo. In some embodiments, the activity of a specified compound can be tested in a cellular assay. Selectivity could also be ascertained in biochemical assays with isolated kinases. Exemplary assays of this type are described in greater detail in the Examples.

d. Compositions and Methods of Administration

The present invention further provides compositions comprising one or more compounds provided herein or a pharmaceutically acceptable salt, ester or prodrug thereof, and a pharmaceutically acceptable carrier or diluent. It will be appreciated that the compounds provided herein in this invention may be derivatized at functional groups to provide prodrug derivatives which are capable of conversion back to the parent compounds in vivo. Examples of such prodrugs include the physiologically acceptable and metabolically labile ester derivatives, such as methoxymethyl esters, methylthiomethyl esters, or pivaloyloxymethyl esters derived from a hydroxyl group of the compound or a carbamoyl moiety derived from an amino group of the compound. Additionally, any physiologically acceptable equivalents of the compounds provided herein, similar to metabolically labile esters or carbamates, which are capable of producing the parent compounds provided herein in vivo, are within the scope of this invention.

As used herein, the term “pharmaceutically acceptable salts” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this invention are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Furthermore, the basic nitrogen-containing groups may be quaternized with agents like lower alkyl halides, such as methyl, ethyl, propyl and butyl chlorides, bromides and iodides; dialkyl sulfates, such as dimethyl, diethyl, dibutyl and diamyl sulfates, long chain halides, such as decyl, lauryl, myristyl and stearyl chlorides, bromides and iodides; aralkyl halides, such as benzyl and phenethyl bromides and others. Water or oil-soluble or dispersible products are thereby obtained.

The compounds utilized in the compositions and methods may also be modified by appending appropriate functionalities to enhance selective biological properties. Such modifications are known in the art and include those which increase biological penetration into a given biological system (e.g., blood, lymphatic system, central nervous system, etc.), increase oral availability, increase solubility to allow administration by injection, alter metabolism and alter rate of excretion.

The pharmaceutical compositions can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain stabilizers, pH modifiers, surfactants, bioavailability modifiers and combinations of these.

The term “unit dosage form” refers to physically discrete units suitable as unitary dosages for human subjects and other mammals, each unit containing a predetermined quantity of drug calculated to produce the desired onset, tolerability, and/or therapeutic effects, in association with a suitable pharmaceutical excipient (e.g., an ampoule). In addition, more concentrated compositions may be prepared, from which the more dilute unit dosage compositions may then be produced. The more concentrated compositions thus will contain substantially more than, e.g., at least 1, 2, 3, 4, 5, 6, 7, 8, 9, 10, or more times the amount of one or more Syk inhibitors.

Methods for preparing such dosage forms are known to those skilled in the art (see, for example, REMINGTON'S PHARMACEUTICAL SCIENCES, 18TH ED., Mack Publishing Co., Easton, Pa. (1990)). In addition, pharmaceutically acceptable salts of the Syk inhibitors of the present invention (e.g., acid addition salts) may be prepared and included in the compositions using standard procedures known to those skilled in the art of synthetic organic chemistry and described, e.g., by J. March, Advanced Organic Chemistry: Reactions, Mechanisms and Structure, 4^{th} Ed. (New York: Wiley-Interscience, 1992).

The compositions typically include a conventional pharmaceutical carrier or excipient and may additionally include other medicinal agents, carriers, adjuvants, diluents, tissue permeation enhancers, solubilizers, and the like. Preferably, the composition will contain about 0.01% to about 90%, preferably about 0.1% to about 75%, more preferably about 0.1% to 50%, still more preferably about 0.1% to 10% by weight of one or more Syk inhibitors, with the remainder consisting of suitable pharmaceutical carrier and/or excipients. Appropriate excipients can be tailored to the particular composition and route of administration by methods well known in the art, e.g., REMINGTON'S PHARMACEUTICAL SCIENCES, supra.

Pharmaceutically acceptable carriers that may be used in these compositions include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols. The compositions can additionally include lubricating agents such as talc, magnesium stearate, and mineral oil; wetting agents; emulsifying agents; suspending agents; preserving agents such as methyl-, ethyl-, and propyl-hydroxy-benzoates; pH adjusting agents such as inorganic and organic acids and bases; sweetening agents; and flavoring agents.

Administration of a composition comprising one or more Syk inhibitors with one or more suitable pharmaceutical excipients as advantageous can be carried out via any of the accepted modes of administration. Thus, administration can be, for example, oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously. The formulations of the invention may be designed as short-acting, fast-releasing, or long-acting. Still further, compounds can be administered in a local rather than systemic means, such as administration (e.g., injection) as a sustained release formulation. According to a representative embodiment, the compositions of this invention are formulated for pharmaceutical administration to a mammal, preferably a human being.

The compositions of the present invention containing one or more Syk inhibitors can be administered repeatedly, e.g., at least 2, 3, 4, 5, 6, 7, 8, or more times, or the composition may be administered by continuous infusion. Suitable sites of administration include, but are not limited to, skin, bronchial, gastrointestinal, anal, vaginal, eye, and ear. The formulations may take the form of solid, semi-solid, lyophilized powder, or liquid dosage forms, such as, for example, tablets, pills, capsules, powders, solutions, suspensions, emulsions, suppositories, retention enemas, creams, ointments, lotions, gels, aerosols, or the like, preferably in unit dosage forms suitable for simple administration of precise dosages.

The pharmaceutical compositions of this invention may be in any orally acceptable dosage form, including tablets, capsules, cachets, emulsions, suspensions, solutions, syrups, elixirs, sprays, boluses, lozenges, powders, granules, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like. In the case of tablets for oral use, carriers that are commonly used include lactose and corn starch. Lubricating agents, such as magnesium stearate, are also typically added. For a capsule form, useful diluents include lactose and dried cornstarch. When aqueous suspensions are required for oral use, the active ingredient is combined with emulsifying and suspending agents. If desired, certain sweetening, flavoring or coloring agents may also be added.

In some embodiments, the compositions take the form of a pill, tablet, or capsule, and thus, the composition can contain, along with one or more Syk inhibitors, a diluent such as lactose, sucrose, dicalcium phosphate, and the like; a disintegrant such as starch or derivatives thereof; a lubricant such as magnesium stearate and the like; and/or a binder such a starch, gum acacia, polyvinylpyrrolidone, gelatin, cellulose and derivatives thereof. A tablet can be made by any compression or molding process known to those of skill in the art. Compressed tablets may be prepared by compressing in a suitable machine the Syk inhibitors in a free-flowing form, e.g., a powder or granules, optionally mixed with accessory ingredients, e.g., binders, lubricants, diluents, disintegrants, or dispersing agents. Molded tablets can be made by molding in a suitable machine a mixture of the powdered Syk inhibitors with any suitable carrier.

Alternatively, the pharmaceutical compositions of this invention may be in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax, polyethylene glycol (PEG), hard fat, and/or hydrogenated cocoglyceride. Compositions suitable for rectal administration may also comprise a rectal enema unit containing one or more Syk inhibitors and pharmaceutically-acceptable vehicles (e.g., 50% aqueous ethanol or an aqueous salt solution) that are physiologically compatible with the rectum and/or colon. The rectal enema unit contains an applicator tip protected by an inert cover, preferably comprised of polyethylene, lubricated with a lubricant such as white petrolatum, and preferably protected by a one-way valve to prevent back-flow of the dispensed formula. The rectal enema unit is also of sufficient length, preferably two inches, to be inserted into the colon via the anus.

Liquid compositions can be prepared by dissolving or dispersing one or more Syk inhibitors and optionally one or more pharmaceutically acceptable adjuvants in a carrier such as, for example, aqueous saline, aqueous dextrose, glycerol, ethanol, and the like, to form a solution or suspension, e.g., for oral, topical, or intravenous administration. Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

The pharmaceutical compositions of this invention may also be in a topical form, especially when the target of treatment includes areas or organs readily accessible by topical application, including diseases of the eye, the skin, or the lower intestinal tract. Suitable topical formulations are readily prepared for each of these areas or organs. For topical administration, the composition containing one or more Syk inhibitors can be in the form of emulsions, lotions, gels, foams, creams, jellies, solutions, suspensions, ointments, and transdermal patches.

Topical application for the lower intestinal tract may be effected in a rectal suppository foimulation or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include mineral oil, sorbitan monostearate, polysorbate 60, cetyl esters, wax, cetyl alcohol, 2-octyldodecanol, benzyl alcohol and water.

The pharmaceutical compositions may also be administered by nasal aerosol or inhalation. For delivery by inhalation, the compositions can be delivered as a dry powder or in liquid form via a nebulizer. Such compositions are prepared according to techniques known in the art of pharmaceutical formulation and may be prepared as solutions in saline, employing benzyl alcohol or other suitable preservatives, absorption promoters to enhance bioavailability, fluorocarbons and/or other conventional solubilizing or dispersing agents.

For ophthalmic use, the pharmaceutical compositions may be formulated as micronized suspensions in isotonic, pH adjusted sterile saline, or, preferably, as solutions in isotonic, pH adjusted sterile saline, either with our without a preservative, such as benzylalkonium chloride. Alternatively, for ophthalmic uses, the pharmaceutical compositions may be formulated in an ointment, such as petrolatum.

For parenteral administration, the compositions can be in the form of sterile injectable solutions and sterile packaged powders. Preferably, injectable solutions are formulated at a pH of about 4.5 to about 7.5.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

The compositions of the present invention can also be provided in a lyophilized form. Such compositions may include a buffer, e.g., bicarbonate, for reconstitution prior to administration, or the buffer may be included in the lyophilized composition for reconstitution with, e.g., water. The lyophilized composition may further comprise a suitable vasoconstrictor, e.g., epinephrine. The lyophilized composition can be provided in a syringe, optionally packaged in combination with the buffer for reconstitution, such that the reconstituted composition can be immediately administered to a patient.

Any of the above dosage forms containing effective amounts are within the bounds of routine experimentation and within the scope of the invention. A therapeutically effective dose may vary depending upon the route of administration and dosage form. The representative compound or compounds of the invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

Besides those representative dosage forms described above, pharmaceutically acceptable excipients and carriers and dosage foul's are generally known to those skilled in the art and are included in the invention. It should be understood that a specific dosage and treatment regimen for any particular patient will depend upon a variety of factors, including the activity of the specific compound employed, the age, body weight, general health, sex and diet of the patient, and the time of administration, rate of excretion, drug combination, judgment of the treating physician and severity of the particular disease being treated. The amount of active ingredient(s) will also depend upon the particular compound and other therapeutic agent, if present, in the composition.

e. Methods of Use

The invention provides methods of inhibiting or decreasing Syk activity as well as treating or ameliorating a Syk associated state, symptom, condition, disorder or disease in a patient in need thereof (e.g., human or non-human). In one embodiment, the Syk associated state, symptom, condition, disorder or disease is mediated, at least in part by Syk kinase activity. In more specific embodiments, the present invention provides a method for treating a condition or disorder mediated at least in part by Syk kinase activity is cardiovascular disease, inflammatory disease or autoimmune disease.

In one embodiment, the invention provides methods for preventing or treating a condition in a mammal mediated at least in part by syk activity comprising the step of administering to the mammal a therapeutically effective amount of a compound of the present invention. Such conditions include, but are not limited, to restenosis, acute coronary syndrome, myocardial infarction, unstable angina, refractory angina, occlusive coronary thrombosis occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolism, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, conditions requiring the fitting of prosthetic devices, and the like.

In a further embodiment, the present invention provides a method for treating thrombosis, immune thrombocytic purura, heparin induced thrombocytopenia, dilated cardiomypathy, sickle cell disease, atherosclerosis, myocardial infarction, vacular inflammation, unstable angina or acute coronary syndromes.

In another embodiment, the present invention also provides a method for treating allergy, asthma, theumatoid arthritis, B Cell mediated disease such as Non-Hodgkin's Lymphoma, anti phospholipids syndrome, lupus, psoriasis, multiple sclerosis, end stage renal disease or chronic lymphocytic leukemia.

In another embodiment, the present invention provides a method for treating hemolytic anemia or immune thrombocytopenic purpura.

In another embodiment, the present invention provides a method for treating vasculitis, including but not limited to: Large vessel vasculitis, such as Giant cell arteritis and Takayasu's arteritis; Medium vessel vasculitis, such as Polyarteritis nodosa (PAN) and Kawasaki Disease; Small vessel vasculitis, such as Wegener's granulomatosis, Churg-Strauss syndrome, Microscopic polyangiitis, Henoch-Schonlein purpura, Cryoglobulinaemic vasculitis, and Cutaneous leucocytoclastic angiitis.

In another embodiment, the present invention provides a method for treating a Auto-immune blistering skin disease including but not limited to: Pemphigus, such as Pemphigus vulgaris, Pemphigus foliaceus, Paraneoplastic pemphigus, and IgA pemphigus; and Subepidermal autoimmune blistering skin disease, such as Bullous pemphigoid, Pemphigoid gestationis, Linear IgA dermatosis, Mucous membrane pemphigoid, Lichen planus pemphigoides, g1/p200 pemphigoid, Epidermolysis bullosa acquisita and Dermatitis herpetiformis.

Therapy using the compounds described herein can be applied alone, or it can be applied in combination with or adjunctive to other common immunosuppressive therapies, such as, for example, the following: mercaptopurine; corticosteroids such as prednisone; methylprednisolone and prednisolone; alkylating agents such as cyclophosphamide; calcineurin inhibitors such as cyclosporine, sirolimus, and tacrolimus; inhibitors of inosine monophosphate dehydrogenase (IMPDH) such as mycophenolate, mycophenolate mofetil, and azathioprine; and agents designed to suppress cellular immunity while leaving the recipient's humoral immunologic response intact, including various antibodies (for example, antilymphocyte globulin (ALG), antithymocyte globulin (ATG), monoclonal anti-T-cell antibodies (OKT3)) and irradiation. These various agents can be used in accordance with their standard or common dosages, as specified in the prescribing information accompanying commercially available forms of the drugs (see also: the prescribing information in the 2006 Edition of The Physician's Desk Reference), the disclosures of which are incorporated herein by reference. Azathioprine is currently available from Salix Pharmaceuticals, Inc., under the brand name AZASAN; mercaptopurine is currently available from Gate Pharmaceuticals, Inc., under the brand name PURINETHOL; prednisone and prednisolone are currently available from Roxane Laboratories, Inc.; Methyl prednisolone is currently available from Pfizer; sirolimus (rapamycin) is currently available from Wyeth-Ayerst under the brand name RAPAMUNE; tacrolimus is currently available from Fujisawa under the brand name PROGRAF; cyclosporine is current available from Novartis under the brand dame SANDIMMUNE and from Abbott under the brand name GENGRAF; IMPDH inhibitors such as mycophenolate mofetil and mycophenolic acid are currently available from Roche under the brand name CELLCEPT and from Novartis under the brand name MYFORTIC; azathioprine is currently available from Glaxo Smith Kline under the brand name IMURAN; and antibodies are currently available from Ortho Biotech under the brand name ORTHOCLONE, from Novartis under the brand name SIMULECT (basiliximab), and from Roche under the brand name ZENAPAX (daclizumab).

In another embodiment, the compounds could be administered either in combination or adjunctively with an inhibitor of a Syk kinase. Syk kinase is a tyrosine kinase known to play a critical role in Fcγ receptor signaling, as well as in other signaling cascades, such as those involving B-cell receptor signaling (Turner et al., (2000), Immunology Today 21:148-154) and integrins beta (1), beta (2), and beta (3) in neutrophils (Mocsai et al., (2002), Immunity 16:547-558). For example, Syk kinase plays a pivotal role in high affinity IgE receptor signaling in mast cells that leads to activation and subsequent release of multiple chemical mediators that trigger allergic attacks. However, unlike the JAK kinases, which help regulate the pathways involved in delayed or cell-mediated Type IV hypersensitivity reactions, Syk kinase helps regulate the pathways involved in immediate IgE-mediated, Type I hypersensitivity reactions. Certain compounds that affect the Syk pathway may or may not also affect the JAK pathways.

Suitable Syk inhibitory compounds are described, for example, in Ser. No. 10/355,543 filed Jan. 31, 2003 (publication no. 2004/0029902); WO 03/063794; Ser. No. 10/631,029 filed Jul. 29, 2003; WO 2004/014382; Ser. No. 10/903,263 filed Jul. 30, 2004; PCT/US2004/24716 filed Jul. 30, 2004 (WO005/016893); Ser. No. 10/903,870 filed Jul. 30, 2004; PCT/US2004/24920 filed Jul. 30, 2004, the disclosures of which are incorporated herein by reference. The described herein and Syk inhibitory compounds could be used alone or in combination with one or more conventional transplant rejection treatments, as described above.

In a specific embodiment, the compounds can be used to treat or prevent these diseases in patients that are either initially non-responsive (resistant) to or that become non-responsive to treatment with a Syk inhibitory compound or one of the other current treatments for the particular disease. The compounds could also be used in combination with Syk inhibitory compounds in patients that are Syk-compound resistant or non-responsive. Suitable Syk-inhibitory compounds with which the compounds can be administered are provided infra

In another embodiment, this invention provides a method of treating a T-cell mediated autoimmune disease, comprising administering to a patient suffering from such an autoimmune disease an amount of a compound effective to treat the autoimmune disease wherein the compound is selected from the compounds of the invention, as described herein, and the compound is administered in combination with or adjunctively to a compound that inhibits Syk kinase with an IC₅₀ in the range of at least 10 μM.

When used to treat or prevent such diseases, the compounds can be administered singly, as mixtures of one or more compounds, or in mixture or combination with other agents useful for treating such diseases and/or the symptoms associated with such diseases. The compounds may also be administered in mixture or in combination with agents useful to treat other disorders or maladies, such as steroids, membrane stabilizers, 5-lipoxygenase (5LO) inhibitors, leukotriene synthesis and receptor inhibitors, inhibitors of IgE isotype switching or IgE synthesis, IgG isotype switching or IgG synthesis, beta.-agonists, tryptase inhibitors, aspirin, cyclooxygenase (COX) inhibitors, methotrexate, anti-TNF drugs, anti CD20 antibody, PD4 inhibitors, p38 inhibitors, PDE4 inhibitors, and antihistamines, to name a few. The compounds can be administered per se in the form of prodrugs or as pharmaceutical compositions, comprising an active compound or prodrug.

Active compounds of the invention typically inhibit the Syk and/or JAK/Stat pathway. The activity of a specified compound as an inhibitor of a Syk kinase can be assessed in vitro or in vivo. In some embodiments, the activity of a specified compound can be tested in a cellular assay.

“Cell proliferative disorder” refers to a disorder characterized by abnormal proliferation of cells. A proliferative disorder does not imply any limitation with respect to the rate of cell growth, but merely indicates loss of normal controls that affect growth and cell division. Thus, in some embodiments, cells of a proliferative disorder can have the same cell division rates as normal cells but do not respond to signals that limit such growth. Within the ambit of “cell proliferative disorder” is neoplasm or tumor, which is an abnormal growth of tissue. Cancer refers to any of various malignant neoplasms characterized by the proliferation of cells that have the capability to invade surrounding tissue and/or metastasize to new colonization sites.

Generally, cell proliferative disorders treatable with the compounds disclosed herein relate to any disorder characterized by aberrant cell proliferation. These include various tumors and cancers, benign or malignant, metastatic or non-metastatic. Specific properties of cancers, such as tissue invasiveness or metastasis, can be targeted using the methods described herein. Cell proliferative disorders include a variety of cancers, including, among others, ovarian cancer, renal cancer, gastrointestinal cancer, kidney cancer, bladder cancer, pancreatic cancer, lung squamous carcinoma, and adenocarcinoma.

In some embodiments, the cell proliferative disorder treated is a hematopoietic neoplasm, which is aberrant growth of cells of the hematopoietic system. Hematopoietic malignancies can have its origins in pluripotent stem cells, multipotent progenitor cells, oligopotent committed progenitor cells, precursor cells, and terminally differentiated cells involved in hematopoiesis. Some hematological malignancies are believed to arise from hematopoietic stem cells, which have the ability for self renewal. For instance, cells capable of developing specific subtypes of acute myeloid leukemia (AML) (Cynthia K. Hahn, Kenneth N. Ross, Rose M. Kakoza, Steven Karr, Jinyan Du, Shao-E Ong, Todd R. Golub, Kimberly Stegmaier, Syk is a new target for AML differentiation, Blood, 2007, 110, Abstract 209) upon transplantation display the cell surface markers of hematopoietic stem cells, implicating hematopoietic stem cells as the source of leukemic cells. Blast cells that do not have a cell marker characteristic of hematopoietic stem cells appear to be incapable of establishing tumors upon transplantation (Blaire et al., 1997, Blood 89:3104-3112). The stem cell origin of certain hematological malignancies also finds support in the observation that specific chromosomal abnormalities associated with particular types of leukemia can be found in normal cells of hematopoietic lineage as well as leukemic blast cells. For instance, the reciprocal translocation t(9q34;22q11) associated with approximately 95% of chronic myelogenous leukemia appears to be present in cells of the myeloid, erythroid, and lymphoid lineage, suggesting that the chromosomal aberration originates in hematopoietic stem cells. A subgroup of cells in certain types of CML displays the cell marker phenotype of hematopoietic stem cells.

Although hematopoietic neoplasms often originate from stem cells, committed progenitor cells or more terminally differentiated cells of a developmental lineage can also be the source of some leukemias. For example, forced expression of the fusion protein Bcr/Abl (associated with chronic myelogenous leukemia) in common myeloid progenitor or granulocyte/macrophage progenitor cells produces a leukemic-like condition. Moreover, some chromosomal aberrations associated with subtypes of leukemia are not found in the cell population with a marker phenotype of hematopoietic stem cells, but are found in a cell population displaying markers of a more differentiated state of the hematopoietic pathway (Turhan et al., 1995, Blood 85:2154-2161). Thus, while committed progenitor cells and other differentiated cells may have only a limited potential for cell division, leukemic cells may have acquired the ability to grow unregulated, in some instances mimicking the self-renewal characteristics of hematopoietic stem cells (Passegue et al., Proc. Natl. Acad. Sci. USA, 2003, 100:11842-9).

In some embodiments, the hematopoietic neoplasm treated is a lymphoid neoplasm, where the abnormal cells are derived from and/or display the characteristic phenotype of cells of the lymphoid lineage. Lymphoid neoplasms can be subdivided into B-cell neoplasms, T and NK-cell neoplasms, and Hodgkin's lymphoma. B-cell neoplasms can be further subdivided into precursor B-cell neoplasm and mature/peripheral B-cell neoplasm. Exemplary B-cell neoplasms are precursor B-lymphoblastic leukemia/lymphoma (precursor B-cell acute lymphoblastic leukemia) while exemplary mature/peripheral B-cell neoplasms are B-cell chronic lymphocytic leukemia/small lymphocytic lymphoma, B-cell prolymphocytic leukemia, lymphoplasmacytic lymphoma, splenic marginal zone B-cell lymphoma, hairy cell leukemia, plasma cell myeloma/plasmacytoma, extranodal marginal zone B-cell lymphoma of MALT type, nodal marginal zone B-cell lymphoma, follicular lymphoma, mantle-cell lymphoma, diffuse large B-cell lymphoma, mediastinal large B-cell lymphoma, primary effusion lymphoma, and Burkitt's lymphoma/Burkitt cell leukemia. T-cell and Nk-cell neoplasms are further subdivided into precursor T-cell neoplasm and mature (peripheral) T-cell neoplasms. Exemplary precursor T-cell neoplasm is precursor T-lymphoblastic lymphoma/leukemia (precursor T-cell acute lymphoblastic leukemia) while exemplary mature (peripheral) T-cell neoplasms are T-cell prolymphocytic leukemia T-cell granular lymphocytic leukemia, aggressive NK-cell leukemia, adult T-cell lymphoma/leukemia (HTLV-1), extranodal NK/T-cell lymphoma, nasal type, enteropathy-type T-cell lymphoma, hepatosplenic gamma-delta T-cell lymphoma, subcutaneous panniculitis-like T-cell lymphoma, Mycosis fungoides/Sezary syndrome, Anaplastic large-cell lymphoma, T/null cell, primary cutaneous type, Peripheral T-cell lymphoma, not otherwise characterized, Angioimmunoblastic T-cell lymphoma, Anaplastic large-cell lymphoma, T/null cell, primary systemic type. The third member of lymphoid neoplasms is Hodgkin's lymphoma, also referred to as Hodgkin's disease. Exemplary diagnosis of this class that can be treated with the compounds include, among others, nodular lymphocyte-predominant Hodgkin's lymphoma, and various classical forms of Hodgkin's disease, exemplary members of which are Nodular sclerosis Hodgkin's lymphoma (grades 1 and 2), Lymphocyte-rich classical Hodgkin's lymphoma, Mixed cellularity Hodgkin's lymphoma, and Lymphocyte depletion Hodgkin's lymphoma. In various embodiments, any of the lymphoid neoplasms that are associated with aberrant Syk activity can be treated with the Syk inhibitory compounds.

In some embodiments, the hematopoietic neoplasm treated is a myeloid neoplasm. This group comprises a large class of cell proliferative disorders involving or displaying the characteristic phenotype of the cells of the myeloid lineage. Myeloid neoplasms can be subdivided into myeloproliferative diseases, myelodysplastic/myeloproliferative diseases, myelodysplastic syndromes, and acute myeloid leukemias. Exemplary myeloproliferative diseases are chronic myelogenous leukemia (e.g., Philadelphia chromosome positive (t(9;22)(qq34;q11)), chronic neutrophilic leukemia, chronic eosinophilic leukemia/hypereosinophilic syndrome, chronic idiopathic myelofibrosis, polycythemia vera, and essential thrombocythemia. Exemplary myelodysplastic/myeloproliferative diseases are chronic myelomonocytic leukemia, atypical chronic myelogenous leukemia, and juvenile myelomonocytic leukemia. Exemplary myelodysplastic syndromes are refractory anemia, with ringed sideroblasts and without ringed sideroblasts, refractory cytopenia (myelodysplastic syndrome) with multilineage dysplasia, refractory anemia (myelodysplastic syndrome) with excess blasts, 5q-syndrome, and myelodysplastic syndrome. In various embodiments, any of the myeloid neoplasms that are associated with aberrant Syk activity can be treated with the Syk inhibitory compounds.

In some embodiments, the compounds can be used to treat Acute myeloid leukemias (AML), which represent a large class of myeloid neoplasms having its own subdivision of disorders. These subdivisions include, among others, AMLs with recurrent cytogenetic translocations, AML with multilineage dysplasia, and other AML not otherwise categorized. Exemplary AMLs with recurrent cytogenetic translocations include, among others, AML with t(8;21)(q22;q22), AML1(CBF-alpha)/ETO, Acute promyelocytic leukemia (AML with t(15;17)(q22;q11-12) and variants, PML/RAR-alpha), AML with abnormal bone marrow eosinophils (inv(16)(p13q22) or t(16;16)(p13;q11), CBFb/MYH11X), and AML with 11q23 (MLL) abnormalities. Exemplary AML with multilineage dysplasia are those that are associated with or without prior myelodysplastic syndrome. Other acute myeloid leukemias not classified within any definable group include, AML minimally differentiated, AML without maturation, AML with maturation, Acute myelomonocytic leukemia, Acute monocytic leukemia, Acute erythroid leukemia, Acute megakaryocytic leukemia, Acute basophilic leukemia, and Acute panmyelosis with myelofibrosis.

The inventive methods comprise administering an effective amount of a compound or composition described herein to a mammal or non-human animal. As used herein, “effective amount” of a compound or composition of the invention includes those amounts that antagonize or inhibit Syk. An amount which antagonizes or inhibits Syk is detectable, for example, by any assay capable of determining Syk activity, including the one described below as an illustrative testing method. Effective amounts may also include those amounts which alleviate symptoms of a Syk associated disorder treatable by inhibiting Syk. Accordingly, “antagonists of Syk” or include compounds which interact with the Syk and modulate, e.g., inhibit or decrease, the ability of a second compound, e.g., another Syk ligand, to interact with the Syk . The Syk binding compounds are preferably antagonists. The language “Syk binding compound” and (e.g., exhibits binding affinity to the receptor) includes those compounds which interact with Syk resulting in modulation of the activity of Syk or JAK, respectively. Syk binding compounds may be identified using an in vitro (e.g., cell and non-cell based) or in vivo method. A description of in vitro methods are provided below.

The amount of compound present in the methods and compositions described herein should be sufficient to cause a detectable decrease in the severity of the disorder, as measured by any of the assays described in the examples. The amount of Syk modulator needed will depend on the effectiveness of the modulator for the given cell type and the length of time required to treat the disorder. In certain embodiments, the compositions of this invention may further comprise another therapeutic agent. When a second agent is used, the second agent may be administered either as a separate dosage form or as part of a single dosage form with the compounds or compositions of this invention. While one or more of the inventive compounds can be used in an application of monotherapy to treat a disorder, disease or symptom, they also may be used in combination therapy, in which the use of an inventive compound or composition (therapeutic agent) is combined with the use of one or more other therapeutic agents for treating the same and/or other types of disorders, symptoms and diseases. Combination therapy includes administration of the two or more therapeutic agents concurrently or sequentially. The agents may be administered in any order. Alternatively, the multiple therapeutic agents can be combined into a single composition that can be administered to the patient. For instance, a single pharmaceutical composition could comprise the compound or pharmaceutically acceptable salt, ester or prodrug thereof according to the formula I, another therapeutic agent (e.g., methotrexate) or a pharmaceutically acceptable salt, ester or prodrug thereof, and a pharmaceutically acceptable excipient or carrier.

In one embodiment, provided is a method of using one or more of the compounds provided herein to treat a variety of disorders, symptoms and diseases (e.g., inflammatory, autoimmune, neurological, neurodegenerative, oncology and cardiovascular). In certain groups of embodiments the inflammatory disease and autoimmune disease is selected from the group consisting of organ transplants, osteoarthritis, irritable bowel disease (IBD), asthma, chronic obstructive pulmonary disease (COPD), systemic lupus erythematosus, multiple sclerosis, rheumatoid arthritis (RA), Crohn's disease, Type I diabetes, conjunctivitis, uveitis, vasculitis and psoriasis. In certain groups of embodiments the inflammatory disease is selected from the group consisting of allergy, asthma, rheumatoid arthritis, B Cell mediated diseases such as Non Hodgkin's Lymphoma, anti phospholipid syndrome, lupus, psoriasis, multiple sclerosis and end stage renal disease. In certain groups of embodiments the cardiovascular disease is selected from the group consisting of immune thrombocytopenic purpura, hemolytic anemia and heparin induced thrombocytopenia. In certain groups of embodiments the inflammatory disease is rheumatoid arthritis. In certain groups of embodiments the sickle cell disease is selected from the group consisting of sickle cell anemia, sickle-hemoglobin C disease, sickle beta-plus thalassemia, and sickle beta-zero thalassemia. In certain groups of embodiments the autoimmune disease is selected from the group consisting of organ transplants, chronic obstructive pulmonary disease (COPD), hemolytic anemia, immune thrombocytopenic purpura (ITP), multiple sclerosis, Sjogren's syndrome Type I diabetes, rheumatoid arthritis, lupus (including systemic lupus erythematosus (SLE), vasculitis, glomerular nephritis (GN), auto-immune-blistering disease, atopic dermatitis(eczema), atherosclerosis, autoimmune neutropenia and psoriasis. In certain groups of embodiments the cell proliferative disorder is leukemia, a lymphoma, myeloproliferative disorders, hematological malignancies, and chronic idiopathic myelofibrosis. In certain groups of embodiments the disorder is acute myeloid leukemia (AML), chronic lymphocytic leukemia (CLL), acute lymphoblastic leukemia (ALL) or non-Hodgkin's lymphoma.

The inventive compounds and their pharmaceutically acceptable salts and/or neutral compositions may be formulated together with a pharmaceutically acceptable excipient or carrier and the resulting composition may be administered in vivo to mammals, such as men, women and animals, to treat a variety of disorders, symptoms and diseases. Furthermore, the inventive compounds can be used to prepare a medicament that is useful for treating a variety of disorders, symptoms and diseases.

All of the compounds of the present invention are potent inhibitors of Syk kinases, exhibiting IC₅₀s in the respective assay in the range of less than 5 μM, with most being in the nanomolar, and several in the sub-nanomolar, range.

f. Kits

Still another aspect of this invention is to provide a kit comprising separate containers in a single package, wherein the inventive pharmaceutical compounds, compositions and/or salts thereof are used in combination with pharmaceutically acceptable carriers to treat states, disorders, symptoms and diseases where Syk plays a role.

### EXAMPLES

The following examples are offered to illustrate, but not to limit, the claimed invention.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1967-2004, Volumes 1-22; Rodd's Chemistry of Carbon Compounds, Elsevier Science Publishers, 1989, Volumes 1-5 and Supplementals; and Organic Reactions, Wiley & Sons: New York, 2005, Volumes 1-65.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C. to about 75° C.

Referring to the examples that follow, compounds of the present invention were synthesized using the methods described herein, or other methods, which are well known in the art.

The compounds and/or intermediates may be characterized by high performance liquid chromatography (HPLC) using a Waters Alliance chromatography system with a 2695 Separation Module (Milford, Mass.). The analytical columns may be C-18 SpeedROD RP-18E Columns from Merck KGaA (Darmstadt, Germany). Alternately, characterization may be performed using a Waters Unity (UPLC) system with Waters Acquity UPLC BEH C-18 2.1 mm×15 mm columns. A gradient elution may be used, typically starting with 5% acetonitrile/95% water and progressing to 95% acetonitrile over a period of 5 minutes for the Alliance system and 1 minute for the Acquity system. All solvents may contain 0.1% trifluoroacetic acid (TFA). Compounds may be detected by ultraviolet light (UV) absorption at either 220 nm or 254 nm. HPLC solvents may be from EMD Chemicals, Inc. (Gibbstown, N.J.). In some instances, purity may be assessed by thin layer chromatography (TLC) using glass backed silica gel plates, such as, for example, EMD Silica Gel 60 2.5 cm×7.5 cm plates. TLC results may be readily detected visually under ultraviolet light, or by employing well known iodine vapor and other various staining techniques.

Mass spectrometric analysis may be performed on one of two Agilent 1100 series LCMS instruments with acetonitrile/water as the mobile phase. One system may use TFA as the modifier and measure in positive ion mode [reported as MH+, (M+1) or (M+H)+] and the other may use either formic acid or ammonium acetate and measure in both positive [reported as MH⁺, (M+1) or (M+H)⁺] and negative [reported as M−, (M−1) or (M−H)⁻] ion modes.

Nuclear magnetic resonance (NMR) analysis may be performed on some of the compounds with a Varian 400 MHz NMR (Palo Alto, Calif.). The spectral reference may be either TMS or the known chemical shift of the solvent.

The purity of some of the invention compounds may be assessed by elemental analysis (Robertson Microlit, Madison, N.J.).

Melting points may be determined on a Laboratory Devices MeI-Temp apparatus (Holliston, Mass.).

Preparative separations may be carried out as needed, using either an Sq16x or an Sg100c chromatography system and prepackaged silica gel columns all purchased from Teledyne Isco, (Lincoln, Nebr.). Alternately, compounds and intermediates may be purified by flash column chromatography using silica gel (230-400 mesh) packing material, or by HPLC using a C-18 reversed phase column. Typical solvents employed for the Isco systems and flash column chromatography may be dichloromethane, methanol, ethyl acetate, hexane, acetone, aqueous hydroxyamine and triethyl amine. Typical solvents employed for the reverse phase HPLC may be varying concentrations of acetonitrile and water with 0.1% trifluoroacetic acid.

### General Methods

The following synthetic reaction schemes are merely illustrative of some methods by which the compounds of the present invention can be synthesized, and various modifications to these synthetic reaction schemes can be made and will be suggested to one skilled in the art having referred to the disclosure contained in this application.

### Example 1

### Preparation of 4-(3-(2H-1,2,3-triazol-2-yl)phenylamino)-2-((1S,2R)-2-aminocyclohexylamino)pyrimidine-5-carboxamide

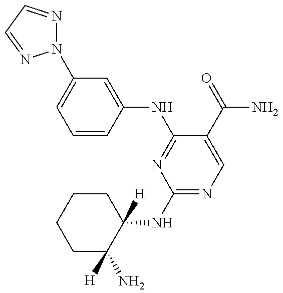 [SMILES: NC(=O)c1cnc(N[C@H]2CCCC[C@H]2N)nc1Nc1cccc(-n2nccn2)c1]

The title compound was prepared according to the synthetic scheme illustrated below:

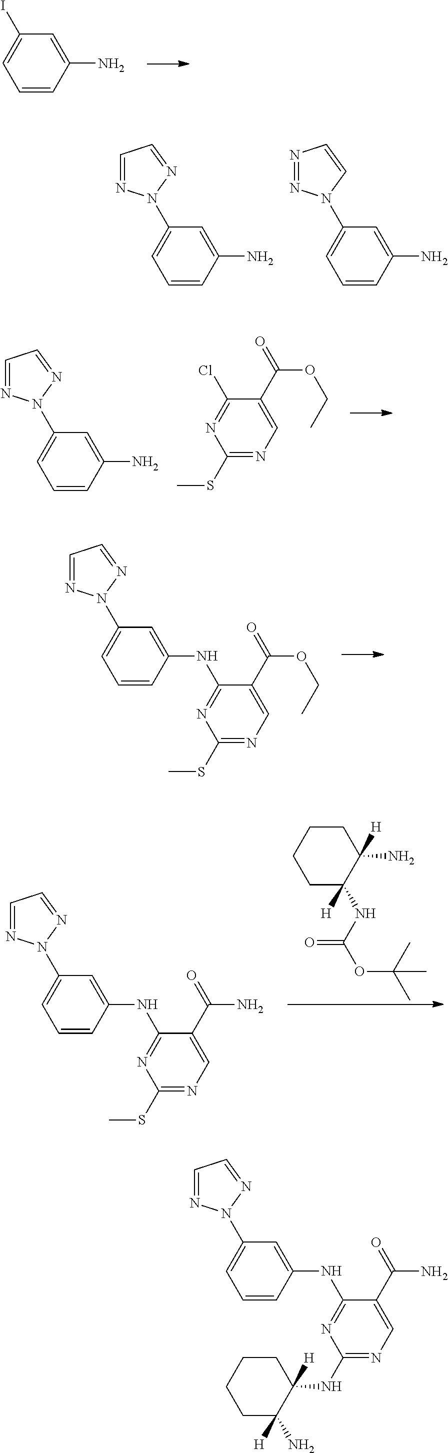

The mixture of 3-iodoaniline (3.70 g, 16.9 mmol), 1,2,3-triazole (3.91 mL, 67.6 mmol), K₃PO₄ (7.17 g, 33.8 mmol), fine powder CuI (1.61 g, 8.45 mmol), ethylenediamine (0.60 mL, 8.45 mmol) in 30 mL dioxane and 15 mL DMSO were refluxed for three days to yield major product 3-(2H-1,2,3-triazol-2-yl)aniline and minor product 3-(1H-1,2,3-triazol-1-yl)aniline in ratio of about 3:1. The mixture was diluted with 400 mL EtOAc, vigorously stirred, filtered through celite, washed with brine twice, concentrated in vacuo, and subjected to flash column to isolate 3-(2H-1,2,3-triazol-2-yl)aniline (1.86 g, 68% yield).

Ethyl 4-chloro-2-(methylthio)pyrimidine-5-carboxylate (5.00 g, 21.5 mmol) was dissolved in 50 mL DMF. To it were added 3-(2H-1,2,3-triazol-2-yl)aniline (4.13 g, 25.8 mmol) and DIEA (7.50 mL, 43.0 mmol). The mixture was stirred at 40° C. for overnight. To it was poured 300 mL water. Solid ethyl 4-(3-(2H-1,2,3-triazol-2-yl)phenylamino)-2-(methylthio)pyrimidine-5-carboxylate crashed out. It was collected by filtration, washed with water. The solid was then dissolved in 100 mL dioxane. To it was added LiOH hydrate (2.80 g, 64.5 mmol) and 50 mL water. The mixture was stirred for overnight. To the mixture was added HCl to adjust the pH to 2. Solid carboxylic acid crashed out. It was isolated by filtration, washed with water and dried in vacuum oven. This solid was dissolved in 100 mL DMF. To it were added EDC.HCl (5.76 g, 30 mmol) and HOBt.H₂O (4.60 g, 30 mmol). The mixture was stirred for 2.5 h. To it was added ammonium hydroxide solution (28%, 9.1 mL, 100 mmol). The mixture was stirred for 2 h. To it was poured 300 mL water. Solid 4-(3-(2H-1,2,3-triazol-2-yl)phenylamino)-2-(methylthio)pyrimidine-5-carboxamide crashed out. It was collected by filtration, washed with water and dried in vacuum oven (5.77 g). 4-(3-(2H-1,2,3-Triazol-2-yl)phenylamino)-2-(methylthio)pyrimidine-5-carboxamide (50 mg, 0.15 mmol) was dissolved in 3 mL NMP. To it was added MCPBA (65%, 48 mg, 0.18 mmol). The mixture was stirred for 1.5 h. To it were added DIEA (78 μL, 0.45 mmol) and tert-butyl (1R,2S)-2-aminocyclohexylcarbamate (64 mg, 0.30 mmol). The mixture was stirred at 90° C. for 2 h. It was diluted with 100 mL EtOAc, washed with 1N NaOH and brine, dried, concentrated in vacuo. The residue was treated with neat TFA at RT for 2 h. It was concentrated and subjected to reverse phase preaparative HPLC to isolate the title compound. MS found for C19H23N9O as (M+H)⁺ 394.4. UV: λ=249 nm.

### Example 2

### Preparation of 4-(3-(2H-1,2,3-triazol-2-yl)phenylamino)-2-(ethylamino)pyrimidine-5-carboxamide

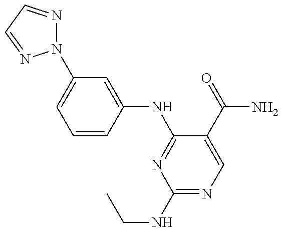 [SMILES: CCNc1ncc(C(N)=O)c(Nc2cccc(-n3nccn3)c2)n1]

4-(3-(2H-1,2,3-Triazol-2-yl)phenylamino)-2-(methylthio)pyrimidine-5-carboxamide (100 mg, 0.31 mmol) was dissolved in 3 mL NMP in a sealed tube. To it was added MCPBA (77%, 103 mg, 0.46 mmol). The mixture was stirred for 30 m at RT. To it was added ethylamine (2.0M in THF, 0.75 mL, 1.5 mmol). The mixture was stirred at 80° C. for 3 h. It was cooled to RT, diluted with 100 mL EtOAc, washed with 1N NaOH and brine, dried, concentrated in vacuo, and subjected to reverse phase preaparative HPLC to isolate the title compound. MS found for C15H16N8O as (M+H)⁺ 325.3. UV: λ=254 nm. Proton NMR: (CD3OD) δ 9.06 (1H, s), 8.48 (1H, s), 7.94 (2H, s), 7.93 (1H, m), 7.56 (1 h, t, J=8.0 Hz), 7.42 (1H, d, J=8.0 Hz), 3.67 (2H, q, J=7.2 Hz), 1.30 (3H, t, J=7.2 Hz) ppm.

### Example 3

### Preparation of 4-(3-(2H-1,2,3-triazol-2-yl)phenylamino)-2-(dimethylamino)pyrimidine-5-carboxamide

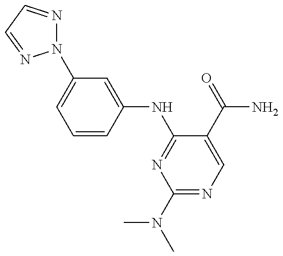 [SMILES: CN(C)c1ncc(C(N)=O)c(Nc2cccc(-n3nccn3)c2)n1]

4-(3-(2H-1,2,3-Triazol-2-yl)phenylamino)-2-(methylthio)pyrimidine-5-carboxamide (100 mg, 0.31 mmol) was dissolved in 3 mL DMF in a sealed tube. To it was added MCPBA (77%, 103 mg, 0.46 mmol). The mixture was stirred for 40 m at RT. To it was added dimethylamine (2.0M in THF, 0.78 mL, 1.6 mmol). The mixture was stirred at 60° C. for overnight. It was cooled to RT, diluted with 100 mL EtOAc, washed with 1N NaOH and brine, dried, concentrated in vacuo, and subjected to reverse phase preaparative HPLC to isolate the title compound. MS found for C15H16N8O as (M+H)⁺ 325.3. UV: λ=259 nm. Proton NMR: (DMSO-d₆) δ 12.25 (1H, s), 8.85 (1H, s), 8.67 (1H, s), 8.46 (1H, s), 8.11 (2H, s), 7.78 (2H, d, J=7.2 Hz), 7.54 (1H, t, J=7.2 Hz), 7.44 (1H, d, J=7.2 Hz), 3.25 (6H, s) ppm.

### Example 4

### Preparation of 4-(3-(2H-1,2,3-triazol-2-yl)phenylamino)-2-((1R,2R)-2-hydroxycyclohexylamino)pyrimidine-5-carboxamide

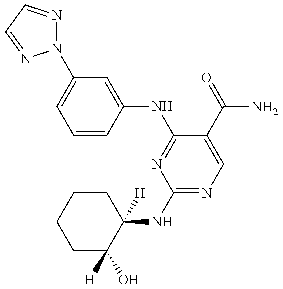 [SMILES: NC(=O)c1cnc(N[C@@H]2CCCC[C@H]2O)nc1Nc1cccc(-n2nccn2)c1]

4-(3-(2H-1,2,3-Triazol-2-yl)phenylamino)-2-(methylthio)pyrimidine-5-carboxamide (300 mg, 0.91 mmol) was dissolved in 15 mL NMP. To it was added MCPBA (77%, 340 mg, 1.38 mmol). The mixture was stirred for 40 m at RT. To it were added DIEA (1.58 mL, 9.1 mmol) and (1R,2R)-2-aminocyclohexanol hydrochloride (415 mg, 2.73 mmol). The mixture was stirred at 90° C. for 2.5 h. It was cooled to RT, diluted with 300 mL EtOAc, washed with 1N NaOH and brine, dried, concentrated in vacuo, and subjected to reverse phase preaparative HPLC to isolate the title compound (330 mg). MS found for C19H22N8O2 as (M+H)⁺ 395.3. UV: λ=254 nm.

### Example 5

### Preparation of 4-(3-(2H-1,2,3-triazol-2-yl)phenylamino)-2-((1R,2S)-2-hydroxycyclohexylamino)pyrimidine-5-carboxamide

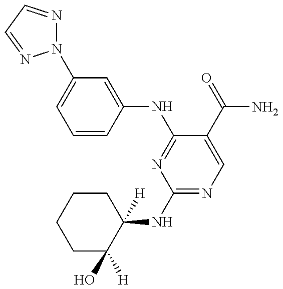 [SMILES: NC(=O)c1cnc(N[C@@H]2CCCC[C@@H]2O)nc1Nc1cccc(-n2nccn2)c1]

4-(3-(2H-1,2,3-Triazol-2-yl)phenylamino)-2-(methylthio)pyrimidine-5-carboxamide (300 mg, 0.91 mmol) was dissolved in 15 mL NMP. To it was added MCPBA (77%, 340 mg, 1.38 mmol). The mixture was stirred for 40 m at RT. To it were added DIEA (1.58 mL, 9.1 mmol) and (1R,2S)-2-aminocyclohexanol hydrochloride (415 mg, 2.73 mmol). The mixture was stirred at 90° C. for 2.5 h. It was cooled to RT, diluted with 300 mL EtOAc, washed with 1N NaOH and brine, dried, concentrated in vacuo, and subjected to reverse phase preaparative HPLC to isolate the title compound (280 mg). MS found for C19H22N8O2 as (M+H)⁺ 395.3. UV: λ=254 nm.

### Example 6

### Preparation of 4-(3-(2H-1,2,3-triazol-2-yl)phenylamino)-2-(1R,2R)-2-ethoxycyclohexylamino)pyrimidine-5-carboxamide

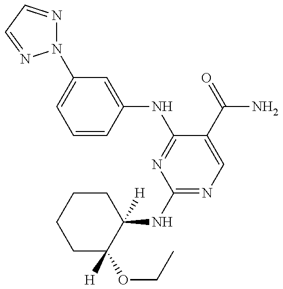 [SMILES: CCO[C@@H]1CCCC[C@H]1Nc1ncc(C(N)=O)c(Nc2cccc(-n3nccn3)c2)n1]

The title compound was prepared according to the synthetic scheme illustrated below:

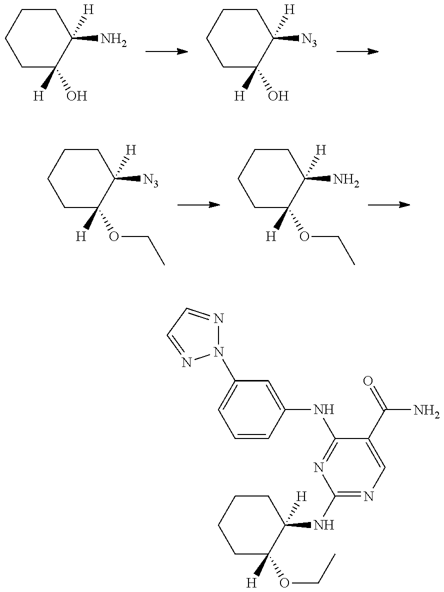

Sodium azide (2.43 g, 18.7 mmol) was dissolved in 7.6 mL water and stirred in ice bath. To it were added 8 mL DCM and then Tf₂O (1.28 mL, 7.6 mmol). The mixture was stirred for 2 h in ice bath. The DCM phase was separated, and the aqueous phase was extracted with DCM twice. The combined DCM phase was washed with sat. NaHCO₃ and water, dried over MgSO₄. (1R,2R)-2-Aminocyclohexanol hydrochloride (302 mg, 2.0 mmol) was dissolved in 5 mL dry DCM and stirred at RT. To it were added DMAP (1.07 g, 8.8 mmol) and then the above-prepared TfN₃/DCM solution dropwise. The mixture was stirred for overnight. It was concentrated in vacuo and subjected to flash column to isolate (1R,2R)-2-azidocyclohexanol (280 mg, a clear oil). Proton NMR: (CDCl₃) δ 3.34 (1H, m), 3.14 (1H, m), 2.87 (1H, s), 1.99 (2H, m), 1.69 (2H, m), 1.31-1.18 (4H, m) ppm.

(1R,2R)-2-Azidocyclohexanol (280 mg, 2.0 mmol) was dissolved in 10 mL NMP. To it was added NaH (60% in mineral oil, 240 mg, 6.0 mmol). The mixture was stirred at RT for 10 m. To it was then added iodoethane (0.80 mL, 10.0 mmol). It was stirred for 1 h, diluted with methanol, concentrated in vacuo. The residue was taken into 200 mL EtOAc, washed with water twice, dried, and passed thru a short silica plug. This EtOAc solution was treated with 300 mg Pd/C (10%, wet) under a hydrogen balloon for overnight. It was filtered through a celite plug and concentrated in vacuo as crude (1R,2R)-2-ethoxycyclohexanamine.

4-(3-(2H-1,2,3-Triazol-2-yl)phenylamino)-2-(methylthio)pyrimidine-5-carboxamide (220 mg, 0.67 mmol) was dissolved in 10 mL NMP. To it was added MCPBA (77%, 180 mg, 0.80 mmol). The mixture was stirred for 40 m at RT. To it were added DIEA (0.58 mL, 3.35 mmol) and the crude (1R,2R)-2-ethoxycyclohexanamine prepared as above described. The mixture was stirred at 90° C. for 1.5 h. It was cooled to RT, diluted with 200 mL EtOAc, washed with 1N NaOH and brine, dried, concentrated in vacuo, and subjected to reverse phase preaparative HPLC to isolate the title compound (240 mg). MS found for C21H26N8O2 as (M+H)⁺ 423.3. UV: λ=254 nm.

### Example 7

### Preparation of 4-(3-(2H-1,2,3-triazol-2-yl)phenylamino)-2-((1R,2S)-2-ethoxycyclohexylamino)pyrimidine-5-carboxamide

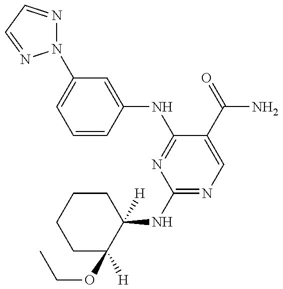 [SMILES: CCO[C@H]1CCCC[C@H]1Nc1ncc(C(N)=O)c(Nc2cccc(-n3nccn3)c2)n1]

The title compound was prepared according to the synthetic scheme illustrated below:

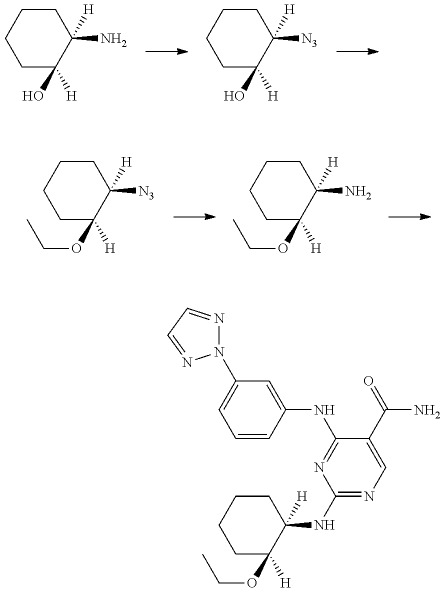

Sodium azide (2.43 g, 18.7 mmol) was dissolved in 7.6 mL water and stirred in ice bath. To it were added 8 mL DCM and then Tf₂O (1.28 mL, 7.6 mmol). The mixture was stirred for 3 h in ice bath. The DCM phase was separated, and the aqueous phase was extracted with DCM twice. The combined DCM phase was washed with sat. NaHCO₃ and water, dried over MgSO₄. (1S,2R)-2-Aminocyclohexanol hydrochloride (302 mg, 2.0 mmol) was dissolved in 5 mL dry DCM and stirred at RT. To it were added DMAP (1.07 g, 8.8 mmol) and then the above-prepared TfN₃/DCM solution dropwise. The mixture was stirred for overnight. It was concentrated in vacuo and subjected to flash column to isolate (1S,2R)-2-azidocyclohexanol (300 mg, a clear oil). Proton NMR: (CDCl₃) δ 3.74 (1H, m), 3.59 (1H, m), 2.72 (1H, s), 1.83 (1H, m), 1.69-1.48 (5H, m), 1.27 (2H, m) ppm.

(1S,2R)-2-Azidocyclohexanol (300 mg, 2.1 mmol) was dissolved in 10 mL NMP. To it was added NaH (60% in mineral oil, 250 mg, 6.3 mmol). The mixture was stirred at RT for 10 m. To it was then added iodoethane (0.80 mL, 10.0 mmol). It was stirred for 1 h, diluted with methanol, concentrated in vacuo. The residue was taken into 200 mL EtOAc, washed with water twice, dried, and passed thru a short silica plug. This EtOAc solution was treated with 300 mg Pd/C (10%, wet) under a hydrogen balloon for overnight. It was filtered through a celite plug and concentrated in vacuo as crude (1S,2R)-2-ethoxycyclohexanamine.

4-(3-(2H-1,2,3-Triazol-2-yl)phenylamino)-2-(methylthio)pyrimidine-5-carboxamide (220 mg, 0.67 mmol) was dissolved in 10 mL NMP. To it was added MCPBA (77%, 180 mg, 0.80 mmol). The mixture was stirred for 40 m at RT. To it were added DIEA (0.58 mL, 3.35 mmol) and the crude (1S,2R)-2-ethoxycyclohexanamine prepared as above described. The mixture was stirred at 90° C. for 2 h. It was cooled to RT, diluted with 200 mL EtOAc, washed with 1N NaOH and brine, dried, concentrated in vacuo, and subjected to reverse phase preaparative HPLC to isolate the title compound (152 mg). MS found for C21H26N8O2 as (M+H)⁺ 423.3. UV: λ=254 nm.

### Example 8

### Preparation of 4-(3-(2H-1,2,3-triazol-2-yl)phenylamino)-2-((1R,2S)-2-azidocyclohexylamino)pyrimidine-5-carboxamide

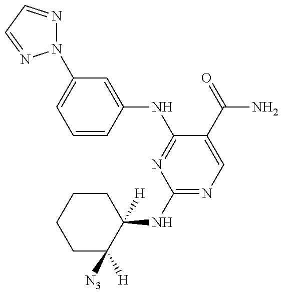 [SMILES: [N-]=[N+]=N[C@H]1CCCC[C@H]1Nc1ncc(C(N)=O)c(Nc2cccc(-n3nccn3)c2)n1]

The title compound was prepared according to the synthetic scheme illustrated below:

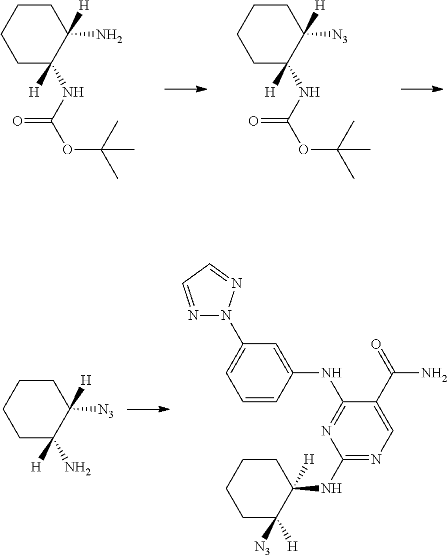 [SMILES: CC(C)(C)OC(=O)N[C@@H]1CCCC[C@@H]1N ; CC(C)(C)OC(=O)N[C@@H]1CCCC[C@@H]1N=[N+]=[N-] ; [N-]=[N+]=N[C@H]1CCCC[C@H]1N ; [N-]=[N+]=N[C@H]1CCCC[C@H]1Nc1ncc(C(N)=O)c(Nc2cccc(-n3nccn3)c2)n1]

Sodium azide (13.25 g, 203.8 mmol) was dissolved in 40 mL water and stirred in ice bath. To it were added 40 mL DCM and then Tf₂O (7.0 mL, 41.4 mmol). The mixture was stirred for 2 h in ice bath. The DCM phase was separated, and the aqueous phase was extracted with DCM twice. The combined DCM phase was washed with sat. NaHCO₃ and water, dried over MgSO₄. tert-butyl (1R,2S)-2-aminocyclohexylcarbamate (2.33 g, 10.9 mmol) was dissolved in 30 mL dry DCM and stirred at RT. To it were added DMAP (5.85 g, 48.0 mmol) and then the above-prepared TfN₃/DCM solution dropwise. The mixture was stirred for overnight. It was concentrated in vacuo and subjected to flash column to isolate tert-butyl (1R,2S)-2-azidocyclohexylcarbamate in quantitative yield. It was treated with 4N HCl in dioxane (25 mL) at RT for 1 h. The mixture was concentrated in vacuo and pumped for overnight to afford (1R,2S)-2-azidocyclohexanamine hydrochloride (1.61 g).

4-(3-(2H-1,2,3-Triazol-2-yl)phenylamino)-2-(methylthio)pyrimidine-5-carboxamide (310 mg, 0.94 mmol) was dissolved in 15 mL NMP. To it was added MCPBA (77%, 460 mg, 1.88 mmol). The mixture was stirred for 30 m at RT. To it were added DIEA (0.98 mL, 5.64 mmol) and (1R,2S)-2-azidocyclohexanamine hydrochloride (250 mg, 1.40 mmol). The mixture was stirred at 90° C. for 5 h. It was cooled to RT, diluted with 200 mL EtOAc, washed with 1N NaOH and brine, dried, concentrated in vacuo, and subjected to reverse phase preaparative HPLC to isolate the title compound (200 mg). MS found for C19H21N11O as (M+H)⁺ 420.4. UV: λ=254 nm.

### Example 9

### Preparation of 2-((1R,2S)-2-(1H-1,2,3-triazol-1-yl)cyclohexylamino)-4-(3-(2H-1,2,3-triazol-2-yl)phenylamino)pyrimidine-5-carboxamide

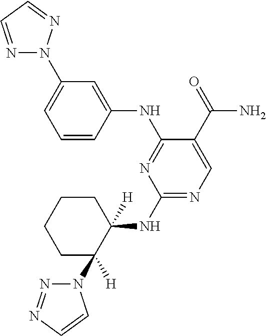 [SMILES: NC(=O)c1cnc(N[C@@H]2CCCC[C@@H]2n2ccnn2)nc1Nc1cccc(-n2nccn2)c1]

4-(3-(2H-1,2,3-Triazol-2-yl)phenylamino)-2-((1R,2S)-2-azidocyclohexylamino)pyrimidine-5-carboxamide (28 mg, 0.06 mmol) was dissolved in 10 mL methanol. To it were added ethynyltrimethylsilane (12 mg, 0.12 mmol), DBU (27 μL, 0.18 mmol) and fine powder CuI (17 mg, 0.09 mmol). The mixture was stirred at 40° C. for overnight. It was acidified with TFA and subjected to reverse phase preparative HPLC to isolate the title compound. MS found for C21H23N11O as (M+H)⁺ 446.4. UV: λ=254 nm.

### Example 10

### Preparation of 4-(3-(2H-1,2,3-triazol-2-yl)phenylamino)-2-((1R,2S)-2-formamidocyclohexylamino)pyrimidine-5-carboxamide

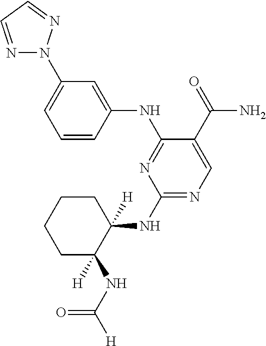 [SMILES: NC(=O)c1cnc(N[C@@H]2CCCC[C@@H]2NC=O)nc1Nc1cccc(-n2nccn2)c1]

4-(3-(2H-1,2,3-Triazol-2-yl)phenylamino)-2-((1R,2S)-2-aminocyclohexylamino)pyrimidine-5-carboxamide was prepared by the same scheme shown in Example 1 for 4-(3-(2H-1,2,3-triazol-2-yl)phenylamino)-2-((1S,2R)-2-aminocyclohexylamino)pyrimidine-5-carboxamide. 4-(3-(2H-1,2,3-Triazol-2-yl)phenylamino)-2-((1R,2S)-2-aminocyclohexylamino)pyrimidine-5-carboxamide (100 mg) was stirred in 5 mL formic acid at 100° C. in a sealed tube for three days to yield the title compound. It was isolated using reverse phase preparative HPLC. MS found for C20H23N9O2 as (M+H)⁺ 422.3. UV: λ=254 nm.

### Example 11

### Preparation of 4-(3-(2H-1,2,3-triazol-2-yl)phenylamino)-2-((1R,2S)-2-(cyanomethylamino)cyclohexylamino)pyrimidine-5-carboxamide

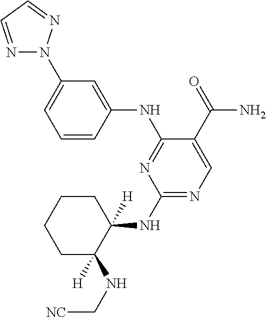 [SMILES: N#CCN[C@H]1CCCC[C@H]1Nc1ncc(C(N)=O)c(Nc2cccc(-n3nccn3)c2)n1]

4-(3-(2H-1,2,3-Triazol-2-yl)phenylamino)-2-((1R,2S)-2-aminocyclohexylamino)pyrimidine-5-carboxamide was prepared by the same scheme shown in Example 1 for 4-(3-(2H-1,2,3-triazol-2-yl)phenylamino)-2-((1S,2R)-2-aminocyclohexylamino)pyrimidine-5-carboxamide. 4-(3-(2H-1,2,3-Triazol-2-yl)phenylamino)-2-((1R,2S)-2-aminocyclohexylamino)pyrimidine-5-carboxamide (80 mg, 0.2 mmol) was dissolved in 3 mL NMP. To it were added DIEA (70 μL, 0.4 mmol) and bromoacetonitrile (26 μL, 0.4 mmol). The mixture was stirred at RT for 1.5 h and diluted with a methylamine/methanol solution. The mixture was concentrated in vacuo, acidified with TFA and subjected to reverse phase preparative HPLC to isolate the title compound. MS found for C21H24N10O as (M+H)⁺ 433.3. UV: λ=249 nm.

### Example 12

### Preparation of 4-(3-(2H-1,2,3-triazol-2-yl)phenylamino)-2-((1R,2S)-2-(2-hydroxyethylamino)cyclohexylamino)pyrimidine-5-carboxamide

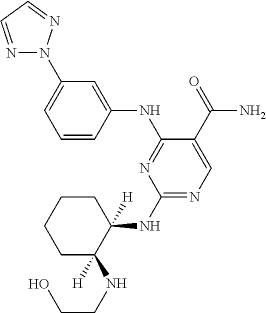 [SMILES: NC(=O)c1cnc(N[C@@H]2CCCC[C@@H]2NCCO)nc1Nc1cccc(-n2nccn2)c1]

4-(3-(2H-1,2,3-Triazol-2-yl)phenylamino)-2-((1R,2S)-2-aminocyclohexylamino)pyrimidine-5-carboxamide was prepared by the same scheme shown in Example 1 for 4-(3-(2H-1,2,3-triazol-2-yl)phenylamino)-2-((1S,2R)-2-aminocyclohexylamino)pyrimidine-5-carboxamide. 4-(3-(2H-1,2,3-Triazol-2-yl)phenylamino)-2-((1R,2S)-2-aminocyclohexylamino)pyrimidine-5-carboxamide (230 mg, 0.58 mmol) was dissolved in 6 mL NMP. To it were added DIEA (300 μL, 1.74 mmol) and (2-bromoethoxy)(tert-butyl)dimethylsilane (500 μL, 2.32 mmol). The mixture was stirred at RT for three days. It was diluted with 200 mL EtOAc, washed with brine three times, dried, concentrated and subjected to flash column to isolate 4-(3-(2H-1,2,3-triazol-2-yl)phenylamino)-2-((1R,2S)-2-(2-(tert-butyldimethylsilyloxy)ethylamino)cyclohexylamino)pyrimidine-5-carboxamide (120 mg, 38%). It was dissolved in 10 mL THF and treated with Bu₄NF (1.0M in THF, 0.66 mL, 0.66 mmol) for 40 m. The mixture was concentrated in vacuo, acidized with TFA and subjected to reverse phase preparative HPLC to isolate the title compound (85 mg). MS found for C21H27N9O₂ as (M+H)⁺ 438.4. UV: λ=249 nm.

### Example 13

### Preparation of 4-(3-(2H-1,2,3-triazol-2-yl)phenylamino)-2-(1R,2R)-2-amino-3,3-difluorocyclohexylamino)pyrimidine-5-carboxamide

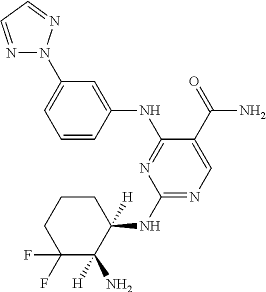 [SMILES: NC(=O)c1cnc(N[C@@H]2CCCC(F)(F)[C@@H]2N)nc1Nc1cccc(-n2nccn2)c1]

The title compound was prepared according to the synthetic scheme illustrated below:

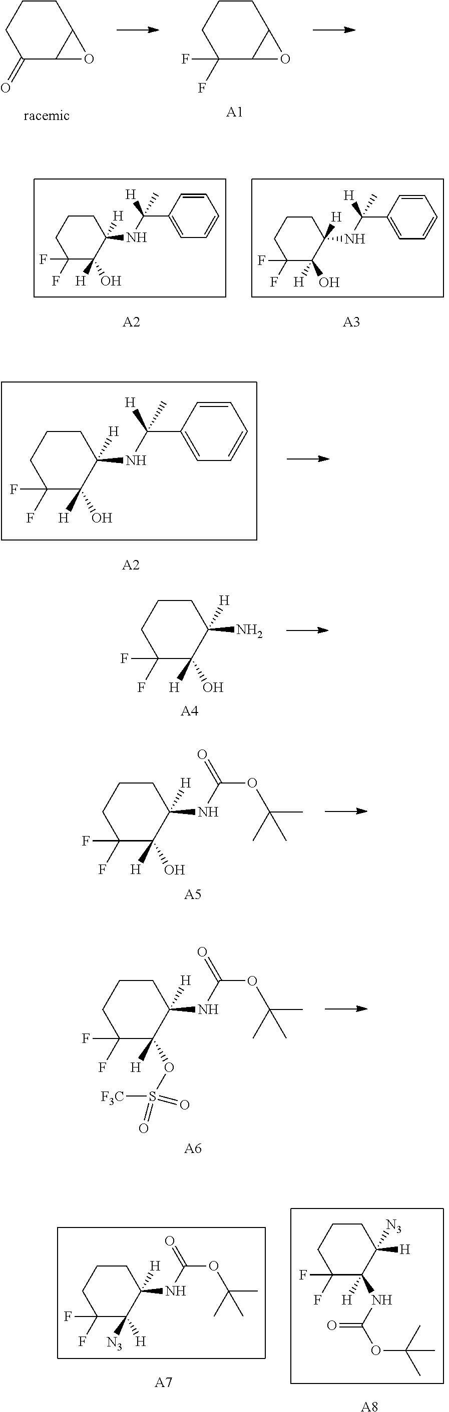

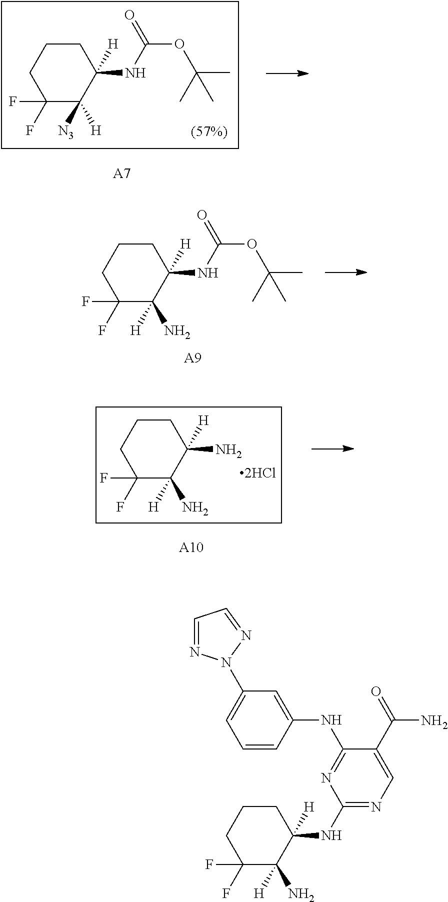

7-Oxabicyclo[4.1.0]heptan-2-one (8.0 mL, 81 mmol) was dissolved in 40 mL dry DCM and stirred in ice bath. To it was added Deoxo-Fluor (32.8 mL, 178 mmol) dropwise. The mixture was allowed to warm up to RT and stirred for overnight to give a mixture of 2,2-difluoro-7-oxabicyclo[4.1.0]heptane (A1) and some remaining 7-oxabicyclo[4.1.0]heptan-2-one. The mixture was cooled to −20° C. and carefully quenched with 5 mL water dropwise. The mixture was diluted with 600 mL DCM and 200 mL water. The organic phase was separated, dried, filtered through a short silica plug and concentrated in vacuo. The residue was then dissolved in 150 mL DCM.

A solution of (R)-(+)-α-methylbenzylamine (12.2 mL, 96 mmol) in 50 mL DCM was prepared and stirred in ice bath. To it was added a solution of trimethylaluminum in hexane (Aldrich #268569, 44 mL, 88 mmol). The mixture was stirred for 1 h. To it was then added the 150 mL DCM solution from previous step. The mixture was stirred at RT for over the weekend to give a mixture of (1S,6R)-2,2-difluoro-6-((R)-1-phenylethylamino)cyclohexanol (A2) and (1R,6S)-2,2-difluoro-6-((R)-1-phenylethylamino)cyclohexanol (A3) in about 1:1 ratio. The mixture was then cooled in ice bath. Powder NaF (16.8 g, 400 mmol) was added. Then the mixture was treated with ice chips slowly. To it was poured 500 mL DCM. The mixture was stirred for 2 h at RT. It was filtered through celite. The filtrate was concentrated in vacuo and subjected to flash column with 0-2.5% MeOH in DCM to isolate (1S,6R)-2,2-difluoro-6-((R)-1-phenylethylamino)cyclohexanol (A2) (6.67 g) and (1R,6S)-2,2-difluoro-6-((R)-1-phenylethylamino)cyclohexanol (A3) (5.70 g). A2 NMR (CDCl₃): 7.39-7.25 (5H, m), 4.00 (1H, q, J=6.8 Hz), 3.53 (1H, ddd), 3.04 (2H, bs), 2.74 (1H, m), 2.11 (1H, m), 1.79 (1H, m), 1.63 (2H, m), 1.44 (3H, d, J=6.4 Hz), 1.40 (1H, m), 1.11 (1H, m) ppm. A3 NMR (CDCl₃): 7.36-7.23 (5H, m), 3.95 (1H, q, J=6.4 Hz), 3.48 (1H, ddd), 2.44 (2H, bs), 2.41 (1H, m), 2.09 (2H, m), 1.72-1.55 (2H, m), 1.38 (3H, d, J=6.8 Hz), 1.31 (1H, m), 1.12 (1H, m) ppm.

(1S,6R)-2,2-Difluoro-6-((R)-1-phenylethylamino)cyclohexanol (A2) (6.67 g) was dissolved in 200 mL EtOAc and 200 mL methanol. To the solution was added 20 wt % palladium hydroxide on carbon (1.65 g). The mixture was shaken on a Parr shaker under 50 psi hydrogen for overnight. The mixture was filtered through celite. The filtrate was concentrated in vacuo to afford (1S,6R)-6-amino-2,2-difluorocyclohexanol (A4) (4.03 g). It was dissolved in 200 mL THF. To it were added triethylamine (18.1 mL, 130 mmol) and BOC anhydride (6.8 g, 31.2 mmol). The mixture was stirred for overnight, concentrated in vacuo and subjected to flash column (10-20% EtOAc in hexane) to isolate tert-butyl (1R,2S)-3,3-difluoro-2-hydroxycyclohexylcarbamate (A5) (5.34 g).

(1R,2S)-3,3-Difluoro-2-hydroxycyclohexylcarbamate (A5) (1.83 g, 7.3 mmol) was dissolved in 50 mL dry DCM. To it was added 15 mL dry pyridine. The mixture was stirred in ice bath. To it was added Tf₂O (4.9 mL, 29 mmol). The reaction was allowed for 15 min and quenched with water. It was further diluted with 100 mL water and 500 mL DCM. The organic phase was separated and washed with water three times, dried, concentrated in vacuo and pumped to dryness to give crude (1S,6R)-6-(tert-butoxycarbonylamino)-2,2-difluorocyclohexyl trifluoromethanesulfonate (A6). It was dissolved in 30 mL NMP. To it was added sodium azide (2.85 g, 43.8 mmol). The mixture was stirred at 100° C. for 3 h. It was cooled to RT. To it was poured 500 mL EtOAc. The mixture was washed with water three times, dried, concentrated in vacuo and subjected to flash column (0-20% EtOAc in hexane) to isolate major product A7, tert-butyl (1R,2R)-2-azido-3,3-difluorocyclohexylcarbamate (1.15 g, 57%), and minor product A8, tert-butyl (1R,6S)-6-azido-2,2-difluorocyclohexylcarbamate (0.18 g, 9%). A7 NMR (CDCl₃): 4.77 (1H, d, J=6.8 Hz), 3.97 (1H, bs), 3.87 (1H, bm), 1.97-1.86 (2H, m), 1.72-1.63 (2H, m), 1.45 (9H, s), 1.36 (2H, m) ppm. A8 NMR (CDCl₃): 4.81 (1H, d, J=8.8 Hz), 3.91 (1H, m), 3.28 (1H, m), 2.21 (1H, m), 2.11 (1H, m), 1.86-1.79 (2H, m), 1.78-1.64 (2H, m), 1.48 (9H, s), 1.43-1.39 (2H, m) ppm.

tert-butyl (1R,2R)-2-azido-3,3-difluorocyclohexylcarbamate (A7) (1.15 g, 4.16 mmol) was dissolved in 250 mL EtOAc. To it was added 2.0 g of 10% Pd/C. A hydrogen balloon was attached to the reaction flask. The mixture was stirred for overnight. It was filtered through celite. The celite cake was washed thoroughly with EtOAc and methanol. The filtrate was concentrated in vacuo and pumped to dryness to afford tert-butyl (1R,2R)-2-amino-3,3-difluorocyclohexylcarbamate (A9) as a white solid. It was then treated with 40 mL 4N HCl in dioxane at RT for 1.5 h to get a thick gel. It was concentrated and pumped overnight to afford (1R,2R)-3,3-difluorocyclohexane-1,2-diamine dihydrochloride (A10) as a light brown solid. 4-(3-(2H-1,2,3-Triazol-2-yl)phenylamino)-2-(methylthio)pyrimidine-5-carboxamide (80 mg, 0.24 mmol) was dissolved in 4 mL NMP. To it was added MCPBA (77%, 88 mg, 0.36 mmol). The mixture was stirred for 30 m at RT. To it were added DIEA (0.34 mL, 1.92 mmol) and (1R,2R)-3,3-difluorocyclohexane-1,2-diamine dihydrochloride (80 mg, 0.36 mmol). The mixture was stirred at 90° C. for 2 h. It was cooled to RT, diluted with 200 mL EtOAc, washed with 1N NaOH and brine, dried, concentrated in vacuo, and subjected to reverse phase preparative HPLC to isolate the title compound (72 mg). MS found for C19H21F2N9O as (M+H)⁺ 430.4. UV: λ=249 nm.

### Example 14

### Preparation of 4-(3-(2H-1,2,3-triazol-2-yl)phenylamino)-2-((1S,6S)-6-amino-2,2-difluorocyclohexylamino)pyrimidine-5-carboxamide

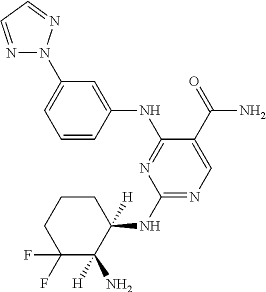 [SMILES: NC(=O)c1cnc(N[C@@H]2CCCC(F)(F)[C@@H]2N)nc1Nc1cccc(-n2nccn2)c1]

The title compound was prepared according to the synthetic scheme illustrated below:

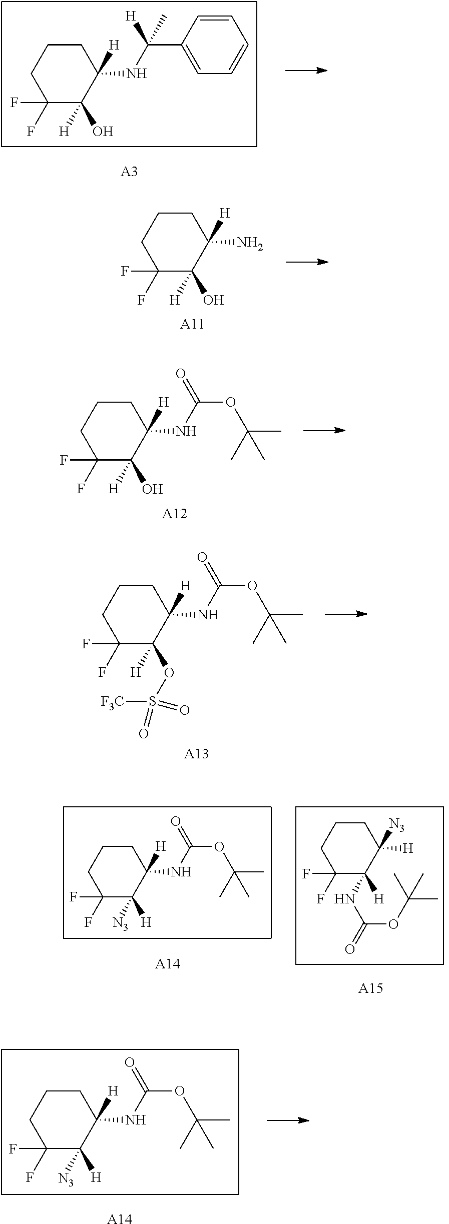

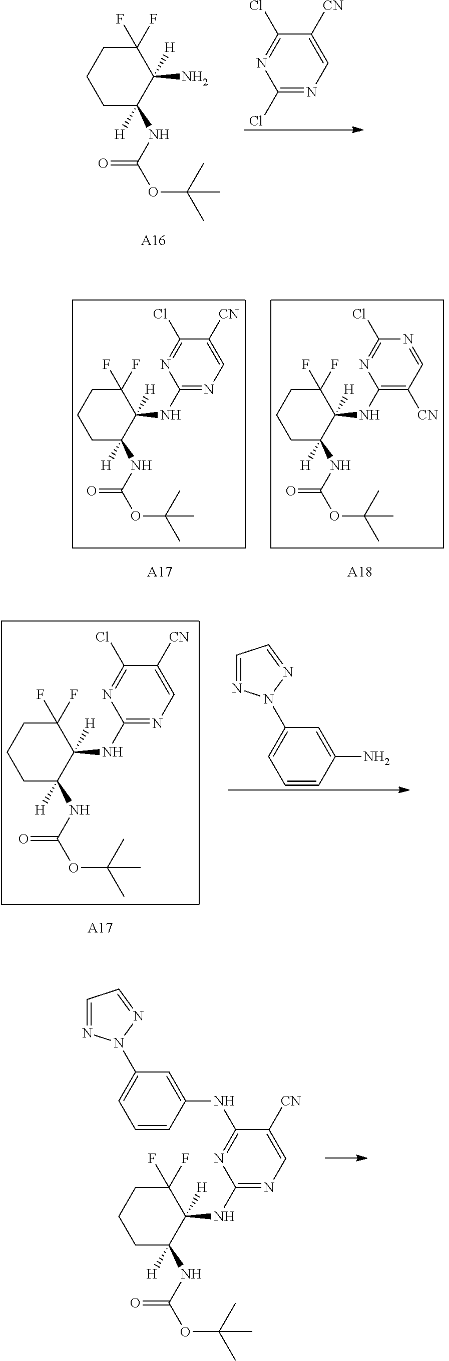

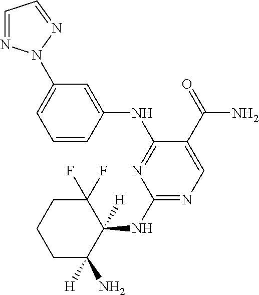 [SMILES: NC(=O)c1cnc(N[C@H]2[C@@H](N)CCCC2(F)F)nc1Nc1cccc(-n2nccn2)c1]

(1R,6S)-2,2-Difluoro-6-((R)-1-phenylethylamino)cyclohexanol (A3) (5.70 g, 22 mmol) was dissolved in 200 mL and 200 mL methanol. To the solution was added 20 wt % palladium hydroxide on carbon (1.50 g). The mixture was shaken on a Parr shaker under 40 psi hydrogen for overnight. The mixture was filtered through celite. The filtrate was concentrated in vacuo to afford (1R,6S)-6-amino-2,2-difluorocyclohexanol (A11). It was dissolved in 200 mL THF. To it were added triethylamine (15.3 mL, 110 mmol) and BOC anhydride (5.8 g, 26.4 mmol). The mixture was stirred for overnight, concentrated in vacuo and subjected to flash column (10-20% EtOAc in hexane) to isolate tert-butyl (1S,2R)-3,3-difluoro-2-hydroxycyclohexylcarbamate (A12) (4.65 g).

tert-butyl (1S,2R)-3,3-difluoro-2-hydroxycyclohexylcarbamate (A12) (3.00 g, 11.9 mmol) was dissolved in 100 mL dry DCM. To it was added 30 mL dry pyridine. The mixture was stirred in ice bath. To it was added Tf₂O (8.0 mL, 47 mmol). The reaction was allowed for 10 min and quenched with water. It was further diluted with 100 mL water and 500 mL DCM. The organic phase was separated and washed with water x3, dried, concentrated in vacuo and pumped to dryness to give crude (1R,6S)-6-(tert-butoxycarbonylamino)-2,2-difluorocyclohexyl trifluoromethanesulfonate (A13). It was dissolved in 36 mL NMP. To it was added sodium azide (4.64 g, 71.4 mmol). The mixture was stirred at 100° C. for 3 h. It was cooled to RT. To it was poured 500 mL EtOAc. The mixture was washed with water three times, dried, concentrated in vacuo and subjected to flash column (0-15% EtOAc in hexane) to isolate major product A14, tert-butyl (1S,2S)-2-azido-3,3-difluorocyclohexylcarbamate (2.17 g, 66%) and minor product A15, tert-butyl (1S,6R)-6-azido-2,2-difluorocyclohexylcarbamate (0.42 g, 13%). A15 NMR (CDCl₃): 4.92 (1H, d, J=8.8 Hz), 3.91 (1H, m), 3.77 (1H, bm), 1.85 (1H, m), 1.80 (1H, m), 1.64-1.53 (2H, m), 1.36 (9H, s), 1.36-1.27 (2H, m) ppm. A14 NMR (CDCl₃): 4.83 (1H, d, J=9.2 Hz), 3.91 (1H, m), 3.28 (1H, m), 2.20 (1H, m), 2.10 (1H, m), 1.84-1.69 (2H, m), 1.47 (9H, s), 1.47-1.42 (2H, m) ppm.

tert-butyl (1S,2S)-2-azido-3,3-difluorocyclohexylcarbamate (A14)(2.17 g, 7.86 mmol) was dissolved in 250 mL EtOAc. To it was added 0.5 g of 10% Pd/C. A hydrogen balloon was attached to the reaction flask. The mixture was stirred for overnight. It was filtered through celite. The celite cake was washed thoroughly with EtOAc and methanol. The filtrate was concentrated in vacuo and pumped to dryness to afford tert-butyl (1S,2S)-2-amino-3,3-difluorocyclohexylcarbamate as a white solid (A16) (1.61 g, 85%).

2,4-Dichloropyrimidine-5-carbonitrile (571 mg, 3.28 mmol) was dissolved in 25 mL NMP. To it were added tert-butyl (1S,2S)-2-amino-3,3-difluorocyclohexylcarbamate (820 mg, 3.28 mmol) and DIEA (1.14 mL, 6.56 mmol). The mixture was stirred at 100° C. for 20 m to afford major product tert-butyl (1S,2S)-2-(4-chloro-5-cyanopyrimidin-2-ylamino)-3,3-difluorocyclohexylcarbamate (A17, UV=268 nm) and minor product tert-butyl (1S,2S)-2-(2-chloro-5-cyanopyrimidin-4-ylamino)-3,3-difluorocyclohexylcarbamate (A18, UV=249, 301 nm) in the ratio of 2.6:1. The mixture was cooled to RT, diluted with EtOAc, washed with brine three times, concentrated in vacuo and subjected to flash column to separate A17 and A18.

The mixture of tert-butyl (1S,2S)-2-(4-chloro-5-cyanopyrimidin-2-ylamino)-3,3-difluorocyclohexylcarbamate (A17) (51 mg, 0.13 mmol), 3-(2H-1,2,3-triazol-2-yl)aniline (43 mg, 0.26 mmol), fine powder Cs₂CO₃ (130 mg, 0.40 mmol), Q-Phos (21 mg, 0.03 mmol) and Pd(dba)₂ (18 mg, 0.03 mmol) in 15 mL toluene was degassed using argon stream and stirred at 105° C. under argon atmosphere for overnight. It was diluted with 100 mL EtOAc, filtered through celite, concentrated in vacuo and subjected to flash column to isolate tert-butyl (1S,2S)-2-(4-(3-(2H-1,2,3-triazol-2-yl)phenylamino)-5-cyanopyrimidin-2-ylamino)-3,3-difluorocyclohexylcarbamate. It was treated with 5 mL TFA and 1 mL concentrate H₂SO₄ at 80° C. for 45 m. It was cooled to RT. To it was added 5 mL water. The mixture was stirred, cooled, filtered and subjected to reverse phase preparative HPLC to isolate the title compound. MS found for C19H21F2N9O as (M+H)⁺ 430.4. UV: λ=249 nm.

### Example 15

### Preparation of 2,2′-(1R,1′R,2S,2′S)-2,2′-(ethane-1,1-diylbis(azanediyl))bis(cyclohexane-2,1-diyl)bis(azanediyl)bis(4-(3-(2H-1,2,3-triazol-2-yl)phenylamino)pyrimidine-5-carboxamide)

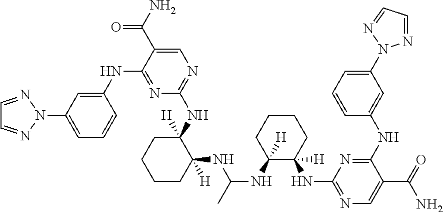

4-(3-(2H-1,2,3-Triazol-2-yl)phenylamino)-2-((1R,2S)-2-aminocyclohexylamino)pyrimidine-5-carboxamide was prepared by the same scheme shown in Example 1 for 4-(3-(2H-1,2,3-triazol-2-yl)phenylamino)-2-((1S,2R)-2-aminocyclohexylamino)pyrimidine-5-carboxamide. 4-(3-(2H-1,2,3-Triazol-2-yl)phenylamino)-2-((1R,2S)-2-aminocyclohexylamino)pyrimidine-5-carboxamide (50 mg) was stirred in 5 mL CHCl₃ with pTSA (10 mg) and CH₃CHO (100 μL) at RT for overnight. The mixture was concentrated in vacuo and subjected to reverse phase preparative HPLC to isolate the title compound (44 mg). MS found for C40H48N18O2 as (M+H)⁺ 813.5. UV: λ=259 nm.

### Example 16

### Preparation of (3S,4S,5R,6S)-6-((1S,2R)-2-(4-(3-(2H-1,2,3-triazol-2-yl)phenylamino)-5-carbamoylpyrimidin-2-ylamino)cyclohexylcarbamoyloxy)-3,4,5-trihydroxytetrahydro-2H-pyran-2-carboxylic acid

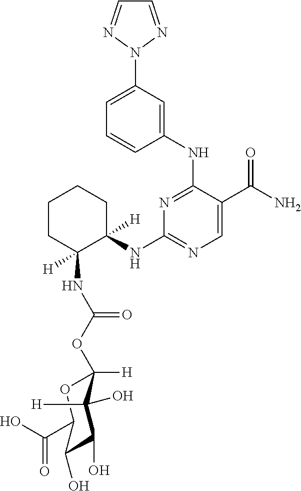

The title compound was prepared according to the synthetic scheme illustrated below:

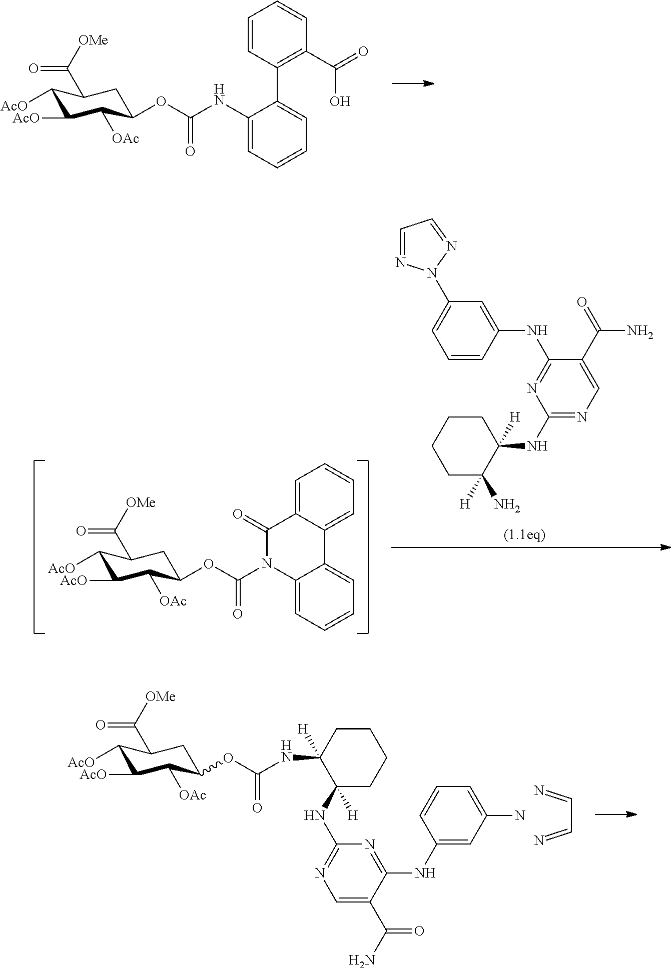

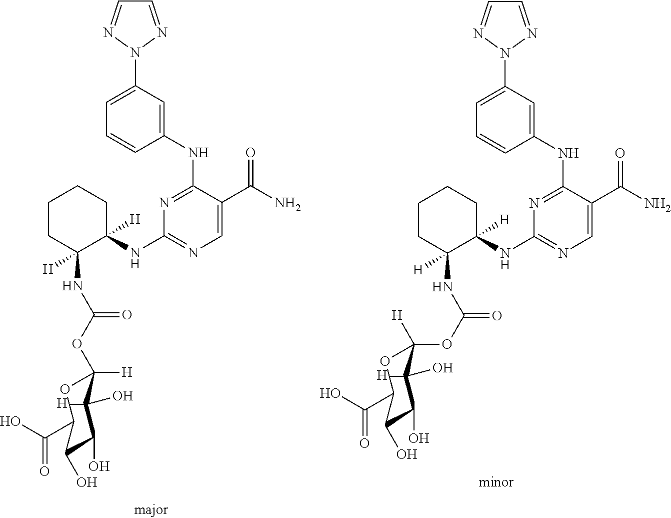

2′-(((2S,3S,4S,5S)-3,4,5-triacetoxy-6-(methoxycarbonyl)tetrahydro-2H-pyran-2-yloxy)carbonylamino)biphenyl-2-carboxylic acid was prepared according to the procedure reported in Tetrahedron Letters (2000, vol. 41, p 9173). This compound (200 mg, 0.35 mmol) was dissolved in 10 mL dry DCM and stirred at RT. To it was added BOP (146 mg, 0.385 mmol). The mixture was stirred for 45 m. To the mixture was dropwise added a slurry of 4-(3-(2H-1,2,3-Triazol-2-yl)phenylamino)-2-((1R,2S)-2-aminocyclohexylamino)pyrimidine-5-carboxamide hydrochloride (165 mg, 0.385 mmol) and DIEA (305 μL, 1.75 mmol) in 10 mL dry DCM. The mixture was stirred at RT for overnight to get the desired adducts as a mixture of alph and beta. The mixture was diluted with EtOAc, washed with water, dried, concentrated and subjected to flash column to isolate the adducts (47 mg, inseparable by flash column). The adduct mixture was dissolved in 15 mL methanol. To it was added 3 mL water. The mixture was stirred at RT. To it was added 1N LiOH aq solution (80 μL). The mixture was stirred for 35 m. Then another 80 μL of the LiOH solution was added. The reaction was quenched with 1N HCl in 20 m. The mixture was concentrated in vacuo and subjected to reverse phase preparative HPLC to isolate the title compound as the major product (17 mg), and also the minor product, (3S,4S,5R,6R)-6-((1S,2R)-2-(4-(3-(2H-1,2,3-triazol-2-yl)phenylamino)-5-carbamoylpyrimidin-2-ylamino)cyclohexylcarbamoyloxy)-3,4,5-trihydroxytetrahydro-2H-pyran-2-carboxylic acid (2 mg). MS found for C26H31N9O9 as (M+H)⁺ 614.4. UV: λ=254 mm.

### Example 17

### Preparation of (3S,4S,5R,6R)-6-((1S,2R)-2-(4-(3-(2H-1,2,3-triazol-2-yl)phenylamino)-5-carbamoylpyrimidin-2-ylamino)cyclohexylcarbamoyloxy)-3,4,5-trihydroxytetrahydro-2H-pyran-2-carboxylic acid

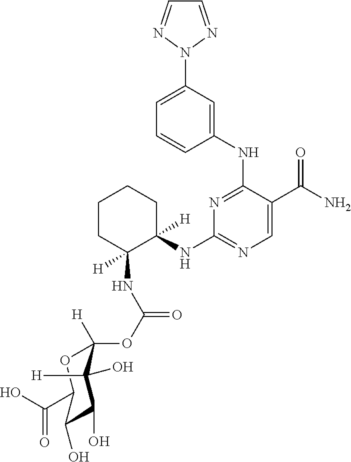

The title compound was prepared as the minor product according to the synthetic scheme illustrated in Example 16 for (3S,4S,5R,6S)-6-((1S,2R)-2-(4-(3-(2H-1,2,3-triazol-2-yl)phenylamino)-5-carbamoylpyrimidin-2-ylamino)cyclohexylcarbamoyloxy)-3,4,5-trihydroxytetrahydro-2H-pyran-2-carboxylic acid. MS found for C26H31N9O9 as (M+H)⁺ 614.4. UV: λ=254 nm.

### Example 18

### Preparation of 4-(3-(2H-1,2,3-triazol-2-yl)phenylamino)-2-(5,6,7,8-tetrahydroquinolin-8-ylamino)pyrimidine-5-carboxamide

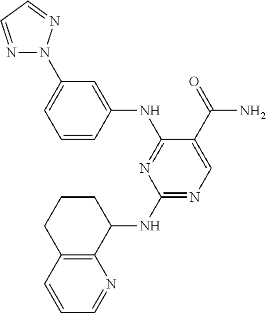 [SMILES: NC(=O)c1cnc(NC2CCCc3cccnc32)nc1Nc1cccc(-n2nccn2)c1]

4-(3-(2H-1,2,3-Triazol-2-yl)phenylamino)-2-(methylthio)pyrimidine-5-carboxamide (100 mg, 0.31 mmol) was dissolved in 6 mL NMP. To it was added MCPBA (77%, 104 mg, 0.47 mmol). The mixture was stirred for 30 m at RT. To it were added DIEA (0.55 mL, 3.1 mmol) and 5,6,7,8-tetrahydroquinolin-8-amine dihydrochloride (210 mg, 0.93 mmol). The mixture was stirred at 90° C. for 1.5 h. It was cooled to RT, diluted with 300 mL EtOAc, washed with 1N NaOH and brine, dried, concentrated in vacuo, and subjected to reverse phase preparative HPLC to isolate the title compound (racemic) (127 mg). MS found for C22H21N9O as (M+H)⁺ 428.3. UV: λ=263 nm.

### Example 19

### Preparation of 4-(3-(2H-1,2,3-triazol-2-yephenylamino)-2-((1S,2S)-2-amino-3,3-difluorocyclohexylamino)pyrimidine-5-carboxamide

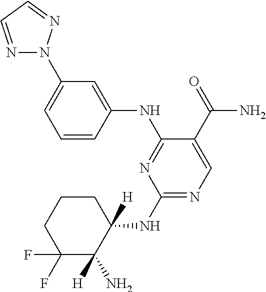 [SMILES: NC(=O)c1cnc(N[C@H]2CCCC(F)(F)[C@H]2N)nc1Nc1cccc(-n2nccn2)c1]

The title compound was prepared according to the synthetic scheme illustrated below:

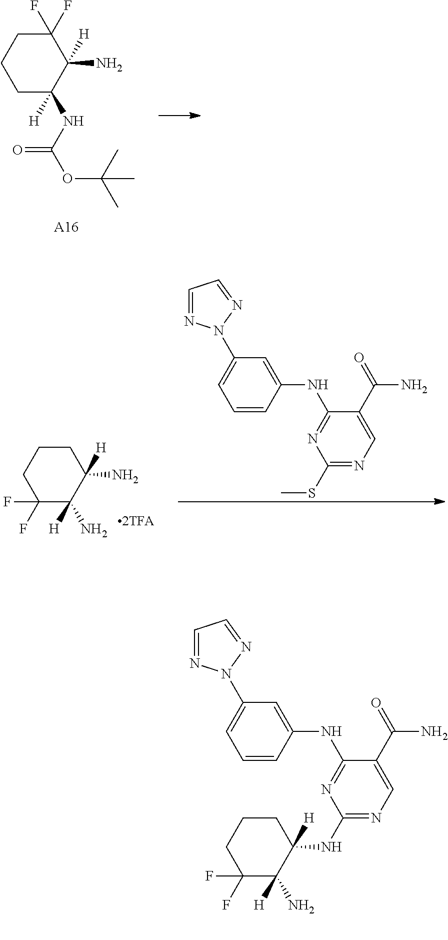

tert-butyl (1S,2S)-2-amino-3,3-difluorocyclohexylcarbamate (A16, see Example 14) was treated with TFA at RT for 1 h. The mixture was concentrated in vacuo to dryness to afford (1S,2S)-3,3-difluorocyclohexane-1,2-diamine di-TFA salt.

4-(3-(2H-1,2,3-Triazol-2-yl)phenylamino)-2-(methylthio)pyrimidine-5-carboxamide (65 mg, 0.2 mmol) was dissolved in 3 mL NMP. To it was added MCPBA (77%, 74 mg, 0.3 mmol). The mixture was stirred for 40 m at RT. To it were added DIEA (0.35 mL, 2.0 mmol) and (1S,2S)-3,3-difluorocyclohexane-1,2-diamine di-TFA salt (0.2 mmol). The mixture was stirred at 90° C. for 2 h. It was cooled to RT and subjected to reverse phase preparative HPLC to isolate the title compound (38 mg). MS found for C19H21F2N9O as (M+H)⁺ 430.4. UV: λ=249 nm.

### Example 20

### Preparation of 2-((1R,2S)-2-aminocyclohexylamino)-4-(4-bromo-3-(2H-1,2,3-triazol-2-yl)phenylamino)pyrimidine-5-carboxamide

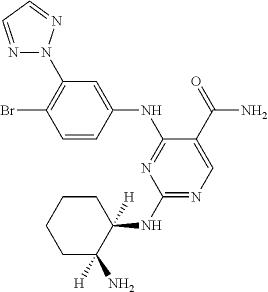 [SMILES: NC(=O)c1cnc(N[C@@H]2CCCC[C@@H]2N)nc1Nc1ccc(Br)c(-n2nccn2)c1]

The title compound was prepared according to the synthetic scheme illustrated below:

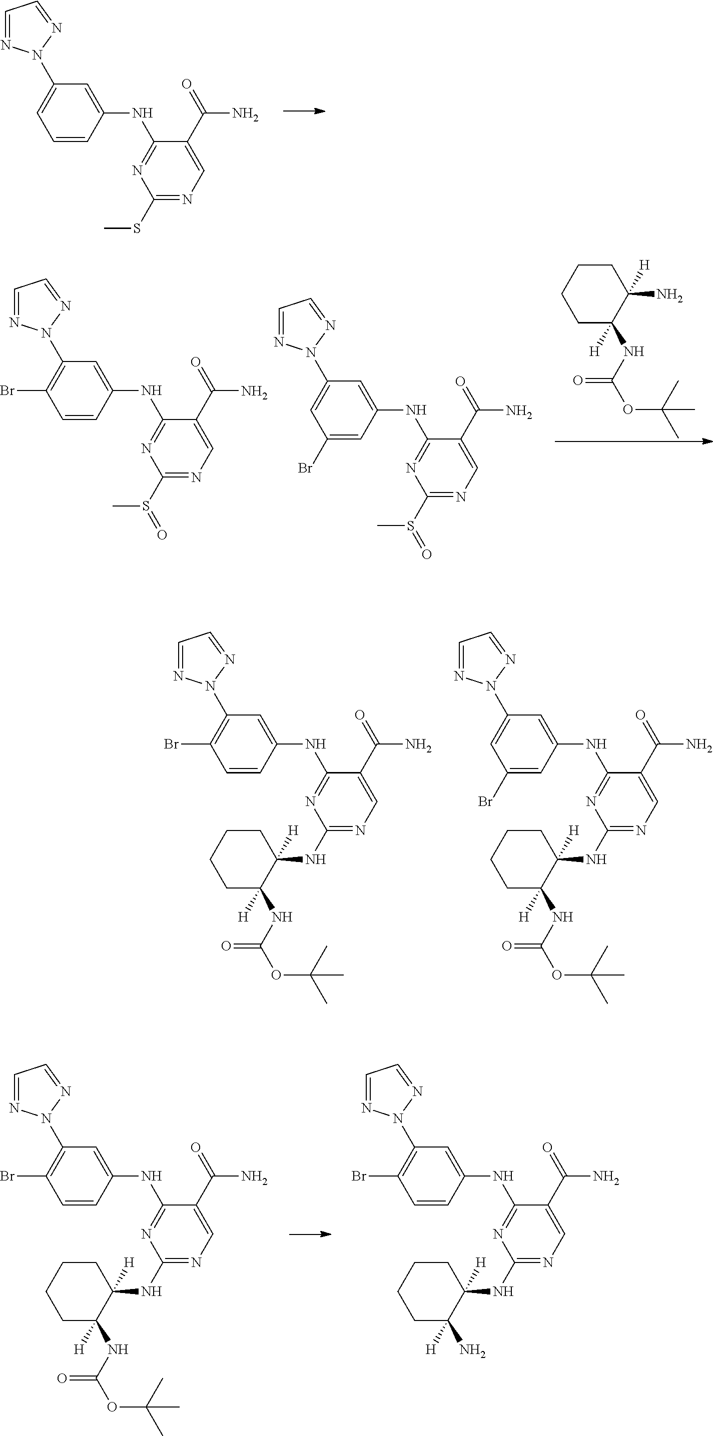

4-(3-(2H-1,2,3-Triazol-2-yl)phenylamino)-2-(methylthio)pyrimidine-5-carboxamide (150 mg, 0.46 mmol) was dissolved in 15 mL DMF. To it was added NBS (122 mg, 0.69 mmol). The mixture was stirred for 12 m. Then another portion of NBS (112 mg, 0.69 mmol) was added. The mixture was stirred for 1 h to afford a mixture of 4-(4-bromo-3-(2H-1,2,3-triazol-2-yl)phenylamino)-2-(methylsulfinyl)pyrimidine-5-carboxamide and 4-(3-bromo-5-(2H-1,2,3-triazol-2-yl)phenylamino)-2-(methylsulfinyl)pyrimidine-5-carboxamide in ratio of about 2.5:1.

To it were added DIEA (0.40 mL, 2.30 mmol) and then tert-butyl (1S,2R)-2-aminocyclohexylcarbamate (200 mg, 0.92 mmol). The mixture was stirred at 90° C. for 3 h. It was diluted with EtOAc, washed with 1N NaOH and brine, concentrated in vacuo and subjected to reverse phase preparative HPLC to isolate the major product, tert-butyl (1S,2R)-2-(4-(4-bromo-3-(2H-1,2,3-triazol-2-yl)phenylamino)-5-carbamoylpyrimidin-2-ylamino)cyclohexylcarbamate, and the minor product, tert-butyl (1S,2R)-2-(4-(3-bromo-5-(2H-1,2,3-triazol-2-yl)phenylamino)-5-carbamoylpyrimidin-2-ylamino)cyclohexylcarbamate.

The major product, tert-butyl (1S,2R)-2-(4-(4-bromo-3-(2H-1,2,3-triazol-2-yl)phenylamino)-5-carbamoylpyrimidin-2-ylamino)cyclohexylcarbamate, was treated with TFA and subjected to reverse phase preparative HPLC to isolate the title compound. MS found for C19H22BrN9O as (M+H)⁺ 472.4. UV: λ=246, 294 nm.

### Example 21

### Preparation of 2-((1R,2S)-2-aminocyclohexylamino)-4-(3-bromo-5-(2H-1,2,3-triazol-2-yl)phenylamino)pyrimidine-5-carboxamide

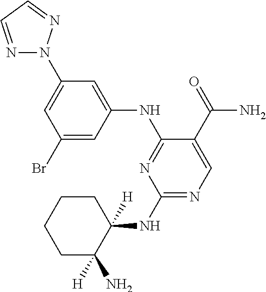 [SMILES: NC(=O)c1cnc(N[C@@H]2CCCC[C@@H]2N)nc1Nc1cc(Br)cc(-n2nccn2)c1]

The minor product shown in Example 20, tert-butyl (1S,2R)-2-(4-(3-bromo-5-(2H-1,2,3-triazol-2-yl)phenylamino)-5-carbamoylpyrimidin-2-ylamino)cyclohexylcarbamate, was treated with TFA and subjected to reverse phase preparative HPLC to isolate the title compound. MS found for C19H22BrN9O as (M+H)⁺ 472.4. UV: λ=253, 272 nm.

### Example 22

### Preparation of 2-((1R,2S)-2-aminocyclohexylamino)-4-(4-chloro-3-(2H-1,2,3-triazol-2-yl)phenylamino)pyrimidine-5-carboxamide

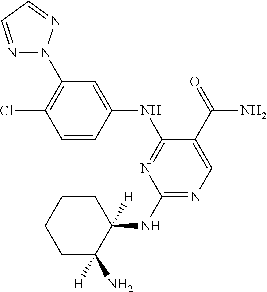 [SMILES: NC(=O)c1cnc(N[C@@H]2CCCC[C@@H]2N)nc1Nc1ccc(Cl)c(-n2nccn2)c1]

The title compound was prepared according to the synthetic scheme illustrated below:

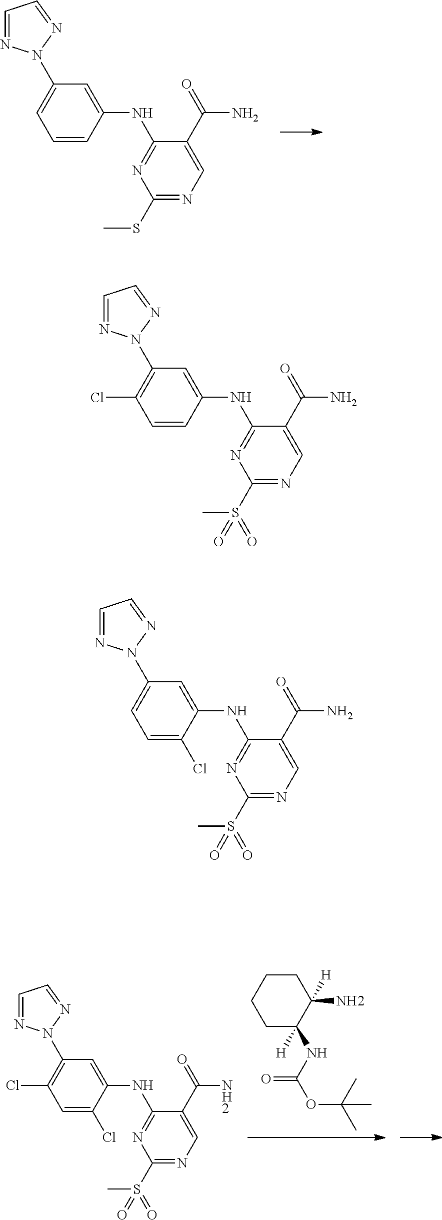

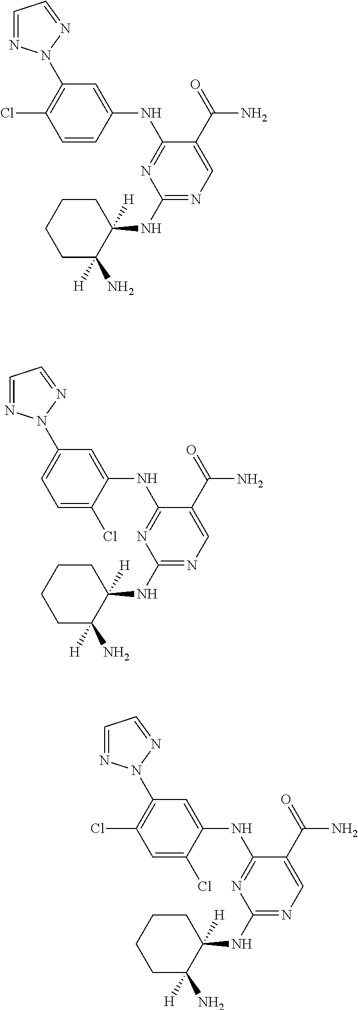 [SMILES: NC(=O)c1cnc(N[C@@H]2CCCC[C@@H]2N)nc1Nc1ccc(Cl)c(-n2nccn2)c1 ; NC(=O)c1cnc(N[C@@H]2CCCC[C@@H]2N)nc1Nc1cc(-n2nccn2)ccc1Cl ; NC(=O)c1cnc(N[C@@H]2CCCC[C@@H]2N)nc1Nc1cc(-n2nccn2)c(Cl)cc1Cl]

4-(3-(2H-1,2,3-Triazol-2-yl)phenylamino)-2-(methylthio)pyrimidine-5-carboxamide (150 mg, 0.46 mmol) was dissolved in 15 mL DMF. To it was added NCS (276 mg, 2.07 mmol). The mixture was stirred for 1 h to afford a mixture of 4-(4-chloro-3-(2H-1,2,3-triazol-2-yl)phenylamino)-2-(methylsulfonyl)pyrimidine-5-carboxamide (major), 4-(2-chloro-5-(2H-1,2,3-triazol-2-yl)phenylamino)-2-(methylsulfonyl)pyrimidine-5-carboxamide (minor) and 4-(2,4-dichloro-5-(2H-1,2,3-triazol-2-yl)phenylamino)-2-(methylsulfonyl)pyrimidine-5-carboxamide (very minor).

To it were added DIEA (0.40 mL, 2.30 mmol) and then tert-butyl (1S,2R)-2-aminocyclohexylcarbamate (200 mg, 0.92 mmol). The mixture was stirred at 90° C. for 2 h. It was diluted with EtOAc, washed with 1N NaOH and brine, concentrated in vacuo. The residue was treated with TFA at RT for 30 m and subjected to reverse phase preparative HPLC to isolate the three products. The title compound, 2-((1R,2S)-2-aminocyclohexylamino)-4-(4-chloro-3-(2H-1,2,3-triazol-2-yl)phenylamino)pyrimidine-5-carboxamide, was the major product. MS found for C19H22ClN9O as (M+H)⁺ 428.4. UV: λ=246, 291 nm.

### Example 23

### Preparation of 2-((1R,2S)-2-aminocyclohexylamino)-4-(2-chloro-3-(2H-1,2,3-triazol-2-yl)phenylamino)pyrimidine-5-carboxamide

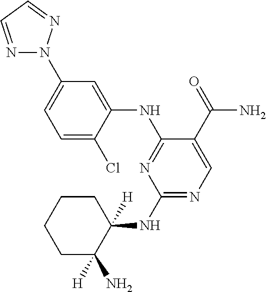 [SMILES: NC(=O)c1cnc(N[C@@H]2CCCC[C@@H]2N)nc1Nc1cc(-n2nccn2)ccc1Cl]

The title compound was isolated from the reaction mixture for 2-((1R,2S)-2-aminocyclohexylamino)-4-(4-chloro-3-(2H-1,2,3-triazol-2-yl)phenylamino)pyrimidine-5-carboxamide (Example 22) as a minor product. MS found for C19H22ClN9O as (M+H)⁺ 428.4. UV: λ=252 nm.

### Example 24

### Preparation of 2-((1R,2S)-2-aminocyclohexylamino)-4-(2,4-dichloro-5-(2H-1,2,3-triazol-2-yl)phenylamino)pyrimidine-5-carboxamide

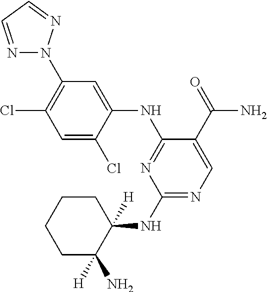 [SMILES: NC(=O)c1cnc(N[C@@H]2CCCC[C@@H]2N)nc1Nc1cc(-n2nccn2)c(Cl)cc1Cl]

The title compound was isolated from the reaction mixture for 2-((1R,2S)-2-aminocyclohexylamino)-4-(4-chloro-3-(2H-1,2,3-triazol-2-yl)phenylamino)pyrimidine-5-carboxamide (Example 22) as a very minor product. MS found for C19H21Cl2N9O as (M+H)⁺ 462.4. UV: λ=249, 295 nm.

### Example 26

### Preparation of 2-(((1R,2R)-2-amino-3,3-difluorocyclohexyl)amino)-4-((3-(pyrimidin-2-yl)phenyl)amino)pyrimidine-5-carboxamide

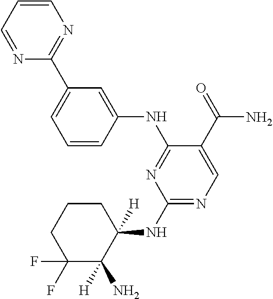 [SMILES: NC(=O)c1cnc(N[C@@H]2CCCC(F)(F)[C@@H]2N)nc1Nc1cccc(-c2ncccn2)c1]

The title compound was prepared according to the similar synthetic scheme illustrated in Example 13 for 4-(3-(2H-1,2,3-triazol-2-yl)phenylamino)-2-((1R,2R)-2-amino-3,3-difluorocyclohexylamino)pyrimidine-5-carboxamide. MS found for C21H22F2N8O as (M+H)⁺ 441.4. UV: λ=244 nm.

### Example 27

### Preparation of 2-(((1R,2R)-2-amino-3,3-difluorocyclohexyl)amino)-4-(phenylamino)pyrimidine-5-carboxamide

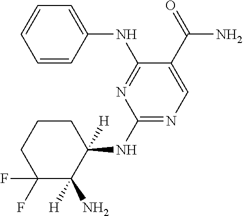 [SMILES: NC(=O)c1cnc(N[C@@H]2CCCC(F)(F)[C@@H]2N)nc1Nc1ccccc1]

The title compound was prepared according to the similar synthetic scheme illustrated in Example 13 for 4-(3-(2H-1,2,3-triazol-2-yl)phenylamino)-2-((1R,2R)-2-amino-3,3-difluorocyclohexylamino)pyrimidine-5-carboxamide. MS found for C17H20F2N6O as (M+H)⁺ 363.3. UV: λ=239, 290 nm.

### Example 28

### Preparation of 2-(((1R,2R)-2-amino-3,3-difluorocyclohexyl)amino)-4-((3-chlorophenyl)amino)pyrimidine-5-carboxamide

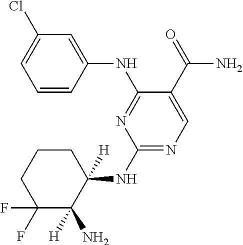 [SMILES: NC(=O)c1cnc(N[C@@H]2CCCC(F)(F)[C@@H]2N)nc1Nc1cccc(Cl)c1]

The title compound was prepared according to the similar synthetic scheme illustrated in Example 13 for 4-(3-(2H-1,2,3-triazol-2-yl)phenylamino)-2-((1R,2R)-2-amino-3,3-difluorocyclohexylamino)pyrimidine-5-carboxamide. MS found for C17H19ClF2N6O as (M+H)⁺ 397.3. UV: λ=239, 288 nm.

### Example 29

### Preparation of 2-(((1R,2R)-2-amino-3,3-difluorocyclohexyl)amino)-4-(benzo[d]thiazol-6-ylamino)pyrimidine-5-carboxamide

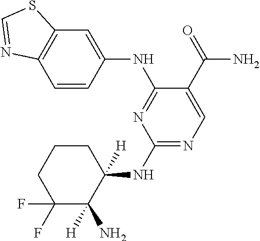 [SMILES: NC(=O)c1cnc(N[C@@H]2CCCC(F)(F)[C@@H]2N)nc1Nc1ccc2ncsc2c1]

The title compound was prepared according to the similar synthetic scheme illustrated in Example 13 for 4-(3-(2H-1,2,3-triazol-2-yl)phenylamino)-2-((1R,2R)-2-amino-3,3-difluorocyclohexylamino)pyrimidine-5-carboxamide. MS found for C18H19F2N7OS as (M+H)⁺ 420.3. UV: λ=240, 297 nm.

### Example 30

### Preparation of 2-(((1R,2R)-2-amino-3,3-difluorocyclohexyl)amino)-4-(benzo[d]thiazol-5-ylamino)pyrimidine-5-carboxamide

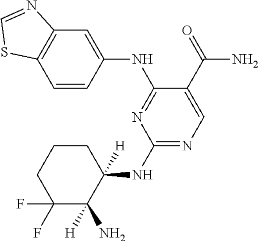 [SMILES: NC(=O)c1cnc(N[C@@H]2CCCC(F)(F)[C@@H]2N)nc1Nc1ccc2scnc2c1]

The title compound was prepared according to the similar synthetic scheme illustrated in Example 13 for 4-(3-(2H-1,2,3-triazol-2-yl)phenylamino)-2-(1R,2R)-2-amino-3,3-difluorocyclohexylamino)pyrimidine-5-carboxamide. MS found for C18H19F2N7OS as (M+H)⁺ 420.3. UV: λ=244, 292 nm.

### Example 31

### Preparation of 2-(((1R,2R)-2-amino-3,3-difluorocyclohexyl)amino)-4-(thieno[2,3-b]pyridin-3-ylamino)pyrimidine-5-carboxamide

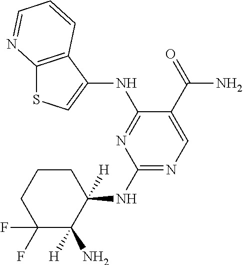 [SMILES: NC(=O)c1cnc(N[C@@H]2CCCC(F)(F)[C@@H]2N)nc1Nc1csc2ncccc12]

The title compound was prepared according to the similar synthetic scheme illustrated in Example 13 for 4-(3-(2H-1,2,3-triazol-2-yl)phenylamino)-2-((1R,2R)-2-amino-3,3-difluorocyclohexylamino)pyrimidine-5-carboxamide. MS found for C18H19F2N7OS as (M+H)⁺ 420.3. UV: λ=240, 301 nm.

### Example 32

### Preparation of 2-(((1R,2R)-2-amino-3,3-difluorocyclohexyl)amino)-4-(m-tolylamino)pyrimidine-5-carboxamide

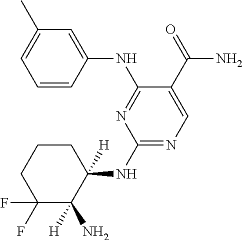 [SMILES: Cc1cccc(Nc2nc(N[C@@H]3CCCC(F)(F)[C@@H]3N)ncc2C(N)=O)c1]

The mixture of 2-((1H-benzo[d][1,2,3]triazol-1-yl)oxy)-4-(m-tolylamino)pyrimidine-5-carboxamide (40 mg, 0.11 mmol), (1R,2R)-3,3-difluorocyclohexane-1,2-diamine dihydrochloride (A10 in Example 13), DIEA (0.12 mL, 0.66 mmol) in 4 mL NMP was stirred at 90° C. for 3 h. It was directly subjected to reverse phase preparative HPLC to isolate the title compound (25 mg). MS found for C18H22F2N6O as (M+H)⁺ 377.4. UV: λ=240,292 nm.

### Example 33

### Preparation of 2-(((1R,2R)-2-amino-3,3-difluorocyclohexyl)amino)-4-((3,5-dimethylphenyl)amino)pyrimidine-5-carboxamide

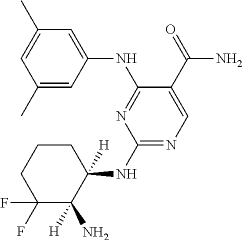 [SMILES: Cc1cc(C)cc(Nc2nc(N[C@@H]3CCCC(F)(F)[C@@H]3N)ncc2C(N)=O)c1]

The title compound was prepared according to the similar synthetic scheme illustrated in Example 32 for 2-(((1R,2R)-2-amino-3,3-difluorocyclohexyl)amino)-4-(m-tolylamino)pyrimidine-5-carboxamide. MS found for C19H24F2N6O as (M+H)⁺ 391.4. UV: λ=240, 292 nm.

### Example 34

### Preparation of 4-((3-(1H-pyrazol-1-yl)phenyl)amino)-2-(((1S,6S)-6-amino-2,2-difluorocyclohexyl)amino)pyrimidine-5-carboxamide

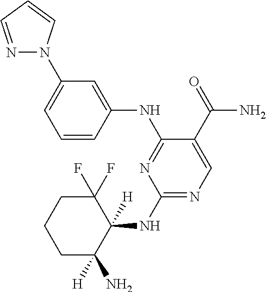 [SMILES: NC(=O)c1cnc(N[C@H]2[C@@H](N)CCCC2(F)F)nc1Nc1cccc(-n2cccn2)c1]

The mixture of tert-butyl (1S,2S)-2-(4-chloro-5-cyanopyrimidin-2-ylamino)-3,3-difluorocyclohexylcarbamate (A17, Example 14) (70 mg, 0.18 mmol), 3-(1H-pyrazol-1-yl)aniline (58 mg, 0.36 mmol), fine powder Cs₂CO₃ (180 mg, 0.54 mmol), Q-Phos (28 mg, 0.04 mmol) and Pd(dba)₂ (23 mg, 0.04 mmol) in 15 mL toluene was degassed using argon stream and stirred at 105° C. under argon atmosphere for overnight. It was diluted with 100 mL EtOAc, filtered through celite, concentrated in vacuo and subjected to flash column to isolate tert-butyl ((1S,2S)-2-((4-((3-(1H-pyrazol-1-yl)phenyl)amino)-5-cyanopyrimidin-2-yl)amino)-3,3-difluorocyclohexyl)carbamate. It was treated with 5 mL TFA and 1 mL concentrate H₂SO₄ at 80° C. for 1 h. It was cooled to RT. To it was added 5 mL water. The mixture was stirred, cooled, filtered and subjected to reverse phase preparative HPLC to isolate the title compound (21 mg). MS found for C20H22F2N8O as (M+H)⁺ 429.4. UV: λ=244 nm.

### Example 35

### Preparation of 2-(((1S,6S)-6-amino-2,2-difluorocyclohexyl)amino)-4-((3-(pyrimidin-2-yl)phenyl)amino)pyrimidine-5-carboxamide

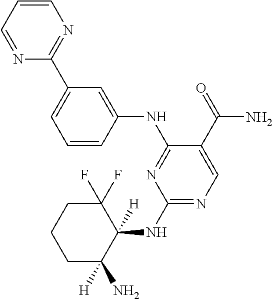 [SMILES: NC(=O)c1cnc(N[C@H]2[C@@H](N)CCCC2(F)F)nc1Nc1cccc(-c2ncccn2)c1]

The mixture of tert-butyl (1S,2S)-2-(4-chloro-5-cyanopyrimidin-2-ylamino)-3,3-difluorocyclohexylcarbamate (A17, Example 14) (70 mg, 0.18 mmol), 3-(pyrimidin-2-yl)aniline (62 mg, 0.36 mmol), fine powder Cs₂CO₃ (180 mg, 0.54 mmol), Q-Phos (28 mg, 0.04 mmol) and Pd(dba)₂ (23 mg, 0.04 mmol) in 15 mL toluene was degassed using argon stream and stirred at 105° C. under argon atmosphere for overnight. It was diluted with 100 mL EtOAc, filtered through celite, concentrated in vacuo and subjected to flash column to isolate tert-butyl ((1S,2S)-2-((5-cyano-4-((3-(pyrimidin-2-yl)phenyl)amino)pyrimidin-2-yl)amino)-3,3-difluorocyclohexyl)carbamate. It was treated with 5 mL TFA and 1 mL concentrate H₂SO₄ at 80° C. for 1 h. It was cooled to RT. To it was added 5 mL water. The mixture was stirred, cooled, filtered and subjected to reverse phase preparative HPLC to isolate the title compound (12 mg). MS found for C21H22F2N8O as (M+H)⁺ 441.4. UV: λ=244 nm.

### Example 36

### Preparation of 2-(((1S,6S)-6-amino-2,2-difluorocyclohexyl)amino)-4-(thieno[2,3-b]pyridin-3-ylamino)pyrimidine-5-carboxamide

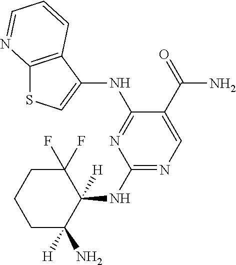 [SMILES: NC(=O)c1cnc(N[C@H]2[C@@H](N)CCCC2(F)F)nc1Nc1csc2ncccc12]

The mixture of tert-butyl (1S,2S)-2-(4-chloro-5-cyanopyrimidin-2-ylamino)-3,3-difluorocyclohexylcarbamate (A17, Example 14) (100 mg, 0.26 mmol), thieno[2,3-b]pyridin-3-amine (78 mg, 0.52 mmol), fine powder Cs₂CO₃ (255 mg, 0.78 mmol), Q-Phos (36 mg, 0.05 mmol) and Pd(dba)₂ (29 mg, 0.05 mmol) in 15 mL toluene was degassed using argon stream and stirred at 105° C. under argon atmosphere for overnight. It was diluted with 100 mL EtOAc, filtered through celite, concentrated in vacuo and subjected to flash column to isolate tert-butyl ((1S,2S)-2-((5-carbamoyl-4-(thieno[2,3-b]pyridin-3-ylamino)pyrimidin-2-yl)amino)-3,3-difluorocyclohexyl)carbamate. It was treated with 1:1 TFA/DCM for 1 h. The mixture was concentrated in vacuo to dryness. The residue was dissolved in 1 mL DMSO and 10 mL methanol. To it were added KOH (100 mg) and then 1 mL H₂O₂ (50%). The mixture was stirred at RT for overnight. It were diluted with acetonitrile, acidified with TFA, concentrated in vacuo and subjected to reverse phase preparative HPLC to isolate the title compound (54 mg). MS found for C18H19F2N7OS as (M+H)⁺ 420.3. UV: λ=240, 301 nm.

### Example 37

### Preparation of 2-(((1S,6S)-6-amino-2,2-difluorocyclohexyl)amino)-4-(pyrazolo[1,5-a]pyridin-3-ylamino)pyrimidine-5-carboxamide

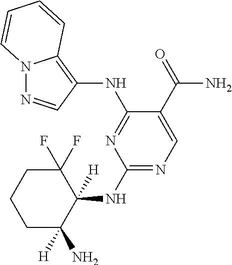 [SMILES: NC(=O)c1cnc(N[C@H]2[C@@H](N)CCCC2(F)F)nc1Nc1cnn2ccccc12]

The mixture of tert-butyl (1S,2S)-2-(4-chloro-5-cyanopyrimidin-2-ylamino)-3,3-difluorocyclohexylcarbamate (A17, Example 14) (100 mg, 0.26 mmol), pyrazolo[1,5-a]pyridin-3-amine dihydrochloride (108 mg, 0.52 mmol), fine powder Cs₂CO₃ (680 mg, 2.08 mmol), Q-Phos (36 mg, 0.05 mmol) and Pd(dba)₂ (29 mg, 0.05 mmol) in 10 mL toluene and 5 mL dioxane was degassed using argon stream and stirred at 105° C. under argon atmosphere for 5 h. It was diluted with 100 mL EtOAc, filtered through celite, concentrated in vacuo and subjected to flash column to isolate tert-butyl ((1S,2S)-2-((5-carbamoyl-4-(pyrazolo[1,5-a]pyridin-3-ylamino)pyrimidin-2-yl)amino)-3,3-difluorocyclohexyl)carbamate. It was treated with 1:1 TFA/DCM for 1 h. The mixture was concentrated in vacuo to dryness. The residue was dissolved in 1 mL DMSO and 10 mL methanol. To it were added KOH (100 mg) and then 1 mL H₂O₂ (50%). The mixture was stirred at RT for overnight. It was diluted with acetonitrile, acidified with TFA, concentrated in vacuo and subjected to reverse phase preparative HPLC to isolate the title compound (27 mg). MS found for C18H20F2N8O as (M+H)⁺ 403.4. UV: λ=226, 325 nm.

### Example 38

### Preparation of 2-(((1S,6S)-6-amino-2,2-difluorocyclohexyl)amino)-4-(p-tolylamino)pyrimidine-5-carboxamide

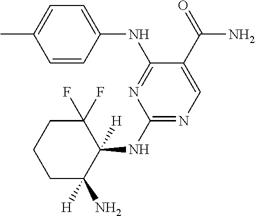 [SMILES: Cc1ccc(Nc2nc(N[C@H]3[C@@H](N)CCCC3(F)F)ncc2C(N)=O)cc1]

The mixture of tert-butyl (1S,2S)-2-(4-chloro-5-cyanopyrimidin-2-ylamino)-3,3-difluorocyclohexylcarbamate (A17, Example 14) (180 mg, 0.46 mmol), p-toluidine (100 mg, 0.92 mmol), fine powder Cs₂CO₃ (450 mg, 1.38 mmol), Q-Phos (71 mg, 0.1 mmol) and Pd(dba)₂ (60 mg, 0.1 mmol) in 20 mL toluene was degassed using argon stream and stirred at 105° C. under argon atmosphere for 5 h. It was diluted with 100 mL EtOAc, filtered through celite, concentrated in vacuo and subjected to flash column to isolate tert-butyl ((1S,2S)-2-((5-cyano-4-(p-tolylamino)pyrimidin-2-yl)amino)-3,3-difluorocyclohexyl)carbamate (120 mg). It was treated with 1:1 TFA/DCM for 30 m. The mixture was concentrated in vacuo to dryness. The residue was dissolved in 1 mL DMSO and 10 mL methanol. To it were added KOH (100 mg) and then 1 mL H₂O₂ (50%). The mixture was stirred at RT for 1.5 h. It was diluted with acetonitrile, acidified with TFA, concentrated in vacuo and subjected to reverse phase preparative HPLC to isolate the title compound (111 mg). MS found for C18H22F2N6O as (M+H)⁺ 377.3. UV: λ=235, 292 nm.

### Example 39

### Preparation of 2-(((1S,6S)-6-amino-2,2-difluorocyclohexyl)amino)-4-(m-tolylamino)pyrimidine-5-carboxamide

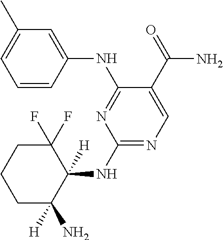 [SMILES: Cc1cccc(Nc2nc(N[C@H]3[C@@H](N)CCCC3(F)F)ncc2C(N)=O)c1]

The title compound was prepared according to the similar procedure described for Example 38, 2-(((1S,6S)-6-amino-2,2-difluorocyclohexyl)amino)-4-(p-tolylamino)pyrimidine-5-carboxamide. MS found for C18H22F2N6O as (M+H)⁺ 377.3. UV: λ=235, 292 nm.

### Example 40

### Preparation of 2-(((1S,2S)-2-amino-3,3-difluorocyclohexyl)amino)-4-(m-tolylamino)pyrimidine-5-carboxamide

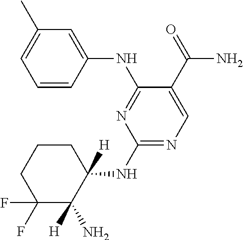 [SMILES: Cc1cccc(Nc2nc(N[C@H]3CCCC(F)(F)[C@H]3N)ncc2C(N)=O)c1]

The mixture of 2-((1H-benzo[d][1,2,3]triazol-1-yl)oxy)-4-(m-tolylamino)pyrimidine-5-carboxamide (80 mg, 0.22 mmol), (1S,2S)-3,3-difluorocyclohexane-1,2-diamine di-TFA salt (shown in Example F19, 0.44 mol), DIEA (0.19 mL, 1.1 mmol) in 4 mL NMP was stirred at 90° C. for 3 h. It was directly subjected to reverse phase preparative HPLC to isolate the title compound (39 mg). MS found for C18H22F2N6O as (M+H)⁺ 377.3. UV: λ=240, 292 nm.

### Example 41

### Preparation of 2-(((1R,2S)-2-aminocyclohexyl)amino)-4-((3-chlorophenyl)amino)pyrimidine-5-carboxamide

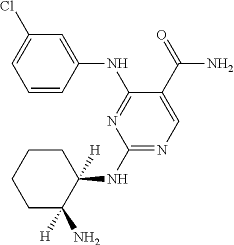 [SMILES: NC(=O)c1cnc(N[C@@H]2CCCC[C@@H]2N)nc1Nc1cccc(Cl)c1]

The title compound was prepared according to the scheme illustrated below:

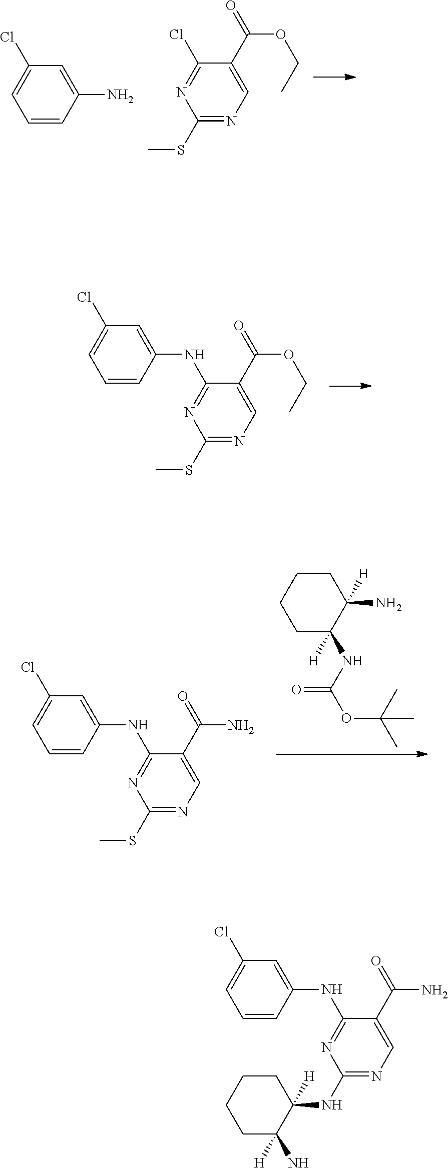 [SMILES: Nc1cccc(Cl)c1 ; CCOC(=O)c1cnc(SC)nc1Cl ; CCOC(=O)c1cnc(SC)nc1Nc1cccc(Cl)c1 ; CSc1ncc(C(N)=O)c(Nc2cccc(Cl)c2)n1 ; CC(C)(C)OC(=O)N[C@H]1CCCC[C@H]1N ; NC(=O)c1cnc(N[C@@H]2CCCC[C@@H]2N)nc1Nc1cccc(Cl)c1]

Ethyl 4-chloro-2-(methylthio)pyrimidine-5-carboxylate (1.00 g, 4.3 mmol) was dissolved in 20 mL DMF. To it were added 3-chloroaniline (0.55 mL, 5.2 mmol) and DIEA (1.50 mL, 8.6 mmol). The mixture was stirred at 80° C. for 3 h. To it were added LiOH (0.42 g, 17.2 mmol), 50 mL THF and 20 mL water. The mixture was stirred at 50° C. for 3 h. It was concentrated in vacuo to remove THF. To the mixture was added HCl to adjust the pH to 2. Solid carboxylic acid crashed out. It was isolated by filtration, washed with water and dried in vacuum oven. This solid was dissolved in 80 mL DMF. To it were added EDC.HCl (4.90 g, 25.5 mmol) and HOBt.H₂O (3.90 g, 25.5 mmol). The mixture was stirred for 1.5 h. To it was added ammonium hydroxide solution (28%, 4.3 mL, 68 mmol). The mixture was stirred for 2 h. To it was poured 300 mL water. Solid 4-((3-chlorophenyl)amino)-2-(methylthio)pyrimidine-5-carboxamide crashed out. It was collected by filtration, washed with water and dried in vacuum oven in quantitative yield.

4-((3-Chlorophenyl)amino)-2-(methylthio)pyrimidine-5-carboxamide (500 mg, 1.7 mmol) was dissolved in 20 mL NMP. To it was added MCPBA (77%, 580 mg, 2.6 mmol). The mixture was stirred for 20 m. To it were added DIEA (1.18 mL, 6.8 mmol) and tert-butyl (1S,2R)-2-aminocyclohexylcarbamate (740 mg, 3.4 mmol). The mixture was stirred at 90° C. for 5 h. It was diluted with 150 mL EtOAc, washed with 1N NaOH and brine, dried, concentrated in vacuo. The residue was treated with 1:1 TFA and DCM at RT for 1 h. It was concentrated and subjected to reverse phase preparative HPLC to isolate the title compound. MS found for C17H21ClN6O as (M+H)⁺ 361.3. UV: λ=244 nm.

### Example 42

### Preparation of 2-(((1R,2S)-2-aminocyclohexyl)amino)-4-(phenylamino)pyrimidine-5-carboxamide

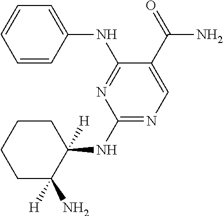 [SMILES: NC(=O)c1cnc(N[C@@H]2CCCC[C@@H]2N)nc1Nc1ccccc1]

The title compound was prepared according to the similar scheme illustrated in Example 41 for 2-(((1R,2S)-2-aminocyclohexyl)amino)-4-((3-chlorophenyl)amino)pyrimidine-5-carboxamide. MS found for C17H22N6O as (M+H)⁺ 327.3. UV: λ=244 nm.

### Example 43

### Preparation of 2-(((1R,2S)-2-aminocyclohexyl)amino)-4-(pyrazolo[1,5-a]pyridin-3-ylamino)pyrimidine-5-carboxamide

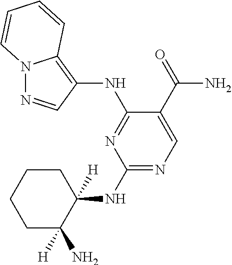 [SMILES: NC(=O)c1cnc(N[C@@H]2CCCC[C@@H]2N)nc1Nc1cnn2ccccc12]

The title compound was prepared according to the similar scheme illustrated in Example 41 for 2-(((1R,2S)-2-aminocyclohexyl)amino)-4-((3-chlorophenyl)amino)pyrimidine-5-carboxamide. MS found for C18H22N8O as (M+H)⁺ 367.4. UV: λ=227, 322 nm.

### Example 44

### Preparation of 2-(((1R,2S)-2-aminocyclohexyl)amino)-4-(thieno[2,3-b]pyridin-3-ylamino)pyrimidine-5-carboxamide

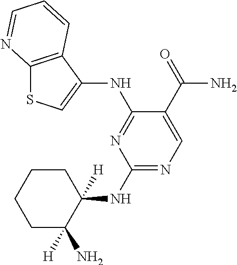 [SMILES: NC(=O)c1cnc(N[C@@H]2CCCC[C@@H]2N)nc1Nc1csc2ncccc12]

The title compound was prepared according to the scheme illustrated below:

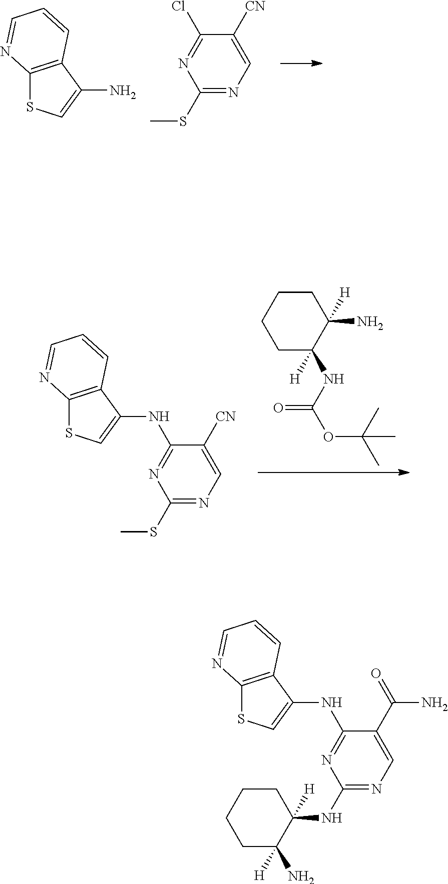 [SMILES: Nc1csc2ncccc12 ; CSc1ncc(C#N)c(Cl)n1 ; CSc1ncc(C#N)c(Nc2csc3ncccc23)n1 ; CC(C)(C)OC(=O)N[C@H]1CCCC[C@H]1N ; NC(=O)c1cnc(N[C@@H]2CCCC[C@@H]2N)nc1Nc1csc2ncccc12]

4-Chloro-2-(methylthio)pyrimidine-5-carbonitrile (410 mg, 2.22 mmol) was dissolved in 10 mL DMF. To it were added thieno[2,3-b]pyridin-3-amine (500 mg, 3.33 mmol) and DIEA (0.80 mL, 4.44 mmol). The mixture was stirred at 50° C. for 1 h. It was concentrated in vacuo and subjected to flash column to isolate 2-(methylthio)-4-(thieno[2,3-b]pyridin-3-ylamino)pyrimidine-5-carbonitrile.

2-(Methylthio)-4-(thieno[2,3-b]pyridin-3-ylamino)pyrimidine-5-carbonitrile (55 mg, 0.18 mmol) was dissolved in 5 mL NMP. To it was added MCPBA (70%, 70 mg, 0.28 mmol). The mixture was stirred at RT for 45 m. To it were added DIEA (0.13 mL, 0.72 mmol) and tert-butyl ((1S,2R)-2-aminocyclohexyl)carbamate (80 mg, 0.36 mmol). The mixture was stirred at 90° C. for 1 h. It was diluted with EtOAc, washed with 1N NaOH and brine, dried, concentrated in vacuo. The residue was treated with 1:1 DCM and TFA at RT for 15 m. It was concentrated in vacuo to dryness. It was dissolved in 3 mL DMSO. To it were added KOH (100 mg) and 1 mL H₂O₂ (50%). The mixture was stirred at RT for 30 m, diluted with acetonitrile, acidified with TFA, concentrated and subjected to reverse phase preparative HPLC to isolate the title compound (18 mg). MS found for C18H21N7OS as (M+H)⁺ 384.3. UV: λ=240, 297 nm.

### Example 45

### Preparation of 2-(((1R,2S)-2-aminocyclohexyl)amino)-4-(5-fluoropyridin-3-yl)amino)pyrimidine-5-carboxamide

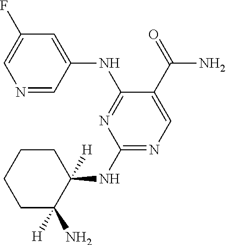 [SMILES: NC(=O)c1cnc(N[C@@H]2CCCC[C@@H]2N)nc1Nc1cncc(F)c1]

The title compound was prepared according to the scheme illustrated below:

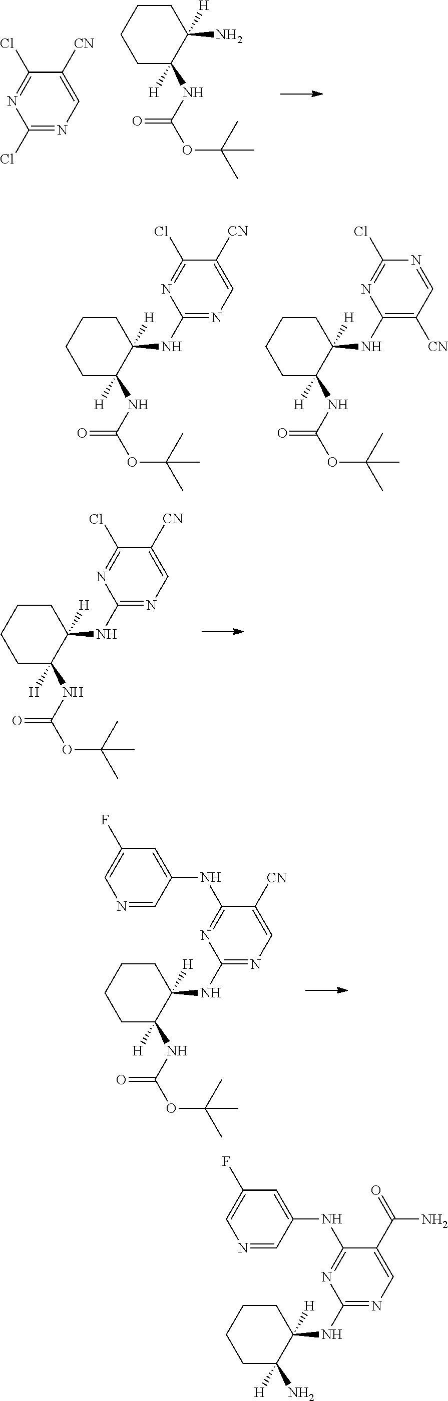

2,4-Dichloropyrimidine-5-carbonitrile (4.46 g, 25.6 mmol) was dissolved in 100 mL DMF. To it were added tert-butyl ((1S,2R)-2-aminocyclohexyl)carbamate (4.99 g, 23.3 mmol) and DIEA (6.1 mL, 35.0 mmol). The mixture was stirred at 45° C. for 20 m to afford two coupling product in about equal amount in quantitative yield. The mixture was concentrated in vacuo, diluted with EtOAc, washed with brine twice, dried, concentrated and subjected to flash column to separate tert-butyl ((1S,2R)-2-((4-chloro-5-cyanopyrimidin-2-yl)amino)cyclohexyl)carbamate.

The mixture of tert-butyl ((1S,2R)-2-((4-chloro-5-cyanopyrimidin-2-yl)amino)cyclohexyl)carbamate (200 mg, 0.57 mmol), 3-amino-5-fluoropyridine (192 mg, 1.71 mmol), fine powder Cs₂CO₃ (930 mg, 2.85 mmol), Q-Phos (43 mg, 0.06 mmol) and Pd(dba)₂ (35 mg, 0.06 mmol) in 30 mL toluene was degassed using argon stream and stirred at 105° C. under argon atmosphere for overnight. It was diluted with 200 mL EtOAc, vigorously stirred, filtered through celite, concentrated in vacuo and subjected to flash column to isolate tert-butyl ((1S,2R)-2-((5-cyano-4-((5-fluoropyridin-3-yl)amino)pyrimidin-2-yl)amino)cyclohexyl)carbamate. It was stirred at 80° C. in 5 mL TFA and 1 mL concentrated H₂SO₄ for 45 m. It was cooled to RT, diluted with 5 mL water, stirred and subjected to reverse phase preparative HPLC to isolate the title compound (31 mg). MS found for C16H20FN7O as (M+H)⁺ 346.3. UV: λ=244, 297 nm.

### Example 46

### Preparation of 2-(((1R,2S)-2-aminocyclohexyl)amino)-4-((5-methylpyridin-3-yl)amino)pyrimidine-5-carboxamide

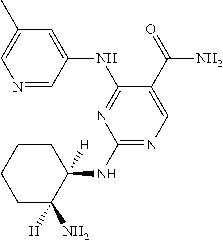 [SMILES: Cc1cncc(Nc2nc(N[C@@H]3CCCC[C@@H]3N)ncc2C(N)=O)c1]

The title compound was prepared according to the similar scheme illustrated in Example 45 for 2-(((1R,2S)-2-aminocyclohexyl)amino)-4-((5-fluoropyridin-3-yl)amino)pyrimidine-5-carboxamide. MS found for C17H23N7O as (M+H)⁺ 342.3. UV: λ=249 nm.

### Example 47

### Preparation of 2-(((1R,2S)-2-aminocyclohexyl)amino)-4-((5-methoxypyridin-3-yl)amino)pyrimidine-5-carboxamide

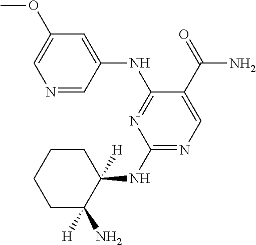 [SMILES: COc1cncc(Nc2nc(N[C@@H]3CCCC[C@@H]3N)ncc2C(N)=O)c1]

The title compound was prepared according to the similar scheme illustrated in Example 45 for 2-(((1R,2S)-2-aminocyclohexyl)amino)-4-((5-fluoropyridin-3-yl)amino)pyrimidine-5-carboxamide. MS found for C17H23N7O2 as (M+H)⁺ 358.3. UV: λ=244, 306 nm.

### Example 48

### Preparation of 4-((5-(1H-pyrazol-1-yl)pyridin-3-yl)amino)-2-(((1R,2S)-2-aminocyclohexyl)amino)pyrimidine-5-carboxamide

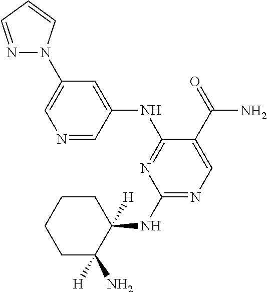 [SMILES: NC(=O)c1cnc(N[C@@H]2CCCC[C@@H]2N)nc1Nc1cncc(-n2cccn2)c1]

The title compound was prepared according to the similar scheme illustrated in Example 45 for 2-(((1R,2S)-2-aminocyclohexyl)amino)-4-((5-fluoropyridin-3-yl)amino)pyrimidine-5-carboxamide. MS found for C19H23N9O as (M+H)⁺ 394.3. UV: λ=254 nm. Preparation of 5-(1H-pyrazol-1-yl)pyridin-3-amine: The mixture of 5-iodopyridin-3-ylamine (1.00 g, 4.6 mmol), pyrazole (0.94 g, 13.8 mmol), fine powder K₃PO₄ (1.95 g, 9.2 mmol), fine powder CuI (270 mg, 0.14 mmol) and ethylenediamine (0.10 mL, 0.14 mmol) in 20 mL dioxane and 5 mL DMSO was stirred at 110° C. in a sealed tube for 1 day. The mixture was cooled to RT, diluted with 200 mL EtOAc, vigorously stirred, filtered through a silica plug, which was rinsed with about 200 mL EtOAc. The filtrate was washed with brine twice, dried, concentrated in vacuo and subjected to flash column using 10% methanol in DCM to afford the desired aniline (1.12 g).

### Example 49

### Preparation of 2-(((1R,2S)-2-aminocyclohexyl)amino)-4-((3-methylisothiazol-5-yl)amino)pyrimidine-5-carboxamide

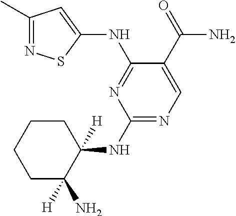 [SMILES: Cc1cc(Nc2nc(N[C@@H]3CCCC[C@@H]3N)ncc2C(N)=O)sn1]

The title compound was prepared according to the similar scheme illustrated in Example 45 for 2-(((1R,2S)-2-aminocyclohexyl)amino)-4-((5-fluoropyridin-3-yl)amino)pyrimidine-5-carboxamide, with 3-methylisothiazol-5-amine to replace 3-amino-5-fluoropyridine. MS found for C15H21N7OS as (M+H)⁺ 348.2. UV: λ=263, 301 nm.

### Example 50

### Preparation of 2-(((3R,4R)-3-aminotetrahydro-2H-pyran-4-yl)amino)-4-((3-chlorophenyl)amino)pyrimidine-5-carboxamide

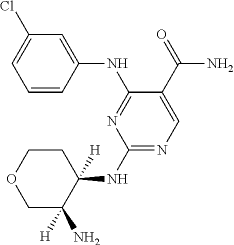 [SMILES: NC(=O)c1cnc(N[C@@H]2CCOC[C@@H]2N)nc1Nc1cccc(Cl)c1]

4-((3-Chlorophenyl)amino)-2-(methylthio)pyrimidine-5-carboxamide (see Example 41) (100 mg, 0.34 mmol) was dissolved in 5 mL NMP. To it was added MCPBA (70%, 126 mg, 0.51 mmol). The mixture was stirred for 50 m. To it were added DIEA (0.18 mL, 1.02 mmol) and tert-butyl ((3R,4R)-4-aminotetrahydro-2H-pyran-3-yl)carbamate (110 mg, 0.51 mmol). The mixture was stirred at 90° C. for 4 h. It was diluted with 100 mL EtOAc, washed with 1N NaOH and brine, dried, concentrated in vacuo, subjected to flash column to isolate tert-butyl ((3R,4R)-4-((5-carbamoyl-4-((3-chlorophenyl)amino)pyrimidin-2-yl)amino)tetrahydro-2H-pyran-3-yl)carbamate. It was treated with 1:1 TFA and DCM at RT for 1.5 h. The mixture was concentrated and subjected to reverse phase preparative HPLC to isolate the title compound (45 mg). MS found for C16H19ClN6O2 as (M+H)⁺ 363.3. UV: λ=241 nm.

### Example 51

### Preparation of 2-(((3R,4R)-3-aminotetrahydro-2H-pyran-4-yl)amino)-4-((4-chlorophenyl)amino)pyrimidine-5-carboxamide

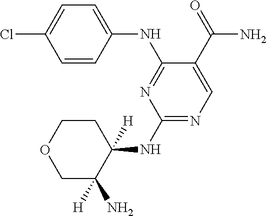 [SMILES: NC(=O)c1cnc(N[C@@H]2CCOC[C@@H]2N)nc1Nc1ccc(Cl)cc1]

The title compound was prepared according to the similar procedure shown in Example 50 for 2-(((3R,4R)-3-aminotetrahydro-2H-pyran-4-yl)amino)-4-(3-chlorophenyl)amino)pyrimidine-5-carboxamide. MS found for C16H19ClN6O2 as (M+H)⁺ 363.3. UV: λ=242, 287 nm.

### Example 52

### Preparation of 2-(((3R,4R)-3-aminotetrahydro-2H-pyran-4-yl)amino)-4-(phenylamino)pyrimidine-5-carboxamide

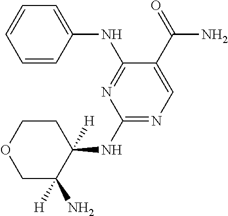 [SMILES: NC(=O)c1cnc(N[C@@H]2CCOC[C@@H]2N)nc1Nc1ccccc1]

The title compound was prepared according to the similar procedure shown in Example 50 for 2-(((3R,4R)-3-aminotetrahydro-2H-pyran-4-yl)amino)-4-((3-chlorophenyl)amino)pyrimidine-5-carboxamide. MS found for C16H20N6O2 as (M+H)⁺ 329.3. UV: λ=240 nm.

### Example 53

### Preparation of 2-(((3R,4R)-3-aminotetrahydro-2H-pyran-4-yl)amino)-4-((3-cyanophenyl)amino)pyrimidine-5-carboxamide

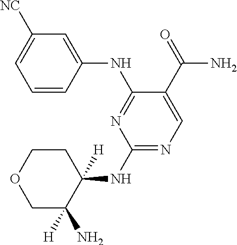 [SMILES: N#Cc1cccc(Nc2nc(N[C@@H]3CCOC[C@@H]3N)ncc2C(N)=O)c1]

The title compound was prepared according to the similar procedure shown in Example 50 for 2-(((3R,4R)-3-aminotetrahydro-2H-pyran-4-yl)amino)-4-((3-chlorophenyl)amino)pyrimidine-5-carboxamide. MS found for C17H19N7O2 as (M+H)⁺ 354.3. UV: λ=240, 282 nm.

### Example 54

### Preparation of 2-(((3R,4R)-3-aminotetrahydro-2H-pyran-4-yl)amino)-4-((4-cyanophenyl)amino)pyrimidine-5-carboxamide

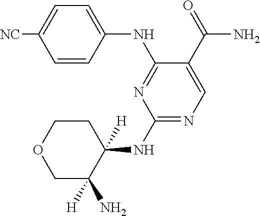

The title compound was prepared according to the similar procedure shown in Example 50 for 2-(((3R,4R)-3-aminotetrahydro-2H-pyran-4-yl)amino)-4-((3-chlorophenyl)amino)pyrimidine-5-carboxamide. MS found for C17H19N7O2 as (M+H)⁺ 354.4. UV: 2=249, 301 nm.

### Example 55

### Preparation of (R)-2-((1-amino-1-oxobutan-2-yl)amino)-4-(thieno[2,3-b]pyridin-3-ylamino)pyrimidine-5-carboxamide

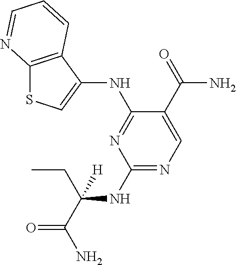 [SMILES: CC[C@@H](Nc1ncc(C(N)=O)c(Nc2csc3ncccc23)n1)C(N)=O]

2-(Methylthio)-4-(thieno[2,3-b]pyridin-3-ylamino)pyrimidine-5-carbonitrile (see Example 44) (50 mg, 0.16 mmol) was dissolved in 4 mL NMP. To it was added MCPBA (70%, 82 mg, 0.33 mmol). The mixture was stirred at RT for 1 h. To it were added DIEA (0.28 mL, 1.6 mmol) and (R)-2-aminobutanamide hydrochloride (111 mg, 0.80 mmol). The mixture was stirred at 120° C. for 1.5 h. It was diluted with EtOAc, washed with 1N NaOH and brine, dried, concentrated in vacuo, and subjected to flash column to isolate (R)-2-((5-cyano-4-(thieno[2,3-b]pyridin-3-ylamino)pyrimidin-2-yl)amino)butanamide. It was dissolved in 1 mL DMSO and 10 mL methanol. To it were added KOH (100 mg) and 1 mL H₂O₂ (50%). The mixture was stirred at RT for 30 m, diluted with acetonitrile, acidified with TFA, concentrated and subjected to reverse phase preparative HPLC to isolate the title compound (16 mg). MS found for C16H17N7O2S as (M+H)⁺ 372.3. UV: 2=240, 306 nm.

### Example 56

### Preparation of (R)-2-((1-amino-1-oxobutan-2-yl)amino)-4-(pyrazolo[1,5-a]pyridin-3-ylamino)pyrimidine-5-carboxamide

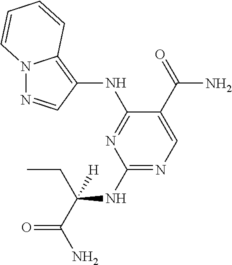 [SMILES: CC[C@@H](Nc1ncc(C(N)=O)c(Nc2cnn3ccccc23)n1)C(N)=O]

2-(Methylthio)-4-(pyrazolo[1,5-a]pyridin-3-ylamino)pyrimidine-5-carboxamide was prepared from pyrazolo[1,5-a]pyridin-3-amine using the similar scheme illustrated in Example 41. 2-(Methylthio)-4-(pyrazolo[1,5-a]pyridin-3-ylamino)pyrimidine-5-carboxamide (120 mg, 0.40 mmol) was dissolved in 5 mL NMP. To it was added MCPBA (70%, 190 mg, 0.80 mmol). The mixture was stirred at RT for 40 m. To it were added DIEA (0.70 mL, 4.0 mmol) and (R)-2-aminobutanamide hydrochloride (280 mg, 2.0 mmol). The mixture was stirred at 120° C. for 2 h. It was diluted with EtOAc, washed with 1N NaOH and brine, dried, concentrated in vacuo, and subjected to reverse phase preparative HPLC to isolate the title compound (65 mg). MS found for C16H18N8O2 as (M+H)⁺ 355.3. UV: λ=226, 325 nm.

### Example 57

### Preparation of (R)-2-((1-amino-1-oxobutan-2-yl)amino)-4-(quinolin-7-ylamino)pyrimidine-5-carboxamide

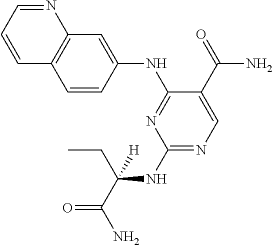 [SMILES: CC[C@@H](Nc1ncc(C(N)=O)c(Nc2ccc3cccnc3c2)n1)C(N)=O]

The title compound was prepared according to the similar procedure shown in Example 56 for (R)-2-((1-amino-1-oxobutan-2-yl)amino)-4-(pyrazolo[1,5-a]pyridin-3-ylamino)pyrimidine-5-carboxamide. MS found for C18H19N7O2 as (M+H)⁺ 366.2. UV: λ=240, 278 nm.

### Example 58

### Preparation of (R)-2-((1-amino-1-oxobutan-2-yl)amino)-4-((4-(thiazol-5-yl)phenyl)amino)pyrimidine-5-carboxamide

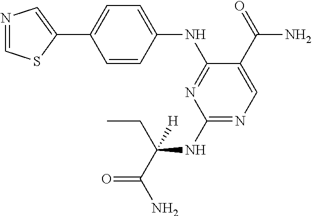 [SMILES: CC[C@@H](Nc1ncc(C(N)=O)c(Nc2ccc(-c3cncs3)cc2)n1)C(N)=O]

The title compound was prepared according to the similar procedure shown in Example 56 for (R)-2-((1-amino-1-oxobutan-2-yl)amino)-4-(pyrazolo[1,5-a]pyridin-3-ylamino)pyrimidine-5-carboxamide. MS found for C18H19N7O2S as (M+H)⁺ 398.3. UV: λ=240, 315 nm.

### Example 59

### Preparation of (R)-4-((4-(1,2,3-thiadiazol-5-yl)phenyl)amino)-2-((1-amino-1-oxobutan-2-yl)amino)pyrimidine-5-carboxamide

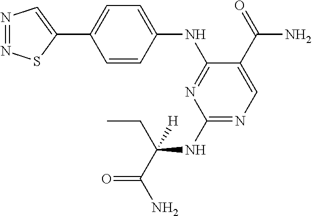 [SMILES: CC[C@@H](Nc1ncc(C(N)=O)c(Nc2ccc(-c3cnns3)cc2)n1)C(N)=O]

The title compound was prepared according to the similar procedure shown in Example 56 for (R)-2-((1-amino-1-oxobutan-2-yl)amino)-4-(pyrazolo[1,5-a]pyridin-3-ylamino)pyrimidine-5-carboxamide. MS found for C17H18N8O2S as (M+H)⁺ 399.3. UV: λ=232, 312 nm.

### Example 60

### Preparation of (R)-2-((1-amino-1-oxobutan-2-yl)amino)-4-((3-methoxyphenyl)amino)pyrimidine-5-carboxamide

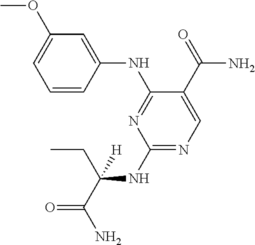 [SMILES: CC[C@@H](Nc1ncc(C(N)=O)c(Nc2cccc(OC)c2)n1)C(N)=O]

The title compound was prepared according to the similar procedure shown in Example 56 for (R)-2-((1-amino-1-oxobutan-2-yl)amino)-4-(pyrazolo[1,5-a]pyridin-3-ylamino)pyrimidine-5-carboxamide. MS found for C16H20N6O3 as (M+H)⁺ 345.3. UV: λ=244, 287 nm.

### Example 61

### Preparation of (R)-2-((1-amino-1-oxopentan-2-yl)amino)-4-((3-methoxyphenyl)amino)pyrimidine-5-carboxamide

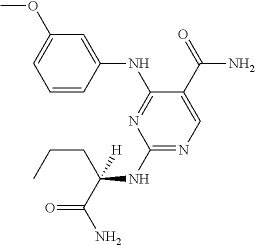 [SMILES: CCC[C@@H](Nc1ncc(C(N)=O)c(Nc2cccc(OC)c2)n1)C(N)=O]

The title compound was prepared according to the similar procedure shown in Example 60 for (R)-2-((1-amino-1-oxobutan-2-yl)amino)-4-((3-methoxyphenyl)amino)pyrimidine-5-carboxamide. MS found for C17H22N6O3 as (M+H)⁺ 359.2. UV: λ=244, 287 nm.

### Example 62

### Preparation of (R)-2-((1-amino-4-methyl-1-oxopentan-2-yl)amino)-4-((3-methoxyphenyl)amino)pyrimidine-5-carboxamide

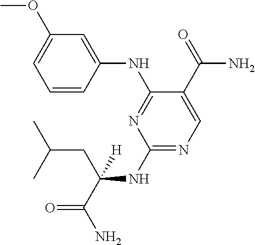 [SMILES: COc1cccc(Nc2nc(N[C@H](CC(C)C)C(N)=O)ncc2C(N)=O)c1]

The title compound was prepared according to the similar procedure shown in Example 60 for (R)-2-((1-amino-1-oxobutan-2-yl)amino)-4-((3-methoxyphenyl)amino)pyrimidine-5-carboxamide. MS found for C18H24N6O3 as (M+H)⁺ 373.3. UV: λ=244, 287 nm.

### Example 63

### Preparation of (R)-2-((1-amino-3-cyclopropyl-1-oxopropan-2-yl)amino)-4-((3-methoxyphenyl)amino)pyrimidine-5-carboxamide

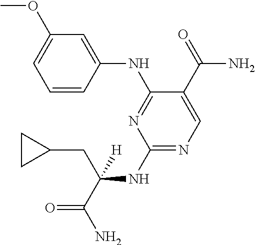 [SMILES: COc1cccc(Nc2nc(N[C@H](CC3CC3)C(N)=O)ncc2C(N)=O)c1]

The title compound was prepared according to the similar procedure shown in Example 60 for (R)-2-((1-amino-1-oxobutan-2-yl)amino)-4-((3-methoxyphenyl)amino)pyrimidine-5-carboxamide. MS found for C18H22N6O3 as (M+H)⁺ 371.3. UV: λ=244, 287 nm.

### Example 64

### Preparation of (R)-2-((2-amino-1-cyclopropyl-2-oxoethyl)amino)-4-((3-methoxyphenyl)amino)pyrimidine-5-carboxamide

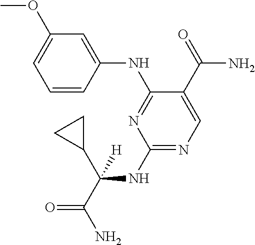 [SMILES: COc1cccc(Nc2nc(N[C@@H](C(N)=O)C3CC3)ncc2C(N)=O)c1]

The title compound was prepared according to the similar procedure shown in Example 60 for (R)-2-((1-amino-1-oxobutan-2-yl)amino)-4-((3-methoxyphenyl)amino)pyrimidine-5-carboxamide. MS found for C17H20N6O3 as (M+H)⁺ 357.3. UV: λ=244, 287 nm.

### Example 65

### Preparation of (R)-2-((1-amino-1-oxobutan-2-yl)amino)-4-((3-methylisothiazol-5-yl)amino)pyrimidine-5-carboxamide

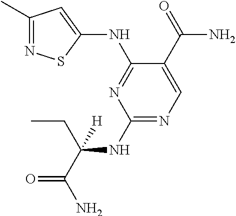 [SMILES: CC[C@@H](Nc1ncc(C(N)=O)c(Nc2cc(C)ns2)n1)C(N)=O]

The title compound was prepared according to the synthetic scheme illustrated below:

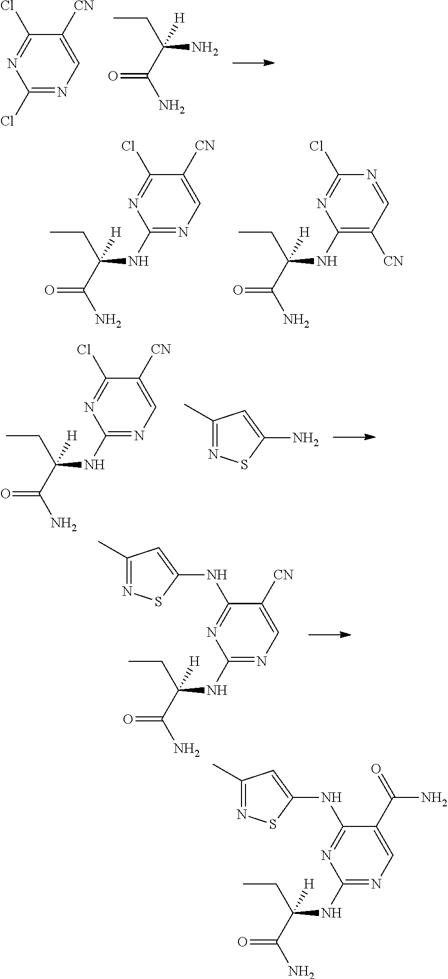

2,4-Dichloropyrimidine-5-carbonitrile (1.00 g, 5.75 mmol) was dissolved in 30 mL DMF. To it were added (R)-2-aminobutanamide hydrochloride (1.20 g, 8.62 mmol) and DIEA (3.0 mL, 17.24 mmol). The mixture was stirred at 50° C. for 10 m to afford two coupling product in about equal amount in quantitative yield. The mixture was concentrated in vacuo, diluted with EtOAc, washed with brine twice, dried, concentrated and subjected to flash column to separate (R)-2-((4-chloro-5-cyanopyrimidin-2-yl)amino)butanamide.

The mixture of (R)-2-((4-chloro-5-cyanopyrimidin-2-yl)amino)butanamide (400 mg, 1.7 mmol), 3-methylisothiazol-5-amine hydrochloride (770 mg, 5.1 mmol), fine powder Cs₂CO₃ (3.88 g, 11.9 mmol), BINAP (212 mg, 0.34 mmol) and Pd(OAc)₂ (77 mg, 0.34 mmol) in 50 mL dioxane was degassed using argon stream and stirred at 105° C. under argon atmosphere for 6 h. It was diluted with 400 mL EtOAc, vigorously stirred, filtered through celite, concentrated in vacuo and subjected to flash column to isolate (R)-2-((4-chloro-5-cyanopyrimidin-2-yl)amino)butanamide (210 mg).

It was dissolve in 4 mL DMSO. To it were added fine powder K₂CO₃ (185 mg, 1.32 mmol) and then 2 mL H₂O₂ (50%). The mixture was stirred at RT for 1 h, diluted with acetonitrile, acidified with TFA and subjected to reverse phase preparative HPLC to isolate the title compound (118 mg). MS found for C13H17N7O2S as (M+H)⁺ 336.1. UV: λ=268, 301 nm.

### Example 66

### Preparation of (R)-4-(1H-indazol-5-ylamino)-2-(1-amino-1-oxobutan-2-ylamino)pyrimidine-5-carboxamide

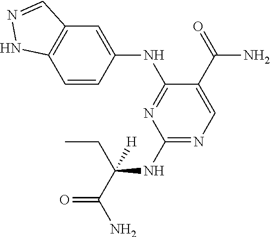 [SMILES: CC[C@@H](Nc1ncc(C(N)=O)c(Nc2ccc3[nH]ncc3c2)n1)C(N)=O]

The title compound was prepared as described in Scheme 1.

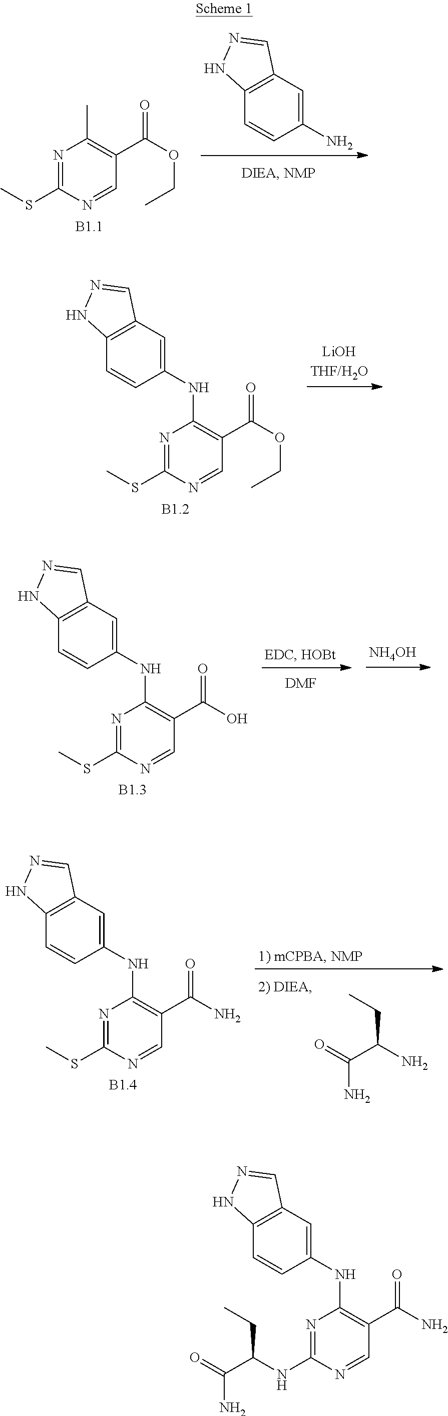

Step 1: Commercially available 4-chloro-2-(methylthio)pyrimidine-5-carboxylic acid ethyl ester was dissolved in NMP with an excess of DIEA. To this was added ˜1.1 eq of 5-aminoindazole. The reaction mixture was stirred at 90° C. for 2 hours. The mixture was cooled and water was added. Solid (crude B1.2) precipitated and was filtered.

Step 2: Ethyl ester B1.2 was dissolved in THF. To it were added lithium hydroxide hydrate and water. The mixture was stirred overnight and to it was carefully added 1N HCl solution until the pH was ˜3. The mixture was concentrated in vacuo to remove THF. Solid crashed out, was filtered, washed with water, and dried in vacuum oven to give compound B1.3 as a crude solid.

Step 3-4: Carboxylic acid B1.3 was dissolved in DMF. To it were added EDC hydrochloride and HOBt hydrate. The mixture was stirred at RT for 30 minutes. To it was then added concentrated ammonium hydroxide. The mixture was stirred for 30 minutes. Water was added, solid precipated and was filtered. The solid was washed with water and dried in a vacuum oven to give crude B1.4.

Step 5: Compound B1.4 was dissolved in ˜3 mL NMP. To it was added ˜2 eq mCPBA. The reaction mixture was stirred at RT for 45 minutes. To it then were added commercially available (R)-2-aminobutanamide HCl and DIEA. The mixture was stirred for 90 minutes at 120° C. bath. This mixture was then subjected to preparative HPLC to isolate the title compound. MS found for C₁₆H₁₈N₈O₂ as (M+H)⁺ 355.3.

### Example 67

### Preparation of (R)-4-(1H-indazol-6-ylamino)-2-(1-amino-1-oxobutan-2-ylamino)pyrimidine-5-carboxamide

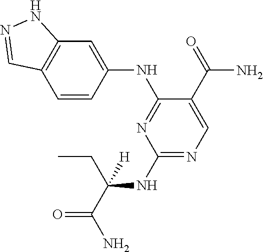 [SMILES: CC[C@@H](Nc1ncc(C(N)=O)c(Nc2ccc3cn[nH]c3c2)n1)C(N)=O]

The title compound was prepared as described in Scheme 1 utilizing 6-aminoindazole instead of 5-aminoindazole. MS found for C₁₆H₁₈N₈O₂ as (M+H)⁺ 355.3.

### Example 68

### Preparation of (R)-2-(1-amino-1-oxobutan-2-ylamino)-4-(quinolin-3-ylamino)pyrimidine-5-carboxamide

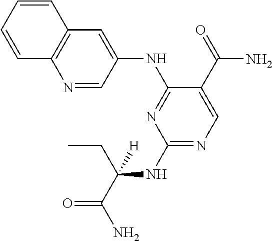 [SMILES: CC[C@@H](Nc1ncc(C(N)=O)c(Nc2cnc3ccccc3c2)n1)C(N)=O]

The title compound was prepared as described in Scheme 1 utilizing 3-aminoquinoline instead of 5-aminoindazole. MS found for C₁₈H₁₉N₇O₂ as (M+H)⁺ 366.3.

### Example 69

### Preparation of (R)-2-(1-amino-3-methyl-1-oxobutan-2-ylamino)-4-(quinolin-6-ylamino)pyrimidine-5-carboxamide

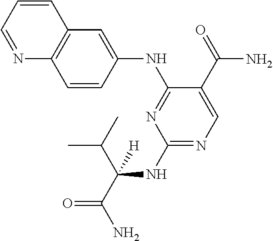 [SMILES: CC(C)[C@@H](Nc1ncc(C(N)=O)c(Nc2ccc3ncccc3c2)n1)C(N)=O]

The title compound was prepared as described in Scheme 1 utilizing 6-aminoquinoline instead of 5-aminoindazole and H-D-Val-NH₂HCl instead of (R)-2-aminobutanamide. MS found for C₁₉H₂₁N₇O₂ as (M+H)⁺ 380.1.

### Example 70

### Preparation of (R)-2-(1-amino-3-methyl-1-oxobutan-2-ylamino)-4-(quinolin-6-ylamino)pyrimidine-5-carboxamide N-oxide

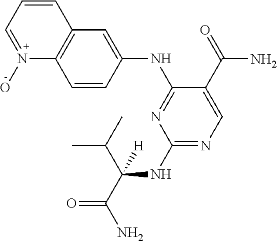

The title compound was prepared as described in Scheme 1 utilizing 6-aminoquinoline instead of 5-aminoindazole and H-D-Val-NH₂HCl instead of (R)-2-aminobutanamide. This material was isolated as a side product due to excess mCPBA. MS found for C₁₉H₂₁N₇O₃ as (M+H)⁺ 396.1.

### Example 71

### Preparation of (R)-2-(1-amino-1-oxobutan-2-ylamino)-4-(isoquinolin-7-ylamino)pyrimidine-5-carboxamide

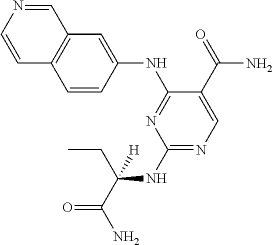 [SMILES: CC[C@@H](Nc1ncc(C(N)=O)c(Nc2ccc3ccncc3c2)n1)C(N)=O]

The title compound was prepared as described in Scheme 1 utilizing 7-aminoisoquinoline instead of 5-aminoindazole. MS found for C₁₈H₁₉N₇O₂ as (M+H)⁺ 366.2.

### Example 72

### Preparation of (R)-2-(2-amino-1-cyclopropyl-2-oxoethylamino)-4-(quinolin-6-ylamino)pyrimidine-5-carboxamide

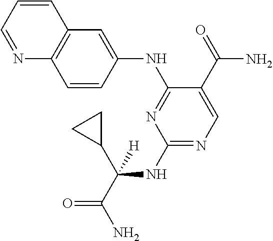 [SMILES: NC(=O)c1cnc(N[C@@H](C(N)=O)C2CC2)nc1Nc1ccc2ncccc2c1]

The title compound was prepared as described in Scheme 1 utilizing 6-aminoquinoline instead of 5-aminoindazole and (R)-2-amino-2-cyclopropylacetamide instead of (R)-2-aminobutanamide MS found for C₁₉H₁₉N₇O₂ as (M+H)⁺ 378.2.

### Example 73

### Preparation of 2-((1R,2S)-2-aminocyclohexylamino)-4-(isoquinolin-7-ylamino)pyrimidine-5-carboxamide

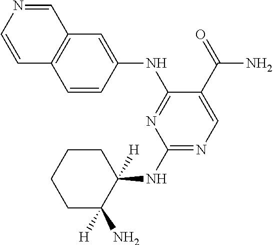 [SMILES: NC(=O)c1cnc(N[C@@H]2CCCC[C@@H]2N)nc1Nc1ccc2ccncc2c1]

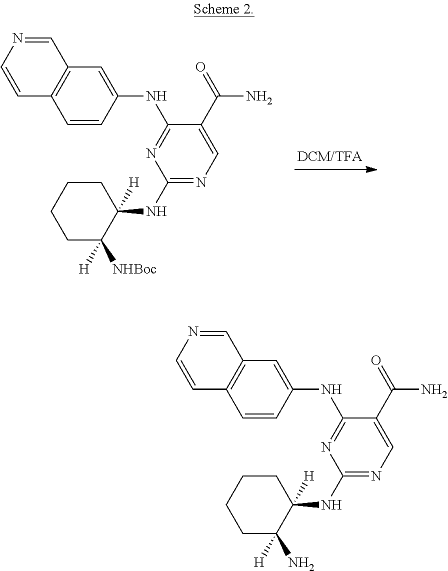 [SMILES: CC(C)(C)OC(=O)N[C@H]1CCCC[C@H]1Nc1ncc(C(N)=O)c(Nc2ccc3ccncc3c2)n1 ; NC(=O)c1cnc(N[C@@H]2CCCC[C@@H]2N)nc1Nc1ccc2ccncc2c1]

The title compound was prepared as described in Scheme 1 utilizing 7-aminoisoquinoline instead of 5-aminoindazole and tert-butyl (1S,2R)-2-aminocyclohexylcarbamate instead of (R)-2-aminobutanamide. Additionally a Boc-deprotection utilizing a DCM/TFA mixture (˜2:1) was undertaken. MS found for C₂₀H₂₃N₇O as (M+H)⁺ 378.3.

### Example 74

### Preparation of (R)-2-(1-amino-3-cyclopropyl-1-oxopropan-2-ylamino)-4-(quinolin-3-ylamino)pyrimidine-5-carboxamide

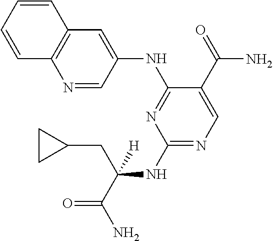 [SMILES: NC(=O)c1cnc(N[C@H](CC2CC2)C(N)=O)nc1Nc1cnc2ccccc2c1]

The title compound was prepared as described in Scheme 1 utilizing 3-aminoquinoline instead of 5-aminoindazole and (R)-2-amino-3-cyclopropylpropanamide instead of (R)-2-aminobutanamide. MS found for C₂₀H₂₁N₇O₂ as (M+H)⁺ 392.3.

### Example 75

### Preparation of 2-((1R,2S)-2-aminocyclohexylamino)-4-(quinolin-7-ylamino)pyrimidine-5-carboxamide

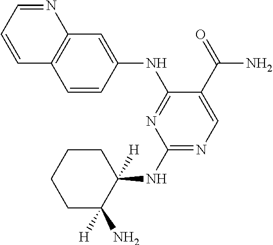 [SMILES: NC(=O)c1cnc(N[C@@H]2CCCC[C@@H]2N)nc1Nc1ccc2cccnc2c1]

The above compound was prepared as described in Scheme 1 utilizing 7-aminoquinoline instead of 5-aminoindazole and tert-butyl (1S,2R)-2-aminocyclohexylcarbamate instead of (R)-2-aminobutanamide. Similar to Scheme 2, a Boc-deprotection utilizing DCM/TFA afforded the title compound. MS found for C₂₀H₂₃N₇O as (M+H)⁺ 378.2.

### Example 76

### Preparation of (S)-2-(2-amino-4,4-difluorobutylamino)-4-(1-ethyl-1H-indol-4-ylamino)pyrimidine-5-carboxamide

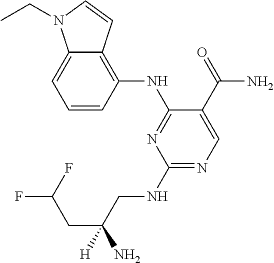 [SMILES: CCn1ccc2c(Nc3nc(NC[C@@H](N)CC(F)F)ncc3C(N)=O)cccc21]

The above compound was prepared as described in Scheme 1 utilizing 1-ethyl-1H-indol-4-amine instead of 5-aminoindazole and (S)-benzyl 1-amino-4,4-difluorobutan-2-ylcarbamate instead of (R)-2-aminobutanamide. The synthesis of and (S)-benzyl 1-amino-4,4-difluorobutan-2-ylcarbamate is detailed in Scheme 3 (below).

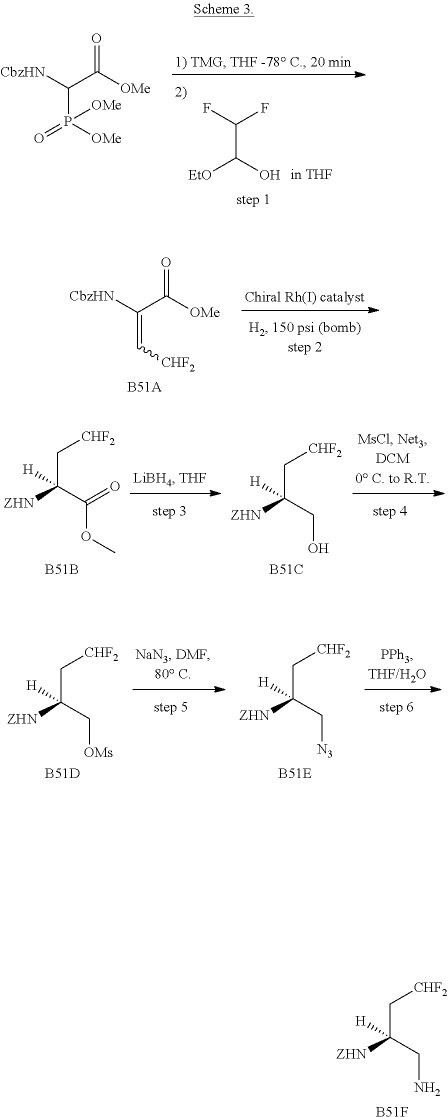

Step 1: To 10.2 g (30.8 mmol) of N-(Benzyloxycarbonyl)phosphonoglycine trimethyl ester in ˜120 mL of THF at −78° C. was added 4.25 mL 1,1,3,3-tetra-methyl-guanidine. The reaction was stirred at −78° C. for 20 minutes and then 4.27 g (33.9 mmol) of 1-ethoxy-2,2-difluoroethanol in ˜15 mL THF was added dropwise. The reaction was stirred at −78° C. for 30 minutes and then was warmed to room temperature. The THF was removed in vacuo and then the resulting residue was dissolved in EtOAc. The organic layer was washed with cold H₂O and the aqueous layer was further extracted with EtOAc. The combined organics were concentrated in vacuo. The resulting crude product (9.2 g) was subjected to normal phase silica chromatography eluting with gradient starting at 10% EtOAc in hexanes, finishing at 20% EtOAc in hexanes. The product B51A (5.20 g) was isolated as a mixture of E and Z isomers. An NMR in CDCl₃ matched spectra reported by Hu et al Tet Lett (2008), 49(5), 901-902.

Step 2: To 4.77 g of B51A in 125 mL MeOH was added 146 mg 1,2-Bis[(2S,5S)-2,5-diethylphospholano]benzene (1,5-cyclooctadiene)rhodium(I) trifluoromethanesulfonate. The resulting solution was degassed with Ar for 5 minutes and then subjected to H₂ at 150 psi for 12 hours. The MeOH was removed to give 4.81 g of crude B51B which was run through a short silica column using 30% EtOAc in hexanes. Isolated product was massed to be 4.72 g.

Step 3: To 4.72 g (16.4 mmol) of B51B in ˜50 mL THF at 0° C. was added LiBH₄ (716 mg, 32.9 mmol). The reaction was allowed to warm to room temperature and was stirred for an additional 30 minutes. The reaction was quenched with saturated NH₄Cl (aq) and EtOAc was added to extract product. The combined organics were washed with brine and concentrated to give 4.23 g of B51C which was used without further purification.

Steps 4 and 5: To B51C dissolved in DCM at 0° C. was added triethylamine. To it was added a solution of MsCl in DCM, dropwise. The mixture was stirred for 2 hours, diluted with more DCM and washed with water. The organic layer was dried (Na₂SO₄) and concentrated in vacuo. This resulting crude material B51D was dissolved in DMF. To it were added NaN₃. The mixture was stirred at 80° C. for 2 h. It was cooled to RT and diluted with EtOAc. The organic layer wash washed with 0.5 N NaOH, water, and brine. The organic solution was dried over MgSO₄ and concentrated. An Isco silica column was run as to isolate pure B51E

Step 6: To 4.16 g (14.5 mmol) of B51E in ˜40 mL THF was added 5.72 g (21.8 mmol) PPh₃ and 6 mL H₂O. The reaction was stirred at 60° C. for 3 hours and then volatiles were removed to give 10.4 g of crude. The crude reaction mixture was subjected to normal phase silica chromatography using a gradient of MeOH in DCM from 0 to 20%, resulting in 3.07 g of pure B51F.

The amine (B51F) was reacted with 4-(1-ethyl-1H-indol-4-ylamino)-2-(methylthio)pyrimidine-5-carboxamide as seen below in Scheme B4, utilizing similar chemistry as previously described in Example 1 (note that the OBt derivative is utilized here and that the mCPBA step is unnecessary). In the final step, deprotection of the Cbz-protected amine was carried out in EtOAc utilizing Pd/C and a balloon filled with H₂. After completion, the mixture was filtered through celite, washed with MeOH and the title compound was purified by rpHPLC. MS found for C₁₉H₂₃F₂N₇O as (M+H)⁺404.2.

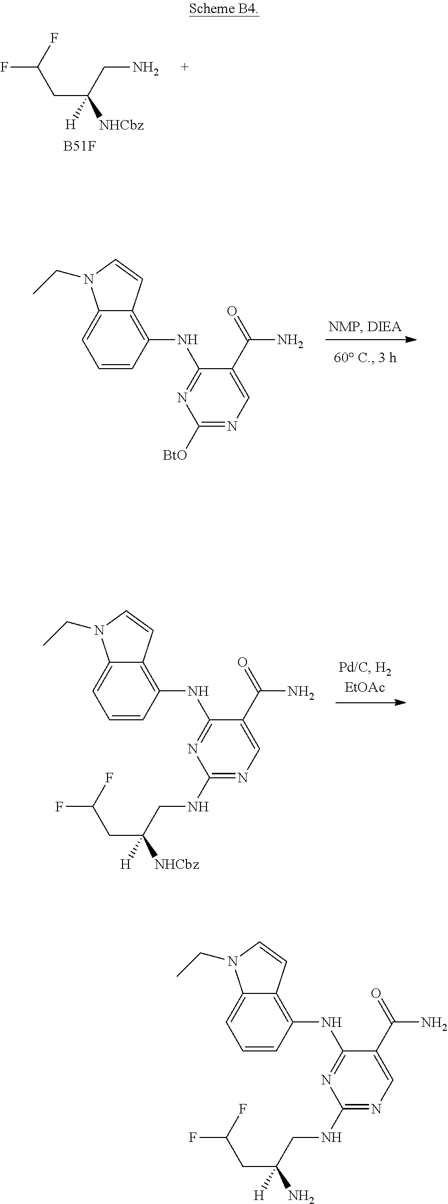

The amine (B51F) was reacted with 2-(1H-benzo[d][1,2,3]triazol-1-yloxy)-4-(1-ethyl-1H-indol-4-ylamino)pyrimidine-5-carboxamide as seen in Scheme B4. Note that since the OBt derivative is utilized here, the mCPBA step is unnecessary. In the final step, deprotection of the Cbz-protected amine was carried out in EtOAc utilizing Pd/C and a balloon filled with H₂. After completion, the mixture was filtered through celite, washed with MeOH and the title compound was purified by rpHPLC. MS found for C₁₉H₂₃F₂N₇O as (M+H)⁺404.2.

### Example 77

### Preparation of (R)-2-(1-amino-3-methyl-1-oxobutan-2-ylamino)-4-(quinolin-7-ylamino)pyrimidine-5-carboxamide

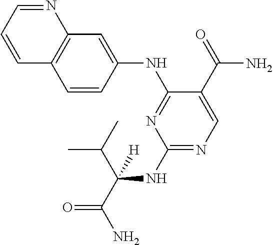 [SMILES: CC(C)[C@@H](Nc1ncc(C(N)=O)c(Nc2ccc3cccnc3c2)n1)C(N)=O]

The above compound was prepared as described in Scheme 5.

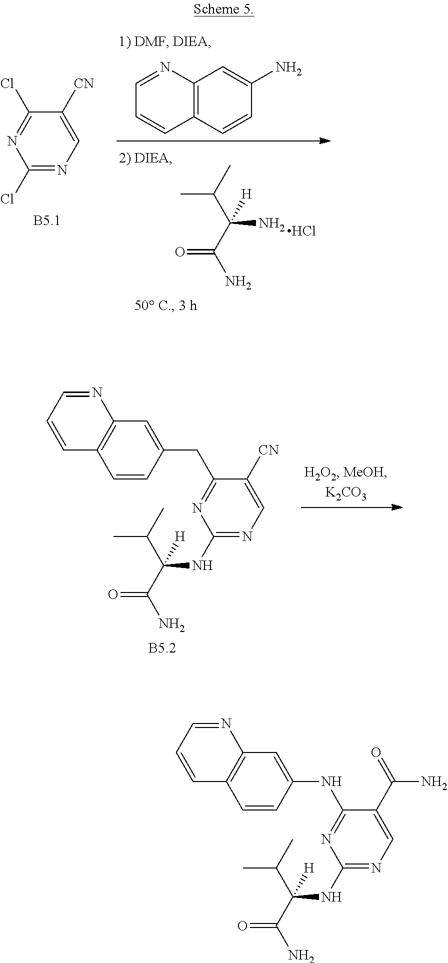

Commercially available B5.1 (1.02 g, 5.86 mmol) was dissolved in ˜30 mL DMF with 1.5 mL DIEA. To this stirring solution was added 850 mg (5.86 mmol, 1 eq) of 7-aminoquinoline. The reaction was stirred at room temperature for 30 minutes. An aliquot (˜0.5 mmol) was taken aside, and to it was added 0.1 mL DIEA and H-D-Val-NH₂HCl. This reaction mixture was stirred for 3 hours at 50° C. and then cooled. Water and DCM was added and the layers were separated. The organic layer was washed with 10% NaHCO₃ and was concentrated. Crude B5.2 was dissolved in ˜10 mL MeOH. To this solution, ˜50 mg K₂CO₃ and ˜1 mL H₂O₂ (40% by wt) were added. The reaction was stirred at 50° C. for 30 minutes and then was concentrated. The crude was purified by rpHPLC to give the title compound. MS found for C₁₉H₂₁N₇O₂ as (M+H)⁺380.2.

### Example 78

### Preparation of (S)-4-(m-toluidino)-2-(2-amino-4,4-difluorobutylamino)pyrimidine-5-carboxamide

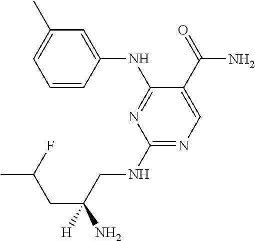 [SMILES: Cc1cccc(Nc2nc(NC[C@@H](N)CC(C)F)ncc2C(N)=O)c1]

The above compound was prepared as described in Scheme 1 utilizing m-toluidine instead of 5-aminoindazole and (S)-benzyl 1-amino-4,4-difluorobutan-2-ylcarbamate (B51F, example 76) instead of (R)-2-aminobutanamide. Additionally a Cbz-deprotection utilizing Pd/C and H₂ as described in Scheme B4 (example B12) was undertaken. MS found for C₁₆H₂₀F₂N₆O as (M+H)⁺351.2.

### Example 79

### Preparation of (S)-4-(3-(2H-1,2,3-triazol-2-yl)phenylamino)-2-(2-amino-4,4-difluorobutylamino)pyrimidine-5-carboxamide

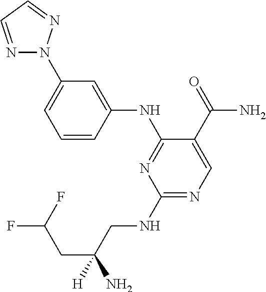 [SMILES: NC(=O)c1cnc(NC[C@@H](N)CC(F)F)nc1Nc1cccc(-n2nccn2)c1]

The above compound was prepared as described in Scheme B4 utilizing 3-(2H-1,2,3-triazol-2-yl)aniline instead of 5-aminoindazole and (S)-benzyl 1-amino-4,4-difluorobutan-2-ylcarbamate (B51F, example 76) instead of (R)-2-aminobutanamide. A Cbz-deprotection utilizing Pd/C and H₂ as described in Scheme B4 (example B12) was undertaken. MS found for C₁₇H₁₉F₂N₉O as (M+H)⁺404.2.

### Example 80

### Preparation of (S)-4-(p-toluidino)-2-(2-aminopropylamino)pyrimidine-5-carboxamide

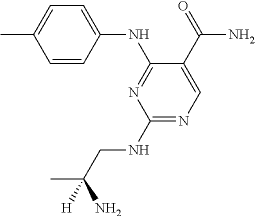 [SMILES: Cc1ccc(Nc2nc(NC[C@H](C)N)ncc2C(N)=O)cc1]

The above compound was prepared as described in Scheme 6.

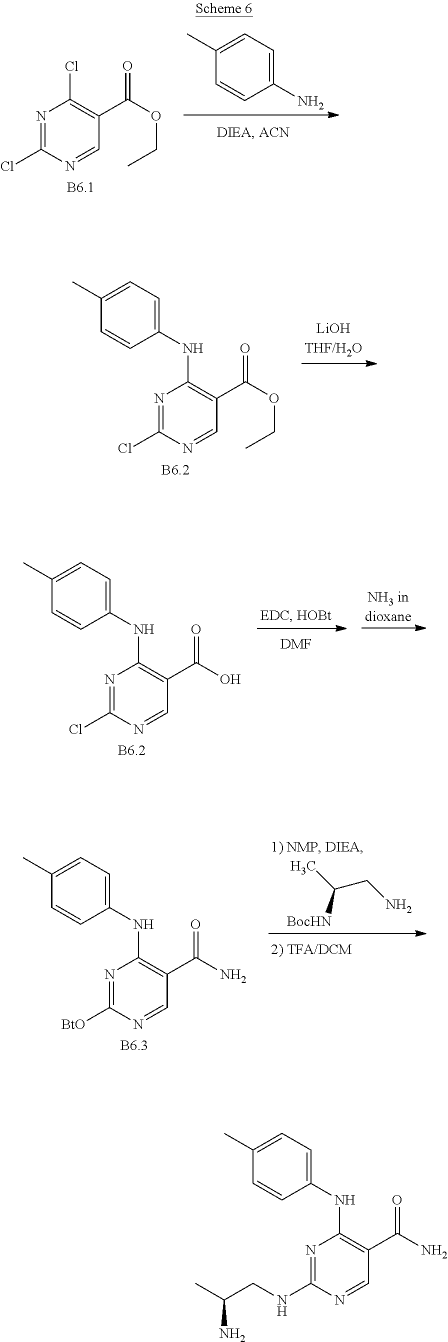

To 1.58 g (7.18 mmol) in ACN was added 920 mg (8.6 mmol) p-toluidine and ˜2 mL DIEA. The reaction was stirred at room temperature for ˜1 hour. The ACN was removed in vacuo until solid precipitated. At this point water (˜50 mL) was added and the solid was filtered. The solid (B6.2) was dried and massed to be 1.85 g (89% yield). To 1.01 g (3.47 mmol) of B6.2 in a 50/50 mixture of THF/H₂O was added 4.16 mmol of LiOH in H₂O (1.2 eq). The reaction was stirred at room temperature for 30 minutes and then was acidified to pH˜2 with 1 M HCl (aq). The volatiles were removed and the resulting solid was filtered to give 613 mg B6.2. To B6.2 in DMF was added 662 mg HOBtH₂O and 708 mg EDCHCl. After stirring for 2 hours and additional 0.5 eq of EDC HCl was added. The reaction was stirred for 1 hour and then NH₃ in dioxane (excess) was added. The reaction was stirred for 45 minutes and then the dioxane was removed in vacuo. Water was added which led to a precipitate. The solid (B6.3) was filtered, washed with water and dried. To ˜200 mg B6.3 in ˜2 mL NMP and 0.3 mL DIEA was added 240 mg (S)-tert-butyl 1-aminopropan-2-ylcarbamate. The reaction mixture was stirred at 90° C. for 2 hours and was then cooled. Water was added, solid precipitated and was filtered. Similar to Scheme 2 (example 73), a Boc-deprotection utilizing DCM/TFA afforded the title compound. MS found for C₁₅H₂₀N₆O as (M+H)⁺301.2.

### Example 81

### Preparation of 4-(p-toluidino)-2-(1R,2S)-2-aminocyclohexylamino)pyrimidine-5-carboxamide

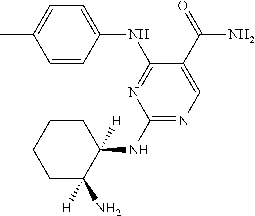 [SMILES: Cc1ccc(Nc2nc(N[C@@H]3CCCC[C@@H]3N)ncc2C(N)=O)cc1]

The title compound was prepared as described in Scheme 6 utilizing tert-butyl (1S,2R)-2-aminocyclohexylcarbamate instead of (5)-tert-butyl 1-aminopropan-2-ylcarbamate. MS found for C₁₈H₂₄N₆O as (M+H)⁺341.2.

### Example 82

### Preparation of (S)-2-(2-amino-4,4-difluorobutylamino)-4-(3-cyanophenylamino)pyrimidine-5-carboxamide

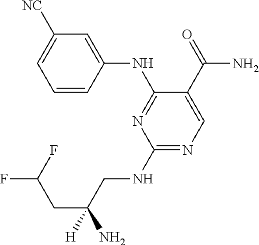 [SMILES: N#Cc1cccc(Nc2nc(NC[C@@H](N)CC(F)F)ncc2C(N)=O)c1]

The above compound was prepared as described in Scheme 6 utilizing a 3-aminobenzonitrile benzotriazole intermediate instead of the p-toluidine benzotriazole intermediate (B6.3) and (S)-benzyl 1-amino-4,4-difluorobutan-2-ylcarbamate (B51F, example 76) instead of (S)-tert-butyl 1-aminopropan-2-ylcarbamate. Here, a Cbz-deprotection utilizing BBr₃ in DCM (as described below in Scheme 7) was undertaken to give the title compound.

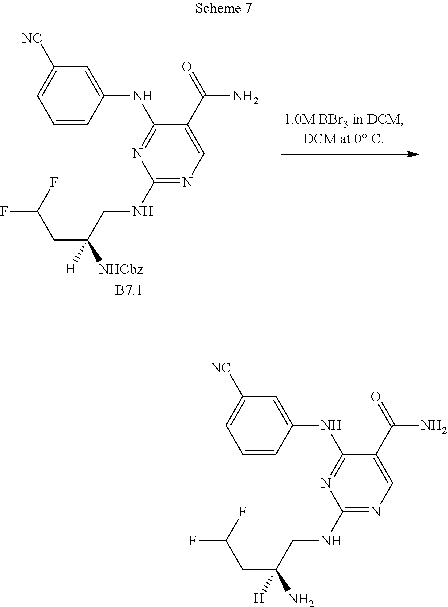 [SMILES: N#Cc1cccc(Nc2nc(NC[C@H](CC(F)F)NC(=O)OCc3ccccc3)ncc2C(N)=O)c1 ; N#Cc1cccc(Nc2nc(NC[C@@H](N)CC(F)F)ncc2C(N)=O)c1]

To ˜100 mg B7.1 in 4 mL DCM at 0° C. was added 0.40 mL of 1.0 M BBr₃ in DCM. The reaction was stirred at 0° C. for 20 minutes and then was concentrated. A 3:7 mixture of ACN/H₂O (10 mL total) was added to the concentrate along with a couple of drops of TFA. The mixture was filtered and then subjected to rpHPLC. MS found for C₁₆H₁₇F₂N₇O as (M+H)⁺362.4.

### Example 83

### Preparation of (S)-4-(p-toluidino)-2-(2-amino-4,4-difluorobutylamino)pyrimidine-5-carboxamide

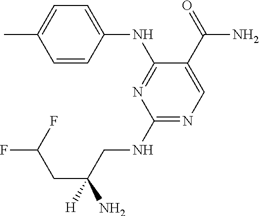 [SMILES: Cc1ccc(Nc2nc(NC[C@@H](N)CC(F)F)ncc2C(N)=O)cc1]

The above compound was prepared as described in Scheme 6 utilizing B6.3 and (S)-benzy(11-amino-4,4-difluorobutan-2-ylcarbamate (B51F, example 76) instead of (S)-tert-butyl 1-aminopropan-2-ylcarbamate. Additionally a Cbz-deprotection utilizing BBr₃ in DCM (Scheme 7, example 82) was undertaken to give the title compound. MS found for C₁₆H₂₀F₂N₆O as (M+H)⁺351.5.

### Example 84

### Preparation of (S)-2-(2-amino-4,4-difluorobutylamino)-4-(1-(2,2,2-trifluoroethyl)-1H-indol-4-ylamino)pyrimidine-5-carboxamide

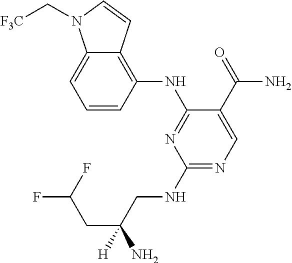 [SMILES: NC(=O)c1cnc(NC[C@@H](N)CC(F)F)nc1Nc1cccc2c1ccn2CC(F)(F)F]

The above compound was prepared as described in Scheme 1 utilizing 1-(2,2,2-trifluoroethyl)-1H-indol-4-amine instead of 5-aminoindazole and (S)-benzyl 1-amino-4,4-difluorobutan-2-ylcarbamate (B51F, example 76) instead of (R)-2-aminobutanamide. The synthesis of 1-(2,2,2-trifluoroethyl)-1H-indol-4-amine is outlined in Scheme 8.

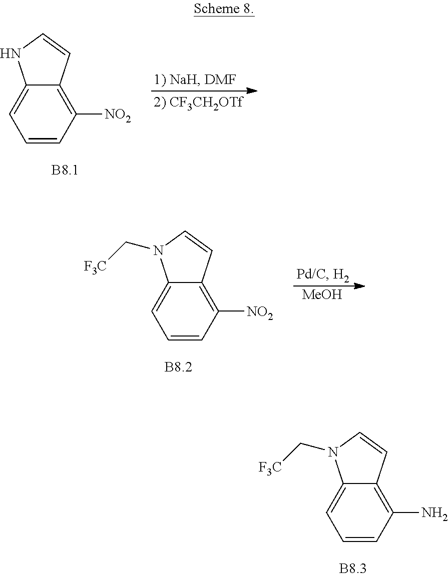 [SMILES: "MeOH" ; O=[N+]([O-])c1cccc2[nH]ccc12 ; O=[N+]([O-])c1cccc2c1ccn2CC(F)(F)F ; Nc1cccc2c1ccn2CC(F)(F)F]

To 720 mg (4.44 mmol) 4-nitroindole (B8.1, commercially available) in ˜25 mL DMF at 0° C. was added 213 mg NaH (60% dispersion in hexanes). The reaction was stirred at 0° C. for 5 minutes and then for 15 minutes at room temperature. The reaction was chilled back down to 0° C. and then 1.24 g (5.33 mmol) of 2,2,2-trifluoroethyl trifluoromethanesulfonate was added dropwise. The reaction mixture was allowed to warm to room temperature and was stirred overnight. The reaction was quenched with 5 mL saturated NaHCO₃ (aq) and then 25 mL water was added. Solid precipitated and was filtered. The filtered solid which still contained some staring material was carried forward. To this crude material (B8.2) dissolved in MeOH was added Pd/C and a balloon filled with H₂. The reaction was stirred overnight before being filtered through celite and concentrated. This yielded crude 1-(2,2,2-trifluoroethyl)-1H-indol-4-amine (B8.3) which was utilized instead of 5-aminoindazole (Scheme 1). Additionally a Cbz-deprotection utilizing BBr₃ in DCM (Scheme 7, example 82) was undertaken to give the title compound. MS found for C₁₆H₂₀F₂N₆O as (M+H)⁺458.2.

### Example 85

### Preparation of (S)-2-(2-amino-4,4-difluorobutylamino)-4-(1-methyl-1H-indol-4-ylamino)pyrimidine-5-carboxamide

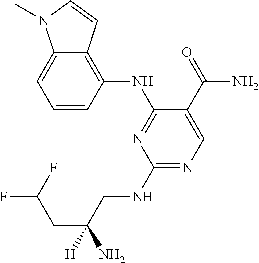 [SMILES: Cn1ccc2c(Nc3nc(NC[C@@H](N)CC(F)F)ncc3C(N)=O)cccc21]

The above compound was prepared as described in Scheme 1 utilizing 1-methyl-1H-indol-4-amine instead of 5-aminoindazole and (S)-benzyl 1-amino-4,4-difluorobutan-2-ylcarbamate (B51F, example 76) instead of (R)-2-aminobutanamide. Additionally a Cbz-deprotection utilizing BBr₃ in DCM (Scheme 7, example 82) was undertaken to give the title compound. MS found for C₁₈H₂₁F₂N₇O as (M+H)⁺390.2.

### Example 86

### Preparation of (S)-2-(2-amino-4,4-difluorobutylamino)-4-(7-fluoro-1-methyl-1H-indol-4-ylamino)pyrimidine-5-carboxamide

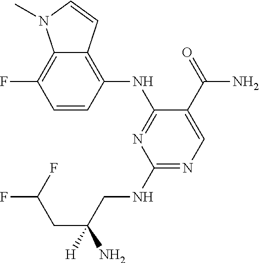 [SMILES: Cn1ccc2c(Nc3nc(NC[C@@H](N)CC(F)F)ncc3C(N)=O)ccc(F)c21]

The above compound was prepared as described in Scheme 1 utilizing 7-fluoro-1-methyl-1H-indol-4-amine instead of 5-aminoindazole and (S)-benzyl 1-amino-4,4-difluorobutan-2-ylcarbamate (B51F, example 76) instead of (R)-2-aminobutanamide. The synthesis of 7-fluoro-1-methyl-1H-indol-4-amine is detailed in Scheme 9 (below).

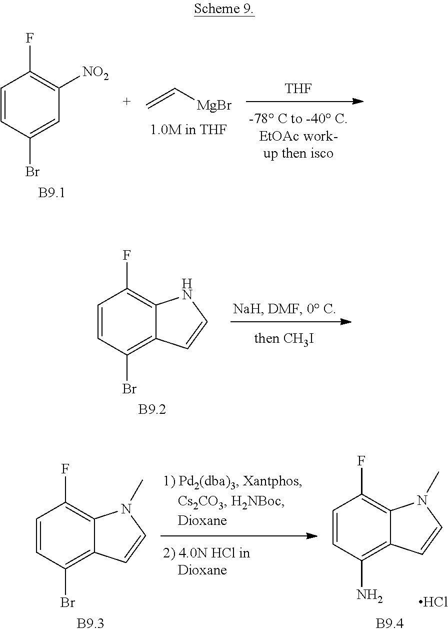

To 2.42 g of 3-bromo-6-fluoronitrobenzene (B9.1) in ˜30 mL THF at −78° C. was added 37 mL 1.0 M vinylmagnesium bromide (in THF). The reaction was stirred at −78° C. for 10 minutes and then at −40° C. for 1.5 hours. The reaction mixture was quenched with saturated NH₄Cl. The layers were separated and the aqueous layer was further extracted with EtOAc. The combined organics were washed with brine and concentrated. The residue was dissolved in DCM and an Isco silica column was run utilizing 100% DCM. Overall, 580 mg B9.2 was obtained. This indole intermediate was alkylated with MeI utilizing analogous procedures as described in Scheme 8. Once B9.3 was in hand, it was reacted with H₂NBoc in dioxane, with Pd₂(dba)₃, Xantphos, and Cs₂CO₃. The reaction mixture was stirred under an argon atmosphere at reflux overnight. The reaction was cooled and dioxane was removed in vacuo. The resulting solid was suspended in DCM and filtered. The filtrate was partitioned with water and the organic layer was removed and concentrated. This resulted in a residue which was treated with 4.0 N HCl in dioxane (excess). The reaction was stirred for 2 hours and then concentrated to give B9.4 which was utilized instead of 5-aminoindazole (Scheme 1). Additionally a Cbz-deprotection utilizing BBr₃ in DCM (Scheme 7, example 82) was undertaken to give the title compound. MS found for C₁₈H₂₀F₃N₇O as (M+H)⁺408.3.

### Example 87

### Preparation of (S)-2-(2-amino-4,4-difluorobutylamino)-4-(1,2-dimethyl-1H-indol-4-ylamino)pyrimidine-5-carboxamide

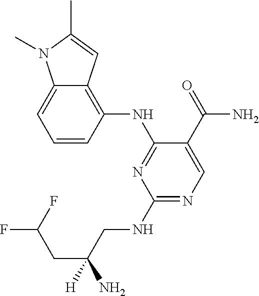 [SMILES: Cc1cc2c(Nc3nc(NC[C@@H](N)CC(F)F)ncc3C(N)=O)cccc2n1C]

The above compound was prepared as described in Scheme 1 utilizing 1,2-dimethyl-1H-indol-4-amine instead of 5-aminoindazole and (S)-benzyl 1-amino-4,4-difluorobutan-2-ylcarbamate (B51F, example 76) instead of (R)-2-aminobutanamide. Additionally a Cbz-deprotection utilizing BBr₃ in DCM (Scheme 7, example 82) was undertaken to give the title compound. MS found for C₁₉H₂₃F₂N₇O as (M+H)⁺404.3.

### Example 88

### Preparation of (R)-2-(2-amino-1-cyclopropylethylamino)-4-(1-ethyl-1H-indol-4-ylamino)pyrimidine-5-carboxamide

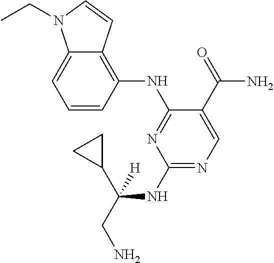 [SMILES: CCn1ccc2c(Nc3nc(N[C@@H](CN)C4CC4)ncc3C(N)=O)cccc21]

The above compound was prepared as described in Scheme 1 utilizing 1-ethyl-1H-indol-4-amine (synthesized analogously as described in Scheme 8) instead of 5-aminoindazole and (R)-2-azido-1-cyclopropylethanamine.HCl instead of (R)-2-aminobutanamide. The synthesis of (R)-2-azido-1-cyclopropylethanamine.HCl is shown below in Scheme 10.

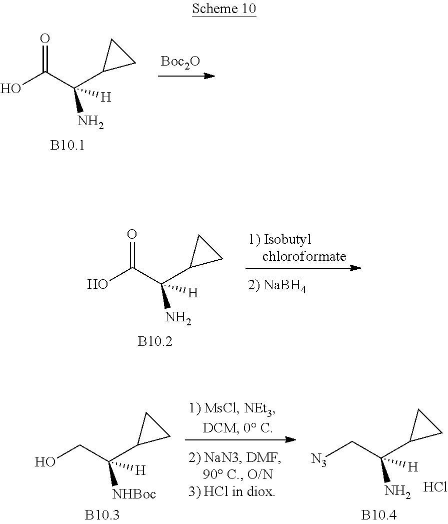

To a stirred solution of B10.1 (5.0 g, 44 mmol) in 5% NaHCO₃ (aq) was added Boc₂O (9.0 g, 45 mmol) in dioxane. The reaction was stirred overnight. An additional 4.0 g Boc₂O was added and the reaction was stirred for 72 hours. The reaction mixture was concentrated and the residue was dissolved in EtOAc. The organic layer was washed with a dilute citric acid solution (pH˜2). This acidic aqueous layer was further extracted with EtOAc. The combined organics were again washed with a dilute citric acid solution and then by a brine wash. The organic layer was dried over MgSO₄ and concentrated to give 8.11 g of B10.2. To 6.5 g (30 mmol) B10.2 in 140 mL THF at −30° C. was added 3.3 mL N-methylmorpholine and 3.93 g isobutyl chloroformate (sequentially). The reaction was stirred at −30° C. for 15 minutes and then 3.47 NaBH₄ was added. Just after the addition of NaBH₄, 5.5 mL H₂O was added. The reaction was stirred for 1 hour and was then diluted with ˜300 mL EtOAc. The reaction mixture was washed with 50 mL 1 N NaHSO₄, 100 mL sat. NaHCO₃, and 100 mL brine. The EtOAc was concentrated and an Isco silica column was run on the crude (0 to 8% MeOH in DCM). Overall, 5.02 g of a clear oil (B10.3) was obtained. Transformation of the alcohol to the azide was achieved utilizing conditions analogous to those described in steps 4 and 5 of Scheme 3 (example 12). A Boc-deprotection utilizing 4.0 N HCl in dioxane afforded B10.4. Note that since the reaction product of the 1-ethyl-1H-indol-4-amine derivative and B10.4 contains an azide, an additional reduction step was required (see example 12, Scheme 3, step 6) to give the title compound. MS found for C₂₀H₂₅N₇O as (M+H)⁺380.2.

### Example 89

### Preparation of (R)-4-(m-toluidino)-2-(1-amino-4,4-difluorobutan-2-ylamino)pyrimidine-5-carboxamide

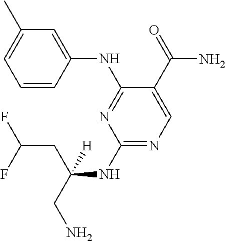 [SMILES: Cc1cccc(Nc2nc(N[C@@H](CN)CC(F)F)ncc2C(N)=O)c1]

The above compound was prepared as described in Scheme 1 utilizing m-toluidine instead of 5-aminoindazole and (R)-tert-butyl 2-amino-4,4-difluorobutylcarbamate instead of (R)-2-aminobutanamide. The synthesis of (R)-tert-butyl 2-amino-4,4-difluorobutylcarbamate is detailed below in Scheme 11.

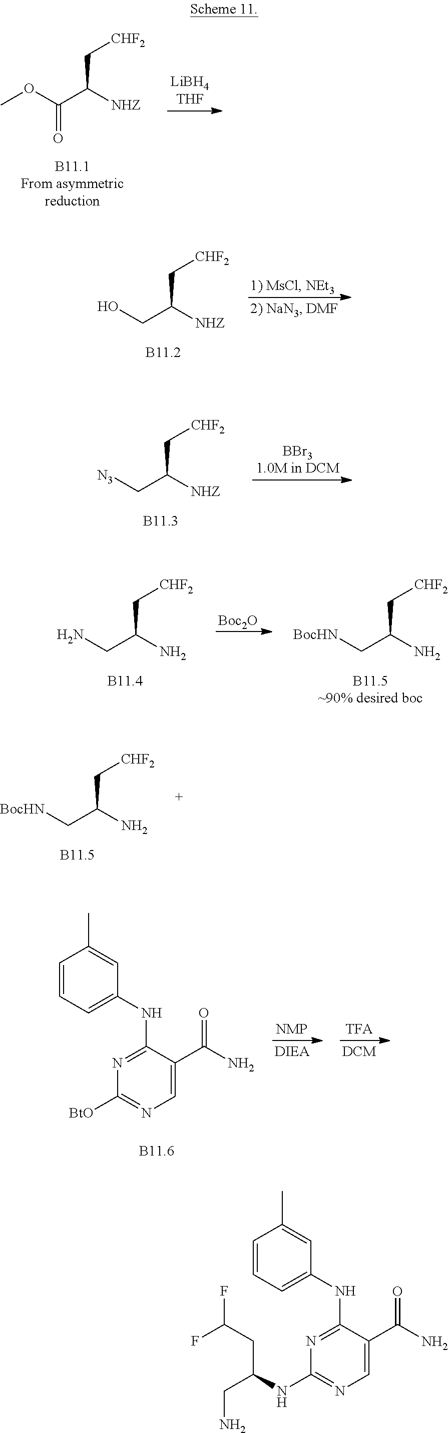

Compound B11.1 was synthesized in analogous manner to that of B51B in Scheme 3. Here however, 1,2-Bis[(2R,5R)-2,5-diethylphospholano]benzene (1,5-cyclooctadiene)rhodium(I) trifluoromethanesulfonate was utilized to give the (R)-substituted product. Also similar to Scheme 3, a LiBH₄ reduction was followed by treatment with MsCl and NaN₃ (two steps) to give azide B11.3, Treatment of B11.3 with 1.1 eq of 1.0 M BBr₃ at 0° C. resulted in incomplete deprotection of the Cbz-protected amine (after stirring for 10 min at 0° C. and 20 min at RT). Therefore an additional 2 mL of 1.0 M BBr₃ was added (at RT). The reaction bubbled violently and the reaction was stirred for an additional 10 minutes at RT. A few drops of H₂O were added and the reaction mixture was concentrated. The concentrate was dissolved in DCM and was treated with water. The layers were separated and the acidic aqueous layer was concentrated to give B11.4. Crude B11.4 was dissolved in NMP and DIEA and Boc₂O was added to it. The reaction was stirred overnight. Water and EtOAc were added to the reaction mixture and the layers were separated. The aqueous layer (pH˜6) was turned basic (to ˜pH=11) with 1 M NaOH and B11.5 was extracted with EtOAc. Brine was used to wash the EtOAc layer which was concentrated and pushed forward. Reaction of B11.5 with B11.6 (made analogously to B6.3) in NMP with DIEA at 90° C. followed by Boc-deprotection utilizing DCM/TFA afforded the title compound. MS found for C₁₆H₂₀F₂N₆O as (M+H)⁺351.2.

### Example 90

### Preparation of (R)-4-(3-(2H-1,2,3-triazol-2-yl)phenylamino)-2-(1-amino-4,4-difluorobutan-2-ylamino)pyrimidine-5-carboxamide

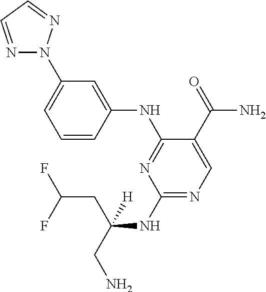 [SMILES: NC[C@@H](CC(F)F)Nc1ncc(C(N)=O)c(Nc2cccc(-n3nccn3)c2)n1]

The above compound was prepared as described in Scheme 1 utilizing 3-(2H-1,2,3-triazol-2-yl)aniline instead of 5-aminoindazole and (R)-tert-butyl 2-amino-4,4-difluorobutylcarbamate (B11.5, example B26) instead of (R)-2-aminobutanamide. Similar to Scheme 2 (example 73), a Boc-deprotection utilizing DCM/TFA afforded the title compound. MS found for C₁₇H₁₉F₂N₉O as (M+H)⁺404.2.

### Example 91

### Preparation of 2-((1R,2R)-2-amino-3,3-difluorocyclohexylamino)-4-(1-methyl-1H-indol-4-ylamino)pyrimidine-5-carboxamide

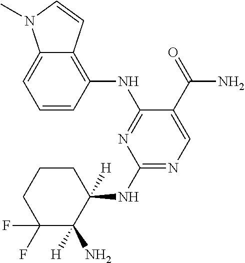 [SMILES: Cn1ccc2c(Nc3nc(N[C@@H]4CCCC(F)(F)[C@@H]4N)ncc3C(N)=O)cccc21]

The title compound was prepared as described in Scheme 1 utilizing 1-methyl-1H-indol-4-amine instead of 5-aminoindazole and (1R,2R)-3,3-difluorocyclohexane-1,2-diamine (synthesis detailed previously) instead of (R)-2-aminobutanamide. MS found for C₂₀H₂₃F₂N₇O as (M+H)⁺416.2.

### Example 92

### Preparation of 2-((1R,2R)-2-amino-3,3-difluorocyclohexylamino)-4-(1-ethyl-1H-indol-4-ylamino)pyrimidine-5-carboxamide

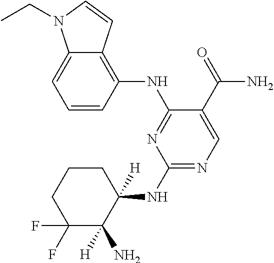 [SMILES: CCn1ccc2c(Nc3nc(N[C@@H]4CCCC(F)(F)[C@@H]4N)ncc3C(N)=O)cccc21]

The title compound was prepared as described in Scheme 1 utilizing 1-ethyl-1H-indol-4-amine instead of 5-aminoindazole and (1R,2R)-3,3-difluorocyclohexane-1,2-diamine (synthesis detailed previously) instead of (R)-2-aminobutanamide. MS found for C₂₁H₂₅F₂N₇O as (M+H)⁺430.3.

### Example 93

### Preparation of 2-((1R,2R)-2-amino-3,3-difluorocyclohexylamino)-4-(1-(2,2,2-trifluoroethyl)-1H-indol-4-ylamino)pyrimidine-5-carboxamide

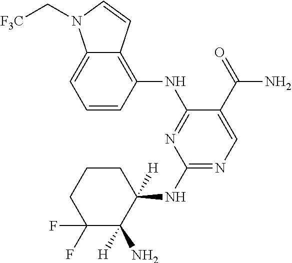 [SMILES: NC(=O)c1cnc(N[C@@H]2CCCC(F)(F)[C@@H]2N)nc1Nc1cccc2c1ccn2CC(F)(F)F]

The title compound was prepared as described in Scheme 1 utilizing 1-(2,2,2-trifluoroethyl)-1H-indol-4-amine (B8.3, Example 21) instead of 5-aminoindazole and (1R,2R)-3,3-difluorocyclohexane-1,2-diamine (synthesis detailed previously) instead of (R)-2-aminobutanamide. MS found for C₂₁H₂₂F₅N₇O as (M+H)⁺484.2.

### Example 94

### Preparation of (S)-2-(2-amino-4,4-difluorobutylamino)-4-(1-methyl-1H-pyrrolo[2,3-b]pyridin-4-ylamino)pyrimidine-5-carboxamide

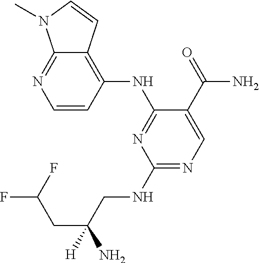 [SMILES: Cn1ccc2c(Nc3nc(NC[C@@H](N)CC(F)F)ncc3C(N)=O)ccnc21]

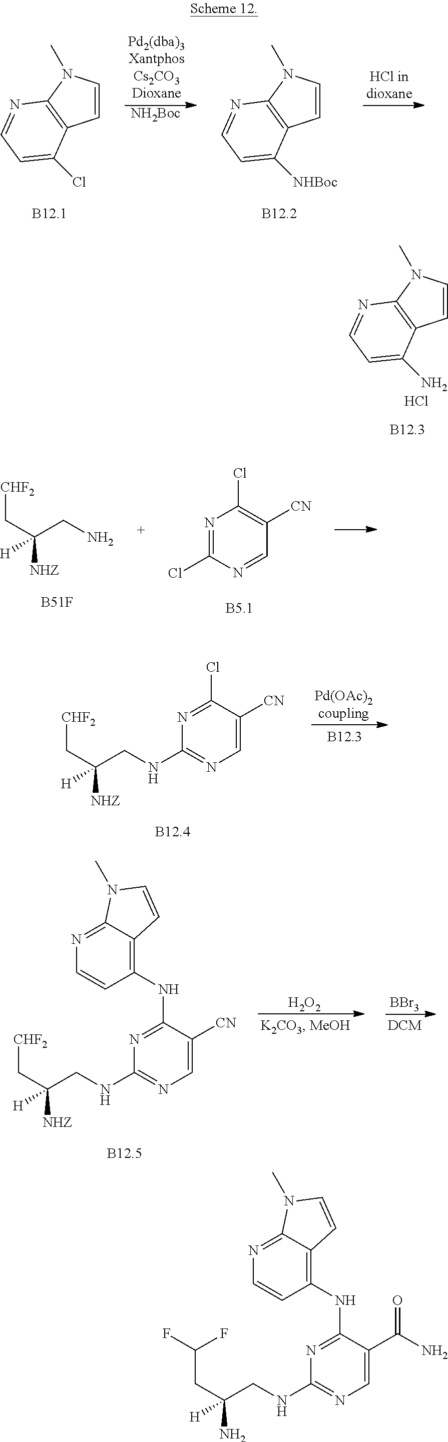

To 100 mg B12.1 (commercially available, 0.60 mmol) was added 117 mg (1.5 eq) H₂NBoc, 393 mg Cs₂CO₃ (2 eq), 52 mg Xantphos (0.15 eq) and 27 mg Pd₂(dba)₃ (0.05 eq). To the above mixture was added degassed (Ar) dioxane. The suspension was further degassed for 5 minutes and then heated at 110° C. under an Ar atmosphere overnight. The reaction mixture was cooled and the dioxane was removed in vacuo. The residue was dissolved in DCM and then the DCM layer was washed with water. The DCM layer was concentrated to give crude B12.2. To crude B12.2 was added 4.0 M HCl in dioxane. The reaction mixture was stirred at room temperature for 3 hours and then the resulting solid was filtered, washed with Et₂O and dried to give B12.3. To 264 mg (1.51 mmol) B5.1 in 2 mL DMF and 0.30 mL DIEA was added 2.5 mL of a ˜1 mM stock solution of B51F in NMP. The reaction mixture was stirred for 15 minutes and then H₂O was added. EtOAc utilized to extract product. Organic layer separated and then washed with 5% NaHCO₃, H₂O, and brine. EtOAc dried over MgSO₄ and concentrated. An Isco silica column (0 to 5% EtOAc in DCM) was run and product B12.4 was isolated (245 mg). To 100 mg B12.4 and B12.3 (89 mg) in 5 mL dioxane was added Cs₂CO₃ (375 mg), rac-BINAP (52 mg), and Pd(OAc)₂ (17 mg). The reaction mixture was degassed for 5 minutes and then heated at 90° C. for 1 hour and then at 110° C. for 30 minutes. The reaction was cooled and concentrated. DCM was added and the suspension was filtered. The filtrate was washed with H₂O and was concentrated. An Isco silica column was run on the crude residue (0 to 7% MeOH in DCM) to afford pure B12.5. Nitrile to amide conversion (as detailed in Scheme 5) and Cbz-deprotection (as detailed in Scheme 7) afforded the title compound. MS found for C₁₇H₂₀F₂N₈O as (M+H)⁺391.2.

### Example 95

### Preparation of (S)-2-(2-amino-4,4-difluorobutylamino)-4-(3-(pyrimidin-2-yl)phenylamino)pyrimidine-5-carboxamide

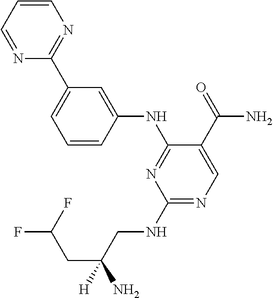 [SMILES: NC(=O)c1cnc(NC[C@@H](N)CC(F)F)nc1Nc1cccc(-c2ncccn2)c1]

The above compound was prepared as described in Scheme 1 utilizing 3-(pyrimidin-2-yl)aniline instead of 5-aminoindazole and (S)-benzyl 1-amino-4,4-difluorobutan-2-ylcarbamate (B51F, example 76) instead of (R)-2-aminobutanamide. Additionally a Cbz-deprotection utilizing BBr₃ in DCM (Scheme 7, example 82) was undertaken to give the title compound. MS found for C₁₉H₂₀F₂N₈O as (M+H)⁺415.4.

### Example 96

### Preparation of 2-((1R,2S)-2-aminocyclohexylamino)-4-(1-methyl-2-oxoindolin-4-ylamino)pyrimidine-5-carboxamide

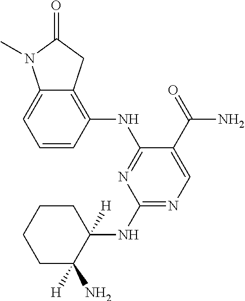 [SMILES: CN1C(=O)Cc2c(Nc3nc(N[C@@H]4CCCC[C@@H]4N)ncc3C(N)=O)cccc21]

The above compound was prepared as described in Scheme 1 utilizing 4-amino-1-methylindolin-2-one instead of 5-aminoindazole and tert-butyl (1S,2R)-2-aminocyclohexylcarbamate instead of (R)-2-aminobutanamide. The synthesis of 4-amino-1-methylindolin-2-one is detailed in Scheme 13.

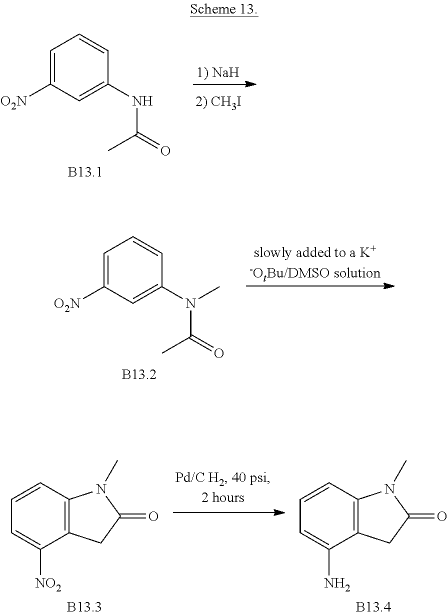

Compound B13.2 was synthesized in a manner similar to that described in Scheme 8. Conversion to B13.3 was achieved by utilizing a procedure described by Makosza et al. in Synthesis, (15), 2203-2206; 2002. Hydrogenation using Pd/C and H₂ in EtOH at 40 psi gave 4-amino-1-methylindolin-2-one (B13.4) which was utilized instead of 5-aminoindazole. Similar to Scheme 2 (example 73), the final step was a Boc-deprotection utilizing DCM/TFA to afford the title compound. MS found for C₂₀H₂₅N₇O₂ as (M+H)⁺396.3.

### Example 97

### Preparation of (S)-2-(2-amino-4,4-difluorobutylamino)-4-(1-(2,2,2-trifluoroethypindolin-4-ylamino)pyrimidine-5-carboxamide

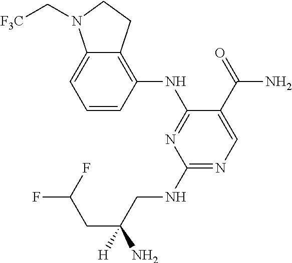 [SMILES: NC(=O)c1cnc(NC[C@@H](N)CC(F)F)nc1Nc1cccc2c1CCN2CC(F)(F)F]

The title compound was synthesized from Example 84 and Et₃SiH in TFA as described in Scheme 14.

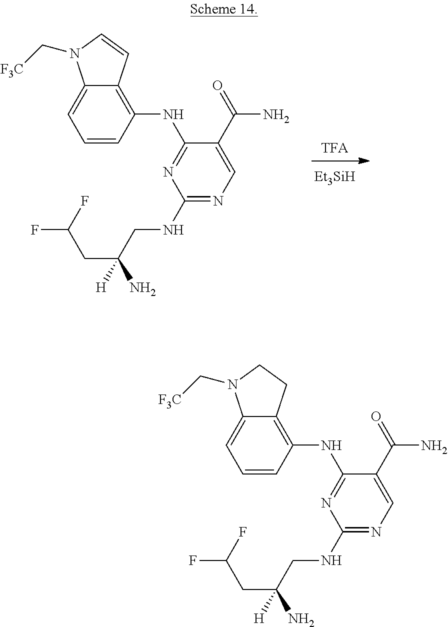 [SMILES: "Et3SiH" ; NC(=O)c1cnc(NC[C@@H](N)CC(F)F)nc1Nc1cccc2c1ccn2CC(F)(F)F ; NC(=O)c1cnc(NC[C@@H](N)CC(F)F)nc1Nc1cccc2c1CCN2CC(F)(F)F]

To 9 mg of (S)-2-(2-amino-4,4-difluorobutylamino)-4-(1-(2,2,2-trifluoroethyl)-1H-indol-4-ylamino)pyrimidine-5-carboxamide in ˜1 mL TFA was added 50 uL Et₃SiH. The reaction mixture was stirred at room temperature for 5 minutes and then 50° C. for 1 hour. Upon cooling, water was added (˜4 mL), the reaction was filtered and prepped via rpHPLC. MS found for C₁₉H₂₂F₅N₇O as (M+H)⁺460.2.

### Example 98

### Preparation of (S)-2-(2-amino-4,4-difluorobutylamino)-4-(1-methylindolin-4-ylamino)pyrimidine-5-carboxamide

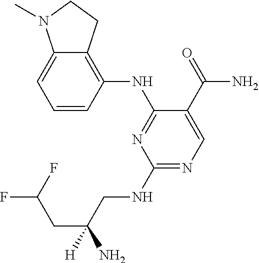 [SMILES: CN1CCc2c(Nc3nc(NC[C@@H](N)CC(F)F)ncc3C(N)=O)cccc21]

The title compound was synthesized from example 85 and Et₃SiH in TFA as described in Scheme 14. MS found for C₁₈H₂₃F₂N₇O as (M+H)⁺392.2.

### Example 99

### Preparation of (R)-2-(2-amino-1-cyclopropylethylamino)-4-(1-ethylindolin-4-ylamino)pyrimidine-5-carboxamide

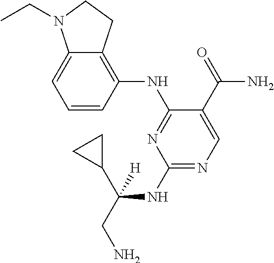 [SMILES: CCN1CCc2c(Nc3nc(N[C@@H](CN)C4CC4)ncc3C(N)=O)cccc21]

The title compound was synthesized from example 88 and Et₃SiH in TFA as described in Scheme 14. MS found for C₂₀H₂₇N₇O as (M+H)⁺382.2.

### Example 100

### Preparation of 2-((1R,2S)-2-aminocyclohexylamino)-4-(2-ethyl-1,3-dimethyl-1H-indol-4-ylamino)pyrimidine-5-carboxamide

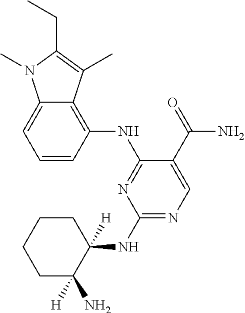 [SMILES: CCc1c(C)c2c(Nc3nc(N[C@@H]4CCCC[C@@H]4N)ncc3C(N)=O)cccc2n1C]

The above compound was prepared as described in Scheme 1 utilizing 2-ethyl-1,3-dimethyl-1H-indol-4-amine instead of 5-aminoindazole and tert-butyl (1S,2R)-2-aminocyclohexylcarbamate instead of (R)-2-aminobutanamide. The synthesis of 2-ethyl-1,3-dimethyl-1H-indol-4-amine is detailed in Scheme 15.

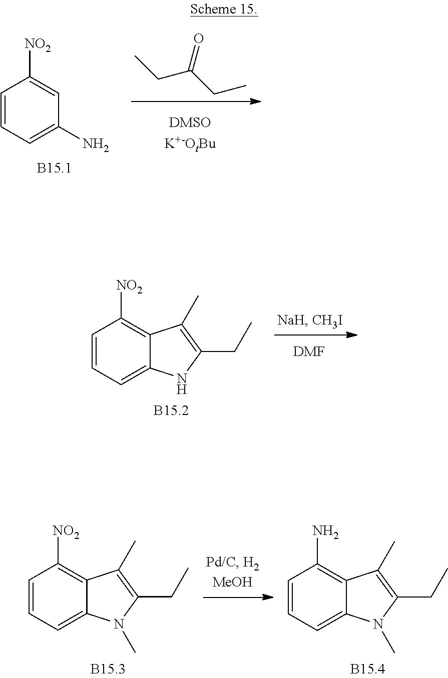

To 3.22 g (23.3 mmol) B15.1 and 4.93 mL (46.6 mmol) 3-pentanone in 50 mL DMSO was added 5.2 g K⁺⁻OₜBu. The reaction mixture was stirred at room temperature for 1 hour and then was pour over ice. Product was extracted with EtOAc (3×150 mL) and washed with H₂O and brine. The organic phase was dried over MgSO₄, filtered and concentrated. An Isco silica column was run on the crude (100% DCM). Overall, 552 mg B15.2 was recovered. Conversion of B15.2 to B15.3 was achieved via methods described in Scheme 8. A hydrogenation of B15.3 in MeOH gave B15.4. Additional steps (reaction with B1.1, etc) as detailed in Scheme 1 were undertaken to give the title compound. MS found for C₂₃H₃₁N₇O as (M+H)⁺422.2.

### Example 101

### Preparation of 2-((1R,2S)-2-aminocyclohexylamino)-4-(3-ethyl-1,2-dimethyl-1H-indol-4-ylamino)pyrimidine-5-carboxamide

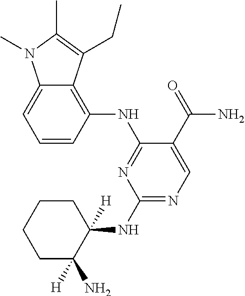 [SMILES: CCc1c(C)n(C)c2cccc(Nc3nc(N[C@@H]4CCCC[C@@H]4N)ncc3C(N)=O)c12]

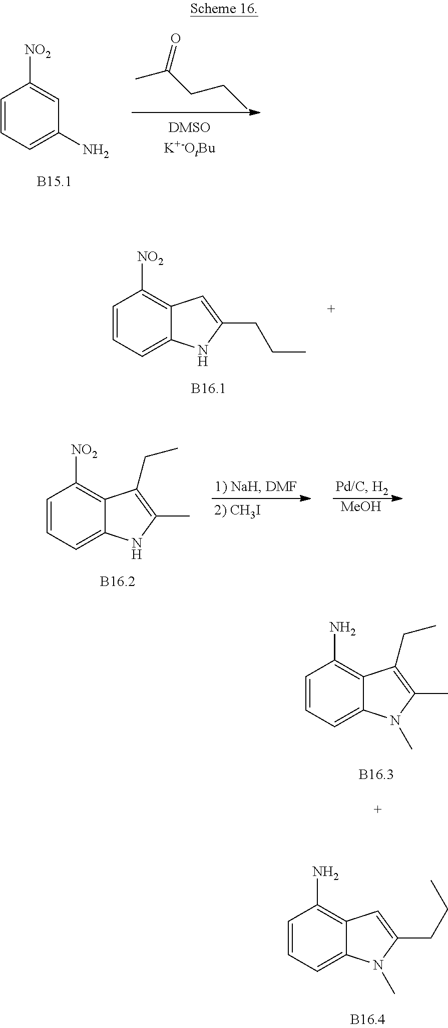

The above compound was prepared as described in Scheme 1 utilizing 3-ethyl-1,2-dimethyl-1H-indol-4-amine (B16.3) instead of 5-aminoindazole and tert-butyl (1S,2R)-2-aminocyclohexylcarbamate instead of (R)-2-aminobutanamide. The synthesis B16.3 is shown in Scheme 16 and is similar to Scheme 15 except that 2-pentanone was utilized instead of 3-pentanone. Note that B16.3 and B16.4 were not separated during the reaction sequence. Additional steps (reaction of B16.3/B16.4 with B1.1, etc) as detailed in Scheme 1 were undertaken to give the title compound. MS found for C₂₃H₃₁N₇O as (M+H)⁺422.3.

### Example 102

### Preparation of 2-((1R,2S)-2-aminocyclohexylamino)-4-(1-methyl-2-propyl-1H-indol-4-ylamino)pyrimidine-5-carboxamide

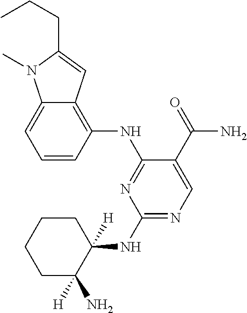 [SMILES: CCCc1cc2c(Nc3nc(N[C@@H]4CCCC[C@@H]4N)ncc3C(N)=O)cccc2n1C]

The title compound was prepared as described in Example B38 (utilizing the B16.3/B16.4 mixture). MS found for C₂₃H₃₁N₇O as (M+H)⁺422.3.

### Example 103

### Preparation of 2-((1R,2S)-2-aminocyclohexylamino)-4-(3-cyano-1-methyl-1H-indol-4-ylamino)pyrimidine-5-carboxamide

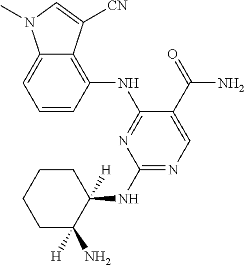 [SMILES: Cn1cc(C#N)c2c(Nc3nc(N[C@@H]4CCCC[C@@H]4N)ncc3C(N)=O)cccc21]

The title compound was prepared as described in Scheme 6 utilizing 4-amino-1-methyl-1H-indole-3-carbonitrile instead of p-toluidine and tert-butyl (1S,2R)-2-aminocyclohexylcarbamate instead of (S)-tert-butyl 1-aminopropan-2-ylcarbamate. The synthesis of 4-amino-1-methyl-1H-indole-3-carbonitrile is detailed in Scheme 17.

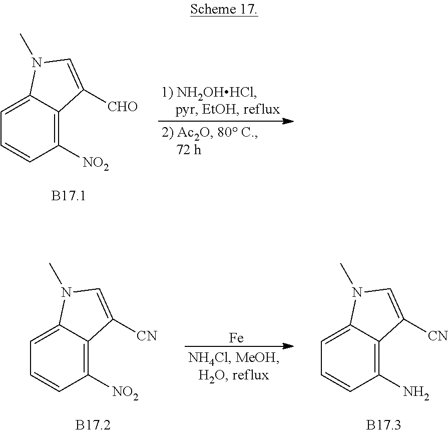

To 590 mg (2.89 mmol) B17.1, 402 mg (5.78 mmol) NH₂OH.HCl, and 2 mL pyridine, was added ˜10 mL EtOH. The reaction mixture was stirred at 80° C. for 1.5 hours. The reaction mixture was cooled and 15 mL 1 M HCl (aq) was added. Solvent was evaporated and the resulting residue was extracted with EtOAc. The organic layer was washed with 1 M HCl (aq) and dried over Na₂SO₄. The organics were removed in vacuo. The crude oxime intermediate was then treated with ˜5 mL Ac₂O and stirred at 80° C. for 72 hours. The reaction mixture was cooled, EtOAc was added followed by sat. NaHCO₃ (aq). Insoluble solid (between the layers) was filtered. The layers were separated and the EtOAc layer was further washed with NaHCO₃ (aq). The organics were removed in vacuo to give 360 mg crude B17.2. To 240 mg B17.2 in ˜20 mL MeOH and 20 mL H₂O was added activated Fe (600 mg) and 400 mg NH₄Cl. The reaction mixture was refluxed for 1 hour and then was diluted with EtOAc and filtered. The organic layer was washed with H₂O and concentrated. An Isco silica column was run on the crude (0 to 2% MeOH in DCM) to separate B17.3 from B17.1. Overall, 114 mg B17.3 obtained. Additional steps (reaction of B17.3 with B6.1, etc) as detailed in Scheme 6 were undertaken to give the title compound. MS found for C₂₁H₂₄N₈O as (M+H)⁺405.2.

### Example 104

### Preparation of 2-(2-amino-3,3-difluorobutylamino)-4-(1-methyl-1H-indol-4-ylamino)pyrimidine-5-carboxamide

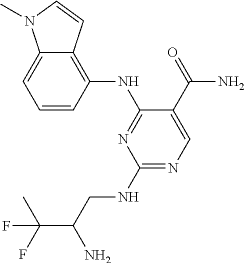 [SMILES: Cn1ccc2c(Nc3nc(NCC(N)C(C)(F)F)ncc3C(N)=O)cccc21]

The above compound was prepared as described in Scheme 1 utilizing 1-methyl-1H-indol-4-amine instead of 5-aminoindazole and 3,3-difluorobutane-1,2-diamine dihydrochloride salt (B18.6) instead of (R)-2-aminobutanamide. The synthesis of 3,3-difluorobutane-1,2-diamine dihydrochloride salt (B18.6) is detailed below in Scheme 18.

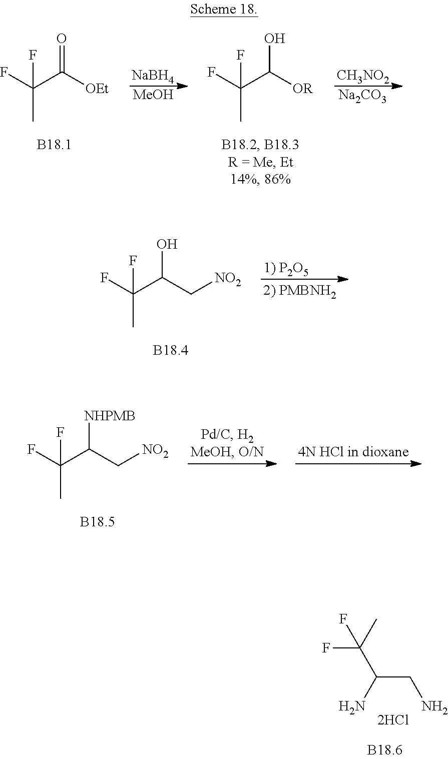

Conversion of B18.1 (5.0 g) to B18.2 (Me) and B18.3 (Et) was conducted according to Parisi et al., JOC, 60(16), 5174-9; 1995. To 2.34 g of the B18.2/B18.3 mixture was added 0.30 g Na₂CO₃ and 3.5 mL CH₃NO₂. The reaction mixture was stirred at room temperature for 16 hours and then was quenched with 1 N HCl (aq). Anhydrous Et₂O was used to extract product. The organic layer was washed with brine, dried over MgSO₄, filtered, and concentrated to give a clear oil, B18.4. To this oil was added P₂O₅ (3×600 mg). This resulted in a gel-like mixture to which ˜10 mL DMSO was added. The reaction mixture (now a yellow solution) was stirred for 1 hour at room temperature. To it was added ˜4 mL PMBNH₂. The reaction was allowed to stir for an additional 1 hour and then H₂O and EtOAc were added. The layers were separated and then the EtOAc layer was washed with 1 N NaOH (aq) and brine. It was dried over MgSO₄ and concentrated in vacuo to give 1.7 g of a golden colored oil. An Isco silica column was run (100% DCM) and B18.5 was isolated. B18.5 was hydrogenated in MeOH and then was filtered through celite. To the filtrate was added 4.0 N HCl in dioxane. The volatiles were removed and B18.6 was obtained as the diHCl salt. Additional steps (reaction with B1.1, etc) as detailed in Scheme 1 were undertaken to give the title compound. MS found for C₁₈H₂₁F₂N₇O as (M+H)⁺390.3.

### Example 105

### Preparation of 4-(m-toluidino)-2-(2-amino-3,3-difluorobutylamino)pyrimidine-5-carboxamide

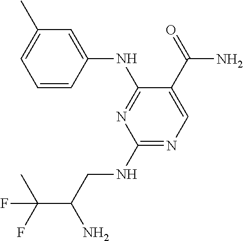 [SMILES: Cc1cccc(Nc2nc(NCC(N)C(C)(F)F)ncc2C(N)=O)c1]

The title compound was prepared as described in Scheme 6 utilizing m-toluidine instead of p-toluidine and 3,3-difluorobutane-1,2-diamine dihydrochloride salt (B18.1) instead of (S)-tert-butyl 1-aminopropan-2-ylcarbamate. MS found for C₁₆H₂₀F₂N₆O as (M+H)⁺351.2.

### Example 106

### Preparation of 4-(3-(1H-pyrazol-1-yl)phenylamino)-2-((1-aminocyclopropyl)methylamino)pyrimidine-5-carboxamide

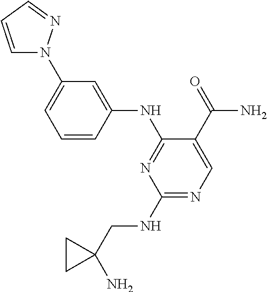 [SMILES: NC(=O)c1cnc(NCC2(N)CC2)nc1Nc1cccc(-n2cccn2)c1]

The title compound was prepared as described in Scheme 6 utilizing 3-(1H-pyrazol-1-yl)aniline instead of p-toluidine and benzyl 1-(aminomethyl)cyclopropylcarbamate instead of (S)-tert-butyl 1-aminopropan-2-ylcarbamate. MS found for C₁₈H₂₀H₈O as (M+H)⁺365.3.

### Example 107

### Preparation of 2-((1R,2S)-2-hydroxycyclohexylamino)-4-(1-methyl-1H-indol-4-ylamino)pyrimidine-5-carboxamide

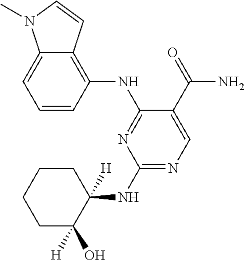 [SMILES: Cn1ccc2c(Nc3nc(N[C@@H]4CCCC[C@@H]4O)ncc3C(N)=O)cccc21]

The above compound was prepared as described in Scheme 1 utilizing 1-methyl-1H-indol-4-amine instead of 5-aminoindazole and (1S,2R)-2-aminocyclohexanol instead of (R)-2-aminobutanamide. MS found for C₂₀H₂₄N₆O₂ as (M+H)⁺381.3.

### Example 108

### Preparation of 2-(cyclohexylamino)-4-(1-methyl-1H-indol-4-ylamino)pyrimidine-5-carboxamide

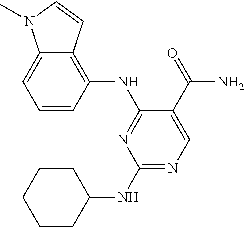 [SMILES: Cn1ccc2c(Nc3nc(NC4CCCCC4)ncc3C(N)=O)cccc21]

The title compound was prepared as described in Scheme 1 utilizing 1-methyl-1H-indol-4-amine instead of 5-aminoindazole and cyclohexylamine instead of (R)-2-aminobutanamide. MS found for C₂₀H₂₄N₆O as (M+H)⁺365.3.

### Example 109

### Preparation of 2-(1-cyanocyclopropylamino)-4-(1-methyl-1H-indol-4-ylamino)pyrimidine-5-carboxamide

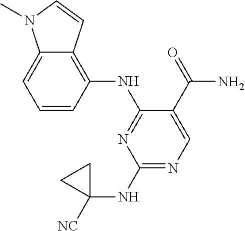 [SMILES: Cn1ccc2c(Nc3nc(NC4(C#N)CC4)ncc3C(N)=O)cccc21]

The title compound was prepared as described in Scheme 1 utilizing 1-methyl-1H-indol-4-amine instead of 5-aminoindazole and 1-aminocyclopropane-1-carbonitrile hydrochloride instead of (R)-2-aminobutanamide. MS found for C₁₈H₁₇N₇O as (M+H)⁺348.2.

### Example 110

### Preparation of (R)-2-(1-cyclopropylethylamino)-4-(1-methyl-1H-indol-4-ylamino)pyrimidine-5-carboxamide

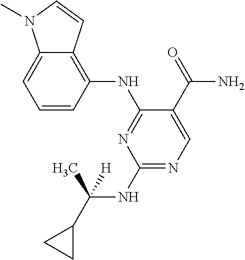 [SMILES: C[C@@H](Nc1ncc(C(N)=O)c(Nc2cccc3c2ccn3C)n1)C1CC1]

The title compound was prepared as described in Scheme 1 utilizing 1-methyl-1H-indol-4-amine instead of 5-aminoindazole and (R)-1-cyclopropylethanamine instead of (R)-2-aminobutanamide. MS found for C₁₉H₂₂N₆O as (M+H)⁺351.3.

### Example 111

### Preparation of 2-(cyclopentylmethylamino)-4-(1-methyl-1H-indol-4-ylamino)pyrimidine-5-carboxamide

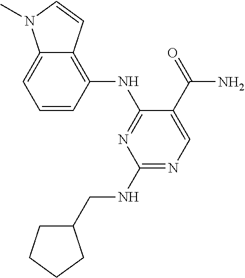 [SMILES: Cn1ccc2c(Nc3nc(NCC4CCCC4)ncc3C(N)=O)cccc21]

The title compound was prepared as described in Scheme 1 utilizing 1-methyl-1H-indol-4-amine instead of 5-aminoindazole and cyclopentylmethanamine instead of (R)-2-aminobutanamide. MS found for C₂₀H₂₄N₆O as (M+H)⁺365.3.

### Example 112

### Preparation of 2-(cyclopropylmethylamino)-4-(1-methyl-1H-indol-4-ylamino)pyrimidine-5-carboxamide

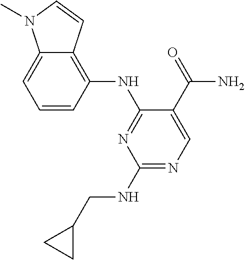 [SMILES: Cn1ccc2c(Nc3nc(NCC4CC4)ncc3C(N)=O)cccc21]

The title compound was prepared as described in Scheme 1 utilizing 1-methyl-1H-indol-4-amine instead of 5-aminoindazole and cyclopropylmethanamine instead of (R)-2-aminobutanamide. MS found for C₁₈H₂₀N₆O as (M+H)⁺337.3.

### Example 113

### Preparation of 4-(1-ethyl-1H-indol-4-ylamino)-2-(2-oxoindolin-5-ylamino)pyrimidine-5-carboxamide

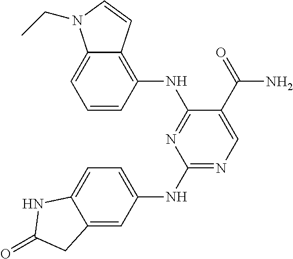 [SMILES: CCn1ccc2c(Nc3nc(Nc4ccc5c(c4)CC(=O)N5)ncc3C(N)=O)cccc21]

The title compound was prepared as described in Scheme 1 utilizing 1-ethyl-1H-indol-4-amine instead of 5-aminoindazole and 5-aminoindolin-2-one instead of (R)-2-aminobutanamide. MS found for C₂₃H₂₁N₇O₂ as (M+H)⁺428.4.

### Example 114

### Preparation of 2-(cyclopropylamino)-4-(1-ethyl-1H-indol-4-ylamino)pyrimidine-5-carboxamide

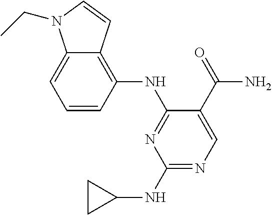 [SMILES: CCn1ccc2c(Nc3nc(NC4CC4)ncc3C(N)=O)cccc21]

The title compound was prepared as described in Scheme 1 utilizing 1-ethyl-1H-indol-4-amine instead of 5-aminoindazole and cyclopropylamine instead of (R)-2-aminobutanamide. MS found for C₁₈H₂₀N₆O₂ as (M+H)⁺337.3.

### Example 115

### Preparation of 4-(1-ethyl-1H-indol-4-ylamino)-2-(isopropylamino)pyrimidine-5-carboxamide

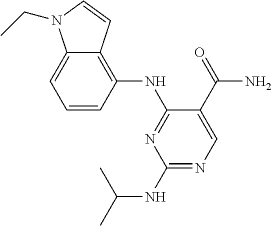 [SMILES: CCn1ccc2c(Nc3nc(NC(C)C)ncc3C(N)=O)cccc21]

The title compound was prepared as described in Scheme 1 utilizing 1-ethyl-1H-indol-4-amine instead of 5-aminoindazole and isopropylamine instead of (R)-2-aminobutanamide. MS found for C₁₈H₂₂N₆O₂ as (M+H)⁺339.3.

### Example 116

### Preparation of 4-(1-ethyl-1H-indol-4-ylamino)-2-(isobutylamino)pyrimidine-5-carboxamide

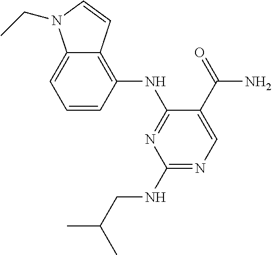 [SMILES: CCn1ccc2c(Nc3nc(NCC(C)C)ncc3C(N)=O)cccc21]

The title compound was prepared as described in Scheme 1 utilizing 1-ethyl-1H-indol-4-amine instead of 5-aminoindazole and isobutylamine instead of (R)-2-aminobutanamide. MS found for C₁₉H₂₄N₆O₂ as (M+H)⁺353.3.

### Example 117

### Preparation of 4-(m-toluidino)-2-(1-(aminomethyl)cyclopropylamino)pyrimidine-5-carboxamide

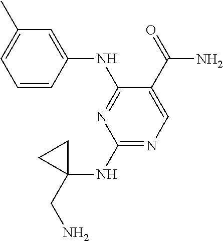 [SMILES: Cc1cccc(Nc2nc(NC3(CN)CC3)ncc2C(N)=O)c1]

The title compound was prepared as described in Scheme 6 utilizing m-toluidine instead of p-toluidine and 1-aminocyclopropane-1-carbonitrile hydrochloride instead of (S)-tert-butyl 1-aminopropan-2-ylcarbamate. This gave intermediate B19.1 which was converted to the product as detailed in Scheme 19.

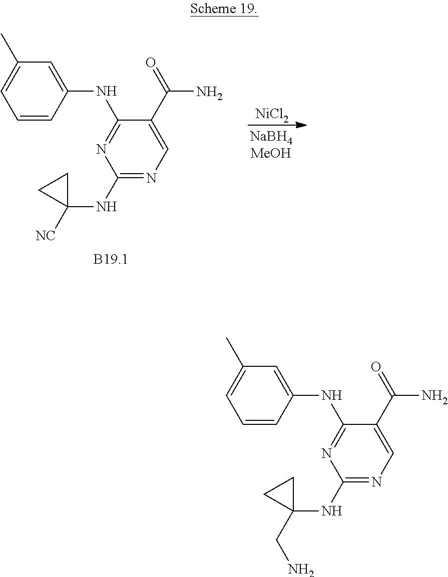

To ˜50 mg B19.1 in 5 mL MeOH was added ˜50 mg NiCl₂ and 50 mg NaBH₄. The reaction mixture was stirred for 5 minutes and then 5 mL 1 N HCl (aq) was added. The reaction was stirred for 10 minutes and then the volatiles were removed. The aqueous layer was turned basic with 1 N NaOH (pH˜12) and was extracted with EtOAc (2×). The combined organic layers were washed with brine and concentrated. A 3:7 mixture of ACN/H₂O (10 mL total) was added to the concentrate along with a couple of drops of TFA. The mixture was filtered and then subjected to rpHPLC. MS found for C₁₆H₂₀N₆O as (M+H)⁺313.3.

### Example 118

### Preparation of 4-(m-toluidino)-2-((1S,3R)-3-aminocyclopentylamino)pyrimidine-5-carboxamide

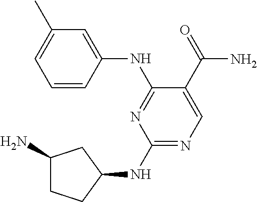 [SMILES: Cc1cccc(Nc2nc(N[C@H]3CC[C@@H](N)C3)ncc2C(N)=O)c1]

The title compound was prepared as described in Scheme 20 utilizing the enantiomer (1S,3R)—N-Boc-1-Aminocyclopentane-3-carboxylic acid instead of (1R,3S)—N-Boc-1-Aminocyclopentane-3-carboxylic acid. MS found for C₁₇H₂₂N₆O as (M+H)⁺327.3.

### Example 119

### Preparation of 4-(m-toluidino)-2-((1R,3S)-3-aminocyclopentylamino)pyrimidine-5-carboxamide

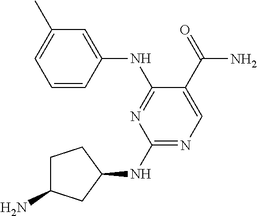 [SMILES: Cc1cccc(Nc2nc(N[C@@H]3CC[C@H](N)C3)ncc2C(N)=O)c1]

The title compound was prepared as described in Scheme 6 utilizing m-toluidine instead of p-toluidine and benzyl (1S,3R)-3-aminocyclopentylcarbamate instead of (S)-tert-butyl 1-aminopropan-2-ylcarbamate. The synthesis of benzyl (1S,3R)-3-aminocyclopentylcarbamate (B20.3) is detailed in Scheme 20.

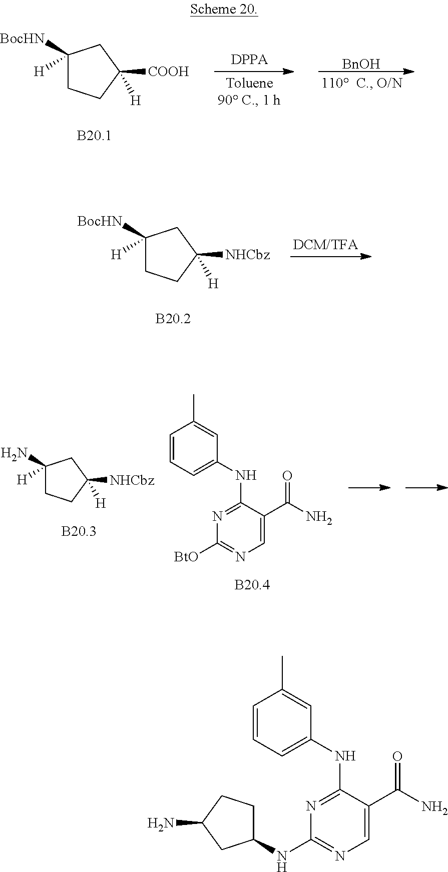

To 1.0 g (4.36 mmol) of commercially available (1R,3S)—N-Boc-1-Aminocyclopentane-3-carboxylic acid (B20.1) in 20 mL toluene was added 1.32 g (4.8 mmol) DPPA. The reaction mixture was stirred at 90° C. for 1 hour. The reaction mixture was cooled to ˜40° C. and BnOH was added (excess, ˜2 mL). The reaction mixture was heated at reflux overnight. The reaction mixture was cooled and diluted with EtOAc as well as ˜30 mL 0.5 M NaOH (aq). The layers were separated and the EtOAc layer was washed with sat. NaHCO₃ and brine. It was then dried over MgSO₄, filtered, and concentrated in vacuo to give crude B20.2. To crude B20.2 was added ˜2 mL DCM and 2 mL TFA. The reaction was stirred for 2 hours and then the volatiles were removed in vacuo to afford B20.3. Next, B20.3 was reacted with B20.4 in NMP and DIEA as described in Scheme 6. Subsequent Cbz-deprotection utilizing BBr₃ in DCM afforded the title compound. MS found for C₁₇H₂₂N₆O as (M+H)⁺327.3.

### Example 120

### Synthesis of (R)-benzyl 1-(1-aminopropyl)cyclopropylcarbamate hydrochloride

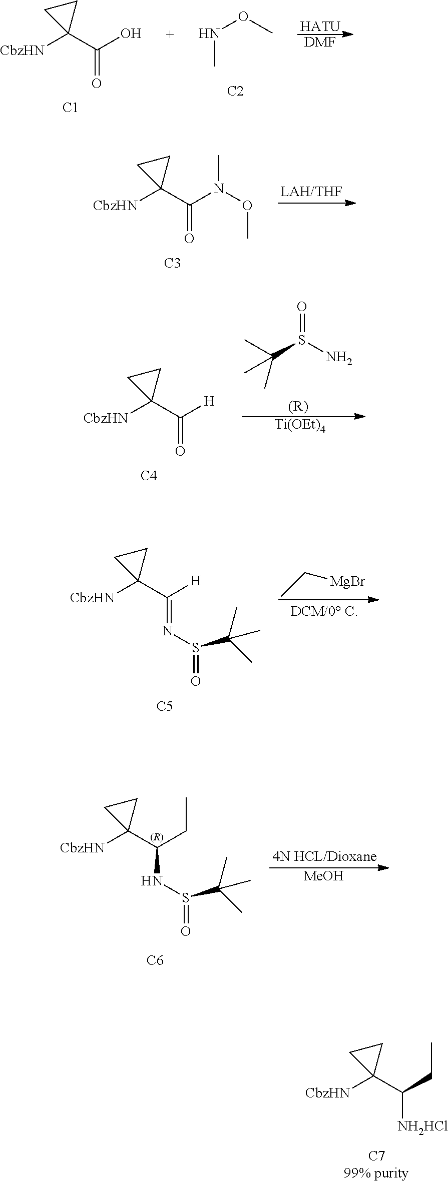

### Step 1

### Synthesis of Benzyl 1-(methoxy(methyl)carbamoyl)cyclopropylcarbamate (C3)

5 g (21.27 mmol) of (C1) and 2.8 g (23.40 mmol) of (C2) were suspended in 30 ml DMF. To this suspension 8.5 g (22.34 mmol) HATU and 17.5 mL (100 mmol) DIPEA was added. Reaction mixture was stirred for 2 hours followed by UPLC (ultra performance liquid chromatography). Reaction completed in 2 hours. Reaction mixture was diluted with EtOAc and 1N NaOH was added to the mixture. Desired compound was extracted in EtOAc 2× (100 mL) Combined organic layers were dried over sodium sulfate and concentrated under vacuum to get desired product. 5.5 g of (C3) was obtained.

### Step 2

### Synthesis of benzyl 1-formylcyclopropylcarbamate (C4)

Benzyl 1-(methoxy(methyl)carbamoyl)cyxlopropylcarbamate (C3) 5.5 g (19.78 mmol) was suspended in 100 ml THF at zero degree. To this suspension 1.5 g (39.56) of LAH was added and reaction mixture was stirred for 1 hour. Reaction was quenched by addition of adding 10% potassiumhydrogensulfate solution. Compound was extracted in DCM 2× (100 ml). Washed with brine and dried over sodium sulfate and concentrated under vacuum to get 3.5 g of the desired product (C4)

### Step 3

### Synthesis of (R,E)-benzyl 1-((tert-butylsulfinylimino)methyl)cyclopropylcarbamate (C5)

3.5 g (16 mmol) of (C4) was suspended in 50 ml THF and 2.3 g (919 mmol) of (R)-2-methylpropane-2-sulfinamide was added to it. To the reaction mixture 7.33 g (32 mmol) of titanium ethoxide was added and stirred for 4 hours. Reaction mixture was quenched with brine and filtered the solid. Filtrate was extracted in DCM, washed with brine and dried over sodium sulfate and concentrated to get oil which was purified by column using DCM:EtOAc (2:1). 3 g of desired product was obtained. (C5)

### Step 4

### Synthesis of benzyl 1-((R)-1-((R)-1,1-dimethylethylsulfinamido)propyl)cyclopropylcarbonate (C6)

3.0 g (9.31 mmol) of 5 was suspended in 50 ml DCM under nitrogen at zero degree. Then 18 ml (54 mmol) of 3M ethyl magnesium bromide solution in ether was added dropwise. Reaction was followed by UPLC. Reaction finished in 2 hours. Reaction was quenched by saturated ammonium chloride solution by drop wise addition in ice bath. Then desired product was extracted in DCM, dried over sodium sulfate and concentrated to get oil. There was 9:1 ratio of isomers. Purified by using flash column using eluent 10% MeOH in DCM. There was no separation. The mixture was suspended in MTBE, and then hexane was used as anti solvent to get precipitates. Solid obtained was filtered which was 99% pure. Filtrate was again suspended in MTBE, similarly more solid as pure product was recovered. 1.5 g of desired product was recovered.

### Step 5

### Synthesis of (R)-benzyl-1-(1-aminopropyl)cyclopropylcarbamate hydrochloride (C7)

Solid obtained (C6) 1.5 g was suspended in 20 ml MeOH and then 4NHCL in dioxane (10 mL) was added to reaction mixture. Stirred for 30 min and concentrated and subjected to high vacuum overnight to get white solid as desired product.

### Example 123

### Preparation of (1R,2R)-3,3-difluorocyclohexane-1,2-diamine

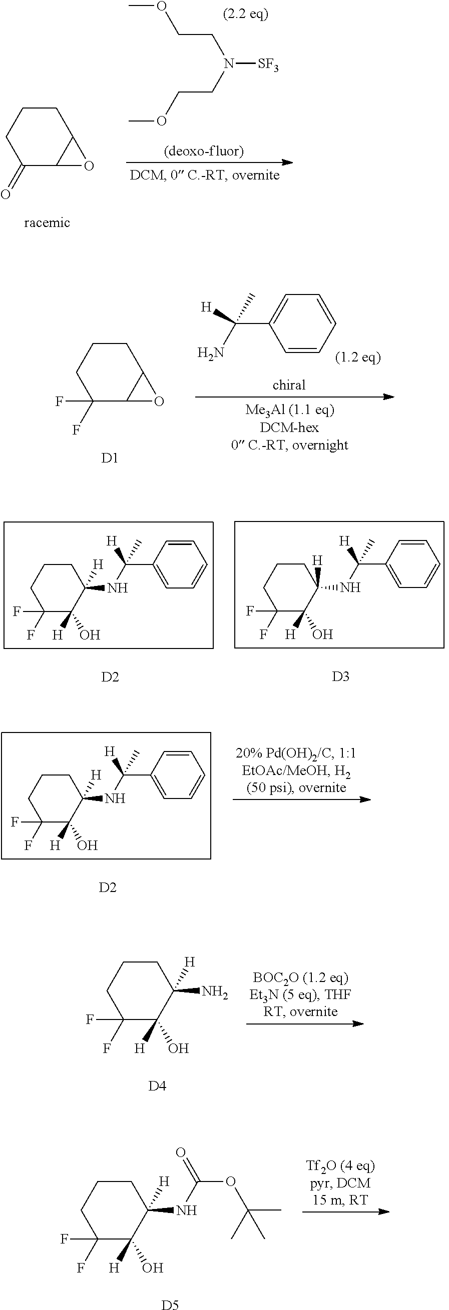

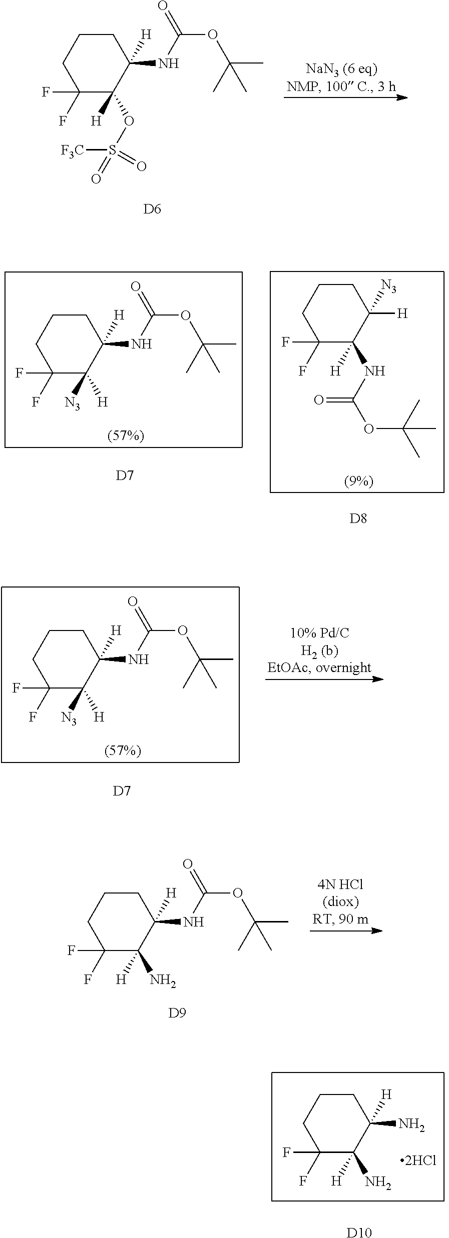

7-Oxabicyclo[4.1.0]heptan-2-one (Aldrich #414522, 8.0 mL, 81 mmol) was dissolved in 40 mL dry DCM and stirred in ice bath. To it was added Deoxo-Fluor (Aldrich #494119, 32.8 mL, 178 mmol) dropwise. The mixture was allowed to warm up to RT and stirred for overnight to give a mixture of compound D1 and some remaining epoxyketone. The mixture was cooled to −20° C. and carefully quenched with 5 mL water dropwise. The mixture was diluted with 600 mL DCM and 200 mL water. The organic phase was separated, dried, filtered through a short (2-inch) silica plug and concentrated in vacuo. The residue was then dissolved in 150 mL DCM. A solution of (R)-(+)-α-methylbenzylamine (Aldrich #115541, 12.2 mL, 96 mmol) in 50 mL DCM was prepared and stirred in ice bath. To it was added a solution of trimethylaluminum in hexane (Aldrich #268569, 44 mL, 88 mmol). The mixture was stirred for 1 h. To it was then added the 150 mL DCM solution from previous step. The mixture was stirred at RT for over the weekend to give a mixture of (D2) and (D3) in about 1:1 ratio. The mixture was then cooled in ice bath. Powder NaF (16.8 g, 400 mmol) was added. Then mixture was treated later with ice chips slowly. To it was poured 500 mL DCM. The mixture was stirred for 2 h at RT. It was filtered through celite. The filtrate was concentrated in vacuo and subjected to flash column with 0-2.5% MeOH in DCM to isolate compound D2 (6.67 g) and compound D3 (5.70 g).A2 NMR (CDCl₃): 7.39-7.25 (5H, m), 4.00 (1H, q, J=6.8 Hz), 3.53 (1H, ddd), 3.04 (2H, bs), 2.74 (1H, m), 2.11 (1H, m), 1.79 (1H, m), 1.63 (2H, m), 1.44 (3H, d, J=6.4 Hz), 1.40 (1H, m), 1.11 (1H, m) ppm. D3 NMR (CDCl₃): 7.36-7.23 (5H, m), 3.95 (1H, q, J=6.4 Hz), 3.48 (1H, ddd), 2.44 (2H, bs), 2.41 (1H, m), 2.09 (2H, m), 1.72-1.55 (2H, m), 1.38 (3H, d, J=6.8 Hz), 1.31 (1H, m), 1.12 (1H, m) ppm.

Compound D2 (6.67 g) was dissolved in 200 mL EtOAc and 200 mL methanol. To the solution was added 20 wt % palladium hydroxide on carbon (Alfa Aesar #212911, 1.65 g). The mixture was shaken on a Parr shaker under 50 psi hydrogen for overnight. The mixture was filtered through celite. The filtrate was concentrated in vacuo to afford compound D4 (4.03 g). It was dissolved in 200 mL THF. To it were added triethylamine (18.1 mL, 130 mmol) and BOC anhydride (6.8 g, 31.2 mmol). The mixture was stirred for overnight, concentrated in vacuo and subjected to flash column (10-20% EtOAc in hexane) to isolate compound D5 (5.34 g). Compound D5 (1.83 g, 7.3 mmol) was dissolved in 50 mL dry DCM. To it was added 15 mL dry pyridine. The mixture was stirred in ice bath. To it was added Tf₂O (4.9 mL, 29 mmol). The reaction was allowed for 15 min and quenched with water. It was further diluted with 100 mL water and 500 mL DCM. The organic phase was separated and washed with water x3, dried, concentrated in vacuo and pumped to dryness to give crude compound D6. It was dissolved in 30 mL NMP. To it was added sodium azide (2.85 g, 43.8 mmol). The mixture was stirred at 100° C. for 3 h. It was cooled to RT. To it was poured 500 mL EtOAc. The mixture was washed with water ×3, dried, concentrated in vacuo and subjected to flash column (0-20% EtOAc in hexane) to isolate the major product D7 (1.15 g, 57%) and the minor product D8 (0.18 g, 9%). D7 NMR (CDCl₃): 4.77 (1H, d, J=6.8 Hz), 3.97 (1H, bs), 3.87 (1H, bm), 1.97-1.86 (2H, m), 1.72-1.63 (2H, m), 1.45 (9H, s), 1.36 (2H, m) ppm. D8 NMR (CDCl₃): 4.81 (1H, d, J=8.8 Hz), 3.91 (1H, m), 3.28 (1H, m), 2.21 (1H, m), 2.11 (1H, m), 1.86-1.79 (2H, m), 1.78-1.64 (2H, m), 1.48 (9H, s), 1.43-1.39 (2H, m) ppm.

Compound D7 (1.15 g, 4.16 mmol) was dissolved in 250 mL EtOAc. To it was added 2.0 g of 10% Pd/C. A hydrogen balloon was attached to the reaction flask. The mixture was stirred for overnight. It was filtered through celite. The celite cake was washed thoroughly with EtOAc and methanol. The filtrate was concentrated in vacuo and pumped to dryness to afford a white solid D9. It was then treated with 40 mL 4N HCl in dioxane at RT for 1.5 h to get a thick gel. It was concentrated and pumped overnight to afford compound D10 as a light brown solid.

### Example 124

### Preparation of 4-(3-(1H-pyrazol-1-yl)phenylamino)-2-((1S,6S,)-6-amino-2,2-difluorocyclohexylamino)pyrimidine-5-carboxamide

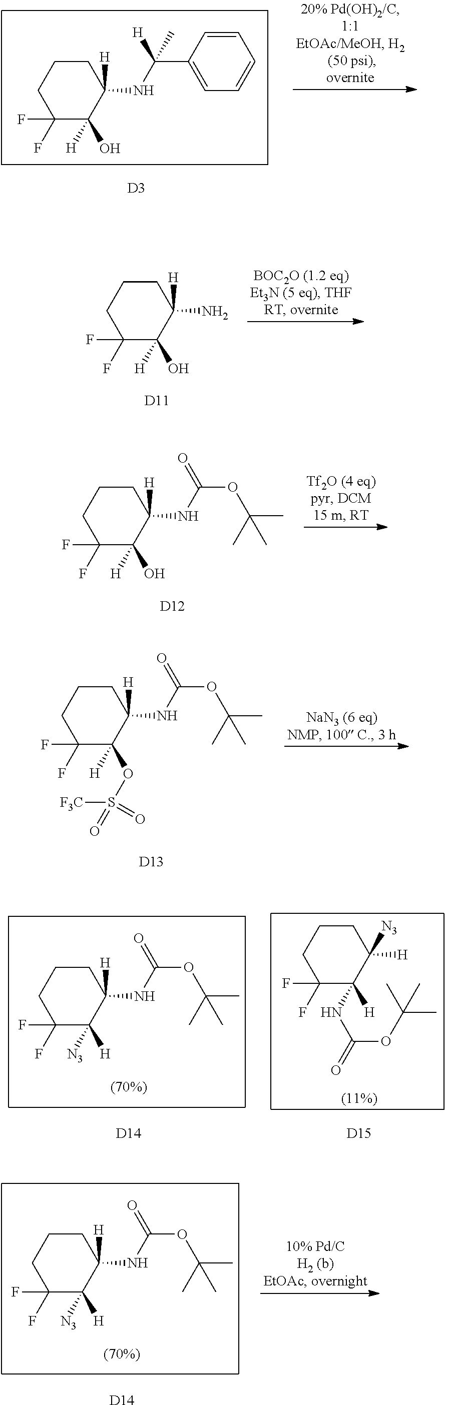

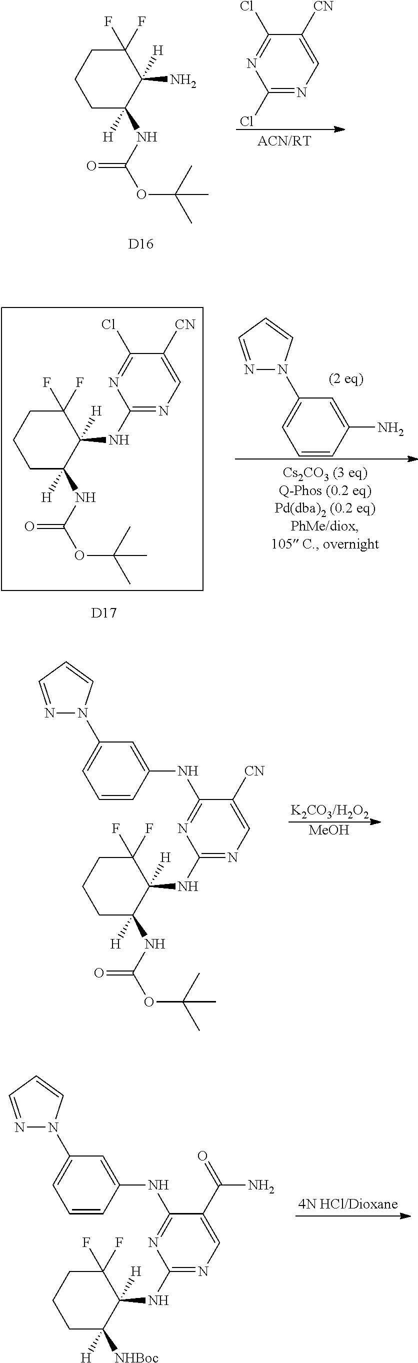

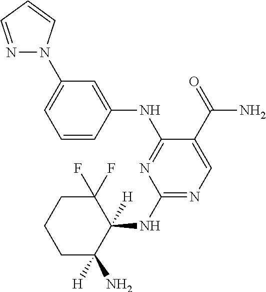 [SMILES: NC(=O)c1cnc(N[C@H]2[C@@H](N)CCCC2(F)F)nc1Nc1cccc(-n2cccn2)c1]

Compound D3 (see Example 122, 5.70 g, 22 mmol) was dissolved in 200 mL and 200 mL methanol. To the solution was added 20 wt % palladium hydroxide on carbon (Alfa Aesar #212911, 1.50 g). The mixture was shaken on a Parr shaker under 40 psi hydrogen for overnight. The mixture was filtered through celite. The filtrate was concentrated in vacuo to afford compound D11. It was dissolved in 200 mL THF. To it were added triethylamine (15.3 mL, 110 mmol) and BOC anhydride (5.8 g, 26.4 mmol). The mixture was stirred for overnight, concentrated in vacuo and subjected to flash column (10-20% EtOAc in hexane) to isolate compound D12 (4.65 g).

Step 2 Compound D12 (3.00 g, 11.9 mmol) was dissolved in 100 mL dry DCM. To it was added 30 mL dry pyridine. The mixture was stirred in ice bath. To it was added Tf₂O (8.0 mL, 47 mmol). The reaction was allowed for 10 min and quenched with water. It was further diluted with 100 mL water and 500 mL DCM. The organic phase was separated and washed with water ×3, dried, concentrated in vacuo and pumped to dryness to give crude compound D13. It was dissolved in 36 mL NMP. To it was added sodium azide (4.64 g, 71.4 mmol). The mixture was stirred at 100° C. for 3 h. It was cooled to RT. To it was poured 500 mL EtOAc. The mixture was washed with water ×3, dried, concentrated in vacuo and subjected to flash column (0-15% EtOAc in hexane) to isolate the major product D14 (2.17 g, 66%) and the minor product D15 (0.42 g, 13%). D15 NMR (CDCl₃): 4.92 (1H, d, J=8.8 Hz), 3.91 (1H, m), 3.77 (1H, bm), 1.85 (1H, m), 1.80 (1H, m), 1.64-1.53 (2H, m), 1.36 (9H, s), 1.36-1.27 (2H, m) ppm. D14 NMR (CDCl₃): 4.83 (1H, d, J=9.2 Hz), 3.91 (1H, m), 3.28 (1H, m), 2.20 (1H, m), 2.10 (1H, m), 1.84-1.69 (2H, m), 1.47 (9H, s), 1.47-1.42 (2H, m) ppm.

Compound D14 (2.17 g, 7.86 mmol) was dissolved in 250 mL EtOAc. To it was added 0.5 g of 10% Pd/C. A hydrogen balloon was attached to the reaction flask. The mixture was stirred for overnight. It was filtered through celite. The celite cake was washed thoroughly with EtOAc and methanol. The filtrate was concentrated in vacuo and pumped to dryness to afford a white solid D16 (1.61 g, 85%).

### Example 125

### (±)-2-(1-(1-aminocyclopropyl)propylamino)-4-(m-tolylamino)pyrimidine-5-carboxamide

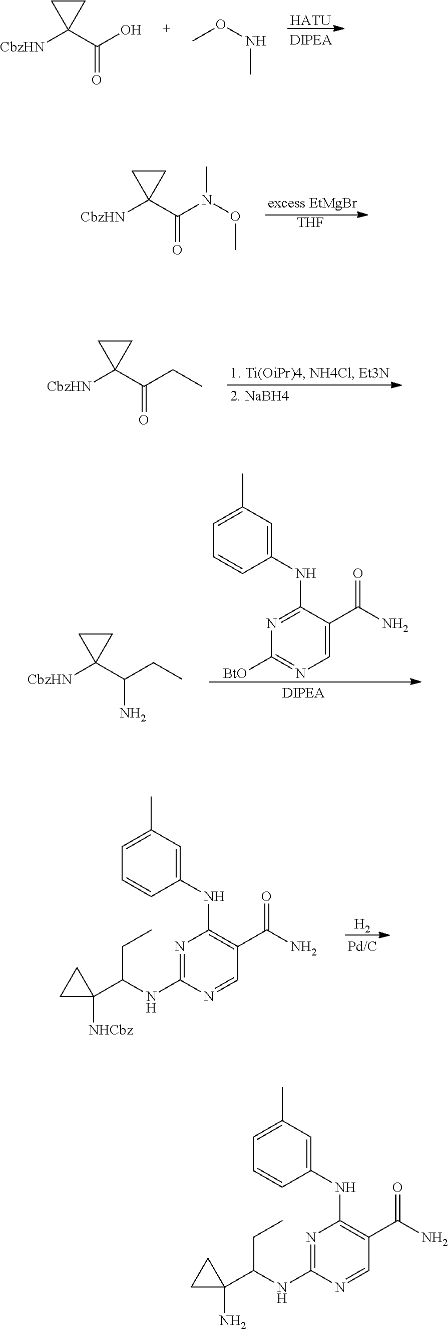

Step 1: To a mixture of (benzyloxycarbonylamino)cyclopropanecarboxylic acid (1.0 g, 4.26 mmol) and N,O-dimethylhydroxyamine hydrogen chloride salt (457 mg, 4.69 mmol) in DMF (7 mL) was added HATU (1.94 g, 5.11 mmol) and DIPEA (3.80 mL). After stirred at room temperature for 2 h, the mixture was diluted with EtOAc, the organic layer was washd with 1N NaOH and brine, dried and concentrated to give crude residue, which was purified by column chromatography to give benzyl 1-(methoxy(methyl)carbamoyl)cyclopropylcarbamate (635 mg).

Step 2: To a solution of benzyl 1-(methoxy(methyl)carbamoyl)cyclopropylcarbamate (635 mg, 2.28 mmol) in THF (12 mL) at ° C. was added EtMgBr (3M, 2.28 mL, 6.85 mmol), the mixture was then warmed up to room temperature and stirred for 2 h, additional EtMgBr was added to drive the reaction to completion. EtOAc and water was added to the mixture, organic layer was separated and washed with brine, dried and concentrated to give crude residue, which was purified by column chromatography to give benzyl 1-propionylcyclopropylcarbamate (405 mg).

Step 3: To a mixture of benzyl 1-propionylcyclopropylcarbamate (200 mg, 0.81 mmol) and Ti(O-iPr)4 (0.475 mL, 1.62 mmol) in EtOH (2 ml) was added NH4Cl (87 mg, 1.62 mmol) and Et3N (0.226 mL, 1.62 mmol). After stirred at room temperature for 15 h, it was added NaBH4 (46 mg, 1.215 mmol) and stirred for additional 48 h. NH4OH was added to the mixture, the resulting precipitate was filtered off, the filtrate was extracted with ether, ether layer was extracted with 1N HCl, the aqueous layer was separated and concentrated to dryness, then it was basified with 1N NaOH, the aqueous layer was extracted with EtOAc, EtOAc layer was combined, washed with brine, dried and concentrated to give benzyl 1-(1-aminopropyl)cyclopropylcarbamte (35 mg).

Step 4: To a suspension of 2-(1H-benzo[d][1,2,3]triazol-1-yloxy)-4-(m-tolylamino)pyrimidine-5-carboxamide (36 mg, 0.1 mmol) in AcCN (1 mL) was added benzyl 1-(1-aminopropyl)cyclopropylcarbamte (35 mg, 0.14 mmol) and DIPEA (0.025 mL, 0.14 mmol). After stirred at 60° C. for 3 h, the solution was concentrated, the residue was diluted with water, the precipitate was collected by filtration to give benzyl 1-(1-(5-carbamoyl-4-(m-tolylamino)pyrimidin-2-ylamino)propyl)cyclopropylcarbamte.

Step 5: To a mixture of benzyl 1-(1-(5-carbamoyl-4-(m-tolylamino)pyrimidin-2-ylamino)propyl)cyclopropylcarbamte in EtOAc (2 mL) was added Pd/C (spatula tip) and charged with H2 (1 atm). Upon completion, Pd/C was filtered off and the filtrate was concentrated to give crude residue, which was purified by preparative HPLC to give 2-(1-(1-aminocyclopropyl)propylamino)-4-(m-tolylamino)pyrimidine-5-carboxamide. MS found for C₁₈H₂₄N₆O as (M+H)⁺341.5, UV: λ=242.8.

### Example 126

### (R)-2-(1-(1-aminocyclopropyl)propylamino)-4-(m-tolylamino)pyrimidine-5-carboxamide

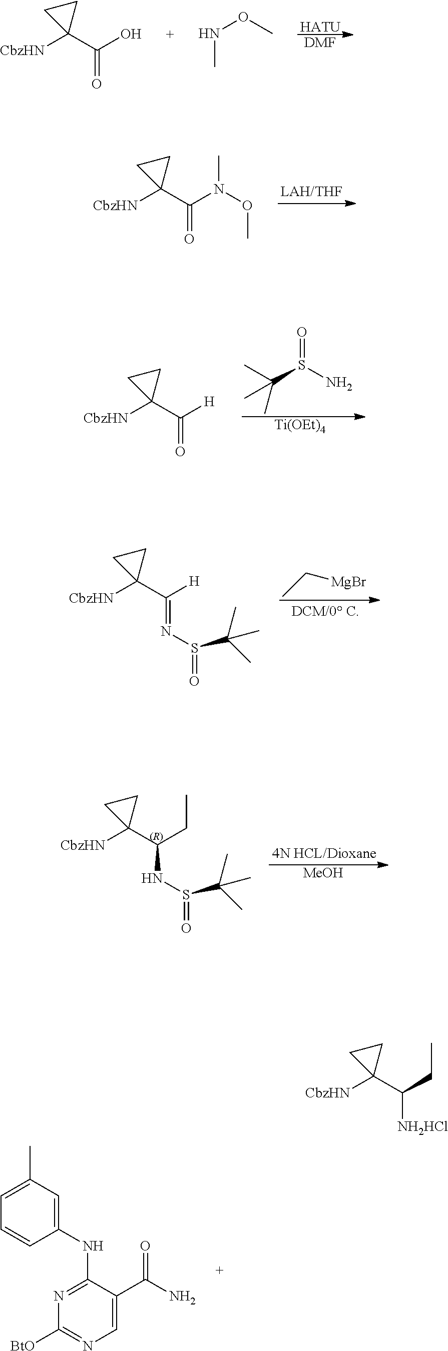

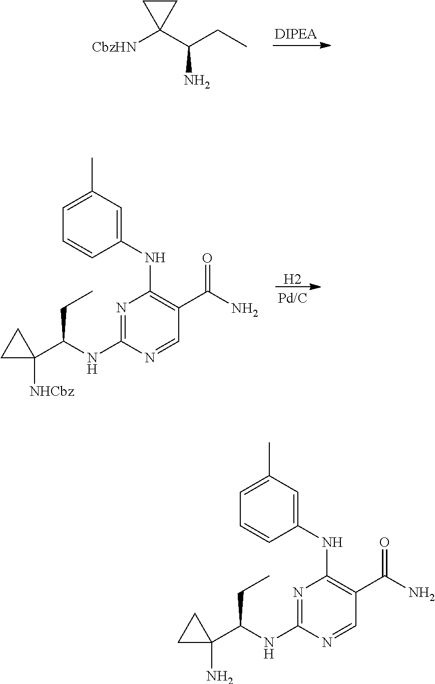 [SMILES: CC[C@@H](N)C1(NC(=O)OCc2ccccc2)CC1 ; CC[C@@H](Nc1ncc(C(N)=O)c(Nc2cccc(C)c2)n1)C1(NC(=O)OCc2ccccc2)CC1 ; "H2" ; CC[C@@H](Nc1ncc(C(N)=O)c(Nc2cccc(C)c2)n1)C1(N)CC1]

### Synthesis of (R)-benzyl 1-(1-aminopropyl)cyclopropylcarbamate hydrochloride

Step 1: To a suspension of (benzyloxycarbonylamino)cyclopropanecarboxylic acid (5 g, 21.27 mmol) and N,O-dimethylhydroxyamine hydrogen chloride salt (2.8 g, 23.40 mmol) DMF (30 ml) was added HATU (8.5 g 22.34 mmol) and DIPEA (17.5 ml, 100 mmol). Reaction mixture was stirred for 2 hours followed by UPLC. Reaction completed in 2 hours. Reaction mixture was diluted with EtOAc and 1N NaOH was added to the mixture. Desired compound was extracted in EtOAc 2× (100 mL) Combined organic layers were dried over sodium sulfate and concentrated under vacuum to get benzyl 1-(methoxy(methyl)carbamoyl)cyclopropylcarbamate (5.5 g).

Step 2: Benzyl 1-(methoxy(methyl)carbamoyl)cyclopropylcarbamate (5.5 g, 19.78 mmol) was suspended in 100 ml THF at 0° C. To this suspension was added LAH (1.5 g, 39.56 mmol) and reaction mixture was stirred for 1 hour. Reaction was quenched by addition of 10% potassium hydrogensulfate solution. Compound was extracted in DCM 2× (100 ml). Washed with brine and dried over sodium sulfate and concentrated under vacuum to get benzyl 1-formylcyclopropylcarbamate (3.5 g).

Step 3: Benzyl 1-formylcyclopropylcarbamate (3.5 g, 16 mmol) was suspended in 50 ml THF and (R)-2-methylpropane-2-sulfinamide (2.3 g, 19 mmol) was added to it. The reaction mixture was added titanium ethoxide (7.33 g, 32 mmol) and stirred for 4 hours. Reaction mixture was quenched with brine and the resulting solid was filtered off. Filtrate was extracted with DCM, DCM layer was combined, washed with brine, dried over sodium sulfate and concentrated to get oil which was purified by column using DCM:EtOAc (2:1) to give (R,E)-benzyl 1-((tert-butylsulfinylimino)methyl)cyclopropylcarbamate (3 g).

Step 4: (R,E)-benzyl 1-((tert-butylsulfinylimino)methyl)cyclopropylcarbamate (3.0 g, 9.3 mmol) was suspended in DCM (50 ml) under Nitrogen at 0° C. Then 3-Methyl magnesium bromide solution in ether (18 ml, 54 mmol) was added drop wise. Reaction was followed by UPLC. Reaction finished in 2 hours. Reaction was quenched by saturated ammonium chloride solution by drop wise addition in ice bath. Then desired product was extracted in DCM, dried over sodium sulfate and concentrated to get oil. There was 9:1 ration of isomers. Purification by using flash column using 10% MeOH in DCM resulted in no separation. The mixture was then suspended in MTBE, and then hexane was used as anti solvent to get precipitates. Solid obtained was filtered which was 99% pure. Filtrate was again suspended in MTBE, similarly more solid as pure product was recovered. 1.5 g of Benzyl 1-((R)-1-((R)-1,1-dimethylethylsulfinamido)propyl)cyclopropylcarbamate was recovered.

Step 5: Benzyl 1-((R)-1-((R)-1,1-dimethylethylsulfinamido)propyl)cyclopropylcarbamate (1.5 g) was suspended in MeOH (20 ml) and then 4N HCl in Dioxane (10 ml) was added to reaction mixture. The mixture was stirred for 30 min and then was concentrated and subjected to high vacuum overnight to get (R)-benzyl-1-(1-aminopropyl)cyclopropylcarbamate hydrochloride as white solid.

Step 6: To a suspension of 2-(1H-benzo[d][1,2,3]triazol-1-yloxy)-4-(m-tolylamino)pyrimidine-5-carboxamide (40 mg, 0.1 mmol) in AcCN (1 mL) was added (R)-benzyl 1-(1-aminopropyl)cyclopropylcarbamte (38 mg, 0.15 mmol) and DIPEA (0.060 mL, 0.33 mmol). After stirred at 60° C. for 3 h, the solution was concentrated, the residue was diluted with water, the precipitate was collected by filtration to give (R)-benzyl 1-(1-(5-carbamoyl-4-(m-tolylamino)pyrimidin-2-ylamino)propyl)cyclopropylcarbamte.

Step 7: To a mixture of (R)-benzyl 1-(1-(5-carbamoyl-4-(m-tolylamino)pyrimidin-2-ylamino)propyl)cyclopropylcarbamte in EtOAc (2 mL) was added Pd/C (spatula tip) and charged with H2 (1 atm). Upon completion, Pd/C was filtered off and the filtrate was concentrated to give crude residue, which was purified by preparative HPLC to give (R)-2-(1-(1-aminocyclopropyl)propylamino)-4-(m-tolylamino)pyrimidine-5-carboxamide (23 mg). MS found for C₁₈H₂₄N₆O as (M+H)⁺ 341.5, UV: λ=241.6.

### Example 127

### (R)-4-(3-(2H-1,2,3-triazol-2-yl)phenylamino)-2-(1-(1-aminocyclopropyl)propylamino)pyrimidine-5-carboxamide

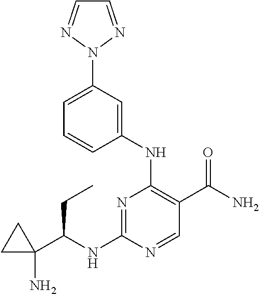 [SMILES: CC[C@@H](Nc1ncc(C(N)=O)c(Nc2cccc(-n3nccn3)c2)n1)C1(N)CC1]

The title compound was synthesized similar to Example 126. MS found for C₁₉H₂₃N₉O as (M+H)⁺ 394.5, UV: λ=248.7.

### Example 128

### (S)-2-(1-(1-aminocyclopropyl)propylamino)-4-(m-tolylamino)pyrimidine-5-carboxamide

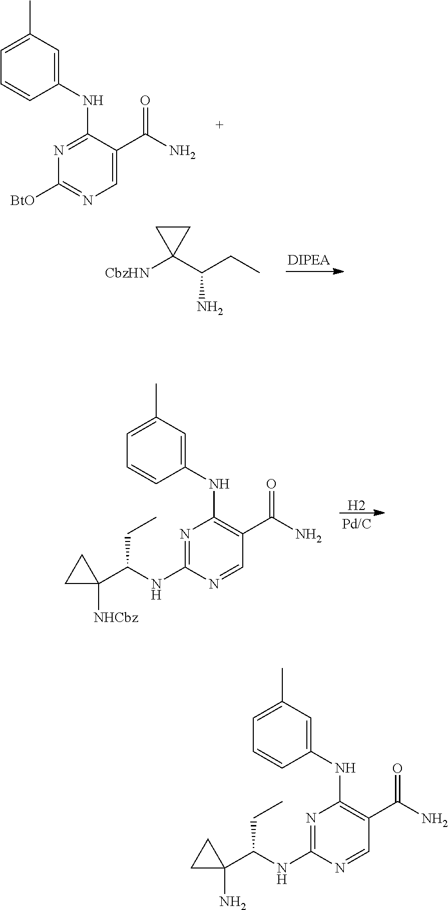

Step 1: To a suspension of 2-(1H-benzo[d][1,2,3]triazol-1-yloxy)-4-(m-tolylamino)pyrimidine-5-carboxamide (54 mg, 0.15 mmol) in AcCN (1.5 mL) was added (S)-benzyl 1-(1-aminopropyl)cyclopropylcarbamte (46 mg, 0.166 mmol) and DIPEA (0.060 mL, 0.33 mmol). After stirred at 60° C. for 3 h, the solution was concentrated, the residue was diluted with water, the precipitate was collected by filtration to give (S)-benzyl 1-(1-(5-carbamoyl-4-(m-tolylamino)pyrimidin-2-ylamino)propyl)cyclopropylcarbamte.

Step 2: To a mixture of (S)-benzyl 1-(1-(5-carbamoyl-4-(m-tolylamino)pyrimidin-2-ylamino)propyl)cyclopropylcarbamte in EtOAc (2 mL) was added Pd/C (spatula tip) and charged with H2 (1 atm). Upon completion, Pd/C was filtered off and the filtrate was concentrated to give crude residue, which was purified by preparative HPLC to give (S)-2-(1-(1-aminocyclopropyl)propylamino)-4-(m-tolylamino)pyrimidine-5-carboxamide (28 mg). MS found for C₁₈H₂₄N₆O as (M+H)⁺ 341.5, UV: λ=240.4.

### Example 129

### (S)-4-(3-(2H-1,2,3-triazol-2-yl)phenylamino)-2-(1-(1-aminocyclopropyl)propylamino)pyrimidine-5-carboxamide

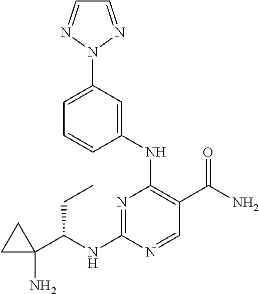 [SMILES: CC[C@H](Nc1ncc(C(N)=O)c(Nc2cccc(-n3nccn3)c2)n1)C1(N)CC1]

The title compound was synthesized similar to Example 128. MS found for C₁₉H₂₃N₉O as (M+H)⁺ 394.5, UV: λ=247.5.

### Example 130

### (R)-2-(1-(1-aminocyclopropyl)propylamino)-4-(p-tolylamino)pyrimidine-5-carboxamide

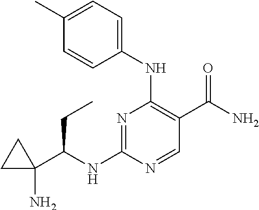 [SMILES: CC[C@@H](Nc1ncc(C(N)=O)c(Nc2ccc(C)cc2)n1)C1(N)CC1]

The title compound was synthesized similar to Example 126. MS found for C₁₈H₂₄N₆O as (M+H)⁺ 341.5. λ=241.6.

### Example 131

### (R)-2-(1-(1-aminocyclopropyl)propylamino)-4-(3-(pyrimidin-2-yl)phenylamino)pyrimidine-5-carboxamide

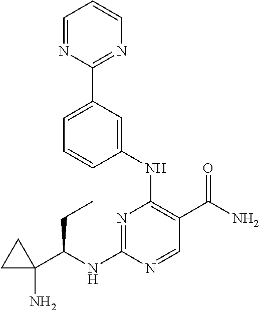 [SMILES: CC[C@@H](Nc1ncc(C(N)=O)c(Nc2cccc(-c3ncccn3)c2)n1)C1(N)CC1]

The title compound was synthesized similar to Example 126. MS found for C₂₁H₂₄N₈O as (M+H)⁺ 405.6, UV: λ=246.3.

### Example 132

### (R)-4-(3-(1H-pyrazol-1-yl)phenylamino)-2-(1-(1-aminocyclopropyl)propylamino)pyrimidine-5-carboxamide

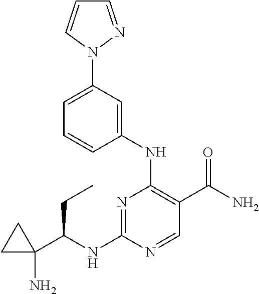 [SMILES: CC[C@@H](Nc1ncc(C(N)=O)c(Nc2cccc(-n3cccn3)c2)n1)C1(N)CC1]

The title compound was synthesized similar to Example 126. MS found for C₂₀H₂₄N₈O as (M+H)⁺ 393.5, UV: λ=247.5.

### Example 133

### (R)-2-(1-(1-aminocyclopropyl)propylamino)-4-(3-(1,4,5,6-tetrahydropyrimidin-2-yl)phenylamino)pyrimidine-5-carboxamide

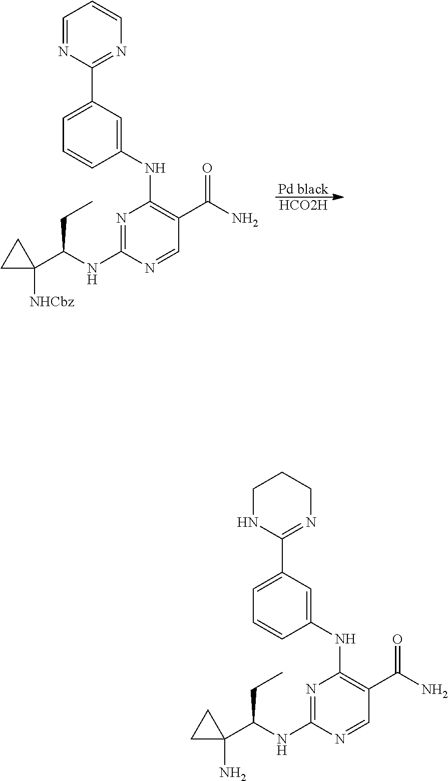 [SMILES: CC[C@@H](Nc1ncc(C(N)=O)c(Nc2cccc(-c3ncccn3)c2)n1)C1(NC(=O)OCc2ccccc2)CC1 ; "HCO2H" ; "Pd black" ; CC[C@@H](Nc1ncc(C(N)=O)c(Nc2cccc(C3=NCCCN3)c2)n1)C1(N)CC1]

To a solution of (R)-benzyl 1-(1-(5-carbamoyl-4-(3-(pyrimidin-2-yl)phenylamino)pyrimidin-2-ylamino)propyl)cyclopropylcarbamte (900 mg) in 5% formic acid in MeOH (40 mL) was added Pd black (350 mg), 10 min later, the solution was concentrated and purified by preparative HPLC to give (R)-2-(1-(1-aminocyclopropyl)propylamino)-4-(3-(1,4,5,6-tetrahydropyrimidin-2-yl)phenylamino)pyrimidine-5-carboxamide. MS found for C₂₁H₂₈N₈O as (M+H)⁺ 409.4, UV: λ=239.3.

### Example 134

### (S)-2-(1-(1-aminocyclopropyl)(cyclopropyl)methylamino)-4-(3-(pyrimidin-2-yl)phenylamino)pyrimidine-5-carboxamide

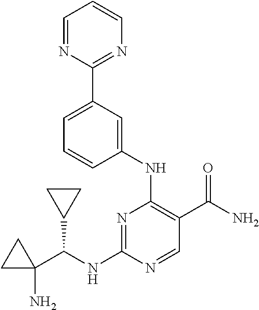 [SMILES: NC(=O)c1cnc(N[C@@H](C2CC2)C2(N)CC2)nc1Nc1cccc(-c2ncccn2)c1]

The title compound was synthesized similar to Example 128. MS found for C₂₂H₂₄N₈O as (M+H)⁺ 417.3, UV: λ=247.5.

### Example 135

### (R)-2-(1-(1-aminocyclopropyl)(cyclopropyl)methylamino)-4-(3-(pyrimidin-2-yl)phenylamino)pyrimidine-5-carboxamide

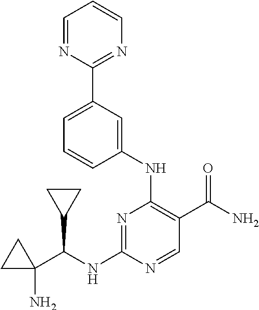 [SMILES: NC(=O)c1cnc(N[C@H](C2CC2)C2(N)CC2)nc1Nc1cccc(-c2ncccn2)c1]

The title compound was synthesized similar to Example 126. MS found for C₂₂H₂₄N₈O as (M+H)⁺ 417.3, UV: λ=251.1.

### Example 136

### (R)-2-(1-(1-aminocyclopropyl)-2-methylpropylamino)-4-(3-(pyrimidin-2-yl)phenylamino)pyrimidine-5-carboxamide

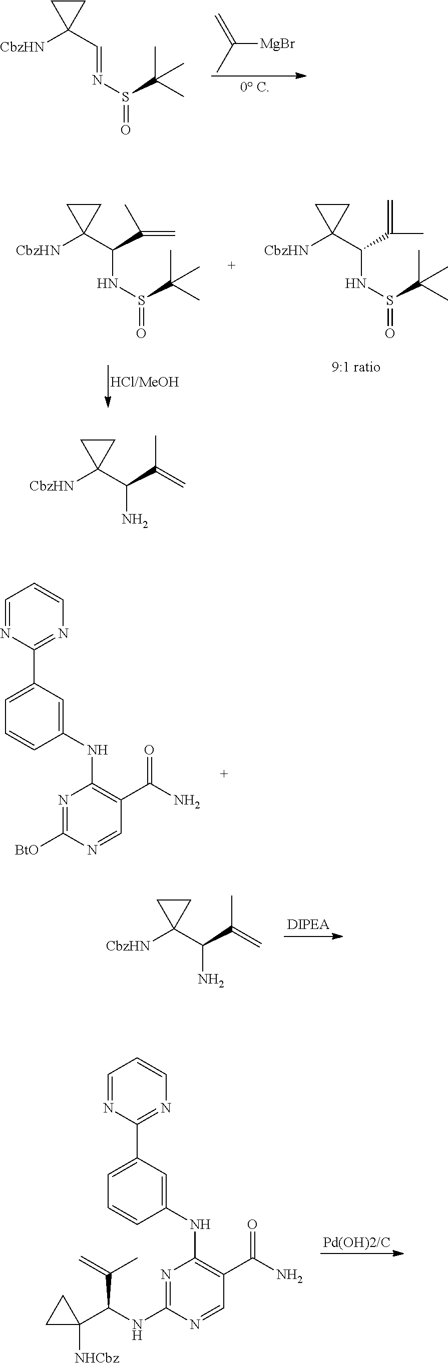

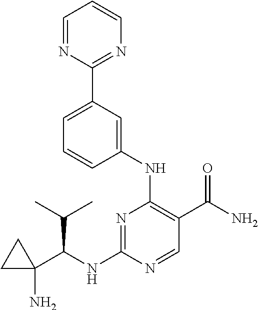 [SMILES: CC(C)[C@@H](Nc1ncc(C(N)=O)c(Nc2cccc(-c3ncccn3)c2)n1)C1(N)CC1]

### Synthesis of (R)-benzyl 1-(1-amino-2-methylallyl)cyclopropylcarbamte

Step 1: To a solution of (R,E)-benzyl 1-((tert-butylsulfinylimino)methyl)cyclopropylcarbamate (200 mg, 0.62 mmol) in DCM (2 ml) under Nitrogen at 0° C. was added a solution of isopropenyl magnesium bromide in THF (0.5 M, 5 mL, 2.5 mmol). After stirred for 1 h, the mixture was quenched with Sat. NH₄Cl, extracted with EtOAc, EtOAc layer was combined, washed with brine, dried and concentrated to give crude oil, which was purified by preparative HPLC to give benzyl 1-((R)-1-((R)-1,1-dimethylethylsulfinamido)-2-methylallyl)cyclopropylcarbamate (120 mg) and benzyl 1-((S)-1-((R)-1,1-dimethylethylsulfinamido)-2-methylallyl)cyclopropylcarbamate (10 mg).

Step 2: To a solution of benzyl 1-((R)-1-((R)-1,1-dimethylethylsulfinamido)-2-methylallyl)cyclopropylcarbamate (150 mg) in MeOH (2 ml) was added 4N HCl in dioxane (0.15 ml). The mixture was stirred for 30 min and then was concentrated and subjected to high vacuum to give (R)-benzyl-1-(1-amino-2-methylallyl)cyclopropylcarbamate hydrochloride as white solid (116 mg).

### Synthesis of (R)-2-(1-(1-aminocyclopropyl)-2-methylpropylamino)-4-(3-(pyrimidin-2-yl)phenylamino)pyrimidine-5-carboxamide

Step 1: To a suspension of 2-(1H-benzo[d][1,2,3]triazol-1-yloxy)-4-(3-(pyrimidin-2-yl)phenylamino)pyrimidine-5-carboxamide (55 mg, 0.12 mmol) in NMP (0.8 mL) was added (R)-benzyl-1-(1-amino2-methylallyl)cyclopropylcarbamate hydrochloride (45 mg, 0.144 mmol) and DIPEA (0.060 mL, 0.33 mmol). After stirred at 60° C. for 3 h, the solution was concentrated, the residue was diluted with water, the precipitate was collected by filtration to give (R)-benzyl 1-(1-(5-carbamoyl-4-(3-(pyrimidine-2-yl)phenylamino)pyrimidin-2-ylamino)-2-methylallyl)cyclopropylcarbamte (60 mg).

Step 2: To a mixture of (R)-benzyl 1-(1-(5-carbamoyl-4-(3-(pyrimidine-2-yl)phenylamino)pyrimidin-2-ylamino)-2-methylallyl)cyclopropylcarbamte (60 mg) in MeOH (2 mL) was added Pd(OH)₂/C (50 mg) and charged with H2 (1 atm). After stirred for 4 h, Pd(OH)₂/C was filtered off and the filtrate was concentrated to give crude residue, which was purified by preparative HPLC to give (R)-2-(1-(1-aminocyclopropyl)-2-methylpropylamino)-4-(3-(pyrimidin-2-yl)phenylamino)pyrimidine-5-carboxamide (17 mg). MS found for C₂₂H₂₆N₈O as (M+H)⁺ 419.3, UV: λ=247.5.

### Example 137

### (R)-4-(3-(2H-1,2,3-triazol-2-yl)phenylamino)-2-(1-(1-aminocyclopropyl)(cyclopropyl)methylamino)pyrimidine-5-carboxamide

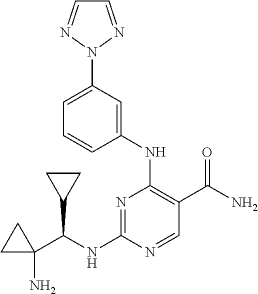 [SMILES: NC(=O)c1cnc(N[C@H](C2CC2)C2(N)CC2)nc1Nc1cccc(-n2nccn2)c1]

The title compound was synthesized similar to Example 126. MS found for C₂₀H₂₃N₉O as (M+H)⁺ 406.3, UV: λ=249.9.

### Example 138

### (R)-4-(3-(2H-1,2,3-triazol-2-yl)phenylamino)-2-(1-(1-aminocyclopropyl)-2-methylpropylamino)pyrimidine-5-carboxamide

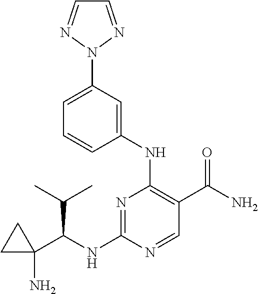 [SMILES: CC(C)[C@@H](Nc1ncc(C(N)=O)c(Nc2cccc(-n3nccn3)c2)n1)C1(N)CC1]

The title compound was synthesized similar to Example 136. MS found for C₂₀H₂₅N₉O as (M+H)⁺ 408.4, UV: λ=249.9.

### Example 139

### (R)-4-(3-(1H-pyrazol-1-yl)phenylamino)-2-(1-(1-aminocyclopropyl)-3-methylbutylamino)pyrimidine-5-carboxamide

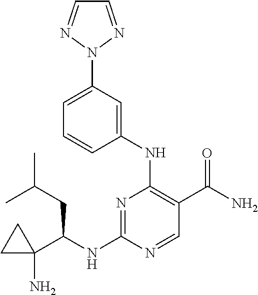 [SMILES: CC(C)C[C@@H](Nc1ncc(C(N)=O)c(Nc2cccc(-n3nccn3)c2)n1)C1(N)CC1]

The title compound was synthesized similar to Example 136. MS found for C₂₂H₂₈N₈O as (M+H)⁺ 421.3, UV: λ=249.9.

### Example 140

### (R)-4-(3-(1H-pyrazol-1-yl)phenylamino)-2-(1-(1-aminocyclopropyl)-2-methylpropylamino)pyrimidine-5-carboxamide

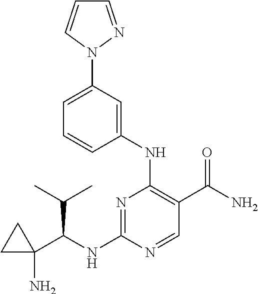 [SMILES: CC(C)[C@@H](Nc1ncc(C(N)=O)c(Nc2cccc(-n3cccn3)c2)n1)C1(N)CC1]

The title compound was synthesized similar to Example 136. MS found for C₂₁H₂₆N₈O as (M+H)⁺ 407.7, UV: λ=205.4, 253.7, 302.9.

### Example 141

### (R)-2-(1-(1-aminocyclopropyl)ethylamino)-4-(3-(pyrimidin-2-yl)phenylamino)pyrimidine-5-carboxamide

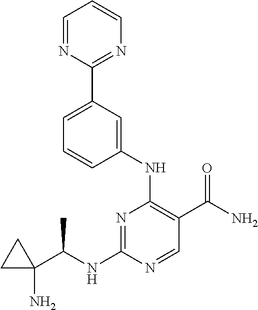 [SMILES: C[C@@H](Nc1ncc(C(N)=O)c(Nc2cccc(-c3ncccn3)c2)n1)C1(N)CC1]

The title compound was synthesized similar to Example 126. MS found for C₂₀H₂₂N₈O as (M+H)⁺ 391.3, UV: λ=257.3, 296.7.

### Example 142

### (R)-4-(3-(2H-1,2,3-triazol-2-yl)phenylamino)-2-(1-(1-aminocyclopropyl)ethylamino)pyrimidine-5-carboxamide

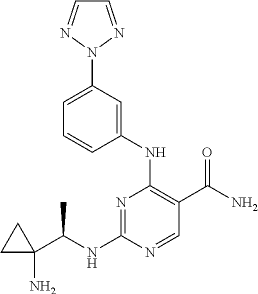 [SMILES: C[C@@H](Nc1ncc(C(N)=O)c(Nc2cccc(-n3nccn3)c2)n1)C1(N)CC1]

The title compound was synthesized similar to Example 126. MS found for C₁₈H₂₁N₉O as (M+H)⁺ 380.3, UV: λ=247.5.

### Example 143

### (R)-4-(3-(1H-pyrazol-1-yl)phenylamino)-2-(1-(1-aminocyclopropyl)ethylamino)pyrimidine-5-carboxamide

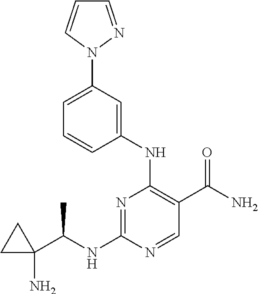 [SMILES: C[C@@H](Nc1ncc(C(N)=O)c(Nc2cccc(-n3cccn3)c2)n1)C1(N)CC1]

The title compound was synthesized similar to Example 126. MS found for C₁₉H₂₂N₈O as (M+H)⁺ 379.3, UV: λ=247.5.

### Example 144

### (R)-2-(1-(1-aminocyclopropyl)-2-methylpropylamino)-4-(m-tolylamino)pyrimidine-5-carboxamide

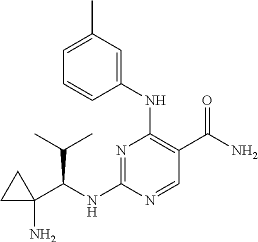 [SMILES: Cc1cccc(Nc2nc(N[C@H](C(C)C)C3(N)CC3)ncc2C(N)=O)c1]

The title compound was synthesized similar to Example 136. MS found for C₁₉H₂₆N₆O as (M+H)⁺ 355.3, UV: λ=239.3.

### Example 145

### (R)-2-(1-(1-aminocyclopropyl)ethylamino)-4-(m-tolylamino)pyrimidine-5-carboxamide

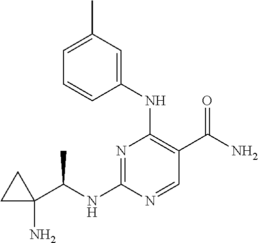 [SMILES: Cc1cccc(Nc2nc(N[C@H](C)C3(N)CC3)ncc2C(N)=O)c1]

The title compound was synthesized similar to Example 126. MS found for C₁₇H₂₁N₆O as (M+H)⁺ 327.2, UV: λ=241.6.

### Example 146

### (R)-2-(1-(1-aminocyclopropyl)ethylamino)-4-(p-tolylamino)pyrimidine-5-carboxamide

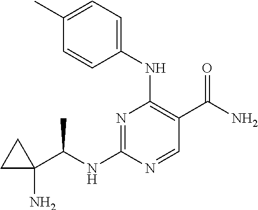 [SMILES: Cc1ccc(Nc2nc(N[C@H](C)C3(N)CC3)ncc2C(N)=O)cc1]

The title compound was synthesized similar to Example 126. MS found for C₁₇H₂₁N₆O as (M+H)⁺ 327.2, UV: λ=242.8.

### Example 147

### (R)-2-(1-(1-aminocyclopropyl)(phenyl)methylamino)-4-(m-tolylamino)pyrimidine-5-carboxamide

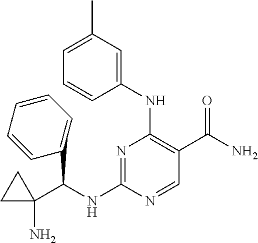 [SMILES: Cc1cccc(Nc2nc(N[C@H](c3ccccc3)C3(N)CC3)ncc2C(N)=O)c1]

The title compound was synthesized similar to Example 126. MS found for C₂₂H₂₄N₆O as (M+H)⁺ 389.3, UV: λ=240, 295.

### Example 148

### (5)-2-(1-(1-aminocyclopropyl)(phenyl)methylamino)-4-(m-tolylamino)pyrimidine-5-carboxamide

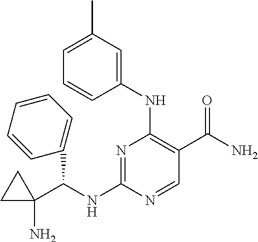 [SMILES: Cc1cccc(Nc2nc(N[C@@H](c3ccccc3)C3(N)CC3)ncc2C(N)=O)c1]

The title compound was synthesized similar to Example 128. MS found for C₂₂H₂₄N₆O as (M+H)⁺ 389.3, UV: λ=240.4, 294.9.

### Example 149

### (R)-4-(3-(2H-1,2,3-triazol-2-yl)phenylamino)-2-(1-(1-aminocyclopropyl)(phenyl)methylamino)pyrimidine-5-carboxamide

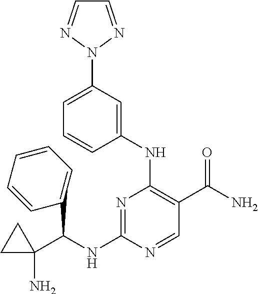 [SMILES: NC(=O)c1cnc(N[C@H](c2ccccc2)C2(N)CC2)nc1Nc1cccc(-n2nccn2)c1]

The title compound was synthesized similar to Example 126. MS found for C₂₃H₂₃N₉O as (M+H)⁺ 442.2, UV: λ=249.9.

### Example 150

### (R)-2-(1-(1-aminocyclopropyl)(phenyl)methylamino)-4-(3-(pyrimidin-2-yl)phenylamino)pyrimidine-5-carboxamide

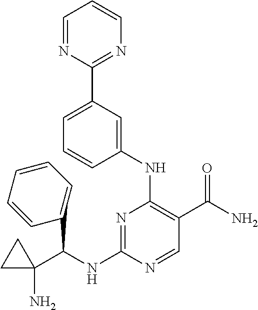 [SMILES: NC(=O)c1cnc(N[C@H](c2ccccc2)C2(N)CC2)nc1Nc1cccc(-c2ncccn2)c1]

The title compound was synthesized similar to Example 126. MS found for C₂₅H₂₄N₈O as (M+H)⁺ 453.3, UV: λ=249.9.

### Example 151

### (R)-4-(3-(1H-pyrazol-1-yl)phenylamino)-2-(1-(1-aminocyclopropyl)(cyclopropyl)methylamino)pyrimidine-5-carboxamide

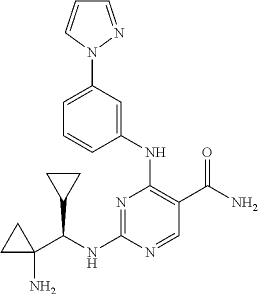 [SMILES: NC(=O)c1cnc(N[C@H](C2CC2)C2(N)CC2)nc1Nc1cccc(-n2cccn2)c1]

The title compound was synthesized similar to Example 126. MS found for C₂₁H₂₄N₈O as (M+H)⁺ 405.4, UV: λ=249.9.

### Example 152

### (R)-2-(1-(1-aminocyclopropyl)(cyclopropyl)methylamino)-4-(quinolin-3-ylamino)pyrimidine-5-carboxamide

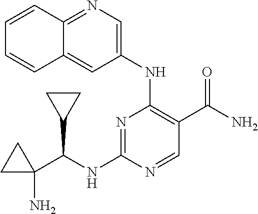 [SMILES: NC(=O)c1cnc(N[C@H](C2CC2)C2(N)CC2)nc1Nc1cnc2ccccc2c1]

The title compound was synthesized similar to Example 126. MS found for C₂₁H₂₃N₇O as (M+H)⁺ 390.3, UV: λ=242.8, 281.9.

### Example 153

### (R)-2-(1-(1-aminocyclopropyl)(cyclopropyl)methylamino)-4-(1,2,3,4-tetrahydroquinolin-6-ylamino)pyrimidine-5-carboxamide

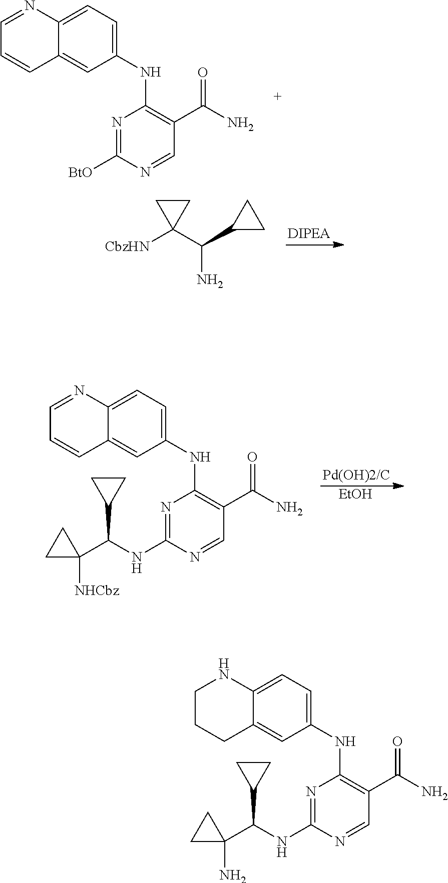

Step 1: To a suspension of 2-(1H-benzo[d][1,2,3]triazol-1-yloxy)-4-(quinolin-6-ylamino)pyrimidine-5-carboxamide (99 mg, 0.25 mmol) in NMP (1.5 mL) was added (R)-benzyl-1-(1-amino-2-cyclopropyl)cyclopropylcarbamate hydrochloride (77 mg, 0.26 mmol) and DIPEA (0.111 mL, 0.625 mmol). After stirred at 75° C. for 3 h, the solution was diluted with water, the precipitate was collected by filtration to give (R)-benzyl 1-(1-(5-carbamoyl-4-(quinolin-6-ylamino)pyrimidin-2-ylamino)-2-cyclopropyl)cyclopropylcarbamte.

Step 2: To a mixture of (R)-benzyl 1-(1-(5-carbamoyl-4-(quinolin-6-ylamino)pyrimidin-2-ylamino)-2-cyclopropyl)cyclopropylcarbamte in EtOH (2 mL) was added Pd(OH)2/C (50 mg) and charged with H2 (1 atm). After stirred for 4 h, Pd(OH)2/C was filtered off and the filtrate was concentrated to give crude residue, which was purified by preparative HPLC to give (R)-2-(1-(1-aminocyclopropyl)(cyclopropyl)methylamino)-4-(1,2,3,4-tetrahydroquinolin-6-ylamino)pyrimidine-5-carboxamide. MS found for C₂₁H₂₇N₇O as (M+H)⁺ 394.3, UV: λ=244.4.

### Example 154

### (R)-2-(1-(1-aminocyclopropyl)propylamino)-4-(quinolin-3-ylamino)pyrimidine-5-carboxamide

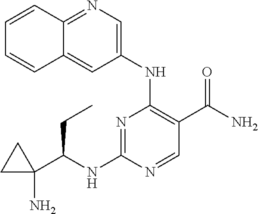 [SMILES: CC[C@@H](Nc1ncc(C(N)=O)c(Nc2cnc3ccccc3c2)n1)C1(N)CC1]

The title compound was synthesized similar to Example 126. MS found for C₂₀H₂₃N₇O as (M+H)⁺ 378.3, UV: λ=242.8, 281.9.

### Example 155

### (R)-2-(1-(1-aminocyclopropyl)(cyclopropyl)methylamino)-4-(quinolin-6-ylamino)pyrimidine-5-carboxamide

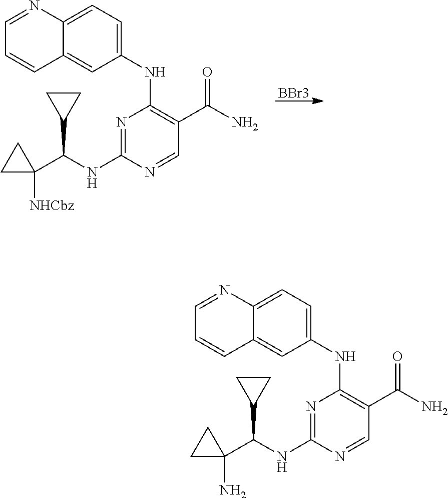

To a mixture of (R)-benzyl 1-(1-(5-carbamoyl-4-(quinolin-6-ylamino)pyrimidin-2-ylamino)-2-cyclopropyl)cyclopropylcarbamte in DCM (2 mL) at 0° C. was added BBr3 (2 eq), after 30 min, the solution was concentrated and the residue was purified by preparative HPLC to give (R)-2-(1-(1-aminocyclopropyl)(cyclopropyl)methylamino)-4-(quinolin-6-ylamino)pyrimidine-5-carboxamide. MS found for C₂₁H₂₃N₇O as (M+H)⁺ 390.3, UV: λ=242.8, 277.1.

### Example 156

### (R)-2-(1-(1-aminocyclopropyl)(cyclopropyl)methylamino)-4-(3-fluorophenylamino)pyrimidine-5-carboxamide

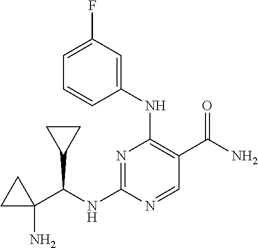 [SMILES: NC(=O)c1cnc(N[C@H](C2CC2)C2(N)CC2)nc1Nc1cccc(F)c1]

The title compound was synthesized similar to Example 126. MS found for C₁₈H₂₁FN₆O as (M+H)⁺ 357.2, UV: λ=245.2.

### Example 157

### (R)-2-(1-(1-aminocyclopropyl)(cyclopropyl)methylamino)-4-(3,5-difluorophenylamino)pyrimidine-5-carboxamide

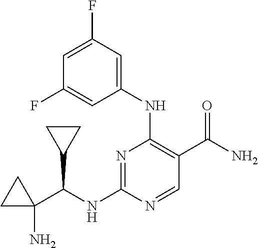 [SMILES: NC(=O)c1cnc(N[C@H](C2CC2)C2(N)CC2)nc1Nc1cc(F)cc(F)c1]

The title compound was synthesized similar to Example 126. MS found for C₁₈H₂₀F₂N₆O as (M+H)⁺ 375.3, UV: λ=247.5.

### Example 158

### (R)-2-(1-(1-aminocyclopropyl)-2-methylpropylamino)-4-(quinolin-3-ylamino)pyrimidine-5-carboxamide

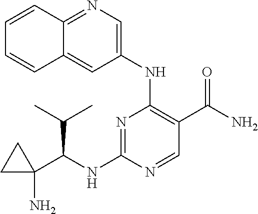 [SMILES: CC(C)[C@@H](Nc1ncc(C(N)=O)c(Nc2cnc3ccccc3c2)n1)C1(N)CC1]

The title compound was synthesized similar to Example 136. MS found for C₂₁H₂₅N₇O as (M+H)⁺ 392.3, UV: λ=244.0.

### Example 159

### (R)-2-(1-(1-aminocyclopropyl)-2-methylpropylamino)-4-(3-fluorophenylamino)pyrimidine-5-carboxamide

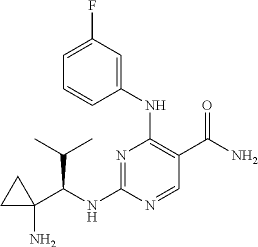 [SMILES: CC(C)[C@@H](Nc1ncc(C(N)=O)c(Nc2cccc(F)c2)n1)C1(N)CC1]

The title compound was synthesized similar to Example 136. MS found for C₁₈H₂₃FN₆O as (M+H)⁺ 359.3, UV: λ=245.2.

### Example 160

### (R)-2-(1-(1-aminocyclopropyl)-2-methylpropylamino)-4-(3,5-difluorophenylamino)pyrimidine-5-carboxamide

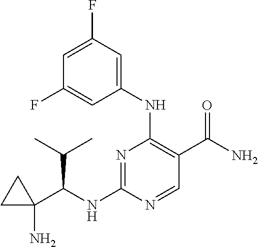 [SMILES: CC(C)[C@@H](Nc1ncc(C(N)=O)c(Nc2cc(F)cc(F)c2)n1)C1(N)CC1]

The title compound was synthesized similar to Example 136. MS found for C₁₈H₂₂F₂N₆O as (M+H)⁺ 377.3, UV: λ=247.5.

### Example 161

### (±)-4-(3-(2H-1,2,3-triazol-2-yl)phenylamino)-2-(1S,2S,6R)-2-amino-6-hydroxycyclohexylamino)pyrimidine-5-carboxamide

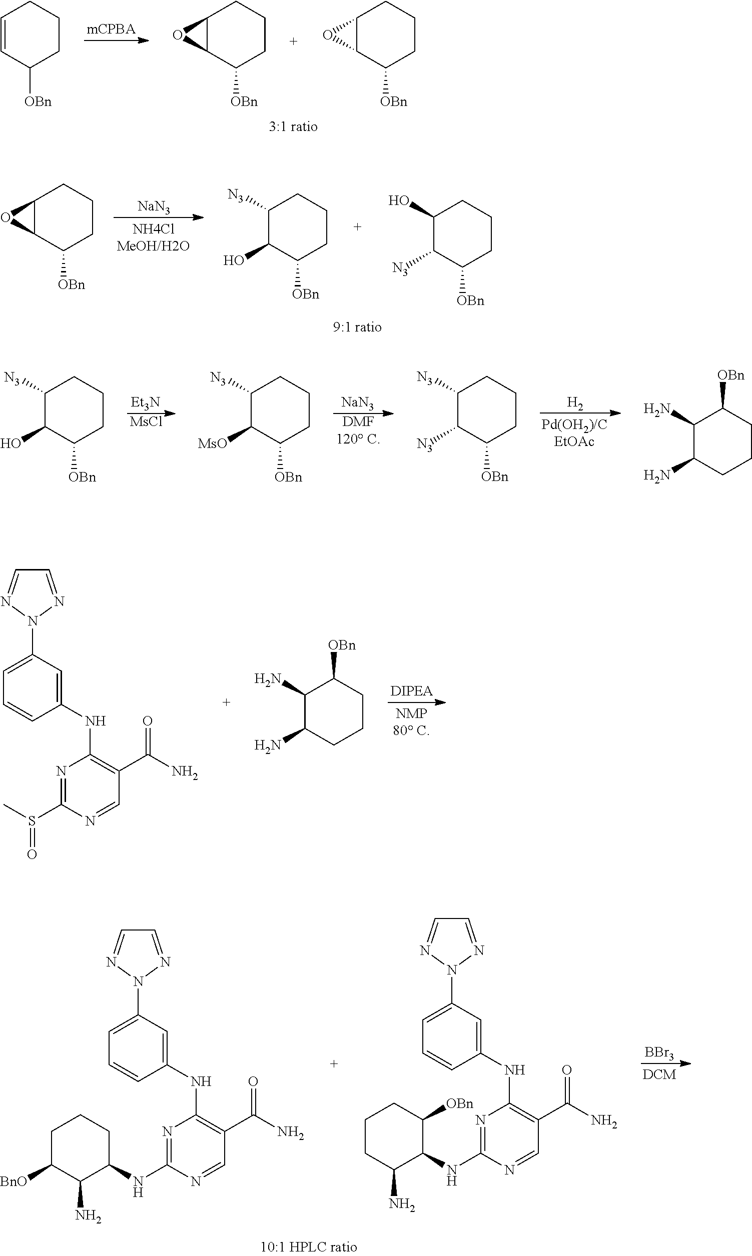

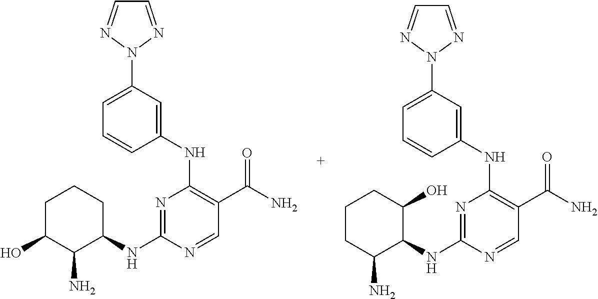 [SMILES: NC(=O)c1cnc(N[C@@H]2CCC[C@H](O)[C@@H]2N)nc1Nc1cccc(-n2nccn2)c1 ; NC(=O)c1cnc(N[C@@H]2[C@H](O)CCC[C@@H]2N)nc1Nc1cccc(-n2nccn2)c1]

### Synthesis of (±)-(1R,2R,3S)-3-(benzyloxy)cyclohexane-1,2-diamine

Step 1: To a solution of ((cyclohex-2-enyloxy)methyl)benzene (1.64 g, 8.68 mmol) in DCM (20 ml) at 0° C. was added mCPBA (65%, 2.53 g, 9.54 mmol). After stirred at room temperature for 15 h, it was diluted with DCM, the organic layer was washed with aqueous Na2S2O3, Sat. NaHCO3, brine, dried and concentrated to give crude residue, which was purified by column chromatography to give (±)-(1R,2S,6S)-2-(benzyloxy)-7-oxabicyclo[4,1,0]heptanes (1.0 g) and (±)-(1R,2S,6R)-2-(benzyloxy)-7-oxabicyclo[4,1,0]heptanes (330 mg).

Step 2: To a solution of (±)-(1R,2S,6S)-2-(benzyloxy)-7-oxabicyclo[4,1,0]heptanes (1.0 g, 4.88 mmol) in MeOH (24 ml) and water (3 ml) was added NaN3 (1.58 g, 24.4 mmol) and NH4Cl (574 mg, 10.74 mmol). The mixture was heated at 80° C. for 15 h, then it was quenched with water, the aqueous layer was extracted with ether, organic layer was combined, washed with brine, dried and concentrated to give crude mixture, which was separated by column chromatography to give (±)-(1S,2R,6S)-2-azido-6-(benzyloxy)cyclohexanol (820 mg).

Step 3: To a solution of (±)-(1S,2R,6S)-2-azido-6-(benzyloxy)cyclohexanol (820 mg, 3.29 mmol) in DCM (15 ml) was added Et3N (0.92 ml, 6.59 mmol) and MsCl (0.384 ml). After stirred at room temperature for 30 min, the reaction was quenched with water, extracted with ether, the organic layer was combined, washed with Sat. NaHCO3, brine, dried and concentrated to give (±)-(1S,2R,6S)-2-azido-6-(benzyloxy)cyclohexane methanesulfonate.

Step 4: To a solution of (±)-(1S,2R,6S)-2-azido-6-(benzyloxy)cyclohexane methanesulfonate (400 mg, 1.22 mmol) in DMF (15 ml) was added NaN3 (400 mg, 6.12 mmol). After stirred at 120° C. for 15 h, the reaction was quenched with water, extracted with ether, ether layer was combined, washed with brine, dried and concentrated to give crude residue, which was purified by column chromatography (Hexanes/EtOAc=9: 1) to give (±)-(((1S,2R,3R)-2,3-diazidocyclohexyloxy)methyl)benzene (180 mg).

Step 5: To a solution of (±)-(((1S,2R,3R)-2,3-diazidocyclohexyloxy)methyl)benzene (180 mg) in tOAc (5 ml) was added Pd/C, charged with H2 (1 atm). After stirred for 5 h, Pd/C was filtered off, the filtrate was concentrated to give (±)-(1R,2R,3S)-3-(benzyloxy)cyclohexane-1,2-diamine (120 mg).

### Synthesis of (±)-4-(3-(2H-1,2,3-triazol-2-yl)phenylamino)-2-((1S,2S,6R)-2-amino-6-hydroxycyclohexylamino)pyrimidine-5-carboxamide

Step 1: To a suspension of 4-(3-(2H-1,2,3-triazol-2-yl)phenylamino)-2-((1S,2S,6R)-2-methylsulfinyl)pyrimidine-5-carboxamide (102 mg, 0.3 mmol) in NMP (1.5 ml) was added (±)-(1R,2R,3S)-3-(benzyloxy)cyclohexane-1,2-diamine (96 mg, 0.44 mmol) and DIPEA (0.133 ml, 0.75 mmol). After heated at 80° C. for 2 h, the reaction was cooled and diluted with water, the precipitate was collected by filtration to give a crude mixture.

Step 2: To a solution of the above mentioned crude mixture in DCM (2 ml) was added BBr3 (excess) at 0° C. After stirred at room temperature for 3 h, the mixture was concentrated and purified by preparative HPLC to give (±)-4-(3-(2H-1,2,3-triazol-2-yl)phenylamino)-2-((1R,2R,3S)-2-amino-3-hydroxycyclohexylamino)pyrimidine-5-carboxamide (61 mg) and (±)-4-(3-(2H-1,2,3-triazol-2-yl)phenylamino)-2-((1S,2S,6R)-2-amino-6-hydroxycyclohexylamino)pyrimidine-5-carboxamide (2 mg). MS found for C₁₉H₂₃N₉O₂ as (M+H)⁺ 410.3, UV: λ=248.7.

### Example 162

### (R)-2-(1-(1-aminocyclopropyl)(cyclopropyl)methylamino)-4-(1-methyl-1H-indazol-4-ylamino)pyrimidine-5-carboxamide

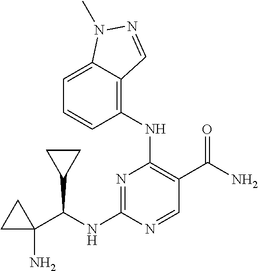 [SMILES: Cn1ncc2c(Nc3nc(N[C@H](C4CC4)C4(N)CC4)ncc3C(N)=O)cccc21]

The title compound was synthesized similar to Example 126. MS found for C₂₀H₂₄N₈O as (M+H)⁺ 393.4, UV: λ=208.6, 241.6.

### Example 163

### (R)-4-(3-(1H-1,2,4-triazol-1-yl)phenylamino)-2-(1-(1-aminocyclopropyl)(cyclopropyl)methylamino)pyrimidine-5-carboxamide

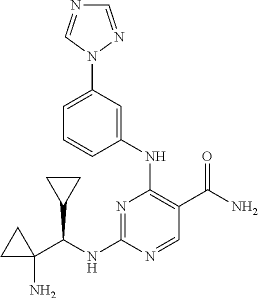 [SMILES: NC(=O)c1cnc(N[C@H](C2CC2)C2(N)CC2)nc1Nc1cccc(-n2cncn2)c1]

The title compound was synthesized similar to Example 126. MS found for C₂₀H₂₃N₉O as (M+H)⁺ 406.4, UV: λ=244.0.

### Example 164

### (R)-4-(3-(2H-1,2,3-triazol-1-yl)phenylamino)-2-(2-oxocyclohexylamino)pyrimidine-5-carboxamide

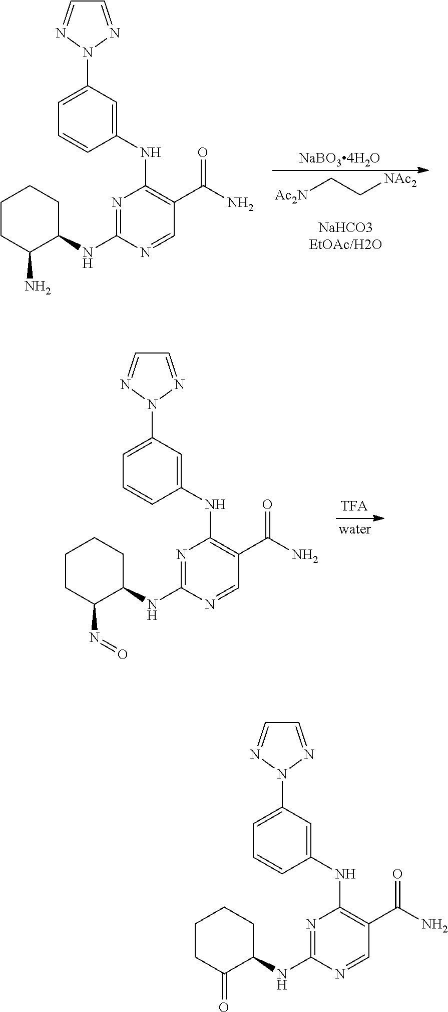

Step 1: To a solution of 4-(3-(2H-1,2,3-triazol-1-yl)phenylamino)-2-((1R,2S)-2-aminocyclohexylamino)pyrimidine-5-carboxamide (590 mg, 1.5 mmol, Onyx WH665Q) in EtOAc (20 ml) and water (20 ml) was added NaHCO3 (1.26 g, 15 mmol, EMD lot 1970C112), NaBO3.4H₂O (2.31 g, 15 mmol, Aldrich 08612MD) and N,N,N,N-tetraacetyl ethylenediamine (856 mg, 3.75 mmol, TCI-EP lot FIF01). After stirred at room temperature for 2 h, it was diluted with EtOAc, organic layer was separated and filtered to get rid of insoluble material, and the filtrate was concentrated to give a crude yellow solid (300 mg).

Step 2: To a suspension of the above crude solid in AcCN/H2O (8 ml, 1:1 volume ratio) was added TFA (6 drops), then it was stirred at room temperature for 15 h, purification of the reaction mixture by preparative HPLC gave (R)-4-(3-(2H-1,2,3-triazol-1-yl)phenylamino)-2-(2-oxocyclohexylamino)pyrimidine-5-carboxamide (45 mg). MS found for C₁₉H₂₀N₈O₂ as (M+H)⁺ 393.3, UV: λ=251.1.

### Example 165

### (±)-4-(3-(2H-1,2,3-triazol-1-yl)phenylamino)-2-((1R,2R)-2-amino-3-oxocyclohexylamino)pyrimidine-5-carboxamide

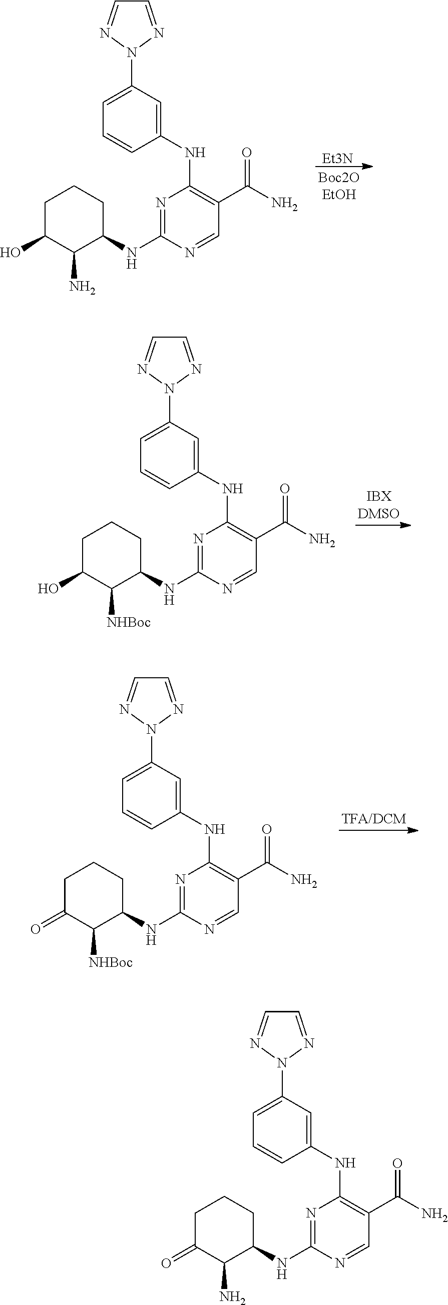 [SMILES: "Et3N" ; NC(=O)c1cnc(N[C@@H]2CCC[C@H](O)[C@@H]2N)nc1Nc1cccc(-n2nccn2)c1 ; CC(C)(C)OC(=O)N[C@H]1[C@@H](O)CCC[C@H]1Nc1ncc(C(N)=O)c(Nc2cccc(-n3nccn3)c2)n1 ; CC(C)(C)OC(=O)N[C@H]1C(=O)CCC[C@H]1Nc1ncc(C(N)=O)c(Nc2cccc(-n3nccn3)c2)n1 ; NC(=O)c1cnc(N[C@@H]2CCCC(=O)[C@@H]2N)nc1Nc1cccc(-n2nccn2)c1]

Step 1: To a solution of (±)-4-(3-(2H-1,2,3-triazol-2-yl)phenylamino)-2-((1R,2R,3S)-2-amino-3-hydroxycyclohexylamino)pyrimidine-5-carboxamide (27 mg, 0.066 mmol) in EtOH (1 ml) was added Et3N (0.018 ml, 0.132 mmol) and Boc2O (14.4 mg, 0.066 mmol). After stirred for 30 min, EtOH was removed under vacuum, and the residue was diluted with H2O, the resulting precipitate was collected by filtration to give (±)-tert butyl (1R,2R,6S)-2-(4-(3-(2H-1,2,3-triazol-2-yl)phenylamino)-5-carbamoylpyrimidin-2-ylamino)-6-hydroxycyclohexylcarbamate (20 mg).

Step 2: To a solution of (±)-tert butyl (1R,2R,6S)-2-(4-(3-(2H-1,2,3-triazol-2-yl)phenylamino)-5-carbamoylpyrimidin-2-ylamino)-6-hydroxycyclohexylcarbamate (20 mg) in DMSO (1 ml) was added IBX (54 mg, 0.20 mmol). After stirred for 48 h, it was diluted with water and was purified by preparative HPLC to give (±)-tert butyl (1R,2R)-2-(4-(3-(2H-1,2,3-triazol-2-yl)phenylamino)-5-carbamoylpyrimidin-2-ylamino)-6-oxocyclohexylcarbamate (8 mg).

Step 3: To a solution of (±)-tert butyl (1R,2R)-2-(4-(3-(2H-1,2,3-triazol-2-yl)phenylamino)-5-carbamoylpyrimidin-2-ylamino)-6-oxocyclohexylcarbamate (8 mg) in DCM (2 ml) was added TFA (2 ml), 10 min later, the reaction was concentrated and the residue was purified by preparative HPLC to give (±)-4-(3-(2H-1,2,3-triazol-1-yl)phenylamino)-2-((1R,2R)-2-amino-3-oxocyclohexylamino)pyrimidine-5-carboxamide (2 mg). MS found for C₁₉H₂₁N₉O₂ as (M+H)⁺ 408.3, UV: λ=248.7.

### Example 166

### (R)-2-(1-(1-aminocyclopropyl)(cyclopropyl)methylamino)-4-(benzo[d]thiazol-5-ylamino)pyrimidine-5-carboxamide

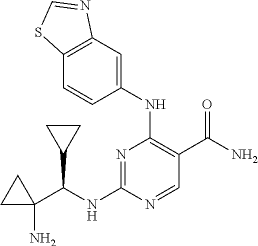 [SMILES: NC(=O)c1cnc(N[C@H](C2CC2)C2(N)CC2)nc1Nc1ccc2scnc2c1]

The title compound was synthesized similar to Example 126. MS found for C₁₉H₂₁N₇OS as (M+H)⁺ 396.5, UV: λ=265.8, 288.0.

### Example 167

### (R)-2-(1-(1-aminocyclopropyl)(cyclopropyl)methylamino)-4-(benzo[d]thiazol-6-ylamino)pyrimidine-5-carboxamide

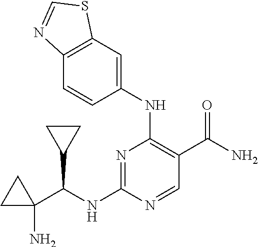 [SMILES: NC(=O)c1cnc(N[C@H](C2CC2)C2(N)CC2)nc1Nc1ccc2ncsc2c1]

The title compound was synthesized similar to Example 126. MS found for C₁₉H₂₁N₇OS as (M+H)⁺ 396.3, UV: λ=241.6.

### Example 168

### 2-((1R,2R)-2-amino-3,3-difluorocyclohexylamino)-4-(quinolin-3-ylamino)pyrimidine-5-carboxamide

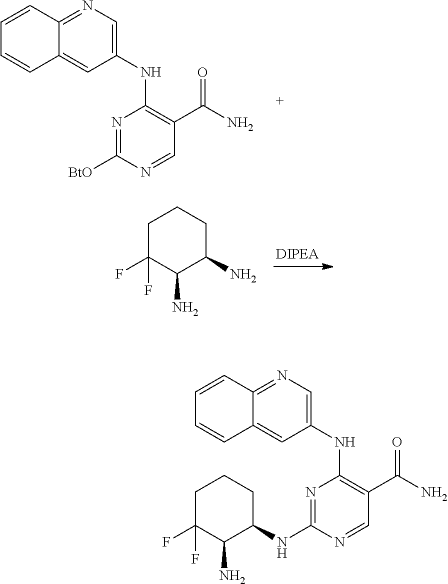

To a suspension of 2-(1H-benzo[d][1,2,3]triazol-1-yloxy)-4-(quinolin-3-ylamino)pyrimidine-5-carboxamide (50 mg, 0.134 mmol) in NMP (0.8 ml) was added (1R,2R)-3,3-difluorocyclohexane-1,2-diamine dihydrochloride (30 mg, 0.134 mmol) and DIPEA (0.072 ml, 0.402 mmol). After heated at 80° C. for 2 h, the mixture was cooled and purified by preparative HPLC to give 2-((1R,2R)-2-amino-3,3-difluorocyclohexylamino)-4-(quinolin-3-ylamino)pyrimidine-5-carboxamide (51 mg). MS found for C₂₀H₂₁F₂N₇O as (M+H)⁺ 414.3, UV: λ=241.6, 280.7.

### Example 169

### 2-((1R,2R)-2-amino-3,3-difluorocyclohexylamino)-4-(quinolin-6-ylamino)pyrimidine-5-carboxamide

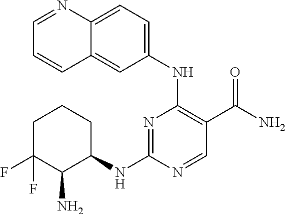 [SMILES: NC(=O)c1cnc(N[C@@H]2CCCC(F)(F)[C@@H]2N)nc1Nc1ccc2ncccc2c1]

The title compound was synthesized similar to Example 170. MS found for C₂₀H₂₁F₂N₇O as (M+H)⁺ 414.3, UV: λ=236.9, 283.1.

### Example 170

### (R)-2-(1-(1-aminocyclopropyl)(cyclopropyl)methylamino)-4-(4-fluorophenylamino)pyrimidine-5-carboxamide

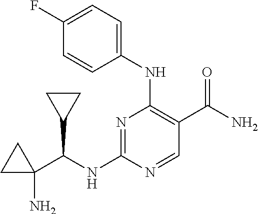 [SMILES: NC(=O)c1cnc(N[C@H](C2CC2)C2(N)CC2)nc1Nc1ccc(F)cc1]

The title compound was synthesized similar to Example 126. MS found for C₁₈H₂₁FN₆O as (M+H)⁺ 357.2, UV: λ=242.8.

### Example 171

### (R)-2-(1-(1-aminocyclopropyl)(cyclopropyl)methylamino)-4-(thieno[2,3-b]pyridine-3-ylamino)pyrimidine-5-carboxamide

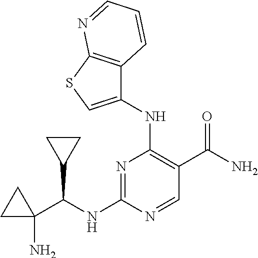 [SMILES: NC(=O)c1cnc(N[C@H](C2CC2)C2(N)CC2)nc1Nc1csc2ncccc12]

The title compound was synthesized similar to Example 126. MS found for C₁₉H₂₁N₇OS as (M+H)⁺ 396.3, UV: λ=238.1, 297.3.

### Example 172

### (±)-2-((1R,2R,3S)-2-amino-3-fluorocyclohexylamino)-4-(m-tolylamino)pyrimidine-5-carboxamide and Example 173 (±)-2-((1S,2S,6R)-2-amino-6-fluorocyclohexylamino)-4-(m-tolylamino)pyrimidine-5-carboxamide

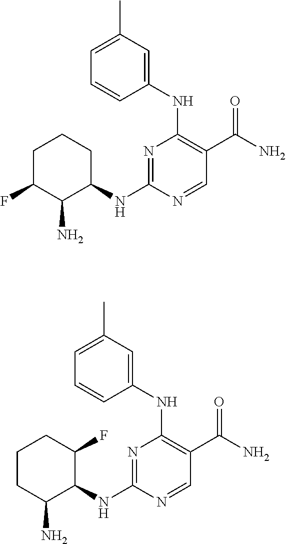 [SMILES: Cc1cccc(Nc2nc(N[C@@H]3CCC[C@H](F)[C@@H]3N)ncc2C(N)=O)c1 ; Cc1cccc(Nc2nc(N[C@@H]3[C@H](F)CCC[C@@H]3N)ncc2C(N)=O)c1]

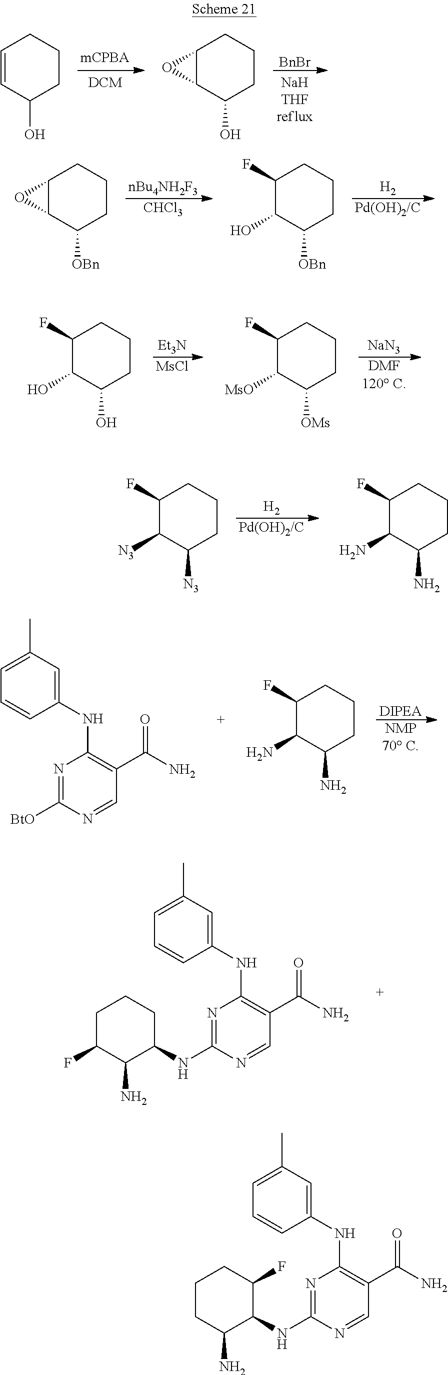

### Synthesis of (±)-(1R,2R,3S)-3-fluorocyclohexane-1,2-diamine

Step 1: To a solution of cyclohex-2-enol (1.0 g) in DCM (70 ml) at 0° C. was added mCPBA (65%, 4.5 g, 17.0 mmol). After stirred at room temperature for 15 h, the solid was filtered off, filter cake was washed with more DCM, the filtrate was washed with aqueous Na2S2O3, Sat. NaHCO3, brine, dried and concentrated to give (±)-(1S,2S,6R)-7-oxabicyclo[4.1.0]hepta-2-ol as crude residue.

Step 2: A solution of (±)-(1S,2S,6R)-7-oxabicyclo[4.1.0]hepta-2-ol in THF (10 ml) was added to a suspension of NaH (1.0 g, 65%) and BnBr (1.54 g) in THF (25 ml) at 55° C. After heating at 55° C. for 5 h, it was poured to ice water, the aqueous layer was extracted with ether, ether layer was combined, washed with brine, dried and concentrated to give crude oil, which was purified by column chromatography to give (±)-(1S,2S,6R)-2-(benzyloxy)-7-oxabicyclo[4.1.0]heptane (640 mg).

Step 3: A mixture of (±)-(1S,2S,6R)-2-(benzyloxy)-7-oxabicyclo[4.1.0]heptanes (640 mg) and tetrabutylammonium dihydrogen trifluoride (3.2 g) was heated at 80° C. for 15 h. The reaction was then dilued with water, extracted with EtOAc, organic layer was combined, washed with brine, dried and concentrated to give crude mixture, which was purified by column chromatography to give (±)-(1S,2S,6S)-2-(benzyloxy)-6-fluorocyclohexanol (590 mg).

Step 4: To a solution of (±)-(1S,2S,6S)-2-(benzyloxy)-6-fluorocyclohexanol (590 mg, 2.62 mmol) in EtOH (10 ml) was added Pd(OH)2/C (200 mg). After completion, Pd(OH)2/C was filtered off, and the filtrate was concentrated to give (±)-(1S,2S,6S)-3-fluorocyclohexane-1,2-diol (350 mg).

Step 5: To a solution of (±)-(1S,2S,6S)-3-fluorocyclohexane-1,2-diol (345 mg, 2.57 mmol) in DCM (8 ml) was added Et3N (1.08 ml, 7.71 mmol) and MSCl (0.5 ml, 6.43 mmol). After stirred at room temperature for 2 h, the reaction was quenched with water, extracted with ether, the organic layer was combined, washed with Sat. NaHCO3, brine, dried and concentrated to give crude residue, which was purified by column chromatography to give (±)-(1S,2S,6S)-3-fluorocyclohexane-1,2-diyl dimethanesulfonate (670 mg).

Step 6: To a solution of (±)-(1S,2S,6S)-3-fluorocyclohexane-1,2-diyl dimethanesulfonate (670 mg, 2.31 mmol) in DMF (30 ml) was added NaN3 (1.5 g, 23 mmol). After stirred at 120° C. for 15 h, the reaction was quenched with water, extracted with ether, ether layer was combined, washed with brine, dried and concentrated to give crude residue, which was purified by column chromatography (Hexanes/EtOAc=9: 1) to give (±)-(1R,2R,3S)-1,2-diazido-3-fluorocyclohexane (300 mg).

Step 7: To a solution of (±)-(1R,2R,3S)-1,2-diazido-3-fluorocyclohexane (300 mg) in EtOAc (6 ml) was added Pd(OH)2/C, charged with H2 (1 atm). After stirred for 2 h, Pd(OH)2/C was filtered off, the filtrate was concentrated to give (±)-(1R,2R,3S)-3-fluorocyclohexane-1,2-diamine (295 mg).

### Synthesis of (±)-2-((1R,2R,3S)-2-amino-3-fluorocyclohexylamino)-4-(m-tolylamino)pyrimidine-5-carboxamide and (±)-2-((1S,2S,6R)-2-amino-6-fluorocyclohexylamino)-4-(m-tolylamino)pyrimidine-5-carboxamide

To a suspension of 2-(1H-benzo[d][1,2,3]triazol-1-yloxy)-4-(m-tolylamino)pyrimidine-5-carboxamide (57 mg, 0.16 mmol) in AcCN (1.5 ml) was added (±)-(1R,2R,3S)-3-fluorocyclohexane-1,2-diamine (40 mg, 0.24 mmol) and DIPEA (0.071 ml, 0.4 mmol). After heating at 75° C. for 3 h, it was diluted with AcCN/H₂O, and was purified by preparative HPLC to give (±)-2-((1R,2R,3S)-2-amino-3-fluorocyclohexylamino)-4-(m-tolylamino)pyrimidine-5-carboxamide (36 mg, MS found for C₁₈H₂₃FN₆O as (M+H)⁺ 359.3, UV: λ=238.1, 286.6) and (±)-2-((1S,2S,6R)-2-amino-6-fluorocyclohexylamino)-4-(m-tolylamino)pyrimidine-5-carboxamide (4 mg, MS found for C₁₈H₂₃FN₆O as (M+H)⁺ 359.3, UV: λ=239.3, 286.6).

### Example 174

### (±)-4-(3-(2H-1,2,3-triazol-1-yl)phenylamino)-2-((1R,2R,3S)-2-amino-3-fluorocyclohexylamino)pyrimidine-5-carboxamide and Example 175 (±)-4-(3-(2H-1,2,3-triazol-1-yl)phenylamino)-2-((1S,2S,6R)-2-amino-6-fluorocyclohexylamino)pyrimidine-5-carboxamide

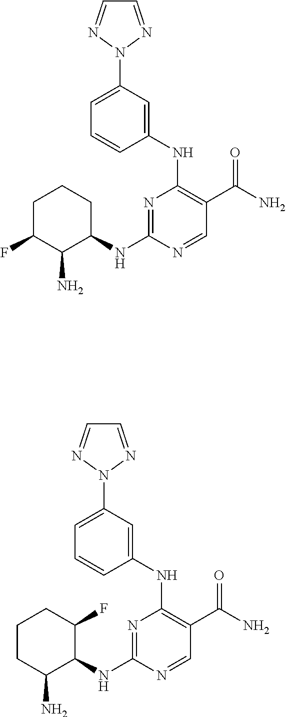 [SMILES: NC(=O)c1cnc(N[C@@H]2CCC[C@H](F)[C@@H]2N)nc1Nc1cccc(-n2nccn2)c1 ; NC(=O)c1cnc(N[C@@H]2[C@H](F)CCC[C@@H]2N)nc1Nc1cccc(-n2nccn2)c1]

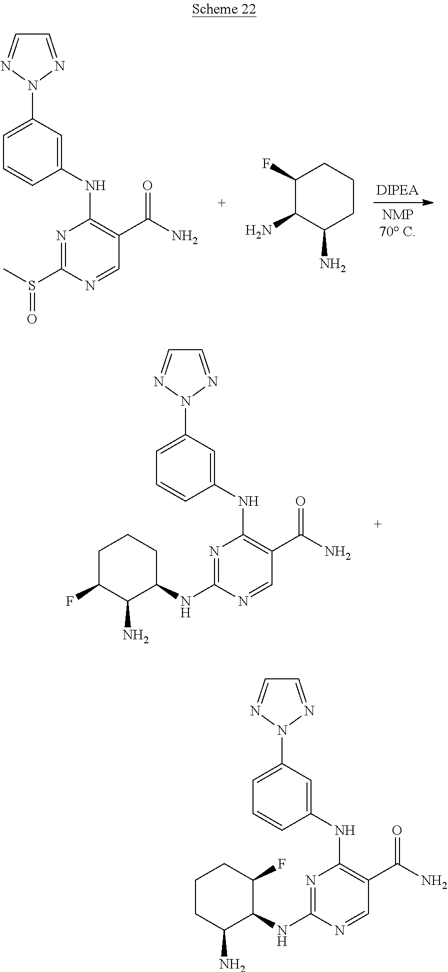 [SMILES: CS(=O)c1ncc(C(N)=O)c(Nc2cccc(-n3nccn3)c2)n1 ; N[C@@H]1[C@H](N)CCC[C@@H]1F ; NC(=O)c1cnc(N[C@@H]2CCC[C@H](F)[C@@H]2N)nc1Nc1cccc(-n2nccn2)c1 ; NC(=O)c1cnc(N[C@@H]2[C@H](F)CCC[C@@H]2N)nc1Nc1cccc(-n2nccn2)c1]

To a suspension of 4-(3-(2H-1,2,3-triazol-2-yl)phenylamino)-2-((1S,2S,6R)-2-methylsulfinyl)pyrimidine-5-carboxamide (55 mg, 0.16 mmol) in NMP (1.0 ml) was added (±)-(1R,2R,3S)-3-fluorocyclohexane-1,2-diamine (40 mg, 0.24 mmol) and DIPEA (0.071 ml, 0.4 mmol). After heated at 80° C. for 3 h, the reaction was cooled and diluted with water, and then was purified by preparative HPLC to give (±)-4-(3-(2H-1,2,3-triazol-1-yl)phenylamino)-2-((1R,2R,3S)-2-amino-3-fluorocyclohexylamino)pyrimidine-5-carboxamide (48 mg, MS found for C₁₉H₂₂FN₉O as (M+H)⁺ 412.4, UV: λ=248.7) and (±)-4-(3-(2H-1,2,3-triazol-1-yl)phenylamino)-2-((1S,2S,6R)-2-amino-6-fluorocyclohexylamino)pyrimidine-5-carboxamide (2 mg, MS found for C₁₉H₂₂FN₉O as (M+H)⁺ 412.4, UV: λ=245.2).

### Example 176

### 4-(3-(2H-1,2,3-triazol-1-yl)phenylamino)-2-((1R,2S)-2-nitrosocyclohexylamino)pyrimidine-5-carboxamide

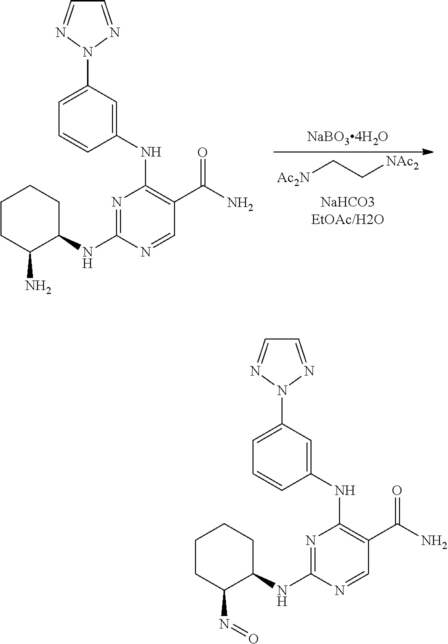

To a solution of 4-(3-(2H-1,2,3-triazol-1-yl)phenylamino)-2-((1R,2S)-2-aminocyclohexylamino)pyrimidine-5-carboxamide (118 mg, 0.3 mmol, Onyx WH665Q) in EtOAc (3 ml) and water (3 ml) was added NaHCO₃ (252 mg, 3 mmol, EMD lot 1970C112), NaBO3.4H2O (462 mg, 3 mmol, Aldrich 08612MD) and N,N,N,N-tetraacetyl ethylenediamine (171 mg, 0.75 mmol, TCI-EP lot FIF01). After stirred at room temperature for 2 h, it was diluted with EtOAc, organic layer was separated and filtered to get rid of insoluble material, and the filtrate was concentrated to give a crude solid, which was first subjected to preparative HPLC followed by preparative TLC (DCM/EtOAc=1/9 and 1% MeOH) to give 4-(3-(2H-1,2,3-triazol-1-yl)phenylamino)-2-((1R,2S)-2-nitrosocyclohexylamino)pyrimidine-5-carboxamid2 (8 mg). MS found for C₁₉H₂₁N₉O₂ as (M+H)⁺ 393.3, UV: λ=251.1.

### Example 177

### 2-((3R,4R)-3-aminotetrahydro-2H-pyran-4-ylamino)-4-(quinolin-3-ylamino)pyrimidine-5-carboxamide

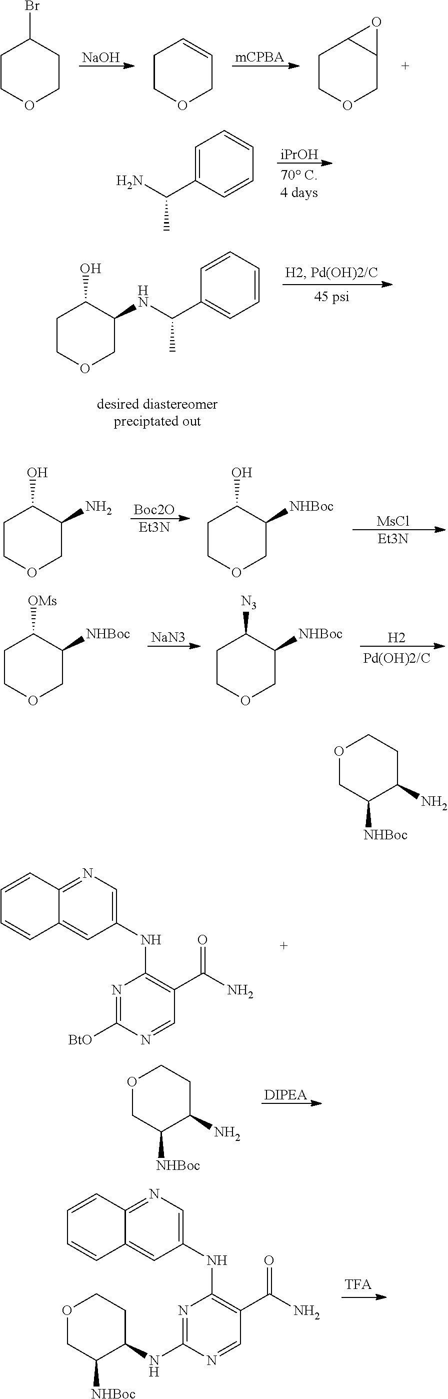

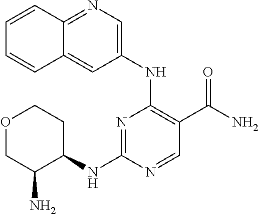 [SMILES: NC(=O)c1cnc(N[C@@H]2CCOC[C@@H]2N)nc1Nc1cnc2ccccc2c1]

### Synthesis of tert-butyl (3R,4R)-4-aminotetrahydro-2H-pyran-3-ylcarbamate

Step 1: To 4-bromotetrahydro-2H-pyran (10 g) in round bottom flask was added 10N NaOH (15 ml). After heated at 90° C. for 24 h, the aqueous layer was separated out leaving 3,6-dihydro-2H-pyran as the crude material.

Step 2: To a solution of 3,6-dihydro-2H-pyran in CHCl₃ (40 ml) at 0° C. was added mCPBA (16 g, 60 mmol). After stirred at 0° C. for 1 h, it was warmed up to room temperature and stirred for 15 h. Solid was filtered off, and the filtrate was diluted with more CHCl₃, organic layer was washed with Sat. NaHCO3, Na2S2O3, brine, dried and concentrated to give 3,7-dioxabicyclo[4.1.0]heptanes (3.3 g).

Step 3: To a solution of 3,7-dioxabicyclo[4.1.0]heptanes (3.3 g) in i-PrOH (18 ml) was added (S)-1-phenylethanamine (3.70 g). After heated at 70° C. for 4 days, the reaction was concentrated and diluted with MTBE and Hexanes, the resulting precipitate was collected by filtration to give (3S,4S)-3-((S)-1-phenylethylamino)tetrahydro-2H-pyran-4-ol (1.47 g).

Step 4: To a solution of (3S,4S)-3-((S)-1-phenylethylamino)tetrahydro-2H-pyran-4-ol (1.47 g, 6.65 mmol) in EtOH (25 ml) was added Pd(OH)2/C (200 mg), charged with H2 (30 psi) in a parr shaker, and was shaked for 15 h, Pd(OH)2/C was filtered off, the filtrate was concentrated to give (3S,4S)-3-aminotetrahydro-2H-pyran-4-ol (600 mg).

Step 5: To a solution of (3S,4S)-3-aminotetrahydro-2H-pyran-4-ol (600 mg, 5.13 mmol) in MeOH (4 ml) was added Et3N (0.1 ml) and a solution of Boc2O (1.2 g, 5.5 mmol) in MeOH (2 ml). After stirred at room temperature for 15 h, it was concentrated and added MTBE (1 ml) and Hexanes (9 ml), the resulting solid was then collected by filtration to give tert-butyl (3S,4S)-4-hydroxytetrahydro-2H-pyran-3-ylcarbamate (970 mg).

Step 6: To a solution of tert-butyl (3S,4S)-4-hydroxytetrahydro-2H-pyran-3-ylcarbamate (970 mg, 4.45 mmol) in DCM (10 ml) at 0° C. was added Et3N (0.752 ml, 5.38 mmol) and MSCl (0.38 ml, 4.89 mmol). After stirred at room temperature for 2 h, the reaction was quenched with water, extracted with ether, the organic layer was combined, washed with Sat. NaHCO3, brine, dried and concentrated to give crude residue, which was purified by short column chromatography to give (3S,4S)-3-(tert-butoxycarbonylamino)tetrahydro-2H-pyran-4-yl methanesulfonate (1.02 g).

Step 7: To a solution of (3S,4S)-3-(tert-butoxycarbonylamino)tetrahydro-2H-pyran-4-yl methanesulfonate (1.02 g, 3.46 mmol) in DMF (5 ml) was added NaOAc (550 mg, 7.0 mmol) and NaN3 (659 mg, 10 mmol). After stirred at 100° C. for 6 h, the reaction was quenched with water, extracted with EtOAc, EtOAc layer was combined, washed with brine, dried and concentrated to give tert-butyl (3R,4R)-4-azidotetrahydro-2H-pyran-3-ylcarbamate as crude solid (700 mg).

Step 8: To a solution of tert-butyl (3R,4R)-4-azidotetrahydro-2H-pyran-3-ylcarbamate (700 mg, 2.89 mmol) in EtOH (10 ml) was added Pd(OH)2/C (200 mg), charged with H2 (1 atm), 6 h later, Pd(OH)2/C was filtered off, and the filtrate was concentrated to give tert-butyl (3R,4R)-4-aminotetrahydro-2H-pyran-3-ylcarbamate (610 mg).

### Synthesis of 2-((3R,4R)-3-aminotetrahydro-2H-pyran-4-ylamino)-4-(quinolin-3-ylamino)pyrimidine-5-carboxamide

Step 1: To a suspension of 2-(1H-benzo[d][1,2,3]triazol-1-yloxy)-4-(quinolin-3-ylamino)pyrimidine-5-carboxamide (85 mg, 0.21 mmol) in NMP (1.2 ml) was added tert-butyl (3R,4R)-4-aminotetrahydro-2H-pyran-3-ylcarbamate (65 mg, 0.3 mmol) and DIPEA (0.053 ml, 0.3 mmol). After heated at 80° C. for 2 h, the mixture was cooled and diluted with water, the precipitate was collected by filtration to give crude solid.

Step 2: To a suspension of the above solid DCM (1 ml) was added TFA (1 ml), after completion, the reaction was concentrated and the residue was purified by preparative HPLC to give 2-((3R,4R)-3-aminotetrahydro-2H-pyran-4-ylamino)-4-(quinolin-3-ylamino)pyrimidine-5-carboxamide. MS found for C₁₉H₂₁N₇O₂ as (M+H)⁺ 380.3, UV: λ=242.8, 279.5.

### Example 178

### 2-((3R,4R)-3-aminotetrahydro-2H-pyran-4-ylamino)-4-(benzo[d]thiazol-5-ylamino)pyrimidine-5-carboxamide

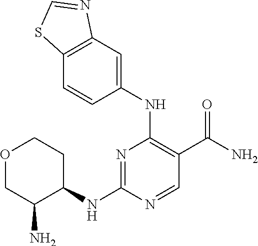 [SMILES: NC(=O)c1cnc(N[C@@H]2CCOC[C@@H]2N)nc1Nc1ccc2scnc2c1]

The title compound was synthesized similar to Example 177. MS found for C₁₇H₁₉N₇O₂S as (M+H)⁺ 386.3, UV: λ=244.0, 286.6.

### Example 179

### 2-((3R,4R)-3-aminotetrahydro-2H-pyran-4-ylamino)-4-(thieno[2,3-b]pyridin-3-ylamino)pyrimidine-5-carboxamide

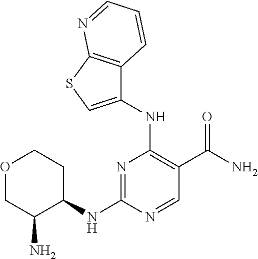 [SMILES: NC(=O)c1cnc(N[C@@H]2CCOC[C@@H]2N)nc1Nc1csc2ncccc12]

The title compound was synthesized similar to Example 177. MS found for C₁₇H₁₉N₇O₂S as (M+H)⁺ 386.3, UV: λ=233.4, 297.3.

### Example 180

### 2-((3R,4R)-3-aminotetrahydro-2H-pyran-4-ylamino)-4-(quinolin-6-ylamino)pyrimidine-5-carboxamide

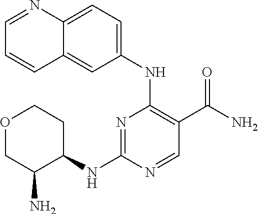 [SMILES: NC(=O)c1cnc(N[C@@H]2CCOC[C@@H]2N)nc1Nc1ccc2ncccc2c1]

The title compound was synthesized similar to Example 177. MS found for C₁₉H₂₁N₇O₂ as (M+H)⁺ 380.3, UV: λ=236.9, 289.0.

### Example 181

### 2-((3R,4R)-3-aminotetrahydro-2H-pyran-4-ylamino)-4-(benzo[d]thiazol-6-ylamino)pyrimidine-5-carboxamide

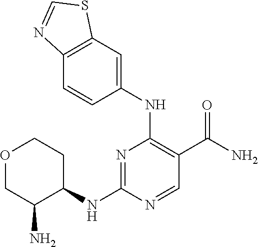 [SMILES: NC(=O)c1cnc(N[C@@H]2CCOC[C@@H]2N)nc1Nc1ccc2ncsc2c1]

The title compound was synthesized similar to Example 177. MS found for C₁₇H₁₉N₇O₂S as (M+H)⁺ 386.3, UV: λ=234.5, 296.1.

### Example 182

### 2-((1R,2R,3S)-2-amino-3-fluorocyclohexylamino)-4-(m-tolylamino)pyrimidine-5-carboxamide

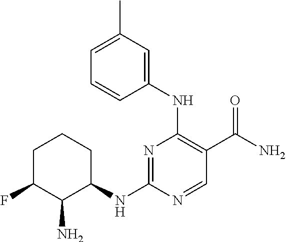 [SMILES: Cc1cccc(Nc2nc(N[C@@H]3CCC[C@H](F)[C@@H]3N)ncc2C(N)=O)c1]

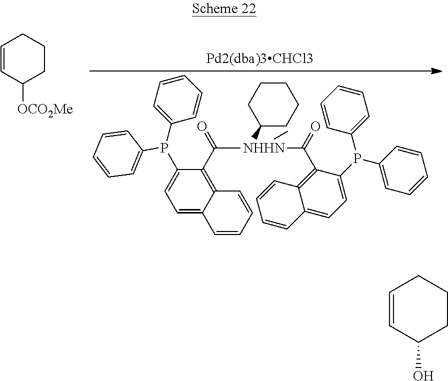

### Synthesis of enantiomerically pure (S)-cyclohexen-2-ol

A solution of cyclohex-2-enyl methyl carbonate (1.41 g) in DCM (4 ml) was added to a mixture of Pd₂ dba₃.CHCl₃ (372 mg, 0.36 mmol) and (1S,2S)-(−)-1,2-diaminocyclohexane-N,N′-bis(2-diphenylphosphino-1-napthoyl) (500 mg, 0.72 mmol) in DCM/water (68 ml/8 ml). After stirred at room temperature for 15 h, it was diluted with more DCM, organic layer was separated and washed with brine, dried and concentrated to give (S)-cyclohexen-2-ol (700 mg). With (S)-cyclohexen-2-ol in hand, the title compound was synthesized same as Example 172. MS found for C₁₈H₂₃FN₆O as (M+H)⁺ 359.3, UV: λ=239.3, 289.0.

### Example 183

### 4-(4-aminocyclohexylamino)-2-(phenylamino)pyrimidine-5-carboxamide

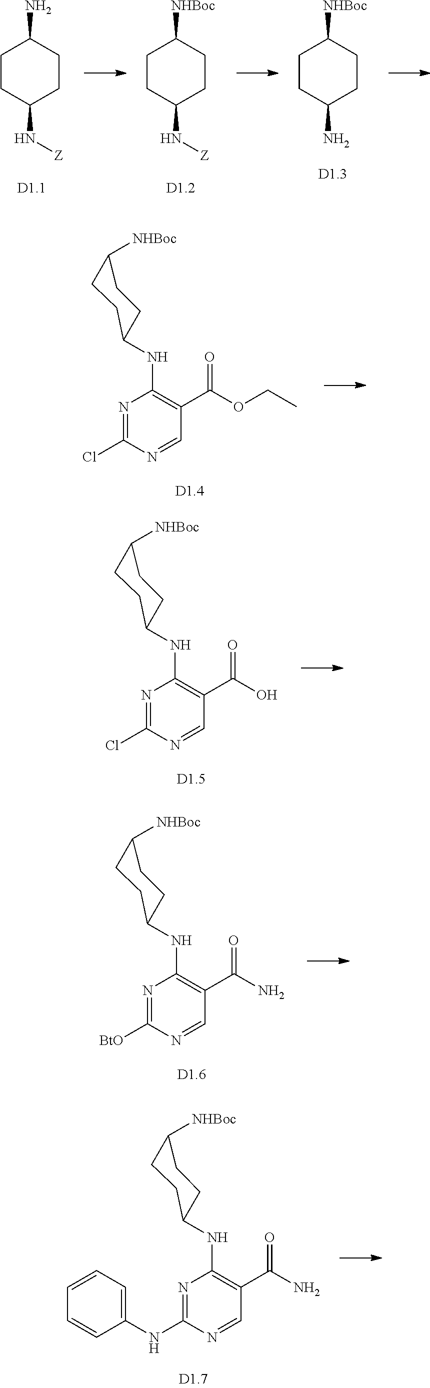

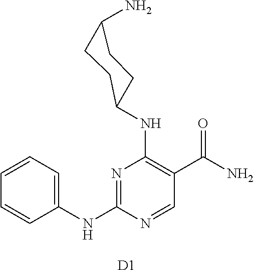 [SMILES: NC(=O)c1cnc(Nc2ccccc2)nc1NC1CCC(N)CC1 ; "D1"]

### Step 1:

Amine D1.1 (Prepared as described in Bauer, Shawn M.; Jia, Zhaozhong J.; Song, Yonghong; Xu, Qing; Mehrotra, Mukund; Rose, Jack W.; Huang, Wolin; Venkataramani, Chandrasekar; Pandey, Anjali. PCT Int. Appl. (2009), WO2009145856A1.) (1.92 g, 7.7 mmol) was diluted with dichloromethane (30 mL) affording an opaque solution. To this was added di-t-butyl dicarbonate (2.0 g, 9.3 mmol) in two portions. The resulting solution was then stirred for approximately 1 hr at which time the reaction was determined to be complete by UPLC. The reaction mixture was then concentrated and dried in vacuo overnight affording 2.47 g of the diprotected diamine as a light pink syrup. MS found for C₁₁H₂₁N₂O₂ as (M−Boc+2H)⁺ 249.2 and (M+Na)⁺ 371.4.

### Step 2:

The amine from the previous step was diluted with 50 mL of methanol and treated with approximately 400 mg of Pd/C (10%, wet) then stirred under an atmosphere of hydrogen overnight. The following morning the reaction was filtered through a short pad of celite to remove the catalyst, concentrated, and used immediately for the next step.

### Step 3:

Amine D1.3 was added as an acetonitrile solution to a stirring solution of ethyl 2,4-dichloropyrimidine-5-carboxylate (Prepared as described in Bauer, Shawn M.; Jia, Zhaozhong J.; Song, Yonghong; Xu, Qing; Mehrotra, Mukund; Rose, Jack W.; Huang, Wolin; Venkataramani, Chandrasekar; Pandey, Anjali. PCT Int. Appl. (2009), WO2009145856A1) (1 g, 4.5 mmol) and DIPEA (1.7 mL, 9.9 mmol) in 15 mL of acetonitrile until all of the dichloropyrimidine was consumed as determined by UPLC. The reaction mixture was then diluted with 1M HCl, then concentrated by rotary evaporation to remove the acetonitrile affording an oil. The mixture was then partitioned with ethyl acetate, the layers separated, and the organic phase extracted an additional time with ethyl acetate. The combined organic layers were concentrated and the resulting syrup used without further purification for the next step. MS found for C₁₈H₂₇ClN₄O₄ as (M+H)⁺ 399.3, 401.3.

### Step 4:

Ester D1.4 was diluted with 15 mL of 1,4-dioxane and 9.75 mL of 1M LiOH and the resulting solution stirred overnight at room temperature. After 2 hrs the reaction was concentrated to approximately 10 mL total volume and acidified with 3M HCl to pH=3. After stirring a gummy solid formed. The liquor was decanted and the solid was resuspended in a small amount of acetonitrile affording a filterable solid which was isolate by filtration and determined to contain the desired carboxylic acid and a small amount of impurity. The material was dried in vacuo and used for the next step. MS found for C₁₆H₂₃ClN₄O₄ as (M−t-Bu+2H)⁺315.2, 317.2.

### Step 5:

Carboxylic acid D1.5 (0.42 g, 1.1 mmol) was dissolved in 5.2 mL of DMF. To this was added HOBt (0.27 g, 1.7 mmol) and EDC (0.33 g, 1.7 mmol). After 10 min the reaction was checked by UPLC which showed formation of the reactive OBt intermediate. Ammonia (0.5M in dioxane, 5.2 mL, 2.6 mmol) was added and the reaction stirred at rt overnight. The following day the reaction was concentrated to remove the dioxane, then diluted to 50 mL total volume. The resulting solid was then isolated by filtration affording the desired amide as a light beige solid. MS found for C₂₂H₂₈N₈O₄ as (M−tBu+2H)⁺ 413.4 and (M−Boc+2H)⁺ 369.4.

### Step 6:

Pyrimidine D1.7 (84 mg, 0.18 mmol) was diluted with NMP (2 mL). To this was added aniline (22 mg, 0.24 mmol) and p-TsOH (46 mg, 0.24 mmol) and the resulting solution stirred until the starting pyrimidine was consumed. The reaction was then diluted with 2 mL of TFA followed by 2 mL of 4M HCl in dioxane, then stirred until deprotection of the Boc amine was complete. The reaction was then concentrated to remove dioxane, then diluted with water and the crude mixture purified by preparative HPLC affording the titled compound after lyophilization. MS found for C₁₇H₂₂N₆O as (M+H)⁺ 327.2. UV: λ=207, 254 nm. ¹H NMR (400 MHz, MeOH-d₄) δ 8.41 (s, 1H), 7.56 (d, 2H), 7.37 (t, 2H), 7.18 (m, 1H), 4.32 (m, 1H), 1.98 (m, 4H), 1.82 (m, 2H), 1.63 (m, 2H).

### Example 184

### 4-(4-aminocyclohexylamino)-2-(p-tolylamino)pyrimidine-5-carboxamide

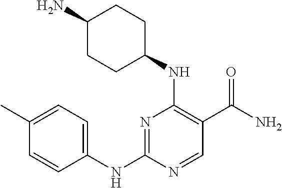 [SMILES: Cc1ccc(Nc2ncc(C(N)=O)c(N[C@H]3CC[C@@H](N)CC3)n2)cc1]

The titled compound was synthesized using a procedure similar to that described in Example 183, using p-anisidine in place of aniline. MS found for C₁₈H₂₄N₆O as (M+H)⁺ 341.2. UV: λ=210, 254 nm. ¹H NMR (400 MHz, MeOH-d₄) δ 8.38 (s, 1H), 7.40 (d, 2H), 7.18 (d, 2H), 4.31 (m, 1H), 2.33 (s, 3H), 1.99 (m, 4H), 1.82 (m, 2H), 1.63 (m, 2H).

### Example 185

### 4-(4-aminocyclohexylamino)-2-(4-methoxyphenylamino)pyrimidine-5-carboxamide

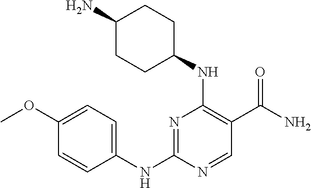 [SMILES: COc1ccc(Nc2ncc(C(N)=O)c(N[C@H]3CC[C@@H](N)CC3)n2)cc1]

The titled compound was synthesized using a procedure similar to that described in Example 183. MS found for C₁₈H₂₄N₆O₂ as (M+H)⁺ 357.2. UV: λ=214, 251, 278 nm. ¹H NMR (400 MHz, MeOH-d₄) δ 8.37 (s, 1H), 7.40 (m, 2H), 6.96 (d, 2H), 4.33 (m, 1H), 3.81 (s, 3H), 2.00 (m, 4H), 1.83 (m, 2H), 1.67 (m, 2H).

### Example 186

### 4-(4-aminocyclohexylamino)-2-(4-chlorophenylamino)pyrimidine-5-carboxamide

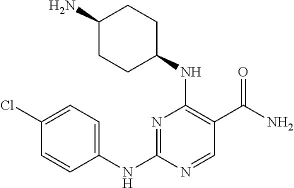 [SMILES: NC(=O)c1cnc(Nc2ccc(Cl)cc2)nc1N[C@H]1CC[C@@H](N)CC1]

The titled compound was synthesized using a procedure similar to that described in Example 183. MS found for C₁₇H₂₁ClN₆O as (M+H)⁺ 361.2, 363.2. UV: λ=210, 256 nm. ¹H NMR (400 MHz, MeOH-d₄) S 8.42 (s, 1H), 7.59 (d, 2H), 7.36 (d, 2H), 4.33 (m, 1H), 1.98 (m, 4H), 1.84 (m, 2H), 1.63 (m, 2H).

### Example 187

### Racemic (1R,3S,4R)-3-amino-4-(5-carbamoyl-4-(m-tolylamino)pyrimidin-2-ylamino)cyclohexanecarboxylic acid was prepared according to Scheme 23

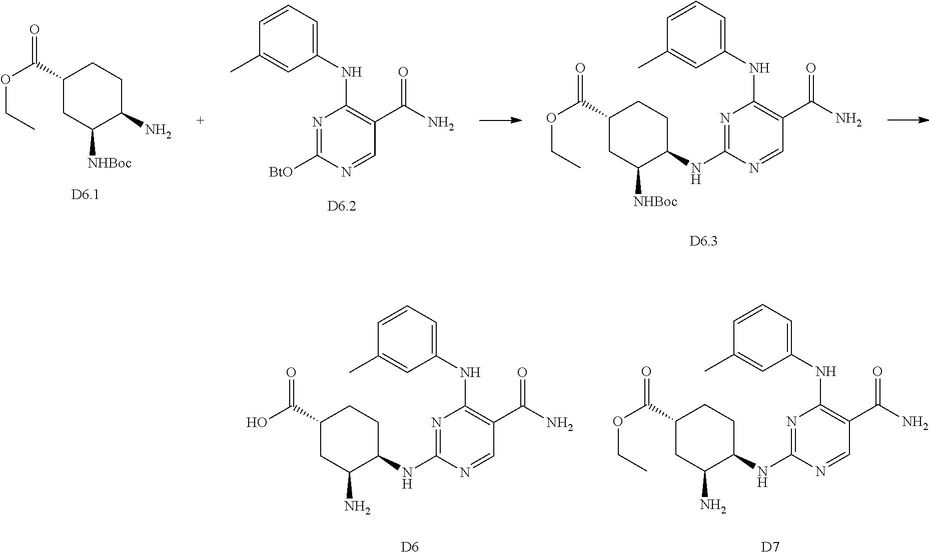

MS found for C₁₉H₂₄N₆O₃ as (M+H)⁺ 385.4. UV: λ=241, 288 nm. ¹H NMR (400 MHz, MeOH-d₄) δ 8.51 (s, 1H). 7.37 (m, 2H), 7.28 (m, 1H), 7.00 (m, 1H), 4.18 (m, 1H), 3.96 (m, 1H), 2.64 (m, 1H), 2.38 (s, 3H), 2.13 (m, 2H), 1.93 (m, 2H), 1.87 (m, 2H)

### Example 188

### Racemic (1R,3S,4R)-ethyl 3-amino-4-(5-carbamoyl-4-(m-tolylamino)pyrimidin-2-ylamino)cyclohexanecarboxylate was prepared according to Scheme 23

MS found for C₂₁H₂₈N₆O₃ as (M+H)⁺ 413.4. UV: λ=241, 290 nm. ¹H NMR (400 MHz, MeOH-d₄) δ 8.52 (s, 1H), 7.41 (m, 2H), 7.29 (t, 1H), 7.05 (m, 1H), 4.13 (m, 3H), 3.92 (m, 1H), 2.63 (m, 1H), 2.38 (s, 3H), 2.10 (m, 2H), 1.63-1.98 (m, 4H), 1.24 (t, 3H).

### Step 1:

Amine D6.1 (Prepared according to Ohta, T.; Satoshi, K.; Toshijaru, Y.; Uoto, K.; Nakamoto, Y. Diamine Derivatives. US Patent Application US2005/0020634A1.) (0.87 g, 3.1 mmol) was combined with intermediate D6.2 (prepared in a manner similar to that described in Example D1 using m-toluidine in place of amine D1.3) (0.92 g, 2.5 mmol) and DIPEA (0.87 mL, 5.0 mmol) in 10 mL of 1,4-dioxane and 2 mL NMP. The mixture was stirred at 70° C. until complete. The reaction was then diluted with ethyl acetate, 1 M HCl, and water. The layers were separated and the aqueous phase extracted once more with ethyl acetate. The combined organic layers were concentrated affording the desired product (D6.3) as a yellow oil.

### Step 2:

150 mg of D6.3 was diluted with 5 mL of 4M HCl in dioxane and stirred at rt overnight. The following day the reaction was diluted with water and purified by preparative HPLC affording carboxylic acid D6 and ethyl ester D7 after lyophilization.

### Example 189

### Racemic 2-((1R,2S,4R)-2-amino-4-(methylcarbamoyl)cyclohexylamino)-4-(m-tolylamino)pyrimidine-5-carboxamide

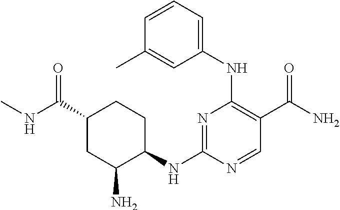 [SMILES: CNC(=O)[C@@H]1CC[C@@H](Nc2ncc(C(N)=O)c(Nc3cccc(C)c3)n2)[C@@H](N)C1]

The titled compound was prepared from intermediate D6.3 using a procedure similar to that described in Example 183, Step 4, followed by Step 5 using methylamine in place of ammonia, followed by deprotection of the Boc protected amine using 4M HCl in dioxane. MS found for C₂₀H₂₇N₇O₂ as (M+H)⁺ 398.4. UV: λ=241, 289 nm. ¹H NMR (400 MHz, MeOH-d₄) δ 8.51 (s, 1H), 7.80 (m, 1H), 7.44 (broad s, 1H), 7.39 (s, 1H), 7.29 (m, 1H), 7.03 (m, 1H), 4.18 (m, 1H), 4.02 (m, 1H), 2.73 (s, 3H), 2.43 (m, 1H), 2.38 (s, 3H), 1.99 (m, 4H), 1.82 (m, 1H), 1.71 (m, 1H).

### Example 190

### Racemic 2-((1R,2S,4R)-2-amino-4-(dimethylcarbamoyl)cyclohexylamino)-4-(m-tolylamino)pyrimidine-5-carboxamide

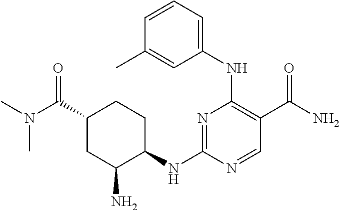 [SMILES: Cc1cccc(Nc2nc(N[C@@H]3CC[C@@H](C(=O)N(C)C)C[C@@H]3N)ncc2C(N)=O)c1]

The titled compound was synthesized using a procedure similar to that described in Example 189. MS found for C₂₁H₂₉N₇O₂ as (M+H)⁺ 412.3. UV: λ=242 nm. ¹H NMR (400 MHz, MeOH-d₄) δ 8.51 (s, 1H), 7.43 (m, 1H), 7.38 (s, 1H), 7.33 (distorted t, 1H), 7.03 (m, 1H), 4.18 (m, 1H), 4.04 (m, 1H), 3.11 (s, 3H), 3.03 (m, 1H), 2.94 (s, 3H), 2.38 (s, 3H), 1.71-2.04 (m, 5H), 1.67 (m, 1H).

### Example 191

### 2-((1R,2S,4R)-2-amino-4-(azetidine-1-carbonyl)cyclohexylamino)-4-(m-tolylamino)pyrimidine-5-carboxamide

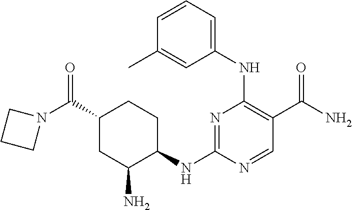 [SMILES: Cc1cccc(Nc2nc(N[C@@H]3CC[C@@H](C(=O)N4CCC4)C[C@@H]3N)ncc2C(N)=O)c1]

The titled compound was synthesized using a procedure similar to that described in Example 190. MS found for C₂₂H₂₉N₇O₂ as (M+H)⁺ 424.4. UV: λ=203, 240, 289 nm. ¹H NMR (400 MHz, MeOH-d₄) δ 8.49 (s, 1H), 7.43 (m, 1H), 7.38 (s, 1H), 7.30 (distorted t, 1H), 7.03 (m, 1H), 4.28 (m, 2H), 4.17 (m, 1H), 4.02 (m, 1H), 4.00 (t, 2H), 2.62 (m, 1H), 2.37 (s, 3H), 2.31 (m, 2H), 1.71-2.03 (m, 5H), 1.64 (m, 1H).

### Example 192

### Racemic 2-((1R,2S,4R)-2-amino-4-(3,3-difluoroazetidine-1-carbonyl)cyclohexylamino)-4-(m-tolylamino)pyrimidine-5-carboxamide

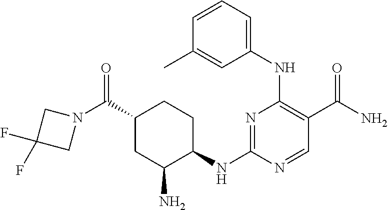 [SMILES: Cc1cccc(Nc2nc(N[C@@H]3CC[C@@H](C(=O)N4CC(F)(F)C4)C[C@@H]3N)ncc2C(N)=O)c1]

The titled compound was synthesized using a procedure similar to that described in Example 190. MS found for C₂₂H₂₇F₂N₇O₂ as (M+H)⁺ 460.5. UV: λ=204, 241, 289 nm.

### Example 193

### Racemic 2-((1R,2S,4R)-2-amino-4-(pyrrolidine-1-carbonyl)cyclohexylamino)-4-(m-tolylamino)pyrimidine-5-carboxamide

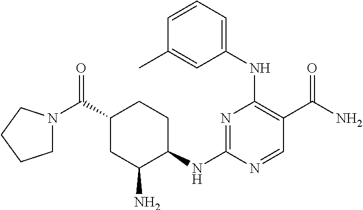 [SMILES: Cc1cccc(Nc2nc(N[C@@H]3CC[C@@H](C(=O)N4CCCC4)C[C@@H]3N)ncc2C(N)=O)c1]

The titled compound was synthesized using a procedure similar to that described in Example 190. MS found for C₂₃H₃₁N₇O₂ as (M+H)⁺ 438.5. UV: λ=213, 241, 290 nm.

### Example 194

### (R)-2-(1-amino-4-methyl-1-oxopentan-2-ylamino)-4-(4-methylthiophen-2-ylamino)pyrimidine-5-carboxamide

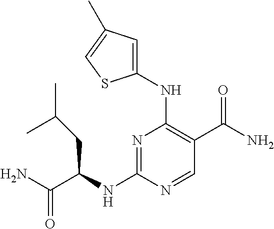 [SMILES: Cc1csc(Nc2nc(N[C@H](CC(C)C)C(N)=O)ncc2C(N)=O)c1]

A solution of 4-methyl thiophene-2-carboxylic acid (1.42 g, 10.0 mmol), TEA (1.50 mL, 10.8 mmol) and diphenyl phosphoryl azide (2.15 mL, 10.0 mmol) in t-BuOH (20 mL) was heated at reflux for 5 h. Excess of t-BuOH was removed in vacuo. Water and Et2O were added. Organic phase was separated, washed with 5% NaHCO3, dried over Na2SO4, concentrated in vacuo. The residue was purified by a silica gel column on an Isco silica column, eluted with 0-10% EtOAc in hexanes to give tert-butyl 4-methylthiophen-2-ylcarbamate as a solid (0.880 g). To a solution of tert-butyl 4-methylthiophen-2-ylcarbamate (0.880 g, 4.13 mmol) in CH₂Cl₂ (8 mL), TFA (6 mL) was added. After being stirred at room temperature for 3 h, the mixture was concentrated in vacuo, and then dried on vacuum to give a crude 4-methylthiophen-2-amine as TFA salt.

A mixture of ethyl 2,4-dichloropyrimidine-5-carboxylate (420 mg, 1.90 mmol), the crude 4-methylthiophen-2-amine TFA salt (613 mg, 2.70 mmol) and DIEA (1.50 mL, 8.62 mmol) in CH3CN (8 mL) was stirred at room temperature for 30 min. Water and EtOAc were added. Organic phase was separated, washed with 1N HCl, then with 5% NaHCO3, dried over Na2SO4, concentrated in vacuo to give ethyl 2-chloro-4-(4-methylthiophen-2-ylamino)pyrimidine-5-carboxylate as a solid (520 mg).

To a solution of ethyl 2-chloro-4-(4-methylthiophen-2-ylamino)pyrimidine-5-carboxylate (520 mg, 1.74 mmol) in THF (7 mL), aq. 1N NaOH (7.00 mL, 7.00 mmol) was added. After being stirred at room temperature for 20 h, the mixture was acidified to pH 1-2 with 6N HCl. Water and EtOAc were added. Organic phase was separated, washed with brine, dried over Na2SO4, concentrated in vacuo to give 2-chloro-4-(4-methylthiophen-2-ylamino)pyrimidine-5-carboxylic acid (422 mg).

To a solution of 2-chloro-4-(4-methylthiophen-2-ylamino)pyrimidine-5-carboxylic acid (422 mg, 1.56 mmol) and HOBt hydrate (479 mg, 3.13 mmol) in DMF (10 mL), EDC (449 mg, 2.34 mmol) was added. The mixture was stirred at room temperature for 1 h. Ammonia (0.5 M in dioxane, 9.0 mL, 4.5 mmol) was added. After being stirred for 20 h, water and EtOAc were added. Organic phase was separated, washed with 5% NaHCO3, dried over Na2SO4, concentrated in vacuo to give 2-(1H-benzo[d][1,2,3]triazol-1-yloxy)-4-(4-methylthiophen-2-ylamino)pyrimidine-5-carboxamide (446 mg).

A solution of 2-(1H-benzo[d][1,2,3]triazol-1-yloxy)-4-(4-methylthiophen-2-ylamino)pyrimidine-5-carboxamide (70 mg, 0.19 mmol), D-leucinamide hydrochloride (52 mg, 0.31 mmol) and DIEA (0.200 mL, 1.15 mmol) in NMP (1 mL) was stirred at 90 C for 2 h. The mixture was then purified by HPLC to give the titled compound (28 mg). MS 363.3 (M+H); UV 201.6, 244.3, 326.1 nm.

### Example 195

### (R)-2-(1-amino-3-methyl-1-oxobutan-2-ylamino)-4-(4-methylthiophen-2-ylamino)pyrimidine-5-carboxamide

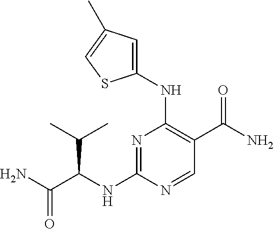 [SMILES: Cc1csc(Nc2nc(N[C@@H](C(N)=O)C(C)C)ncc2C(N)=O)c1]

A solution of 2-(1H-benzo[d][1,2,3]triazol-1-yloxy)-4-(4-methylthiophen-2-ylamino)pyrimidine-5-carboxamide (70 mg, 0.19 mmol), D-valinamide hydrochloride (56 mg, 0.36 mmol) and DIEA (0.200 mL, 1.15 mmol) in NMP (1 mL) was stirred at 90 C for 2 h. The mixture was then purified by HPLC to give the titled compound (35 mg). MS 349.3 (M+H); UV 202.9, 251.0, 324.8 nm.

### Example 196

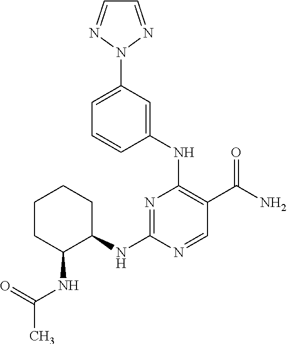 [SMILES: CC(=O)N[C@H]1CCCC[C@H]1Nc1ncc(C(N)=O)c(Nc2cccc(-n3nccn3)c2)n1]

The title compound was synthesized in a manner similar to that described above.

### Example 197

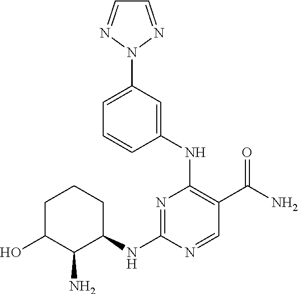 [SMILES: NC(=O)c1cnc(N[C@@H]2CCCC(O)[C@@H]2N)nc1Nc1cccc(-n2nccn2)c1]

The title compound was synthesized in a manner similar to that described above.

### Example 198

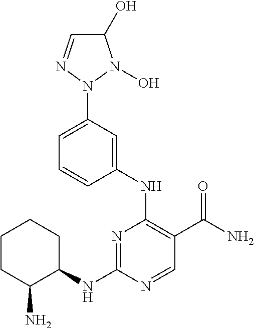 [SMILES: NC(=O)c1cnc(N[C@@H]2CCCC[C@@H]2N)nc1Nc1cccc(N2N=CC(O)N2O)c1]

The title compound was synthesized in a manner similar to that described above.

### Example 199

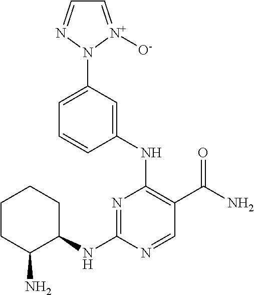 [SMILES: NC(=O)c1cnc(N[C@@H]2CCCC[C@@H]2N)nc1Nc1cccc(-n2ncc[n+]2[O-])c1]

The title compound was synthesized in a manner similar to that described above.

### Example 200

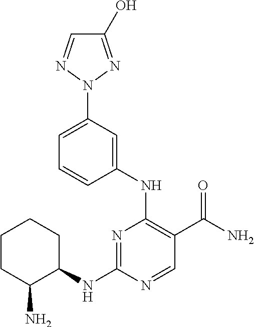 [SMILES: NC(=O)c1cnc(N[C@@H]2CCCC[C@@H]2N)nc1Nc1cccc(-n2ncc(O)n2)c1]

The title compound was synthesized in a manner similar to that described above.

### Example 201

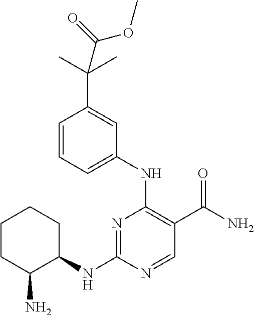 [SMILES: COC(=O)C(C)(C)c1cccc(Nc2nc(N[C@@H]3CCCC[C@@H]3N)ncc2C(N)=O)c1]

The title compound was synthesized in a manner similar to that described above.

### Example 202

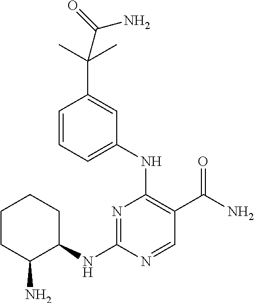 [SMILES: CC(C)(C(N)=O)c1cccc(Nc2nc(N[C@@H]3CCCC[C@@H]3N)ncc2C(N)=O)c1]

The title compound was synthesized in a manner similar to that described above.

### Example 203

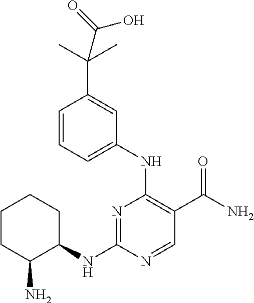 [SMILES: CC(C)(C(=O)O)c1cccc(Nc2nc(N[C@@H]3CCCC[C@@H]3N)ncc2C(N)=O)c1]

The title compound was synthesized in a manner similar to that described above.

### Example 204

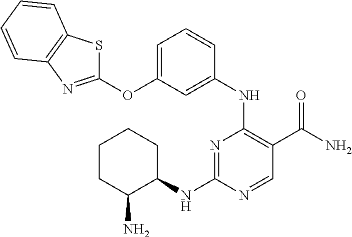 [SMILES: NC(=O)c1cnc(N[C@@H]2CCCC[C@@H]2N)nc1Nc1cccc(Oc2nc3ccccc3s2)c1]

The title compound was synthesized in a manner similar to that described above.

### Example 205

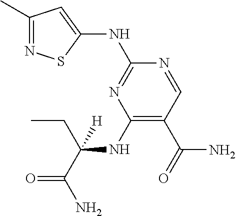 [SMILES: CC[C@@H](Nc1nc(Nc2cc(C)ns2)ncc1C(N)=O)C(N)=O]

The title compound was synthesized in a manner similar to that described above.

### Example 206

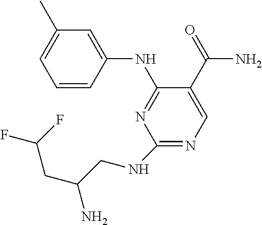 [SMILES: Cc1cccc(Nc2nc(NCC(N)CC(F)F)ncc2C(N)=O)c1]

The title compound was synthesized in a manner similar to that described above.

### Example 207

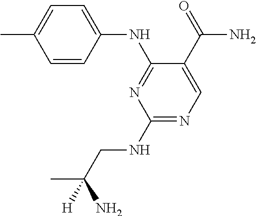 [SMILES: Cc1ccc(Nc2nc(NC[C@H](C)N)ncc2C(N)=O)cc1]

The title compound was synthesized in a manner similar to that described above.

### Example 208

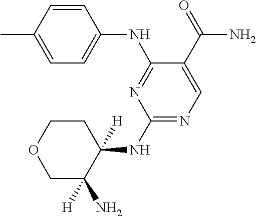 [SMILES: Cc1ccc(Nc2nc(N[C@@H]3CCOC[C@@H]3N)ncc2C(N)=O)cc1]

The title compound was synthesized in a manner similar to that described above.

### Example 209

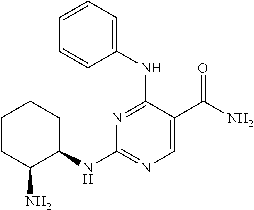 [SMILES: NC(=O)c1cnc(N[C@@H]2CCCC[C@@H]2N)nc1Nc1ccccc1]

The title compound was synthesized in a manner similar to that described above.

### Example 210

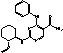 [SMILES: C=N[C@H]1CCCC[C@H]1Nc1ncc(C(N)=O)c(Nc2ccccc2)n1]

The title compound was synthesized in a manner similar to that described above.

### Example 211

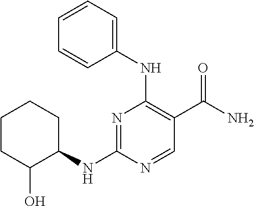 [SMILES: NC(=O)c1cnc(N[C@@H]2CCCCC2O)nc1Nc1ccccc1]

The title compound was synthesized in a manner similar to that described above.

### Example 212

### Preparation of (R)-4-((1-methyl-1H-indol-4-yl)amino)-2-(pyrrolidin-3-ylamino)pyrimidine-5-carboxamide

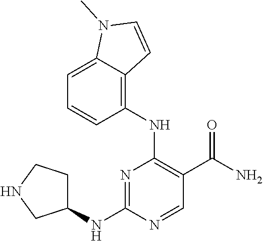 [SMILES: Cn1ccc2c(Nc3nc(N[C@@H]4CCNC4)ncc3C(N)=O)cccc21]

The title compound was synthesized in a manner similar to that described in Example 41. MS found for C18H21N7O as (M+H)⁺ 352.3. UV: λ=219, 241 nm. ¹H NMR: (CD₃OD) δ 8.46 (1H, s), 7.82 (1H, br), 7.32-7.21 (3H, m), 6.56 (1H, dd, J=0.8 Hz, 3.2 Hz), 4.62 (1H, m), 3.86 (3H, s), 3.52-3.35 (4H, m), 2.43-2.32 (1H, m), 2.24-2.15 (1H, m) ppm.

### Example 213

### Preparation of (R)-2-(pyrrolidin-3-ylamino)-4-(m-tolylamino)pyrimidine-5-carboxamide

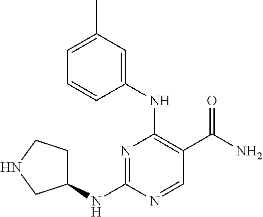 [SMILES: Cc1cccc(Nc2nc(N[C@@H]3CCNC3)ncc2C(N)=O)c1]

The title compound was synthesized in a manner similar to that described in Example 41. MS found for C16H20N6O as (M+H)⁺ 313.3. UV: λ=242, 294 nm. ¹H NMR: (CD₃OD) δ 8.48 (1H, s), 7.46 (1H, d, J=8.0 Hz), 7.41 (1H, s), 7.31 (1H, t, J=8.0 Hz), 7.07 (1H, d, J=7.6 Hz), 4.64 (1H, m), 3.56-3.40 (4H, m), 2.44-2.32 (1H, m), 2.26-2.18 (1H, m) ppm.

### Example 214

### Preparation of (R)-2-(pyrrolidin-3-ylamino)-4-(quinolin-6-ylamino)pyrimidine-5-carboxamide

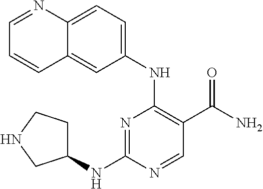 [SMILES: NC(=O)c1cnc(N[C@@H]2CCNC2)nc1Nc1ccc2ncccc2c1]

The title compound was synthesized in a manner similar to that described in Example 41. MS found for C18H19N7O as (M+H)⁺ 350.2. UV: λ=240, 283 nm. ¹H NMR: (CD₃OD) δ 9.01 (1H, dd, J=1.6 Hz, 5.2 Hz), 8.85 (1H, br), 8.64 (1H, s), 8.58 (1H, br), 8.28-8.17 (2H, m), 7.90 (1H, br), 4.72 (1H, br), 3.64-3.42 (4H, m), 2.50-2.40 (1H, m), 2.30-2.20 (1H, m) ppm.

### Example 215

### Preparation of (S)-2-(piperidin-3-ylamino)-4-(quinolin-3-ylamino)pyrimidine-5-carboxamide

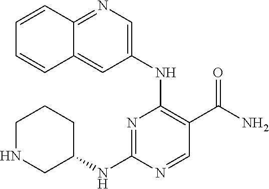 [SMILES: NC(=O)c1cnc(N[C@H]2CCCNC2)nc1Nc1cnc2ccccc2c1]

The title compound was synthesized in a manner similar to that described in Example 41. MS found for C19H21N7O as (M+H)⁺ 364.2. UV: λ=243, 280 nm. ¹H NMR: (CD₃OD) δ 9.38 (2H, m), 8.60 (1H, br), 8.50 (1H, br), 8.03 (2H, m), 7.82 (1H, m), 7.75 (1H, m), 4.23 (1H, m), 3.59 (1H, m), 3.32-3.00 (3H, m), 2.24-2.03 (2H, m), 1.90-1.74 (2H, m) ppm.

### Example 216

### Preparation of 2-((2-oxopyrrolidin-3-yl)amino)-4-(quinolin-3-ylamino)pyrimidine-5-carboxamide

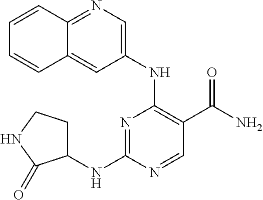 [SMILES: NC(=O)c1cnc(NC2CCNC2=O)nc1Nc1cnc2ccccc2c1]

The title compound was synthesized in a manner similar to that described in Example 60. MS found for C18H17N7O2 as (M+H)⁺ 364.2. UV: λ=244, 299 nm. ¹H NMR: (CD₃OD) δ 9.36 (1H, br), 8.76 (1H, br), 8.58 (1H, s), 8.08 (1H, d, J=8.4 Hz), 8.02 (1H, d, J=8.0 Hz), 7.85 (1H, m), 7.73 (1H, m), 4.73 (1H, m), 3.52-3.20 (2H, m), 2.58-2.43 (1H, m), 2.27-2.16 (1H, m) ppm.

### Example 217

### Preparation of (2S,4R)-4-((5-carbamoyl-4-(quinolin-3-ylamino)pyrimidin-2-yl)amino)pyrrolidine-2-carboxylic acid

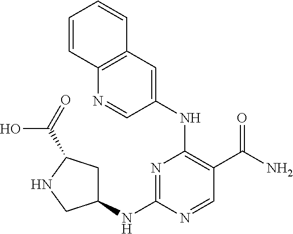 [SMILES: NC(=O)c1cnc(N[C@H]2CN[C@H](C(=O)O)C2)nc1Nc1cnc2ccccc2c1]

The title compound was synthesized in a manner similar to that described in Example 41. MS found for C19H19N7O3 as (M+H)⁺ 394.2. UV: λ=242, 281 nm.

### Example 218

### Preparation of 2-(((3R,5S)-5-(methylcarbamoyl)pyrrolidin-3-yl)amino)-4-(m-tolylamino)pyrimidine-5-carboxamide

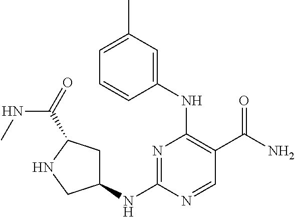 [SMILES: CNC(=O)[C@@H]1C[C@@H](Nc2ncc(C(N)=O)c(Nc3cccc(C)c3)n2)CN1]

The title compound was synthesized in a manner similar to that described in Example 41. MS found for C18H23N7O2 as (M+H)⁺ 370.3. UV: λ=241, 287 nm.

### Example 219

### Preparation of 2-(((3R,5S)-5-(dimethylcarbamoyl)pyrrolidin-3-yl)amino)-4-(m-tolylamino)pyrimidine-5-carboxamide

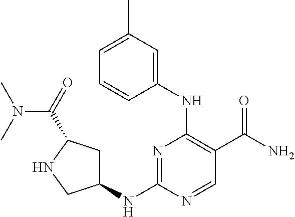 [SMILES: Cc1cccc(Nc2nc(N[C@H]3CN[C@H](C(=O)N(C)C)C3)ncc2C(N)=O)c1]

The title compound was synthesized in a manner similar to that described in Example 41. MS found for C19H25N7O2 as (M+H)⁺ 384.3. UV: λ=241, 292 nm.

### Example 220

### Preparation of 2-(((3R,5R)-5-(hydroxymethyl)pyrrolidin-3-yl)amino)-4-((1-methyl-1,2,3,4-tetrahydroquinolin-6-yl)amino)pyrimidine-5-carboxamide

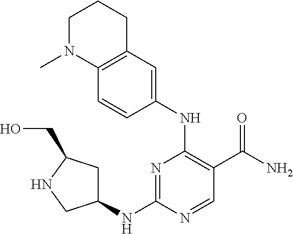 [SMILES: CN1CCCc2cc(Nc3nc(N[C@H]4CN[C@@H](CO)C4)ncc3C(N)=O)ccc21]

The title compound was synthesized in a manner similar to that described below.

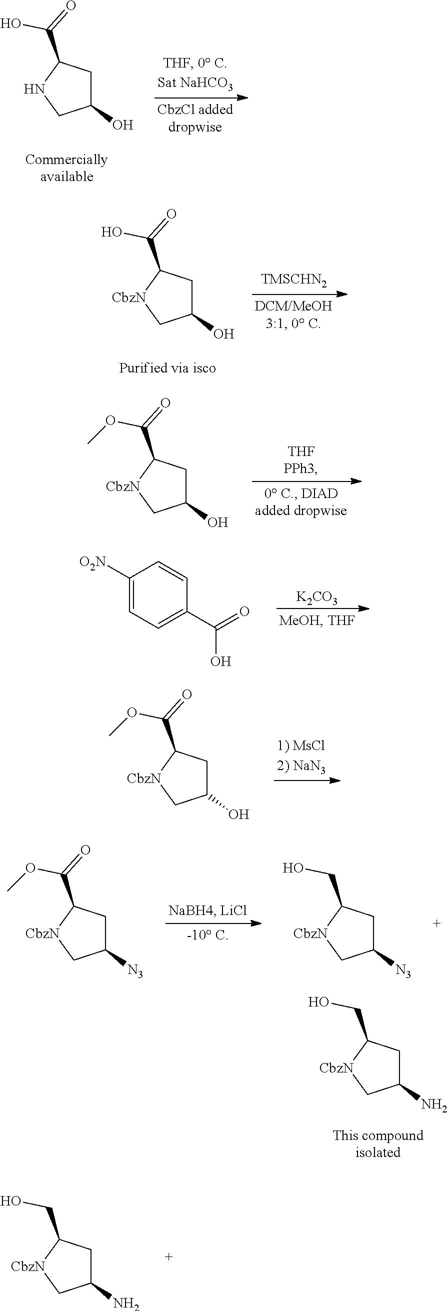

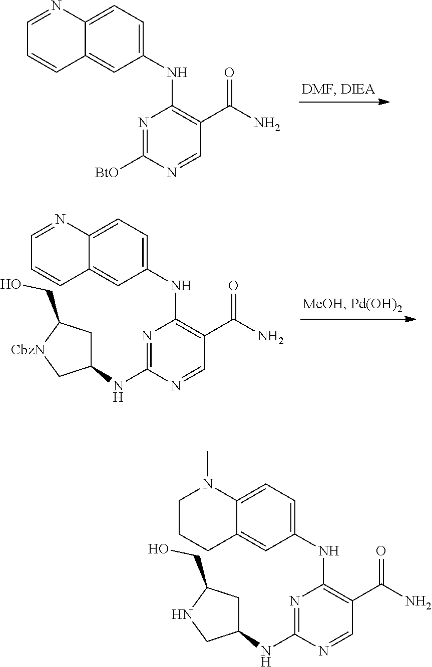

MS found for C20H27N7O2 as (M+H)⁺ 398.3. UV: λ=244, 287 nm. ¹H NMR: (CD₃OD) δ 8.49 (1H, s), 7.40 (2H, br), 6.80 (1H, br), 4.61 (1H, m), 4.02-3.64 (4H, m), 2.97 (3H, s), 2.88 (3H, m), 2.74 (1H, m), 2.60 (1H, m) 2.18-2.00 (3H, m) ppm.

### Example 221

### Preparation of (R)-4-(3-(2H-1,2,3-triazol-2-yl)phenyeamino)-2-(pyrrolidin-3-ylamino)pyrimidine-5-carboxamide

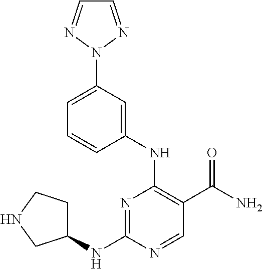 [SMILES: NC(=O)c1cnc(N[C@@H]2CCNC2)nc1Nc1cccc(-n2nccn2)c1]

The title compound was synthesized in a manner similar to that described in Example 41. MS found for C17H19N9O as (M+H)⁺ 366.3. UV: λ=249 nm. ¹H NMR: (CD₃OD) δ 8.97 (1H, s), 8.52 (1H, s), 7.95 (2H, s), 7.92 (1H, m), 7.51 (1H, t, J=8.0 Hz), 7.29 (1H, m), 3.80-3.30 (4H, m), 2.61-2.48 (1H, m), 2.36-2.21 (1H, m) ppm.

### Example 222

### Preparation of (R)-4-(3-(pyrimidin-2-yl)phenyl)amino)-2-(pyrrolidin-3-ylamino)pyrimidine-5-carboxamide

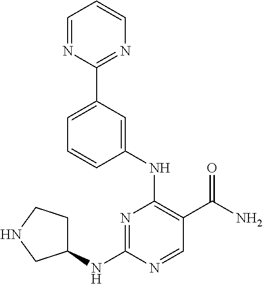 [SMILES: NC(=O)c1cnc(N[C@@H]2CCNC2)nc1Nc1cccc(-c2ncccn2)c1]

The title compound was synthesized in a manner similar to that described in Example 41. MS found for C19H20N8O as (M+H)⁺ 377.3. UV: λ=249 nm. ¹H NMR: (CD₃OD) δ 9.03 (1H, br), 8.87 (2H, d, J=5.2 Hz), 8.52 (1H, s), 8.29 (1H, br), 7.54 (1H, m), 7.39 (1H, t, J=4.8 Hz), 4.82 (1H, m), 3.64-3.40 (4H, m), 2.50-2.41 (1H, m), 2.36-2.22 (1H, m) ppm.

### Example 223

### Preparation of (R)-4-(3-(2H-1,2,3-triazol-2-yl)phenyl)amino)-2-((1-ethylpyrrolidin-3-yl)amino)pyrimidine-5-carboxamide

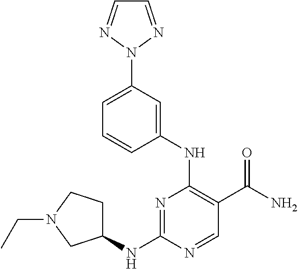 [SMILES: CCN1CC[C@@H](Nc2ncc(C(N)=O)c(Nc3cccc(-n4nccn4)c3)n2)C1]

The title compound was synthesized in a manner similar to that described in Example 41. MS found for C19H23N9O as (M+H)⁺ 394.2. UV: λ=249 nm. ¹H NMR: (CD₃OD) δ 8.83 (1H, br), 8.48 (1H, s), 7.89 (2H, s), 7.77 (1H, d, J=6.8 Hz), 7.43 (1H, t, J=8.4 Hz), 7.23 (1H, br), 4.02 (1H, m), 3.82-3.64 (2H, m), 3.5-.3.40 (1H, m), 3.16 (2H, m), 2.62 (1H, m), 2.26-2.09 (2H, m), 1.24 (3H, m) ppm.

### Example 224

### Preparation of (R)-4-((3-(2H-1,2,3-triazol-2-yl)phenyl)amino)-2-(1-(cyanomethyl)pyrrolidin-3-yl)amino)pyrimidine-5-carboxamide

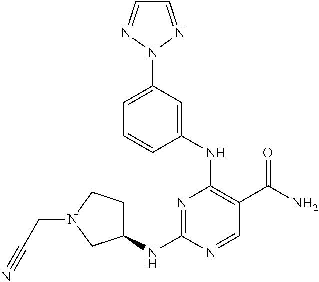 [SMILES: N#CCN1CC[C@@H](Nc2ncc(C(N)=O)c(Nc3cccc(-n4nccn4)c3)n2)C1]

The title compound was synthesized in a manner similar to that described in Example 41. MS found for C19H20N10O as (M+H)⁺ 405.2. UV: λ=251 nm. ¹H NMR: (CD₃OD) δ 9.00 (1H, br), 8.39 (1H, s), 7.88-7.84 (3H, m), 7.47 (1H, t, J=8.0 Hz), 7.25 (1H, d, 8.4 Hz), 4.80 (1H, m), 3.79 (2H, s), 3.02-2.94 (2H, m), 2.81 (1H, m), 2.62 (1H, m), 2.43 (1H, m), 1.84 (1H, m) ppm.

### Example 225

### Preparation of 4-((3-(2H-1,2,3-triazol-2-yl)phenyl)amino)-2-(3R,5S)-5-(hydroxymethyl)pyrrolidin-3-yl)amino)pyrimidine-5-carboxamide

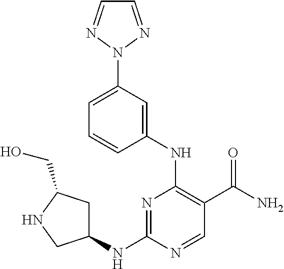 [SMILES: NC(=O)c1cnc(N[C@H]2CN[C@H](CO)C2)nc1Nc1cccc(-n2nccn2)c1]

The title compound was synthesized in a manner similar to that described in Example 41 and

### Example 220

The cis-4-Hydroxy-L-proline derivative was utilized here however (chemistry to intermediate similar to that in Example 220). MS found for C18H21N9O2 as (M+H)⁺ 396.2. UV: λ=249 nm. ¹H NMR: (CD₃OD) δ 9.03 (1H, br), 8.59 (1H, s), 8.01 (2H, s), 7.91 (1H, d, J=7.2 Hz), 7.56 (1H, t, J=8.0 Hz), 7.32 (1H, m), 4.80 (1H, m), 3.94-3.73 (4H, m), 3.40 (1H, dd), 2.71 (1H, m), 2.02 (1H, m) ppm.

### Example 226

### Preparation of (R)-4-((3-(2H-1,2,3-triazol-2-yl)phenyl)amino)-2-(5-methylenepyrrolidin-3-yl)amino)pyrimidine-5-carboxamide

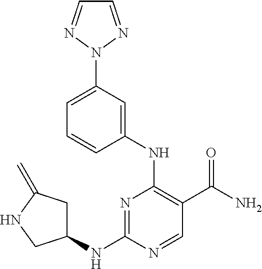 [SMILES: C=C1C[C@@H](Nc2ncc(C(N)=O)c(Nc3cccc(-n4nccn4)c3)n2)CN1]

The title compound was synthesized in as a side product in Example 229. MS found for C18H19N9O as (M+H)⁺ 378.3. UV: λ=255 nm.

### Example 227

### Preparation of (R)-4-(3-(2H-1,2,3-triazol-2-yl)phenyl)amino)-2-((5-methyl-2,3-dihydro-1H-pyrrol-3-yl)amino)pyrimidine-5-carboxamide

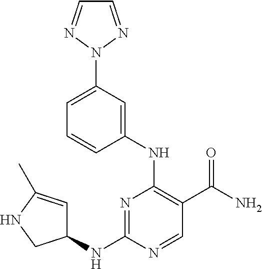 [SMILES: CC1=C[C@@H](Nc2ncc(C(N)=O)c(Nc3cccc(-n4nccn4)c3)n2)CN1]

The title compound was recovered as a side product in Example 229. MS found for C18H19N9O as (M+H)⁺ 378.2. UV: λ=265 nm.

### Example 228

### Preparation of (R)-4-(3-(2H-1,2,3-triazol-2-yl)phenyl)amino)-2-((1-formylpyrrolidin-3-yl)amino)pyrimidine-5-carboxamide

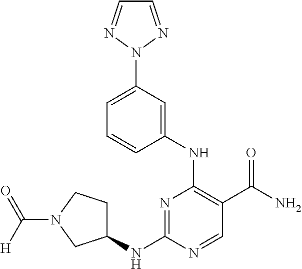 [SMILES: NC(=O)c1cnc(N[C@@H]2CCN(C=O)C2)nc1Nc1cccc(-n2nccn2)c1]

The title compound was synthesized in a manner similar to that described in Example 41. MS found for C18H19N9O2 as (M+H)⁺ 394.3. UV: λ=250 nm.

### Example 229

### Preparation of 4-((3-(2H-1,2,3-triazol-2-yl)phenyl)amino)-2-(((3R,5R)-5-(fluoromethyl)pyrrolidin-3-yl)amino)pyrimidine-5-carboxamide

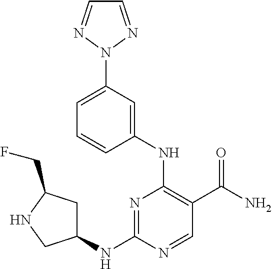 [SMILES: NC(=O)c1cnc(N[C@H]2CN[C@@H](CF)C2)nc1Nc1cccc(-n2nccn2)c1]

The title compound was synthesized in a manner similar to that described in Example 220. Note that here as in Example 225, the cis-4-Hydroxy-L-proline derivative was originally utilized.

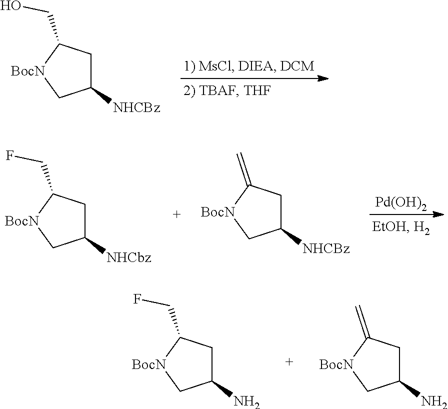

MS found for C18H20FN9O as (M+H)⁺ 398.3. UV: λ=249 nm.

### Example 230

### Preparation of (R)-4-(3-(2H-1,2,3-triazol-2-yl)phenyl)amino)-2-((5-oxopyrrolidin-3-yl)amino)pyrimidine-5-carboxamide

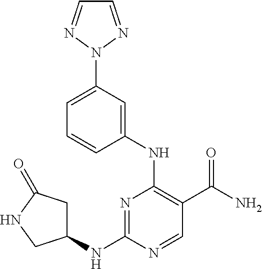 [SMILES: NC(=O)c1cnc(N[C@H]2CNC(=O)C2)nc1Nc1cccc(-n2nccn2)c1]

The title compound was synthesized in a manner similar to that described in Example 60. MS found for C17H17N9O2 as (M+H)⁺ 380.3. UV: λ=250 nm. ¹H NMR: (CD₃OD) δ 9.22 (1H, s), 8.58 (1H, s), 7.96 (3H, m), 7.66 (1H, t, J=8.0 Hz), 7.30 (1H, d, J=8.0 Hz), 5.19 (1H, m), 3.92 (1H, m), 3.40 (1H, m), 2.95 (1H, m), 2.43 (1H, dd) ppm.

### Example 231

### Preparation of 2-(((1R,2S)-2-aminocyclohexyl)amino)-4-((4-iodo-3-methylisothiazol-5-yl)amino)pyrimidine-5-carboxamide

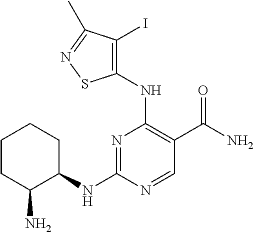 [SMILES: Cc1nsc(Nc2nc(N[C@@H]3CCCC[C@@H]3N)ncc2C(N)=O)c1I]

The title compound was synthesized in a manner seen below utilizing material from Example 49 as starting material.

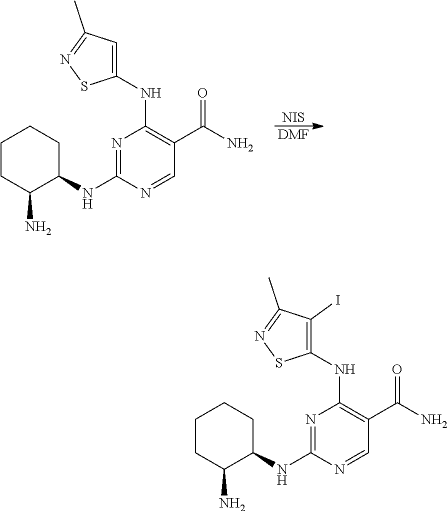

MS found for C15H20IN7OS as (M+H)⁺ 474.3. UV: λ=202, 271, 316 nm. ¹H NMR: (CD₃OD) δ 8.69 (1H, s), 4.66 (1H, br), 3.78 (1H, br), 2.48 (3H, s), 2.04-1.60 (8H, m) ppm.

### Example 232

### Preparation of 2-(((1R,2S)-2-aminocyclohexyl)amino)-4-((4-chloro-3-methylisothiazol-5-yl)amino)pyrimidine-5-carboxamide

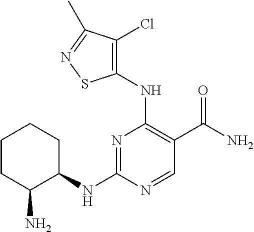 [SMILES: Cc1nsc(Nc2nc(N[C@@H]3CCCC[C@@H]3N)ncc2C(N)=O)c1Cl]

The title compound was synthesized in a manner seen below utilizing material from Example 49 as starting material.

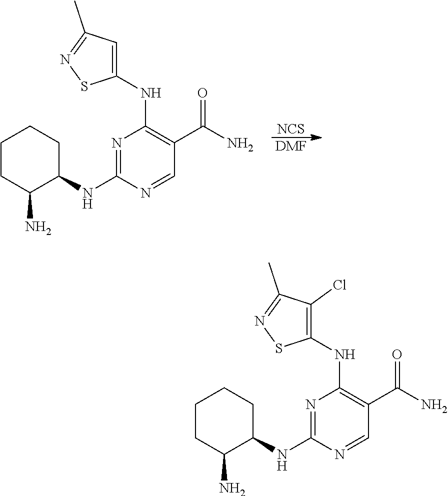

MS found for C15H20ClN7OS as (M+H)⁺ 382.2, 384.4. UV: λ=203, 268, 311 nm. ¹H NMR: (CD₃OD) δ 8.70 (1H, s), 4.61 (1H, br), 3.83 (1H, br), 2.39 (3H, s), 2.02-1.59 (8H, m) ppm.

### Example 233

### 2-((1S,2R,4S)-2-amino-4-(azetidine-1-carbonyl)-cyclohexylamino)-4-(m-tolylamino)-pyrimidine-5-carboxamide

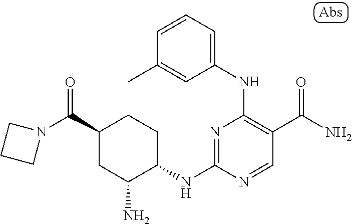 [SMILES: Cc1cccc(Nc2nc(N[C@H]3CC[C@H](C(=O)N4CCC4)C[C@H]3N)ncc2C(N)=O)c1]

The title compound was enantiomerically resolved from Example 191 using Supercritical Fluid Chromatography. EE=99%. MS found for C22H29N7O2 as (M+H)⁺ 424.4. UV: λ=208.3 241.2 287.2 nm.

### Example 234

### 2-((1R,2S,4R)-2-amino-4-(azetidine-1-carbonyl)-cyclohexylamino)-4-(m-tolylamino)-pyrimidine-5-carboxamide

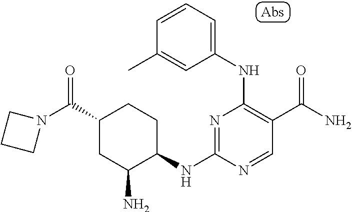 [SMILES: Cc1cccc(Nc2nc(N[C@@H]3CC[C@@H](C(=O)N4CCC4)C[C@@H]3N)ncc2C(N)=O)c1]

The title compound was enantiomerically resolved from Example 191 using Supercritical Fluid Chromatography. EE=99%. MS found for C22H29N7O2 as (M+H)⁺ 424.4. UV: λ=240.6 287.8 nm.

The title compound was synthesized in a manner similar to that described above. MS found for C22H29N7O2 as (M+H)⁺ 424.4. UV: λ=240.6 287.8 nm.

### Example 235

### 4-(3-(2H-1,2,3-triazol-2-yl)phenylamino)-2-((1R,2R,3S)-2-amino-3-fluorocyclohexylamino)pyrimidine-5-carboxamide

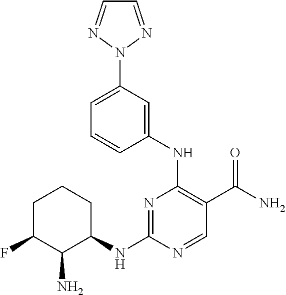 [SMILES: NC(=O)c1cnc(N[C@@H]2CCC[C@H](F)[C@@H]2N)nc1Nc1cccc(-n2nccn2)c1]

The title compound was synthesized in a manner similar to that described in Example 174. MS found for C19H22FN9O as (M+H)⁺ 412.3. UV: λ=249.1 nM. ¹H NMR: (400 MHz, CD₃OD) δ (ppm) 9.08 (bs, 1H), 8.52 (s, 1H), 7.94 (s, 2H), 7.78 (d, J=8.0 Hz, 1H), 7.46 (t, J=8.0 Hz, 1H), 7.27 (d, J=8.0 Hz, 1H), 4.68 (m, 1H), 4.52 (m, 2H), 1.98 (m, 1H), 1.77 (m, 4H), 1.41 (m, 1H).

### Example 236

### 2-((1R,2R,3S)-2-amino-3-fluorocyclohexylamino)-4-(3-(pyrimidin-2-yl)phenylamino)pyrimidine-5-carboxamide

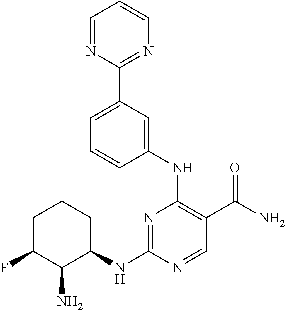 [SMILES: NC(=O)c1cnc(N[C@@H]2CCC[C@H](F)[C@@H]2N)nc1Nc1cccc(-c2ncccn2)c1]

The title compound was synthesized in a manner similar to that described in Example 174. MS found for C21H23FN8O as (M+H)⁺ 423.3. UV: λ=249.1 nM. ¹H NMR: (CD₃OD) δ (ppm) 8.92 (bs, 1H), 8.77 (d, J=4.4 Hz, 1H), 8.41 (s, 1H), 8.03 (d, J=7.6 Hz, 1H), 7.49 (m, 1H), 7.37 (t, J=8.0 Hz, 1H), 7.28 (t, J=4.8 Hz, 1H), 4.65 (m, 1H), 4.51 (m, 1H), 4.36 (m, 1H), 1.88 (m, 1H), 1.58 (m, 4H), 1.25 (m, 1H).

### Example 237

### 2-((1R,2R,3S)-2-amino-3-fluorocyclohexylamino)-4-(quinolin-6-ylamino)pyrimidine-5-carboxamide

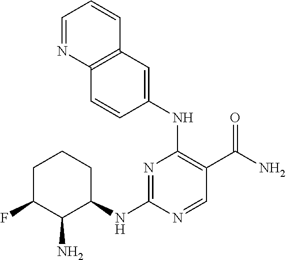 [SMILES: NC(=O)c1cnc(N[C@@H]2CCC[C@H](F)[C@@H]2N)nc1Nc1ccc2ncccc2c1]

The title compound was synthesized in a manner similar to that described in Example 174. MS found for C20H22FN7O as (M+H)⁺ 396.3. UV: λ=239.7, 282.3 nM. ¹H NMR: (CD₃OD) δ (ppm) 8.64 (bs, 1H), 8.44 (m, 1H), 8.37 (m, 1H), 8.19 (m, 1H), 7.87 (q, J=8.8 Hz, 2H), 7.42 (dd, J=4.0, 8.0 Hz, 1H), 4.65 (m, 1H), 4.51 (m, 1H), 4.22 (m, 1H), 1.72 (m, 5H), 1.35 (m, 1H).

### Example 238

### 4-(3-(1H-pyrazol-1-yl)phenylamino)-2-((1R,2R,3S)-2-amino-3-fluorocyclohexylamino)pyrimidine-5-carboxamide

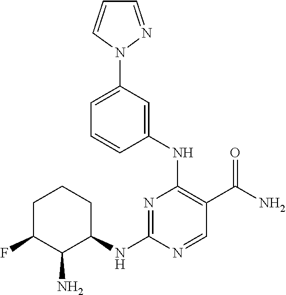 [SMILES: NC(=O)c1cnc(N[C@@H]2CCC[C@H](F)[C@@H]2N)nc1Nc1cccc(-n2cccn2)c1]

The title compound was synthesized in a manner similar to that described in Example 174. MS found for C20H23FN8O as (M+H)⁺ 411.3. UV: λ=249.1. ¹H NMR: (CD₃OD) δ (ppm) 8.58 (bs, 1H), 8.41 (s, 1H), 8.14 (d, J=2.0 Hz, 1H), 7.64 (d, J=1.6 Hz, 1H), 7.33 (q, J=8.0 Hz, 1H), 7.28 (d, J=8.0 Hz, 1H), 7.21 (m, 1H), 6.45 (t, J=2.0 Hz, 1H), 4.51 (m, 2H), 4.26 (m, 1H), 1.84 (m, 1H), 1.59 (m, 4H), 1.21 (m, 1H).

### Example 239

### 2-((1R,2R,3S)-2-amino-3-fluorocyclohexylamino)-4-(benzo[d]thiazol-6-ylamino)pyrimidine-5-carboxamide

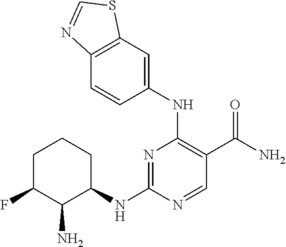 [SMILES: NC(=O)c1cnc(N[C@@H]2CCC[C@H](F)[C@@H]2N)nc1Nc1ccc2ncsc2c1]

The title compound was synthesized in a manner similar to that described in Example 174. MS found for C18H20FN7OS as (M+H)⁺ 402.2. UV: λ=239.7, 296.6. ¹H NMR: (CD₃OD) δ (ppm) 9.04 (s, 1H), 8.57 (s, 1H), 8.14 (d, J=2.0 Hz, 1H), 7.64 (d, J=1.6 Hz, 1H), 7.33 (q, J=8.0 Hz, 1H), 7.28 (d, J=8.0 Hz, 1H), 7.21 (m, 1H), 6.45 (t, J=2.0 Hz, 1H), 4.51 (m, 2H), 4.26 (m, 1H), 1.84 (m, 1H), 1.59 (m, 4H), 1.21 (m, 1H).

### Example 240

### 2-((1R,2R,3S)-2-amino-3-fluorocyclohexylamino)-4-(1-methyl-1H-indazol-4-ylamino)pyrimidine-5-carboxamide

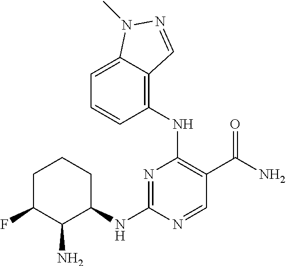 [SMILES: Cn1ncc2c(Nc3nc(N[C@@H]4CCC[C@H](F)[C@@H]4N)ncc3C(N)=O)cccc21]

The title compound was synthesized in a manner similar to that described in Example 174. MS found for C19H23FN8O as (M+H)⁺ 399.3. UV: λ=230.2, 253.8.

### Example 241

### 2-((1R,2R,3S)-2-amino-3-fluorocyclohexylamino)-4-(quinolin-3-ylamino)pyrimidine-5-carboxamide

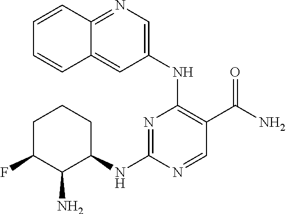 [SMILES: NC(=O)c1cnc(N[C@@H]2CCC[C@H](F)[C@@H]2N)nc1Nc1cnc2ccccc2c1]

The title compound was synthesized in a manner similar to that described in Example 174. MS found for C20H22FN7O as (M+H)⁺ 396.2. UV: λ=244.4, 282.3.

### Example 242

### (±)-4-(3-(2H-1,2,3-triazol-2-yl)phenylamino)-2-((1R,2R,3R)-2-amino-3-fluorocyclohexylamino)pyrimidine-5-carboxamide

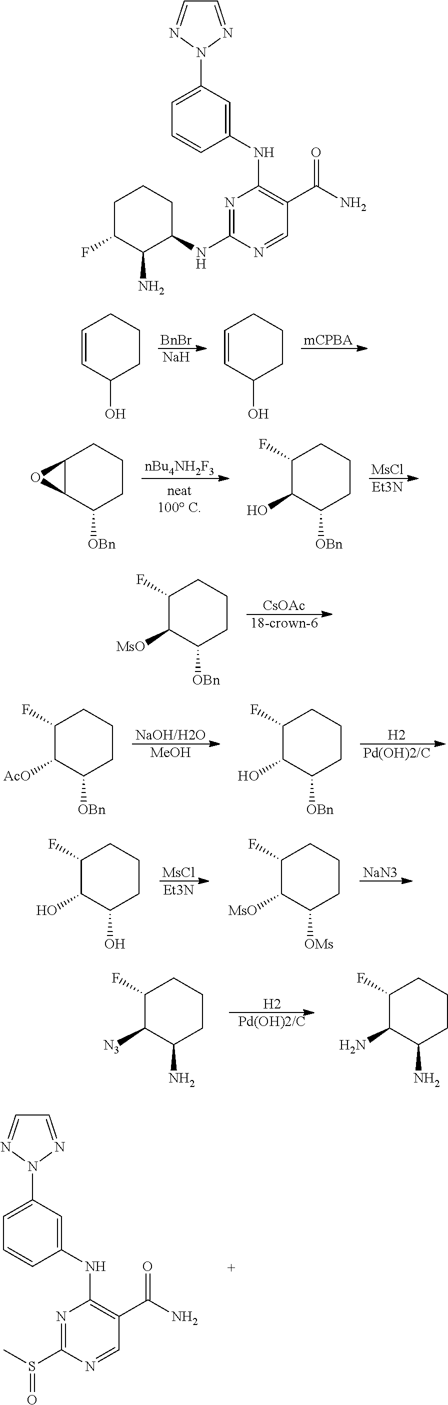

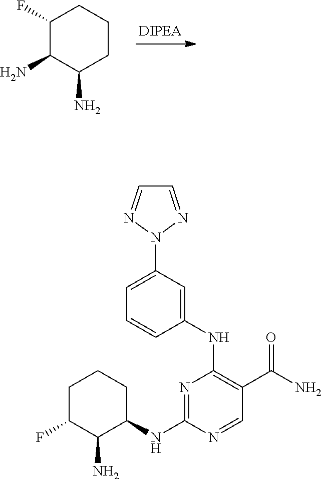 [SMILES: N[C@@H]1[C@H](N)CCC[C@H]1F ; NC(=O)c1cnc(N[C@@H]2CCC[C@@H](F)[C@@H]2N)nc1Nc1cccc(-n2nccn2)c1]

### Synthesis of (±)-(1R,2R,3R)-3-fluorocyclohexane-1,2-diamine

Step 1: A solution of cyclohex-2-enol (2.5 g, 2.55 mmol) in THF (20 mL) was added to a suspension of NaH (1.88 g, 5.10 mmol) and BnBr (3.03 mL, 2.55 mmol) in THF (40 mL) at 55° C., then the mixture was heated at 55° C. for 15 h, cooled to room temperature, poured to ice water, and extracted with Ether, the organic layer was washed with brine, dried and concentrated to give crude oil, which was purified by column chromatography to give ((cyclohex-2-enyloxy)methyl)benzene (4.08 g).

Step 2: To a solution of ((cyclohex-2-enyloxy)methyl)benzene (4.08 g) in DCM (60 mL) was added mCPBA (6.30 g, 65%, 23.75 mmol) and NaHCO3 (2.0 g, 23.75 mmol) at 0° C., after stirred for 15 h, the solid was filtered off and the filtrate was washed with Sat. NaHCO3, brine, dried and concentrated to give crude oil, which was purified by column chromatography (Hexanes/EtOAc=100:0 to 90:10) to give (±)-(1S,2S,6S)-2-(benzyloxy)-7-oxabicyclo[4.1.0]heptanes (1.8 g).

Step 3: A mixture of (±)-(1S,2S,6S)-2-(benzyloxy)-7-oxabicyclo[4.1.0]heptanes (440 mg, 2 mmol) and nBu₄NH₂F3 (1.25 g) was heated at 100° C. for 15 h, the mixture was then cooled and diluted with EtOAc, washed with water, brine, dried and concentrated to give crude oil, which was purified by column chromatography (Hexanes/EtOAc=80:20) to give (±)-(1R,2S,6R)-2-(benzyloxy)-6-fluorocyclohexanol (250 mg).

Step 4: To a solution of (±)-(1R,2S,6R)-2-(benzyloxy)-6-fluorocyclohexanol (560 mg, 2.49 mmol) in DCM at 0° C. was added Et3N (1.04 ml, 7.47 mmol) and MsCl (0.388 ml, 5.0 mmol). After stirring at room temperature for 15 h, it was diluted with DCM, the organic layer was washed with water, brine, dried and concentrated to give crude oil, which was purified by column chromatography to give (±)-(1R,2S,6R)-2-(benzyloxy)-6-fluorocyclohexyl methanesulfonate (586 mg).

Step 5: To a solution of (±)-(1R,2S,6R)-2-(benzyloxy)-6-fluorocyclohexyl methanesulfonate (586 mg, 1.94 mmol) in DMF (30 ml) was added CsOAc (1.12 g) and 18-Crown-6 (1.02 g, 3.88 mmol), after heated at 100° C. for 15 h, it was diluted with EtOAc, organic layer was washed with water and brine, dired and concentrated to give crude product, which was separated by column chromatography to give (±)-(1S,2S,6R)-2-(benzyloxy)-6-fluorocyclohexyl acetate (225 mg).

Step 6: To a solution of (±)-(1S,2S,6R)-2-(benzyloxy)-6-fluorocyclohexyl acetate (225 mg, 0.84 mmol) in MeOH (3 ml) was added NaOH (1N, 1.68 ml, 1.68 mmol). After stirring at room temperature for 2 h, the mixture was diluted with EtOAc and 1N HCl, organic layer was separated and the aqueous layer was further extracted with EtOAc, organic layer was combined, dried and concentrated to give crude (±)-(1S,2S,6R)-2-(benzyloxy)-6-fluorocyclohexanol (182 mg).

Step 7: To a solution of (±)-(1S,2S,6R)-2-(benzyloxy)-6-fluorocyclohexanol (182 mg) in EtOH (3 ml) was added Pd(OH)2/C (100 mg), charged with H2, the mixture was stirred at room temperature for 2 h, Pd(OH)2/C was filtered off and the filtrate was concentrated to give (±)-(1S,2S,3R)-3-fluorocyclohexane-1,2-diol.

Step 8: To a solution of (±)-(1S,2S,3R)-3-fluorocyclohexane-1,2-diol from step 7 in DCM (3 ml) at 0° C. was added Et3N (0.454 ml, 3.2 mmol) and MsCl (0.186 ml, 2.4 mmol). After stirred at room temperature for 3 h, the mixture was diluted with DCM, washed with water, brine, dried and concentrated to give crude product, which was purified by column chromatography (Hexanes/EtOAc=100:0 to 60:40) to give (±)-(1S,2S,3R)-3-fluorocyclohexane-1,2-diyl dimethanesulfonate (130 mg).

Step 9: To a solution of (±)-(1S,2S,3R)-3-fluorocyclohexane-1,2-diyl dimethanesulfonate (130 mg, 0.45 mmol) in DMF (6 ml) was added NaN3 (292 mg, 4.50 mmol), the mixture was heated at 120° C. for 15 h, the mixture was cooled and diluted with ether and brine, organic layer was separated, dried and concentrated to give crude product, which was purified by column chromatography (Hexanes/EtOAc=100:0 to 90:10) to give (±)-(1R,2R,3R)-1,2-diazido-3-fluorocyclohexane (30 mg).

Step 10: To a solution of (±)-(1R,2R,3R)-1,2-diazido-3-fluorocyclohexane (30 mg) in EtOH (2 ml) was added Pd(OH)2/c and charged with H2, after stirred at room temperature for 4 h, pd(OH)2/C was filtered off and the filtrate was concentrated to give (±)-(1R,2R,3R)-3-fluorocyclohexane-1,2-diamine (16 mg).

### Synthesis of (±)-4-(3-(2H-1,2,3-triazol-2-yl)phenylamino)-2-((1R,2R,3R)-2-amino-3-fluorocyclohexylamino)pyrimidine-5-carboxamide

To a solution of 4-(3-(2H-1,2,3-triazol-2-yl)phenylamino)-2-(methylsulfinyl)pyrimidine-5-carboxamide (30 mg, 0.1 mmol) in NMP (1 ml) was added 1R,2R,3R)-3-fluorocyclohexane-1,2-diamine (16 mg, 0.12 mmol) and DIPEA, after heated at 70° C. for 2 h, it was dilted with water and AcCN and then purified by preparative HPLC to give (±)-4-(3-(2H-1,2,3-triazol-2-yl)phenylamino)-2-((1R,2R,3R)-2-amino-3-fluorocyclohexylamino)pyrimidine-5-carboxamide. MS found for C19H22FN9O as (M+H)⁺ 412.3. UV: λ=260.4, 296.1.

### Example 243

### Racemic 2-((1R,2S,5S)-2-amino-5-carbamoylcyclohexylamino)-4-(m-tolylamino)pyrimidine-5-carboxamide

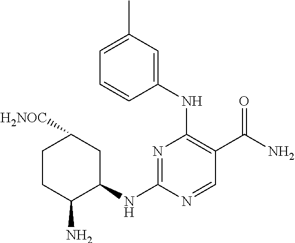 [SMILES: Cc1cccc(Nc2nc(N[C@@H]3C[C@@H](C(N)=O)CC[C@@H]3N)ncc2C(N)=O)c1]

The title compound was synthesized in a manner similar to that described in Scheme 23 using the azidoamine precursor to D6.1 described in Ohta, T.; Satoshi, K.; Toshijaru, Y.; Uoto, K.; Nakamoto, Y. Diamine Derivatives. US Patent Application US2005/0020634A1. MS found for C19H25N7O2 as (M+H)⁺ 384.4. UV: λ=241, 290 nm.

### Example 244

### 4-(trans-4-aminocyclohexylamino)-2-(p-tolylamino)pyrimidine-5-carboxamide

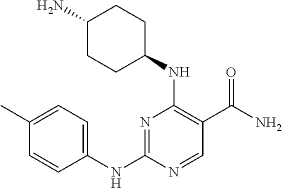 [SMILES: Cc1ccc(Nc2ncc(C(N)=O)c(N[C@H]3CC[C@H](N)CC3)n2)cc1]

The title compound was synthesized in a manner similar to that described in Example 183. MS found for C18H24N6O as (M+H)⁺ 341.3. UV: λ=222, 254 nm. ¹H NMR: (CD₃OD) δ 8.35 (s, 1H), 7.43 (d, 2H, 8 Hz), 7.23 (d, 2H, 8 Hz), 3.98 (m, 1H), 3.17 (m, 1H), 2.34 (s, 3H), 2.21 (m, 2H), 2.13 (m, 2H), 1.50 (t, 4H, 8.8 Hz).

### Example 245

### 4-(trans-4-aminocyclohexylamino)-2-(4-methoxyphenylamino)pyrimidine-5-carboxamide

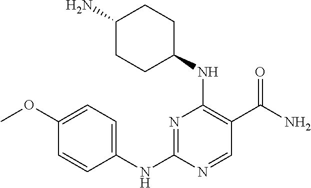 [SMILES: COc1ccc(Nc2ncc(C(N)=O)c(N[C@H]3CC[C@H](N)CC3)n2)cc1]

The title compound was synthesized in a manner similar to that described in Example 183. MS found for C18H24N6O2 as (M+H)⁺ 357.3. UV: λ=254 nm. ¹H NMR: (CD₃OD) δ 8.33 (s, 1H), 7.42 (d, 2H, 6.8 Hz), 6.98 (d, 2H, 8.8 Hz), 3.94 (m, 1H), 3.82 (s, 3H), 3.15 (m, 1H), 2.19 (m, 2H), 2.12 (m, 2H), 1.48 (m, 4H).

### Example 246

### 4-(trans-4-aminocyclohexylamino)-2-(m-tolylamino)pyrimidine-5-carboxamide

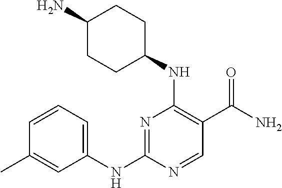 [SMILES: Cc1cccc(Nc2ncc(C(N)=O)c(N[C@H]3CC[C@@H](N)CC3)n2)c1]

The title compound was synthesized in a manner similar to that described in Example 183. MS found for C18H24N6O as (M+H)⁺ 341.3. UV: λ=205, 254 nm. ¹H NMR: (CD₃OD) δ 8.40 (s, 1H), 7.41 (s, 1H), 7.32 (distorted d, 1H), 7.27 (t, 1H, 7.2 Hz), 7.03 (d, 1H), 4.35 (m, 1H), 2.36 (s, 3H), 1.91-2.08 (m, 4H), 1.84 (m, 2H), 1.66 (m, 2H).

### Example 247

### 4-(trans-4-aminocyclohexylamino)-2-(3-methoxyphenylamino)pyrimidine-5-carboxamide

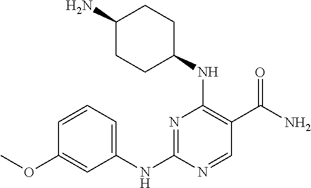 [SMILES: COc1cccc(Nc2ncc(C(N)=O)c(N[C@H]3CC[C@@H](N)CC3)n2)c1]

The title compound was synthesized in a manner similar to that described in Example 183. MS found for C18H24N6O2 as (M+H)⁺ 357.3. UV: λ=252 nm. ¹H NMR: (CD₃OD) δ8.42 (s, 1H), 7.30 (m, 1H), 7.27 (t, 1H, 7.6 Hz), 7.09 (d, 1H, 7.6 Hz), 6.75 (distorted d, 1H, 6 Hz), 4.37 (m, 1H), 3.81 (s, 3H), 3.36 (m, 1H), 2.00 (m, 4H), 1.84 (m, 2H), 1.67 (m, 2H).

### Example 248

### 4-(trans-4-aminocyclohexylamino)-2-(6-methoxypyridin-3-ylamino)pyrimidine-5-carboxamide

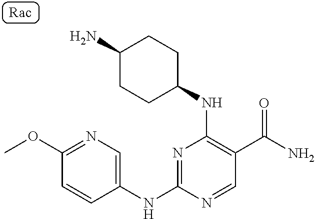 [SMILES: COc1ccc(Nc2ncc(C(N)=O)c(N[C@H]3CC[C@@H](N)CC3)n2)cn1]

The title compound was synthesized in a manner similar to that described in Example 183. MS found for C17H23N7O2 as (M+H)⁺ 358.3. UV: λ=214, 252 nm. ¹H NMR: (CD₃OD) δ 8.42 (s, 1H), 8.33 (s, 1H), 7.82 (d, 1H, 9 Hz), 6.86 (d, 1H, 8.8 Hz), 4.28 (m, 1H), 3.82 (s, 3H), 3.26 (m, 1H), 1.97 (m, 4H), 1.81 (m, 2H), 1.62 (m, 2H).

### Example 249

### 2-(((1R,2S)-2-aminocyclohexyl)amino)-4-((3,5-di(1H-pyrazol-1-yl)phenyl)amino)pyrimidine-5-carboxamide

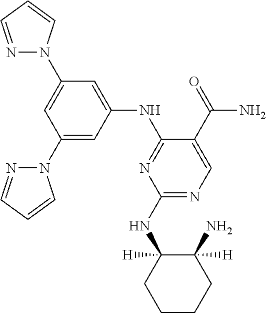 [SMILES: NC(=O)c1cnc(N[C@@H]2CCCC[C@@H]2N)nc1Nc1cc(-n2cccn2)cc(-n2cccn2)c1]

The mixture of 3,5-dibromoaniline (1.16 g, 4.6 mmol), pyrazole (1.88 g, 27.6 mmol), K₃PO₄ (3.90 g, 18.4 mmol), CuI (176 mg, 0.92 mmol), ethylenediamine (61 μL, 0.92 mmol) in 20 mL dioxane and 5 mL DMSO in a sealed tube were stirred at 120° C. for two days. The mixture was cooled to RT and diluted with 300 mL chloroform. The slurry was vigorously stirred and filtered through celite. The filtrate was washed with brine three times, dried over MgSO₄, concentrated in vacuo and subjected to flash column to isolate major product, 3,5-di(1H-pyrazol-1-yl)aniline (720 mg), and minor product, 3-bromo-5-(1H-pyrazol-1-yl)aniline (400 mg). With this aniline, the title compound was synthesized in a manner similar to that described in Example 2-((1R,2S)-2-aminocyclohexylamino)-4-(4-bromo-3-(2H-1,2,3-triazol-2-yl)phenylamino)pyrimidine-5-carboxamide. MS found for C23H26N10O as (M+H)⁺ 459.6. UV: λ=254 nm. ¹H NMR: (CD₃OD) δ 8.53 (1H, s), 8.35 (2H, d, J=2.4 Hz), 8.10 (2H, d, J=2.0 Hz), 7.92 (1H, s), 7.73 (2H, d, J=1.6 Hz), 6.52 (2H, m), 4.55 (1H, m), 3.59 (1H, m), 1.81-1.42 (8H, m) ppm.

### Example 250

### (R)-2-((1-amino-1-oxobutan-2-yl)amino)-4-((3,5-di(1H-pyrazol-1-yl)phenyl)amino)pyrimidine-5-carboxamide

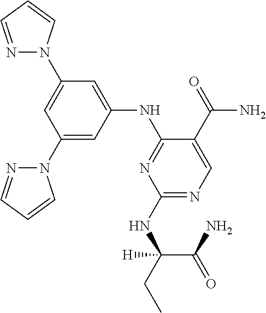

The title compound was synthesized in a manner similar to that described in Example (R)-2-((1-amino-1-oxobutan-2-yl)amino)-4-(pyrazolo[1,5-a]pyridin-3-ylamino)pyrimidine-5-carboxamide. MS found for C21H22N10O2 as (M+H)⁺ 447.5. UV: λ=254 nm. ¹H NMR: (CD₃OD) δ 8.54 (1H, s), 8.42 (2H, d, J=2.8 Hz), 8.21 (2H, d, J=1.6 Hz), 7.96 (1H, m), 7.81 (2H, d, J=1.2 Hz), 6.60 (2H, m), 4.74 (1H, m), 2.01 (1H, m), 1.90 (1H, m), 1.10 (3H, t, J=8.0 Hz) ppm.

### Example 251

### 2-(3-((2-(((1R,2S)-2-aminocyclohexyl)amino)-5-carbamoylpyrimidin-4-yl)amino)phenyl)-2H-1,2,3-triazole 1-oxide

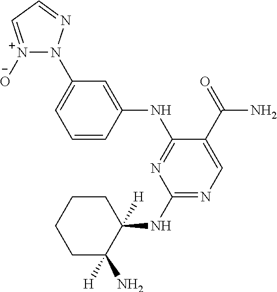 [SMILES: NC(=O)c1cnc(N[C@@H]2CCCC[C@@H]2N)nc1Nc1cccc(-n2ncc[n+]2[O-])c1]

Glyoxal (40%, Aldr 128465, 2.0 mL, 14 mmol) was diluted with 20 mL water. To it were added hydroxylamine hydrochloride (0.96 g, 13.8 mmol) and sodium carbonate (1.54 g, 14.5 mmol). Stirred at RT for 20 m. To it was added 40 mL MeOH and stirred in ice bath. To it 3-bromophenylhydrazine (Aldr 153958, 3.10 g, 13.8 mmol) was added in small portions. The mixture was stirred at RT for 30 m. CuSO₄.5H₂O (19.5 g, 78 mmol) was then added, along with 100 mL water and 100 mL pyridine. The mixture was stirred at 90° C. for overnight. It was concentrated in vacuo to remove most pyridine. The residue was triturated with 6N HCl till pH about 3. It was extracted with EtOAc 200 mL x5. The extracts were combined, dried over MgSO₄, concentrated in vacuo and subjected to flash column with 0-15% etOAc in DCM to isolate 2-(3-bromophenyl)-2H-1,2,3-triazole 1-oxide (400 mg). ¹H NMR: (CDCl₃) δ 8.17 (1H, d, J=2.0 Hz), 7.96 (1H, d, J=7.2 Hz), 7.74 (1H, s), 7.60 (1H, d, J=6.8 Hz), 7.48 (1H, s), 7.41 (1H, t, J=8.0 Hz) ppm.

2-(3-Bromophenyl)-2H-1,2,3-triazole 1-oxide (340 mg, 1.4 mmol), H₂N—BOC (820 mg, 7.0 mmol), Cs₂CO₃ (2.28 g, 7.0 mmol), Pd₂(dba)₃ (260 mg, 0.28 mmol) and XantPhos (320 mg, 0.56 mmol) was mixed in 30 mL dioxane. It was degassed with argon stream, stirred at 110° C. for overnight. The mixture was diluted with EtOAc, washed with brine x3, dried, concentrated and subjected to flash column with 20-66% EtOAc in hexane to isolate 2-(3-((tert-butoxycarbonyl)amino)phenyl)-2H-1,2,3-triazole 1-oxide (110 mg). It was treated with DCM/TFA (3:1) at RT for 90 m. The reaction was quenched with NH₃/MeOH, concentrated and subjected to reverse phase preparative HPLC to isolate 2-(3-aminophenyl)-2H-1,2,3-triazole 1-oxide.

2-(3-Aminophenyl)-2H-1,2,3-triazole 1-oxide (11 mg, 0.06 mmol) was dissolved in 2 mL NMP. To it were added DIEA (44 μL, 0.12 mmol) and then 2,4-dichloropyrimidine-5-carboxamide (36 mg, 0.18 mmol). The mixture was stirred at RT for overnight. To it were added tert-butyl ((1S,2R)-2-aminocyclohexyl)carbamate (40 mg, 0.18 mmol) and DIEA (32 μL, 0.18 mmol). The mixture was stirred at 90° C. for 90 m. It was diluted with EtOAc, washed with brine ×3, dried, concentrated, subjected to flash column with 0-15% MeOH in DCM to isolate 2-(3-((2-(((1R,2S)-2-((tert-butoxycarbonyl)amino)cyclohexyl)amino)-5-carbamoylpyrimidin-4-yl)amino)phenyl)-2H-1,2,3-triazole 1-oxide. It was treated with 4 mL DCM and 1 mL TFA at RT for 1 h. The reaction was quenched with NH₃/MeOH, concentrated and subjected to reverse phase preparative HPLC to isolate the title compound. MS found for C19H23N9O2 as (M+H)⁺ 410.5. UV: λ=244 nm.

### Example 252

### 2-(((1R,2S)-2-aminocyclohexyl)amino)-4-((3-(4-hydroxy-2H-1,2,3-triazol-2-yl)phenyl)amino)pyrimidine-5-carboxamide

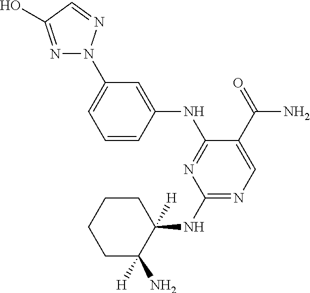 [SMILES: NC(=O)c1cnc(N[C@@H]2CCCC[C@@H]2N)nc1Nc1cccc(-n2ncc(O)n2)c1]

2-(3-Nitrophenyl)-2H-1,2,3-triazole 1-oxide was prepared in a manner similar to that of 2-(3-bromophenyl)-2H-1,2,3-triazole 1-oxide.). ¹H NMR: (CDCl₃) δ 8.92 (1H, t, J=2.0 Hz), 8.39 (1H, m), 8.37 (1H, m), 8.00 (1H, d, J=1.2 Hz), 7.86 (1H, d, J=0.8 Hz), 7.85 (1H, t, J=8.4 Hz) ppm. 2-(3-Nitrophenyl)-2H-1,2,3-triazole 1-oxide (200 mg, 0.97 mmol) was stirred in 5 mL AcCl at RT for 1 h. It was concentrated in vacuo and subjected to flash column with 0-35% EtOAc in hexane to isolate 2-(3-nitrophenyl)-2H-1,2,3-triazol-4-yl acetate (154 mg, 64%).). ¹H NMR: (CDCl₃) δ 8.85 (1H, t, J=2.0 Hz), 8.33 (1H, m), 8.18 (1H, m), 7.89 (1H, s), 7.66 (1H, t, J=8.4 Hz), 2.40 (3H, s) ppm. UV=249, 282 nm.

2-(3-Nitrophenyl)-2H-1,2,3-triazol-4-yl acetate (28 mg, 0.11 mmol) was dissolved in 30 mL EtOAc. 10% Pd/C (28 mg) was added, and a hydrogen balloon was amounted over. The hydrogenation reaction was allowed for overnight at RT. The mixture was filtered through celite, which was then thoroughly washed with MeOH. The filtrate was concentrated in vacuo, and the crude aniline product was used to prepare the title compound in a manner similar to that described in Example 2-(3-((2-(((1R,2S)-2-aminocyclohexyl)amino)-5-carbamoylpyrimidin-4-yl)amino)phenyl)-2H-1,2,3-triazole 1-oxide. MS found for C19H23N9O2 as (M+H)⁺ 410.6. UV: λ=249, 282 nm.

### Example 253

### 2-(3-((2-(((1R,2S)-2-aminocyclohexyl)amino)-5-carbamoylpyrimidin-4-yl)amino)phenyl)-2H-1,2,3-triazol-4-yl acetate

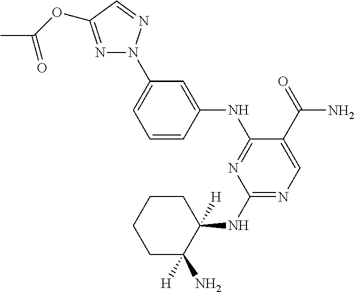 [SMILES: CC(=O)Oc1cnn(-c2cccc(Nc3nc(N[C@@H]4CCCC[C@@H]4N)ncc3C(N)=O)c2)n1]

The title compound was isolated as a byproduct during the preparation of Example 2-(((1R,2S)-2-aminocyclohexyl)amino)-4-((3-(4-hydroxy-2H-1,2,3-triazol-2-yl)phenyl)amino)pyrimidine-5-carboxamide. MS found for C21H25N9O3 as (M+H)⁺ 452.5. UV: λ=254 nm.

### Example 254

### 2-(((1R,2S)-2-aminocyclohexyl)amino)-4-((3-(oxazol-2-yl)phenyl)amino)pyrimidine-5-carboxamide

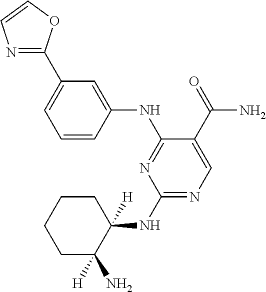 [SMILES: NC(=O)c1cnc(N[C@@H]2CCCC[C@@H]2N)nc1Nc1cccc(-c2ncco2)c1]

The title compound was synthesized in a manner similar to that described in Example 2-((1R,2S)-2-aminocyclohexylamino)-4-(4-bromo-3-(2H-1,2,3-triazol-2-yl)phenylamino)pyrimidine-5-carboxamide. MS found for C20H23N7O2 as (M+H)⁺ 394.5. UV: λ=249 nm. ¹H NMR: (CD₃OD) δ 8.69 (1H, s), 8.59 (1H, s), 8.07 (1H, s), 7.91 (1H, d, J=8.0 Hz), 7.61 (1H, t, J=8.0 Hz), 7.55 (1H, d, J=8.4 Hz), 7.38 (1H, s), 4.53 (1H, m), 3.67 (1H, m), 1.92-1.55 (8H, m) ppm.

### Example 255

### 2-(((1R,2S)-2-aminocyclohexyl)amino)-4-((4-(oxazol-2-yl)phenyl)amino)pyrimidine-5-carboxamide

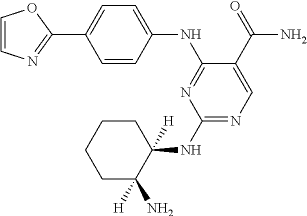 [SMILES: NC(=O)c1cnc(N[C@@H]2CCCC[C@@H]2N)nc1Nc1ccc(-c2ncco2)cc1]

The title compound was synthesized in a manner similar to that described in Example 2-((1R,2S)-2-aminocyclohexylamino)-4-(4-bromo-3-(2H-1,2,3-triazol-2-yl)phenylamino)pyrimidine-5-carboxamide. MS found for C20H23N7O2 as (M+H)⁺ 394.6. UV: λ=244, 316 nm. ¹H NMR: (CD₃OD) δ 8.58 (1H, s), 8.11 (2H, d, J=8.0 Hz), 8.02 (1H, s), 7.82 (2H, d, J=8.4 Hz), 7.33 (1H, s), 4.41 (1H, m), 3.74 (1H, m), 1.88-1.62 (8H, m) ppm.

### Example 256

### 4-((3-(2H-1,2,3-triazol-2-yl)phenyl)amino)-2-(((1R,2S)-2-(hydroxymethyl)cyclohexyl)amino)pyrimidine-5-carboxamide (racemic)

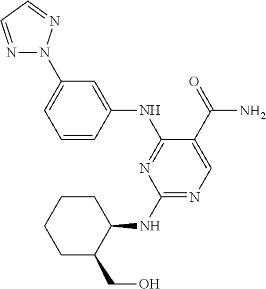 [SMILES: NC(=O)c1cnc(N[C@@H]2CCCC[C@@H]2CO)nc1Nc1cccc(-n2nccn2)c1]

The title compound was synthesized in a manner similar to that described in Example 4-(3-(2H-1,2,3-triazol-2-yl)phenylamino)-2-((1S,2R)-2-aminocyclohexylamino)pyrimidine-5-carboxamide. MS found for C20H24N8O2 as (M+H)⁺ 409.6. UV: λ=254 nm. ¹H NMR: (CD₃OD) δ 8.95 (1H, m), 8.50 (1H, s), 7.96 (2H, s), 7.95 (1H, m), 7.57 (1H, t, J=8.0 Hz), 7.44 (1H, d, J=6.0 Hz), 4.60 (1H, m), 3.71 (1H, m), 3.54 (1H, m), 2.03 (1H, m), 1.88 (1H, m), 1.74-1.40 (7H, m) ppm.

The in vitro and in vivo human Syk activities of the inventive compounds can be determined by various procedures known in the art, such as a test for their ability to inhibit the activity of human plasma Syk. The potent affinities for human Syk inhibition exhibited by the inventive compounds can be measured by an IC₅₀ value (in nM). The IC₅₀ value is the concentration (in nM) of the compound required to provide 50% inhibition of human Syk proteolytic activity. The smaller the IC₅₀ value, the more active (potent) is a compound for inhibiting Syk activity.

An in vitro assay for detecting and measuring inhibition activity against Syk is as follows:

### Inhibition of Syk Tyrosine Phosphorylation Activity

Potency of candidate molecules for inhibiting Syk tyrosine phosphorylation activity is assessed by measuring the ability of a test compound to inhibit Syk-mediated tyrosine phosphorylation of a Syk-specific substrate.

SYK tyrosine phosphorylation activity is measured using the LANCE™ Technology developed by Perkin Elmer Life and Analytical Sciences (Boston, Mass.). LANCE™ refers to homogeneous time resolved fluorometry applications using techniques such as time-resolved fluorescence resonance energy transfer assay (TR-FRET) (see generally for procedures in Perkin Elmer Application Note—How to Optimize a Tyrosine Kinase Assay Using Time Resolved Fluorescence-Based LANCE Detection, wwww.perkinelmer.com/lifesciences). The assay principle involves detection of a phosphorylated substrate using energy transfer from a phosphospecific europium-labeled antibody to streptavidin-allophycocyanin as an acceptor.

To test the ability of candidate molecules to inhibit SYK tyrosine phosphorylation activity, molecules are reconstituted in 30% DMSO and serially diluted 1:3 with the final dilution containing DMSO in the absence of the candidate molecule. The final DMSO concentration in the assay is 3%. Kinase assays are performed as a two part reaction. The first reaction is a kinase reaction and which comprises of a candidate molecule, full length active recombinant SYK enzyme (Millipore, Calif.) and biotin-labeled SYK-specific substrate biotin-DEEDYESP-OH. The second reaction involves termination of the kinase reaction and the simultaneous addition of the detection reagents—europium-labeled anti-phosphotyrosine reagent (Eu-W1024-PY100, Perkin Elmer, Boston, Mass.) and Streptavidin-Allophycocyanin detection reagent (SA-APC, Prozyme, Calif.). The kinase reaction is performed in a black U-bottom 96-well microtitre plate. The final reaction volume is 50 μL and contains a final concentration of 1 nM active SYK enzyme, 550 nM SYK-substrate, and 100 μM ATP diluted in a buffer containing 50 mM Tris pH 7.5, 5 mM MgCl₂, and 1 mM DTT. The reaction is allowed to proceed for 1 hour at room temperature. The quench buffer contains 100 mM Tris pH 7.5, 300 mM NaCl₂, 20 mM EDTA, 0.02% Brij35, and 0.5% BSA. The detection reagents are added to the reaction mixture at the following dilutions-1:500 for Eu-W1024-PY100 and 1:250 for SA-APC. The kinase reaction is terminated by the addition of 50 μL quench buffer containing the detection reagents. The detection is allowed to proceed for 1 hr at room temperature. Detection of the phosphorlated substrate in the absence and presence of inhibitors is measured in the TR-FRET instrument, Analyst HT (Molecular Probes, Sunnyvale, Calif.) and the condition for measurements are set up using CriterionHost Release 2.0 (Molecular Probes, Sunnyvale, Calif.). The settings used are a follows: excitation 360 nm, emission 665-7.5 nm, beam splitter 350 nm 50/50, flash 100 pulses, delay 60 us, integration 400 us, z-height 2 mm. Inhibition of SYK-tyrosine kinase activity is calculated as the maximum response observed in the presence of inhibitor, compared to that in the absence of inhibitor. IC₅₀s were derived by non-linear regression analysis.

Intracellular phospho-flow cytometry can be used to test compound inhibition of Syk activity in the non-Hodgkin's lymphoma cell line Ramos. 1×10⁶ cells in log phase growth were aliqoted; Syk kinase is activated by incubating cells for 10 minutes with 3 μg/ml antibody specific to the B cell receptor. Directly following, cells are fixed in 1% paraformaldehyde for 5 minutes at room temperature, washed in phosphate buffered saline, and then permeablized by incubation for 2 hours in ice cold methanol. Cells are again washed in phosphate buffered saline, then incubated for 30 minutes with antibody specific for phosphorylated Erk (Y204), which are indicators of Syk kinase activity. All antibodies used are purchased from BD Pharmingen (San Jose, Calif.). After incubation with antibodies, cells are again washed and subjected to flow cytometry.

Syk has been implicated experimentally in B cell development, proliferation, and survival. Moreover, Syk is implicated as an oncogene. Expression of constitutively active Syk in adoptively transferred bone marrow cells induces leukemia in mice, and over-activity of Syk is associated with a variety of lymphomas in humans Given the role of Syk in B cell biology, its selective inhibition may be sufficient to provide clinical benefit in B cell proliferative disorders, while reducing toxicities that may arise due to suppression of other off-target kinases.

The anti-proliferative effects of compounds on non-Hodgkin's lymphoma B cell lines SUDHL-4, SUDHL-6, and Toledo can also assessed. SUDHL-4 and SUDHL-6 require B cell receptor signaling for growth and survival, while the Toledo cell line (serving here as a negative control) does not. Cells are aliquoted into each well of a 96-well plate and incubated with increasing concentrations of compound for 72 hours, after which cell survival and proliferation is determined using the MTT assay (Chemicon International, Inc., Temecula, Calif.) following protocols supplied by the manufacturer.

Induction of apoptosis in non-Hodgkin's lymphoma B cell lines SUDHL-4, SUDHL-6, and Toledo is assessed by measuring the apoptotis marker Caspase 3. Cells were incubated with 1, 3, or 10 μM compound for 24, 48, and 72 hours. At the conclusion of each time point, cells were processed for flow cytometry analysis using the Monoclonal Rabbit Anti-Active Caspase-3 Antibody Kit and related protocols (BD Pharmingen). Data from two independent experiments are presented in Table 1, representing the percent of total cells undergoing apoptosis following incubation with compounds under the indicated conditions.

Syk activity is not only required for B cell signaling, proliferation, and survival, as shown, but is also critical for cellular activation upon cross-linking of the B cell receptor. B cell activation leads to increased cell surface expression of several proteins involved in cell signaling, antigen presentation, and adhesion. Among these, CD80, CD86, and CD69 are commonly measured to determine B cell activation status. Primary mouse B cells isolated from spleen can be aliquoted and incubated with increasing concentrations of compound (0.05 to 2 μM) in the presence of goat anti-mouse IgD (eBiosciences, Inc., San Diego, Calif.) for 20 hours to cross-link the B cell receptor. Cells are washed and incubated for 30 minutes on ice with antibodies specific for the CD80, CD86, and CD69 B cell activation markers. B cells are identified from the pooled population by staining with the B cell marker CD45RO. All antibodies are purchased from BD Pharmingen.

In the table below, activity in the Syk assays is provided as follows: +++++=IC₅₀<0.0010 μM; ++++=0.0010 μM<IC₅₀<0.010 μM, +++=0.010 μM<IC₅₀<0.10 μM, ++=0.10 μM<IC₅₀<1 μM, +=IC₅₀>1 μM.

TABLE 1

| Example | |
|---|---|
| No. | Syk IC50 |
| 1 | ++ |
| 2 | ++ |
| 3 | ++ |
| 4 | ++ |
| 5 | ++ |
| 6 | ++ |
| 7 | ++ |
| 8 | ++ |
| 9 | ++ |
| 10 | ++ |
| 11 | ++ |
| 12 | ++ |
| 13 | ++ |
| 14 | ++++ |
| 15 | ++ |
| 18 | ++ |
| 19 | ++ |
| 20 | +++ |
| 21 | +++ |
| 22 | ++++ |
| 23 | +++ |
| 24 | +++ |
| 26 | ++ |
| 27 | ++ |
| 28 | +++ |
| 29 | +++ |
| 30 | ++ |
| 31 | +++ |
| 32 | +++ |
| 33 | ++ |
| 34 | ++++ |
| 35 | +++ |
| 36 | +++ |
| 37 | +++ |
| 38 | +++ |
| 39 | +++ |
| 40 | ++ |
| 41 | ++++ |
| 42 | ++++ |
| 43 | ++++ |
| 44 | ++++ |
| 45 | +++ |
| 46 | +++ |
| 47 | +++ |
| 48 | +++ |
| 49 | ++++ |
| 50 | +++ |
| 51 | +++ |
| 52 | ++ |
| 53 | +++ |
| 54 | ++ |
| 55 | +++ |
| 56 | +++ |
| 57 | +++ |
| 58 | +++ |
| 59 | +++ |
| 60 | ++ |
| 61 | ++ |
| 62 | +++ |
| 63 | ++ |
| 64 | ++ |
| 65 | +++ |
| 66 | +++ |
| 67 | +++ |
| 68 | +++ |
| 69 | +++ |
| 70 | ++ |
| 71 | +++ |
| 73 | +++ |
| 74 | +++ |
| 75 | +++ |
| 76 | ++++ |
| 77 | ++ |
| 78 | +++ |
| 79 | +++ |
| 80 | ++ |
| 81 | +++ |
| 82 | ++ |
| 83 | ++ |
| 84 | ++++ |
| 85 | ++++ |
| 86 | +++ |
| 87 | +++ |
| 88 | ++++ |
| 89 | +++ |
| 90 | ++++ |
| 91 | +++ |
| 92 | +++ |
| 93 | +++ |
| 94 | ++++ |
| 95 | ++ |
| 96 | ++ |
| 97 | +++ |
| 98 | +++ |
| 99 | +++ |
| 100 | ++ |
| 101 | ++ |
| 102 | +++ |
| 103 | ++ |
| 104 | +++ |
| 105 | ++ |
| 106 | +++ |
| 107 | ++ |
| 108 | ++ |
| 109 | ++ |
| 110 | ++ |
| 111 | ++ |
| 112 | ++ |
| 113 | +++ |
| 114 | ++ |
| 115 | ++ |
| 116 | ++ |
| 117 | +++ |
| 118 | ++ |
| 119 | ++ |
| 125 | ++++ |
| 126 | +++++ |
| 127 | +++++ |
| 128 | ++ |
| 129 | ++ |
| 130 | ++++ |
| 131 | ++++ |
| 132 | ++++ |
| 133 | ++ |
| 134 | ++ |
| 135 | ++++ |
| 136 | ++++ |
| 137 | ++++ |
| 138 | ++++ |
| 139 | ++++ |
| 140 | ++++ |
| 141 | ++++ |
| 142 | ++++ |
| 143 | ++++ |
| 144 | ++++ |
| 145 | ++++ |
| 146 | +++ |
| 147 | +++ |
| 148 | ++ |
| 149 | +++ |
| 150 | +++ |
| 151 | ++++ |
| 152 | ++++ |
| 153 | +++ |
| 154 | ++++ |
| 155 | ++++ |
| 156 | ++++ |
| 157 | +++ |
| 158 | ++++ |
| 159 | ++++ |
| 160 | +++ |
| 161 | +++ |
| 162 | ++++ |
| 163 | ++++ |
| 164 | ++ |
| 165 | ++ |
| 166 | +++ |
| 167 | ++++ |
| 168 | ++ |
| 169 | +++ |
| 170 | ++ |
| 171 | ++++ |
| 172 | ++ |
| 173 | ++ |
| 174 | +++ |
| 175 | +++ |
| 176 | ++ |
| 177 | +++ |
| 178 | +++ |
| 179 | +++ |
| 180 | ++++ |
| 181 | +++ |
| 182 | +++ |
| 183 | +++ |
| 184 | +++ |
| 185 | +++ |
| 186 | +++ |
| 187 | ++++ |
| 188 | +++ |
| 189 | ++++ |
| 190 | ++++ |
| 191 | ++++ |
| 192 | +++ |
| 193 | +++ |
| 194 | +++ |
| 195 | ++++ |
| 196 | ++ |
| 197 | +++ |
| 201 | ++ |
| 202 | ++ |
| 203 | ++ |
| 204 | +++ |
| 205 | ++ |
| 206 | +++ |
| 207 | ++ |
| 208 | +++ |
| 212 | +++ |
| 213 | ++ |
| 214 | +++ |
| 215 | ++ |
| 216 | + |
| 217 | + |
| 218 | + |
| 219 | + |
| 220 | +++ |
| 221 | ++++ |
| 222 | +++ |
| 223 | + |
| 224 | ++ |
| 225 | ++ |
| 226 | ++ |
| 227 | ++ |
| 228 | + |
| 229 | ++ |
| 230 | ++ |
| 231 | +++ |
| 232 | +++ |
| 233 | |
| 234 | |
| 235 | +++ |
| 236 | +++ |
| 237 | +++ |
| 238 | +++ |
| 239 | +++ |
| 240 | +++ |
| 241 | ++ |
| 242 | +++ |
| 243 | ++ |
| 244 | ++ |
| 245 | ++ |
| 246 | +++ |
| 247 | +++ |
| 248 | ++ |
| 249 | ++++ |
| 250 | ++ |
| 251 | +++ |
| 252 | ++++ |
| 253 | + |
| 254 | ++++ |
| 255 | +++ |
| 256 | ++ |

The present invention provides a number of embodiments. It is apparent that the examples may be altered to provide other embodiments of this invention. Therefore, it will be appreciated that the scope of this invention is to be defined by the appended claims rather than by the specific embodiments, which have been represented by way of example.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety. From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound of Formula (I): 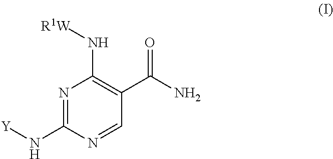
or a pharmaceutically acceptable salt thereof, wherein
W is selected from the group consisting of
(a) C₃₋₈cycloalkyl, optionally substituted with from 1 to 4 substituents independently selected from the group consisting of C₁₋₈ alkyl, amino, hydroxy, C₁₋₈alkylcarbonyl, aminocarbonyl, C₁₋₈alkoxycarbonylamino, arylC₁₋₈alkoxycarbonylamino, aryl and heterocyclylC₁₋₈alkylene;
(b) C₁₋₈ alkyl, optionally substituted with from 1 to 4 substituents independently selected from the group consisting of amino, oxo, C₁₋₈alkoxy, C₂₋₈alkynyl, cyano, aminocarbonyl, C₁₋₈haloalkylene, hydroxy, halogen, C₃₋₈cycloalkyl, and aryl;
(c) C₁₋₈ alkylC₃₋₈heterocyclyl, optionally substituted with from 1 to 4 substituents independently selected from the group consisting of C₁₋₈alkyl, C₁₋₈alkylcarbonyl, C₁₋₈alkylsulfonyl; and aminocarbonyl;
(d) aryl, optionally substituted with from 1 to 4 substituents independently selected from the group consisting of C₁₋₈alkyl, C₂₋₈alkenyl, C₂₋₈alkynyl, C₁₋₈haloalkylene, carboxy, acyl, acylamino, cyano, amino, aminocarbonyl, aminosulfonyl, sulfonyl, nitro, hydroxy, C₁₋₈alkoxy, aryloxy, halo, sulfonylamino, C₃₋₈cycloalkyl, aryl, heterocyclyl C₁₋₈alkylsulfonyl, C₁₋₈alkylcarbonylheterocyclyl and heteroaryl;
(e) heteroaryl, optionally substituted with from 1 to 4 substituents independently selected from the group consisting of C₁₋₈alkyl, C₁₋₈alkylcarbonyl, aminocarbonyl, C₁₋₈alkoxycarbonyl, amino, C₁₋₈ alkoxycarbonylamino, arylC₁₋₈alkoxycarbonylamino, hydroxy, C₁₋₈ alkoxy, C₁₋₈alkylsulfonyl, oxo, halo, aryl and heterocyclylC₁₋₈alkylene;
(f) C₃₋₈heterocyclyl, optionally substituted with from 1 to 4 substituents independently selected from the group consisting of C₁₋₈alkyl, C₁₋₈alkoxycarbonyl and oxo;
R¹ is selected from the group consisting of H, C₁₋₈ alkyl, amino, aminocarbonyl, hydroxy, C₁₋₈ alkoxy, C₁₋₈ haloalkylene, C₂₋₈ alkenyl, C₂₋₈ alkynyl, oxo, cyano, C₁₋₈ alkoxycarbonyl, C₃₋₈ cycloalkyl, aryl and heterocyclyl; and each heterocyclyl is optionally substituted with from 1 to 4 substituents selected from the group consisting of C₁₋₈ alkyl, halo, oxo, amino, C₁₋₈alkoxy, C₁₋₈alkylcarbonyl, arylC₁₋₈ alkoxycarbonyl, aminocarbonyl, arylC₁₋₈ alkylenecarbonyl and C₁₋₈ alkylsulfonyl;
Y is selected from the group consisting of 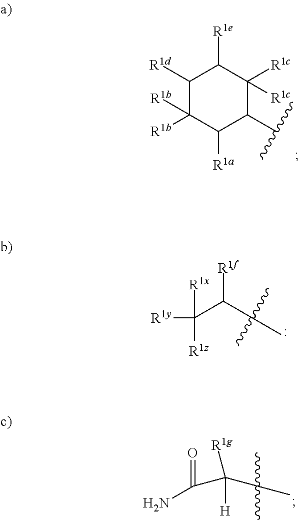
and
d) heterocyclyl, optionally substituted with from 1 to 4 substituents independently selected from the group consisting of C₁₋₈alkyl, C₁₋₈alkenyl, amino, cyanoC₁₋₈alkylene, aminocarbonyl, alkylaminocarbonyl, dialkylaminocarbonyl, oxoC₁₋₈alkylene, hydroxyalkyl, carboxy, haloC₁₋₈alkylene, cyano and oxo and halo
e) phenyl, optionally substituted with from 1 to 4 substituents independently selected from the group consisting of alkyl, alkoxy and halo;
f) pyridyl, optionally substituted with from 1 to 4 substituents independently selected from the group consisting of alkoxy;
g) indinlyl, optionally substituted with from 1 to 4 substituents independently selected from the group consisting of hydroxyl and oxo;
R^{1a} is selected from the group consisting of oxo, hydroxy, alkoxy, NH₂, N₃, triazinyl, HC(O)NH—, NCCH₂NH—, HOCH₂CH₂NH—, R^{1u}OCONH—, R^{1v}NHCH(CH₃)NH—, N⁺(O⁻) H₂, N(O), N(═CH₂), R^{1w}OC(O)NH—, and C₁₋₈alkylC(O)NH—;
R^{1b} is selected from the group consisting of H, hydroxyl, fluoro, combined to form an oxo group, or one R^{1b} is combined with R^{1a} to form a pyridyl ring and the other R^{1b} is null;
R^{1c} is selected from the group consisting of H, fluoro, hydroxyl, alkoxy, benzyloxy;
R^{1d} is independently selected from H, alkoxycarbonyl, alkylaminocarbonyl, dialkylaminocarbonyl, and heterocyclylcarbonyl;
R^{1e} is independently selected from H and aminocarbonyl;
R^{1u} is selected from the group consisting of H, alkyl, and heterocyclyl optionally substituted with one to four substitutents independently selected from the group consisting of oxo, hydroxy, and carboxy;
R^{1v} is a sugar moiety;
R^{1w} is a moiety of formula V attached via a covalent bond at R^{1a} wherein Y is 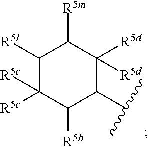
each R^{1f} is selected from the group consisting of H, C₁₋₈alkyl, C₁₋₈haloalkylene, phenyl, C₃₋₈cycloalkyl, hydroxyC₁₋₈alkylene, NH₂, C₁₋₈alkylamino, C₁₋₈ alkoxycarbonylaminoC₁₋₈ alkylene, C₃₋₈cycloalkylC₁₋₈ alkylene, heteroaryl, alkylene, C₁₋₈alkylsulfonylC₁₋₈ alkylene, aminocarbonyl, C₁₋₈alkoxyC₁₋₈alkyl, haloC₁₋₈alkylene, aryl and heterocyclyl; wherein the aryl is optionally substituted by hydroxy, C₁₋₈alkoxy, halo or haloC₁₋₈alkylene;
R^{1g} is independently selected from the group consisting of H, C₁₋₈alkyl, C₃₋₈cycloalkyl, and C₃₋₈cycloalkylC₁₋₈ alkylene;
R^{1x} is H, alkyl, haloalkyl or combined with R^{1y} to form a cycloalkyl group;
R^{1y} is selected from the group consisting of H, C₁₋₈alkyl, C₁₋₈alkylamino, amino aminoC₁₋₈alkylene, carboxy, C₁₋₈alkylaminoC₁₋₈alkylene, C₁₋₈alkoxyC₁₋₈alkylene, hydroxyC₁₋₈alkylene; carboxyC₁₋₈alkylene, C₃₋₈cycloalkylC₁₋₈alkylene, aryloxyC₁₋₈alkylene, arylC₁₋₈alkylene, heteroarylC₁₋₈alkylene, and hydroxyC₁₋₈alkoxy; or
R^{1y} may be combined with R^{1f} or R^{1x} and the atoms to which they are attached to form a C₃₋₈ cycloalkyl or heterocyclyl ring optionally substituted with one to three groups independently selected from hydroxy, halo, oxo and amino;
R^{1z} is selected from the group consisting of H, amino, C₁₋₈alkylamino, hydroxycarbonylamino, C₁₋₈alkoxycarbonylamino, arylC₁₋₈alkoxycarbonylamino and hydroxy;
and the wavy line indicates the point of attachment to the rest of the molecule
wherein the wavy line indicates the point of attachment to the rest of the molecule.

2.-9. (canceled)

10. A compound of Formula (II) or a pharmaceutically acceptable salt thereof: 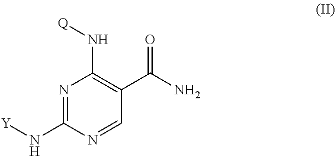
wherein
Q is selected from the group consisting of:
a) heteroaryl optionally substituted with one to five R^{3a} groups;
b) cycloalkyl optionally substituted with one to five R^{3a} groups;
c) heterocyclyl optionally substituted with one to five R^{3a} groups; and
d) aryl substituted with R^{3b} and optionally substituted with one to four R^{3a} groups;
R^{3b} is -is selected from the group consisting of C₁₋₈ alkyl, C₃₋₈ cycloalkylC₁₋₈ alkyl, C₁₋₈ alkoxy, C₃₋₈ cycloalkoxy, hydroxyC₁₋₈ alkyl, C₁₋₈ alkoxyalkyl, haloC₁₋₈ alkyl, haloC₁₋₈ alkoxy, amino, C₁₋₈ alkylamino, diC₁₋₈ alkylamino, halo, haloC₁₋₈ alkylaminocarbonyl, C₁₋₈alkylaminocarbonyl, diC₁₋₈ alkylaminocarbonyl, aminocarbonyl, heterocyclylcarbonyl, C₁₋₈ alkylcarbonylamino, C₁₋₈ alkylsulfonyl, aminosulfonyl, C₃₋₈ cycloalkyl, C₁₋₈ alkylcarbonylpiperadinyl, morpholinyl, phenyl, and heteroaryl optionally substituted with one to three R^{3c} groups;
R^{3a} and R^{3c} are independently selected from the group consisting of C₁₋₈ alkyl, C₂₋₈ alkenyl, C₂₋₈alkynyl, C₃₋₈ cycloalkylC₁₋₈ alkylene, C₁₋₈ alkoxy, C₃₋₈ cycloalkoxy, hydroxyC₁₋₈ alkylene, C₁₋₈ alkoxyalkylene, haloC₁₋₈ alkylene, haloC₁₋₈ alkoxy, amino, hydroxyl, C₁₋₈ alkylamino, diC₁₋₈ alkylamino, C₁₋₈alkylthio, oxo, halo, cyano, haloC₁₋₈ alkylaminocarbonyl, C₁₋₈alkylaminocarbonyl, diC₁₋₈ alkylaminocarbonyl, aminocarbonyl, heterocyclylcarbonyl, C₁₋₈ alkylcarbonylamino, C₁₋₈ alkylsulfonyl, aminosulfonyl, C₃₋₈ cycloalkyl, C₁₋₈ alkylcarbonylpiperadinyl, heterocyclyl, phenyl, heteroaryl, heteroarylsulfinyl; C₁₋₈arylalkylene, aminoC₁₋₈alkylene;
Y is selected from the group consisting of 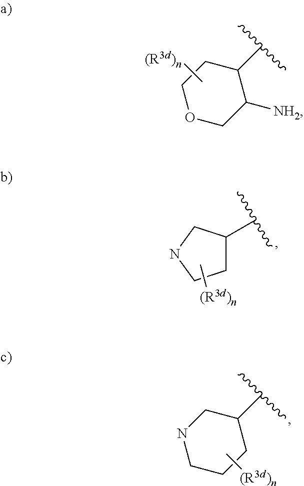
R^{3d} is independently selected from the group consisting of C₁₋₈alkyl, C₁₋₈alkylcarbonyl, cyanoC₁₋₈alkylene, hydroxyC₁₋₈alkylene, haloC₁₋₈alkylene, halo, and amino, and n is 0, 1, 2, 3, 4, or 5;
R^{3e} is selected from the group consisting of hydrogen, cycloalkyl, cycloalkylC₁₋₈alkyl, and C₁₋₈alkyl, wherein R^{3e} is optionally substituted with one to five groups independently selected from halo, C₁₋₈alkyl, and amino;
R^{3f} is hydrogen or together with R^{3e} and the carbon atom to which they are attached to form a cycloalkyl ring;
R^{3g} is C₁₋₈alkyl optionally substituted with one to three halo substituents.

11. A compound of Formula (IIa) 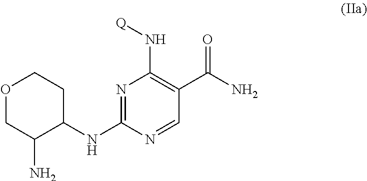
or a pharmaceutically acceptable salt thereof, wherein
Q is phenyl or heteroaryl optionally substituted with R^{2a}, wherein heteroaryl is selected from the group consisting of pyrimidinyl, indolyl, benzothiazolyl, thieno[2,3-b]pyridinyl, and quinolinyl; and
R^{2a} is independently selected from the group consisting of C₁₋₈alkyl, haloC₁₋₈alkylene, halo, and cyano.

12. A compound of Formula (IIb) 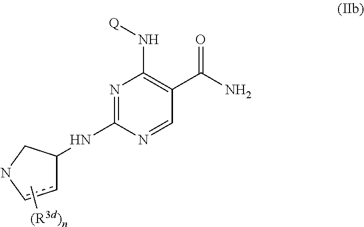
or a pharmaceutically acceptable salt thereof, wherein
Q is phenyl or heteroaryl optionally substituted with R^{2a}, wherein heteroaryl is selected from the group consisting of triazoyl, pyrimidinyl, indolyl, benzothiazolyl, thieno[2,3-b]pyridinyl, and quinolinyl; and
R^{2a} is independently selected from the group consisting of H, C₁₋₈alkyl, cyanoC₁₋₈alkylene, aminocarbonyl, alkylaminocarbonyl, dialkylaminocarbonyl, oxoC₁₋₈alkylene, hydroxyalkyl, carboxy, haloC₁₋₈alkylene, cyano and oxo and halo.

13. A compound of claim 10, or a pharmaceutically acceptable salt thereof, wherein Y is selected from the group consisting of

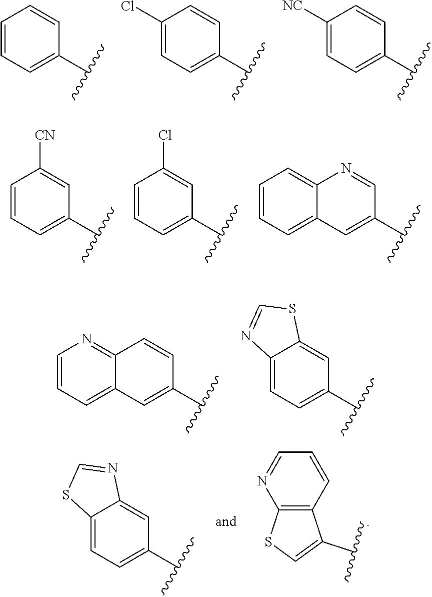

14. A compound of Formula (IIIa), (IIIb), (IIIc) and (IIId): 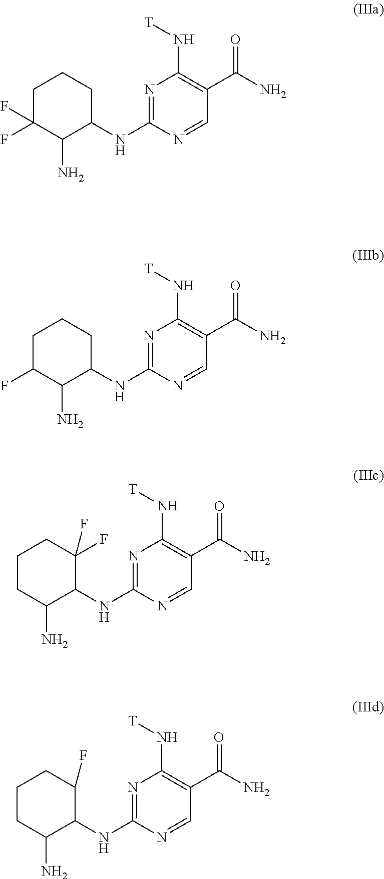
or a pharmaceutically acceptable salt thereof, wherein
T is selected from the group consisting of phenyl, pyrimidinyl, indolyl, indazoyl, benzothiazolyl, thieno[2,3-b]pyridinyl, pyrazolo[1,5-a]pyridine and quinolinyl, wherein T is optionally substituted with one to three R^{3a} substituents; and
R^{3a} is independently selected from the group consisting of C₁₋₈alkyl, haloC₁₋₈alkylene, halo, cyano, pyrimidyl, and pyrazoyl.

15. A compound of claim 14 or a pharmaceutically acceptable salt thereof, wherein T is quinolinyl.

16. A compound of claim 14 or a pharmaceutically acceptable salt thereof, wherein T is selected from the group consisting of

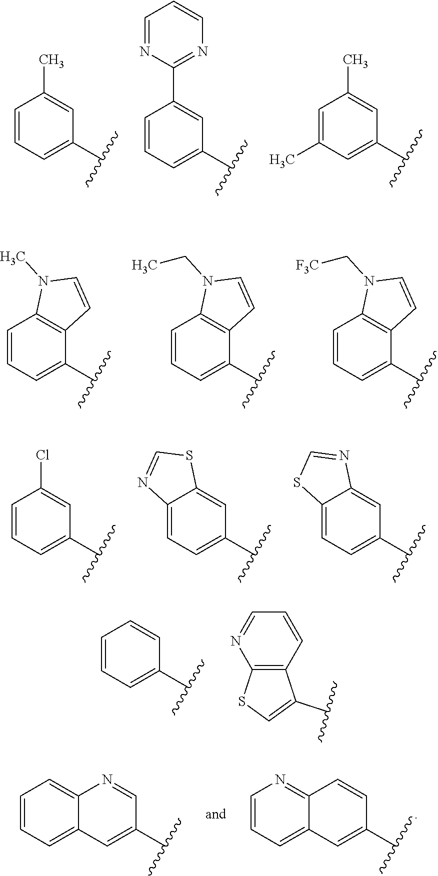

17. A compound of Formula (IV) 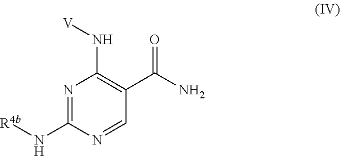
or a pharmaceutically acceptable salt thereof, wherein
V is selected from the group consisting of
(a) phenyl, optionally substituted with from 1 to 4 R^{4a}; and
(b) heteroaryl, optionally substituted with from 1 to 4 R^{4a};
each R^{4a} is independently selected from the group consisting of C₁₋₈alkyl, C₁₋₈alkoxy, cyano, hydroxyl, oxo, halo, haloC₁₋₈alkyl and heteroaryl;
R^{4b} is selected from the group consisting of 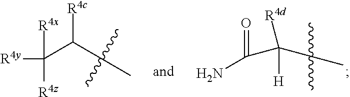
each R^{4c} is selected from the group consisting of H, C₁₋₈alkyl, C₁₋₈haloalkylene, phenyl, C₃₋₈cycloalkyl, hydroxyC₁₋₈alkylene, NH₂, C₁₋₈alkylamino, C₁₋₈ alkoxycarbonylaminoC₁₋₈ alkylene, C₃₋₈cycloalkylC₁₋₈alkylene, heteroaryl, C₁₋₈alkylthioC₁₋₈ alkylene, C₁₋₈alkylsulfonylC₁₋₈ alkylene, aminocarbonyl, C₁₋₈alkoxyC₁₋₈alkyl, haloC₁₋₈alkylene, aryl and heterocyclyl; wherein the aryl is optionally substituted by hydroxy, C₁₋₈alkoxy, halo or haloC₁₋₈alkylene;
R^{4d} is independently selected from the group consisting of H, C₁₋₈alkyl, C₃₋₈cycloalkyl, and C₃₋₈cycloalkylC₁₋₈ alkylene;
R^{4x} is H, alkyl, haloalkyl or combined with R^{4y} to form a cycloalkyl group;
R^{4y} is selected from the group consisting of H, C₁₋₈alkyl, C₁₋₈alkylamino, amino aminoC₁₋₈alkylene, carboxy, C₁₋₈alkylaminoC₁₋₈alkylene, C₁₋₈alkoxyC₁₋₈alkylene, hydroxyC₁₋₈alkylene; carboxyC₁₋₈alkylene, C₃₋₈cycloalkylC₁₋₈alkylene, aryloxyC₁₋₈alkylene, arylC₁₋₈alkylene, heteroarylC₁₋₈alkylene, and hydroxyC₁₋₈alkoxy; or
R^{4y} may be combined with R^{4c} or R^{4X} and the atoms to which they are attached to form a C₃₋₈ cycloalkyl or heterocyclyl ring optionally substituted with one to three groups independently selected from hydroxy, halo, oxo and amino;
R^{4z} is selected from the group consisting of H, amino, C₁₋₈alkylamino, hydroxycarbonylamino, C₁₋₈alkoxycarbonylamino, arylC₁₋₈alkoxycarbonylamino and hydroxy;
and the wavy line indicates the point of attachment to the rest of the molecule.

18. A compound of claim 17 wherein R^{4b} is cyclohexyl substituted with amino and further optionally substituted with one to three halo substituents.

19. A compound of claim 17 or a pharmaceutically acceptable salt thereof, wherein R^{4b} is

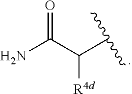

20. A compound of claim 19 or a pharmaceutically acceptable salt thereof, wherein R^{4b} is C₁₋₈alkyl or haloC₁₋₈alkylene.

21. A compound of claim 17 or a pharmaceutically acceptable salt thereof, wherein heteroaryl is selected from the group consisting of: thienyl, thiazoyl, thiadiazoyl, isothiazoyl, pyrazoyl, triazoyl, pyrimidinyl, tetrahydroprimidinyl, indolyl, indolinyl, indazoyl, benzothiazolyl, thieno[2,3-b]pyridinyl, pyrazolo[1,5-a]pyridine, 1H-pyrrolo[2,3-b]pyridine, isoquinolinyl, tetrahydroquinolinyl and quinolinyl.

22. A compound of Formula (V) 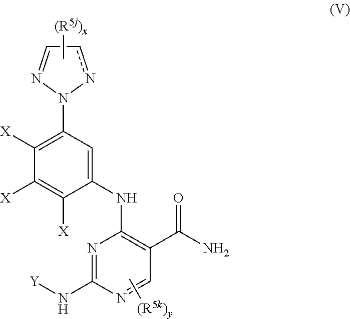
or a pharmaceutically acceptable salt thereof, wherein
X is independently, H or halogen;
Y is CH₃CH₂NH—; (CH₃)₂N—, CH₂CH(NH₂)CH₂CHCF₂; 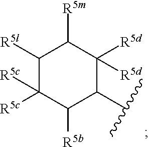
R^{5b} is selected from the group consisting of oxo, hydroxy, alkoxy, NH₂, N₃, triazinyl, HC(O)NH—, NCCH₂NH—, HOCH₂CH₂NH—, R^{5x}OCONH—, R^{5z}NHCH(CH₃)NH—, N⁺(O⁻) H₂, N(O), N(═CH₂), R^{5e}OC(O)NH—, and C₁₋₈alkylC(O)NH—;
R^{5c} is selected from the group consisting of H, hydroxyl, fluoro, combined to form an oxo group, or one R^{5c} is combined with R^{5b} to form a pyridyl ring and the other R^{5c} is null;
R^{5d} is selected from the group consisting of H, fluoro, hydroxyl, alkoxy, benzyloxy;
R^{5e} is selected from the group consisting of H, alkyl, and heterocyclyl optionally substituted with one to four substitutents independently selected from the group consisting of oxo, hydroxy, and carboxy;
R^{5f} is selected from the group consisting of hydrogen, hydroxyl and acetoxy;
R^{5j} is independently selected from oxo, hydroxyl and acetoxy;
R^{5k} is independently selected from oxo, hydroxyl and acetoxy;
R^{5l} is independently selected from H, alkoxycarbonyl, alkylaminocarbonyl, dialkylaminocarbonyl, and heterocyclylcarbonyl;
R^{5m} is independently selected from H and aminocarbonyl;
R^{5x} is a sugar moiety;
R^{5z} is a moiety of formula V attached via a covalent bond at R^{5b} wherein Y is 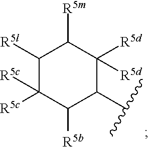
x is 0, 1, or 2;
y is 0, 1, or 2; and
- - - represents a single or double bond;
provided that when R^{5b} is amino and x and y are 0, then R^{5C} and R^{5d} are not both hydrogen.

23.-31. (canceled)

32. A composition comprising a compound or a pharmaceutically acceptable salt thereof of claim 1 in combination with a pharmaceutically acceptable carrier or diluent.

33. A method for inhibiting Syk kinase or a signal transduction pathway mediated at least in part by Syk kinase activity comprising the step of contacting a cell with a compound or a pharmaceutically acceptable salt thereof of claim 1.

34. A method for treating a condition or disorder mediated at least in part by Syk kinase activity in a subject comprising the step of administering to a subject in need of such treatment a therapeutically effective amount of a composition of claim 30.

35.-43. (canceled)

44. A kit comprising a composition of claim 32, packaging and instructions for use.
